(19) United States
(12) Patent Application Publication
(10) Pub. No.: US 2025/0276972 A1
(43) Pub. Date: Sep. 4, 2025

(54) COMPOUNDS AS CASEIN KINASE INHIBITORS

(71) Applicant: GRITSCIENCE BIOPHARMACEUTICALS CO.,LTD., Beijing (CN)

(72) Inventors: Enxing ZHOU, Beijing (CN); Yuan LIU, Beijing (CN); Hanping WANG, Beijing (CN); Guanglong WU, Beijing (CN); Ning SHAO, Beijing (CN)

(21) Appl. No.: 18/265,689

(22) PCT Filed: Dec. 14, 2021
(86) PCT No.: PCT/CN2021/137686

§ 371 Date: Jun. 7, 2023

(30) Foreign Application Priority Data
Dec. 15, 2020 (WO) PCT/CN2020/136541

(51) Int. Cl. C07D 487/04; A61K 31/4985; A61K 31/506; C07B 59/00; C07D 519/00

(52) U.S. Cl. CPC C07D 487/04; A61K 31/4985; A61K 31/506; C07B 59/002; C07D 519/00

(57) ABSTRACT

Provided are casein kinase inhibitors, or pharmaceutically acceptable salts thereof. Corresponding pharmaceutical compositions, methods of synthesis, and intermediates are also provided.

### CROSS REFERENCE TO RELATED APPLICATIONS

This application is a national application of PCT/CN2021/137686, filed on Dec. 14, 2021. The contents of PCT/CN2021/137686 are all hereby incorporated by reference.

### BACKGROUND OF THE INVENTION

The circadian clock links our daily cycles of sleep and activity to the external environment. Deregulation of the clock is implicated in a number of human disorders, including depression, seasonal affective disorder, and metabolic disorders. For example, the circadian clock may regulate multiple downstream rhythms, such as those in sleep and awakening, body temperature, and hormone secretion (Ko and Takahashi, Hum Mol Gen 15: R271-R277). Furthermore, diseases such as depression, seasonal affective disorder, and metabolic disorders, may have a circadian origin (Barnard and Nolan. PLoS Genet. 2008 May; 4(5): e1000040).

Casein kinase (CK) is closely related Ser-Thr protein kinases that serve as key clock regulators that dramatically alter the circadian period. There is a continuing need for CK inhibitors in the treatment of diseases.

### SUMMARY OF THE INVENTION

The present disclosure provides a series of compounds as potent inhibitors of casein kinase.

In one aspect, the present application provides a compound having the structure of formula (I),

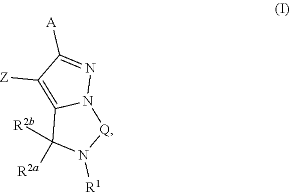

or a pharmaceutically acceptable salt, or prodrug thereof, or a solvate or hydrate of any of the forgoing,
each A and Z is independently selected from the group consisting of optionally substituted (C₆-C₁₄ aryl, and optionally substituted C₂-C₉ heteroaryl;
Q is selected from the group consisting of optionally substituted —CH₂—, optionally substituted —CH₂—CH₂—, optionally substituted —CH═CH—, and optionally substituted —CH₂—CH₂—CH₂—;
R¹ is selected from the group consisting of hydrogen, protium, deuterium, tritium, halogen, cyano, nitro, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl optionally substituted (C₂-C₆)alkenyl, optionally substituted (C₂-C₆)alkynyl, optionally substituted (C₁-C₁₀)carbocycle, optionally substituted (C₂-C₉)heterocycle, optionally substituted (C₆-C₁₀) aryl, and optionally substituted (C₁-C₉)heteroaryl;
each R^{2a} and R^{2b} is independently absent or is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl,
or R^{2a} and R^{2b} combined with the atoms to which they are attached form an optionally substituted ring.

In some embodiments,

wherein, when R¹ is H, Z is not selected from the group consisting of:

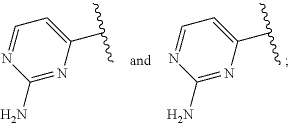 [SMILES: *c1ccnc(N)n1 ; *c1ccnc(N)n1]

when R¹ is —CH₃, Z is not selected from the group consisting of:

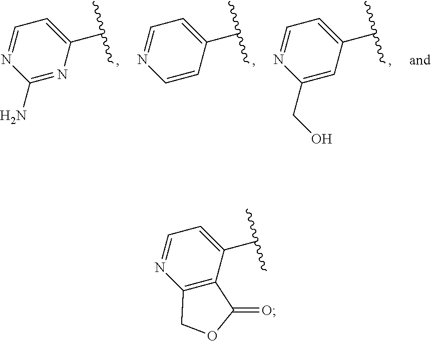 [SMILES: *c1ccnc(N)n1 ; *c1ccncc1 ; *c1ccnc(CO)c1 ; *c1ccnc2c1C(=O)OC2]

when R¹ is selected from the group consisting of: —C(═O)C₂H₅, —C(═O)—CH—(CH₃)₂, —C(═O)—CH₂—CN, —C(═O)—NH—CH₃,

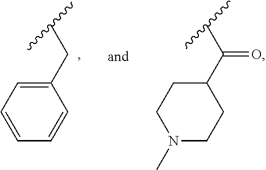

### Z is not

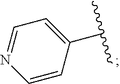 [SMILES: *c1ccncc1]

when R¹ is

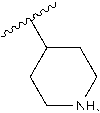 [SMILES: *C1CCNCC1]

### Z is not

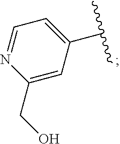 [SMILES: *c1ccnc(CO)c1]

when R¹ is

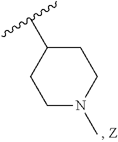 [SMILES: *C1CCN(C)CC1]

is not

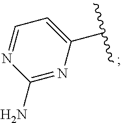 [SMILES: *c1ccnc(N)n1]

when R¹ is —C(═O)—CH₃, Z is not selected from the group consisting of:

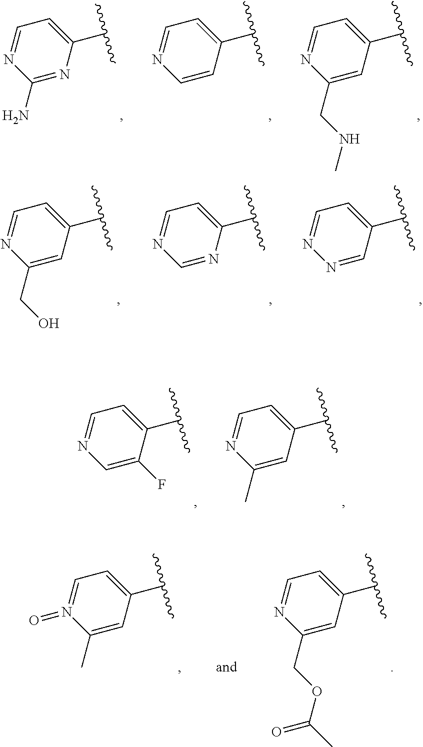

In some embodiments, wherein,
R¹ is selected from the group consisting of deuterium, optionally substituted-methyl-(C₁-C₉)heteroaryl, optionally substituted-halomethyl, optionally substituted deuterated methyl, optionally substituted-methyl-(C₃-C₁₀)carbocycle, optionally substituted-methyl-thio, optionally substituted (C₂-C₆)alkyl, optionally substituted —(C₁) acyl-(C₁-C₉)heteroaryl, optionally substituted —(C₁) acyl-(C₆-C₁₀) aryl, optionally substituted —(C₁) acyl-tetrahydrofuran, optionally substituted —(C₁) acyl-(C₃-C₁₀)carbocycle, optionally substituted-halo(C₂) acyl, optionally substituted —(C₂) acyl-hydroxy, optionally substituted (C₂)thioacyl, optionally substituted —(C₃) acyl-amino, optionally substituted —C(═O)—(CH₂)₂—CH₃, optionally substituted —(C₅-C₆) acyl, optionally substituted oxetane, optionally substituted tetrahydropyran, optionally substituted tetrahydrothiopyran, optionally substituted sulfonyl, optionally substituted phosphorous-containing group, optionally substituted silicon-containing group, optionally substituted thio, optionally substituted (C₂-C₆)alkenyl, optionally substituted (C₃-C₁₀)carbocycle, and optionally substituted (C₁-C₉)heteroaryl.

In some embodiments, wherein,
R¹ is selected from the group consisting of optionally substituted (C₂-C₆)alkyl, optionally substituted —(C₅-C₆) acyl, optionally substituted oxetane, optionally substituted tetrahydropyran, optionally substituted tetrahydrothiopyran, optionally substituted sulfonyl, optionally substituted thio, optionally substituted (C₂-C₆)alkenyl, optionally substituted (C₃-C₁₀)carbocycle, and optionally substituted (C₁-C₉)heteroaryl.

In some embodiments, wherein,
R¹ is optionally substituted (C₂-C₆)alkyl.

In some embodiments, wherein,
R¹ is selected from the group consisting of optionally substituted oxetane, optionally substituted tetrahydropyran, and optionally substituted tetrahydrothiopyran.

In some embodiments, wherein,
R¹ is optionally substituted sulfonyl.

In some embodiments, wherein,
R¹ is optionally substituted thio.

In some embodiments, wherein,
R¹ is optionally substituted (C₂-C₆)alkenyl.

In some embodiments, wherein,
R¹ is optionally substituted (C₃-C₁₀)carbocycle.

In some embodiments, wherein,
R¹ is optionally substituted (C₁-C₆)heteroaryl.

In some embodiments, wherein, said R¹ is substituted with one or more R³, each R³ is independently absent or is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl.

In some embodiments, wherein, each R³ is independently selected from the group consisting of hydrogen, deuterium, halogen, cyano, ═O, optionally substituted hydroxy, optionally substituted thio, optionally substituted amino, optionally substituted (C₁-C₆)alkyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl.

In some embodiments, wherein, each R³ is independently selected from the group consisting of hydrogen, optionally substituted hydroxy, optionally substituted thio, optionally substituted amino, optionally substituted (C₁-C₆)alkyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl.

In some embodiments, wherein, each R³ is independently selected from the group consisting of hydrogen, optionally substituted (C₁-C₆)alkyl, and optionally substituted (C₆-C₁₀) aryl.

In some embodiments, wherein, each R³ is independently selected from the group consisting of hydrogen, optionally substituted methyl, and optionally substituted phenyl.

In some embodiments, wherein, said R³ is substituted with one or more R⁴, each R⁴ is independently absent or is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl.

In some embodiments, wherein, each R⁴ is independently selected from the group consisting of hydrogen, halogen, optionally substituted (C₁-C₆) acyl, and optionally substituted (C₁-C₆)alkyl.

In some embodiments, wherein, each R⁴ is independently selected from the group consisting of hydrogen and halogen.

In some embodiments, wherein, each R⁴ is independently selected from the group consisting of hydrogen and F.

In some embodiments, wherein, said R^{2a} is hydrogen and said R^{2b} is hydrogen.

In some embodiments, wherein, said R^{2a} is ═O and said R^{2b} is absent.

In some embodiments, wherein, Q is optionally substituted —CH₂—CH₂—.

In some embodiments, wherein, said Z is optionally substituted C₆-C₁₄ heteroaryl.

In some embodiments, wherein, said Z is selected from the group consisting of optionally substituted pyridine and optionally substituted pyrimidine.

In some embodiments, wherein, said Z is substituted with one or more R⁵, each R⁵ is independently absent or is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl.

In some embodiments, wherein, each R⁵ is independently selected from the group consisting of hydrogen, optionally substituted amino, optionally substituted (C₁-C₆)alkyl, and optionally substituted (C₂-C₆)heterocycle.

In some embodiments, wherein, each R⁵ is independently selected from the group consisting of hydrogen, optionally substituted amino, and optionally substituted (C₁-C₆)alkyl

In some embodiments, wherein, each R⁵ is independently selected from the group consisting of hydrogen, optionally substituted amino, and optionally substituted methyl.

In some embodiments, wherein, said R⁵ is substituted with one or more R⁶, each R⁶ is independently absent or is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl.

In some embodiments, wherein, each R⁶ is independently selected from the group consisting of hydrogen, optionally substituted hydroxy, optionally substituted (C₁-C₆) acyl, and optionally substituted (C₁-C₉)heteroaryl.

In some embodiments, wherein, each R⁶ is independently selected from the group consisting of hydrogen and optionally substituted (C₂-C₆) acyl.

In some embodiments, wherein, said A is optionally substituted C₆-C₁₄ aryl.

In some embodiments, wherein, said A is optionally substituted phenyl.

In some embodiments, wherein, said A is substituted with one or more R⁷, each R⁷ is independently absent or is independently selected from the group consisting of hydrogen, protium, deuterium, tritium, halogen, cyano, nitro, ═S, ═O, N), optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl.

In some embodiments, wherein, each R⁷ is halogen.

In some embodiments, wherein, each R⁷ is F.

In another aspect, the present application provides a compound or a pharmaceutically acceptable salt, prodrug, or metabolite thereof, or a solvate or hydrate of any of the foregoing, wherein, said compound is selected from the group consisting of:

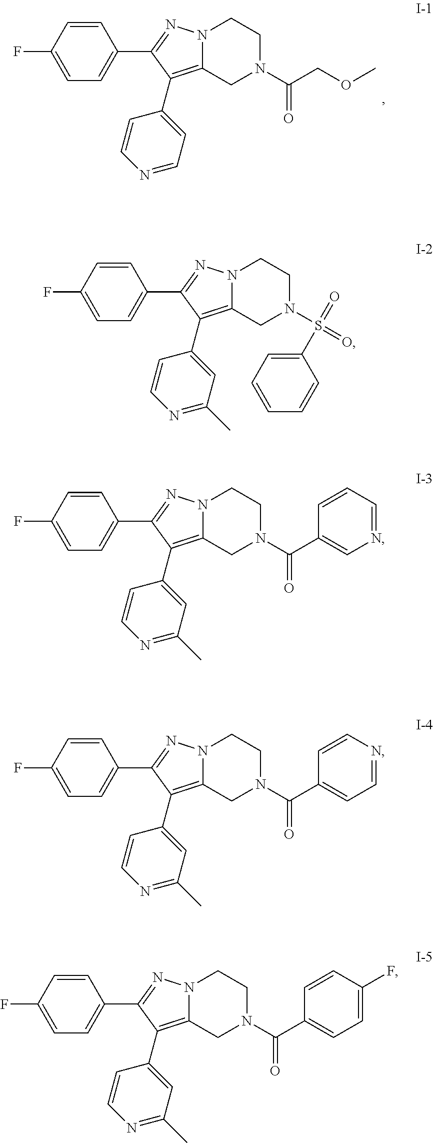 [SMILES: COCC(=O)N1CCn2nc(-c3ccc(F)cc3)c(-c3ccncc3)c2C1 ; Cc1cc(-c2c(-c3ccc(F)cc3)nn3c2CN(S(=O)(=O)c2ccccc2)CC3)ccn1 ; Cc1cc(-c2c(-c3ccc(F)cc3)nn3c2CN(C(=O)c2cccnc2)CC3)ccn1 ; Cc1cc(-c2c(-c3ccc(F)cc3)nn3c2CN(C(=O)c2ccncc2)CC3)ccn1 ; Cc1cc(-c2c(-c3ccc(F)cc3)nn3c2CN(C(=O)c2ccc(F)cc2)CC3)ccn1]

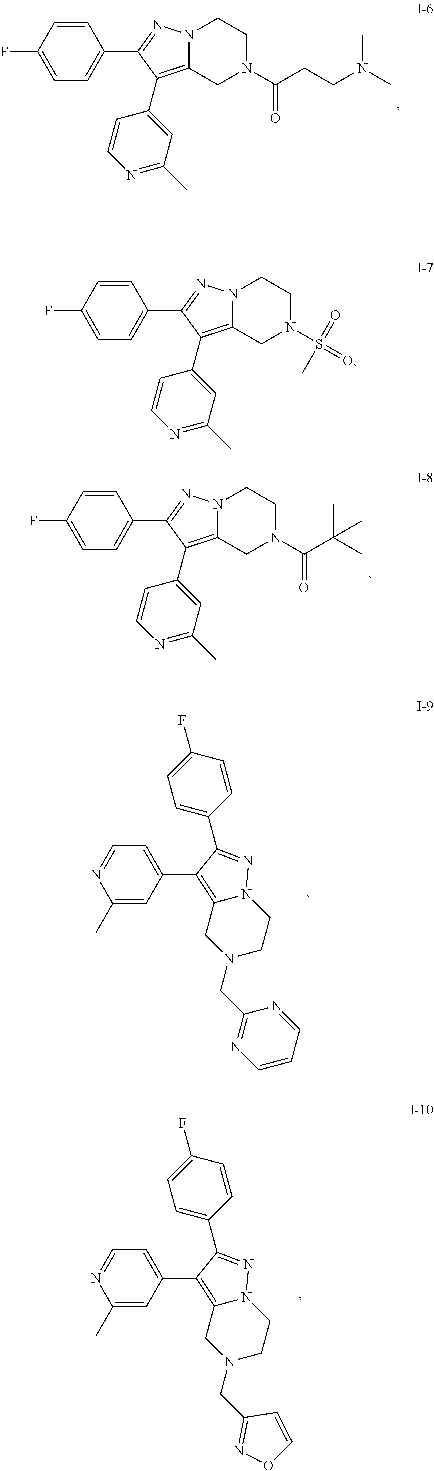

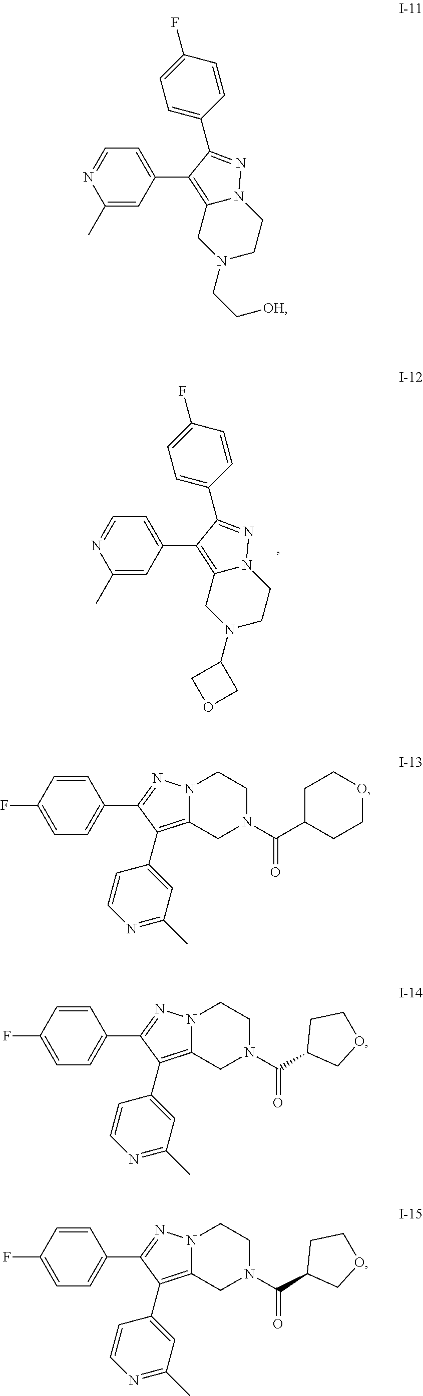 [SMILES: Cc1cc(-c2c(-c3ccc(F)cc3)nn3c2CN(CCO)CC3)ccn1 ; Cc1cc(-c2c(-c3ccc(F)cc3)nn3c2CN(C2COC2)CC3)ccn1 ; Cc1cc(-c2c(-c3ccc(F)cc3)nn3c2CN(C(=O)C2CCOCC2)CC3)ccn1 ; Cc1cc(-c2c(-c3ccc(F)cc3)nn3c2CN(C(=O)[C@@H]2CCOC2)CC3)ccn1 ; Cc1cc(-c2c(-c3ccc(F)cc3)nn3c2CN(C(=O)[C@H]2CCOC2)CC3)ccn1]

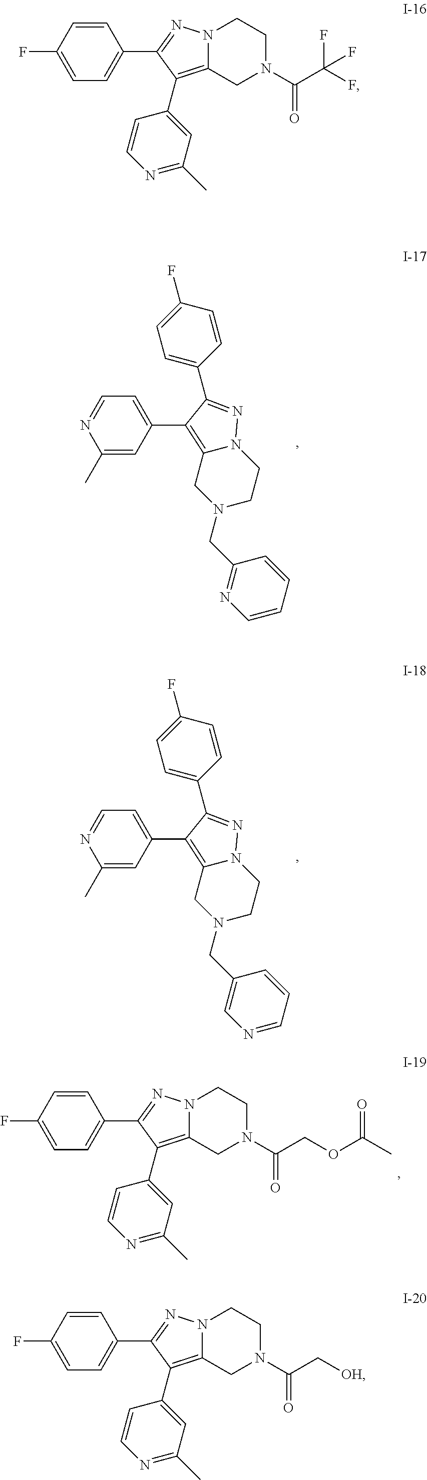 [SMILES: Cc1cc(-c2c(-c3ccc(F)cc3)nn3c2CN(C(=O)C(F)(F)F)CC3)ccn1 ; Cc1cc(-c2c(-c3ccc(F)cc3)nn3c2CN(Cc2ccccn2)CC3)ccn1 ; Cc1cc(-c2c(-c3ccc(F)cc3)nn3c2CN(Cc2cccnc2)CC3)ccn1 ; CC(=O)OCC(=O)N1CCn2nc(-c3ccc(F)cc3)c(-c3ccnc(C)c3)c2C1 ; Cc1cc(-c2c(-c3ccc(F)cc3)nn3c2CN(C(=O)CO)CC3)ccn1]

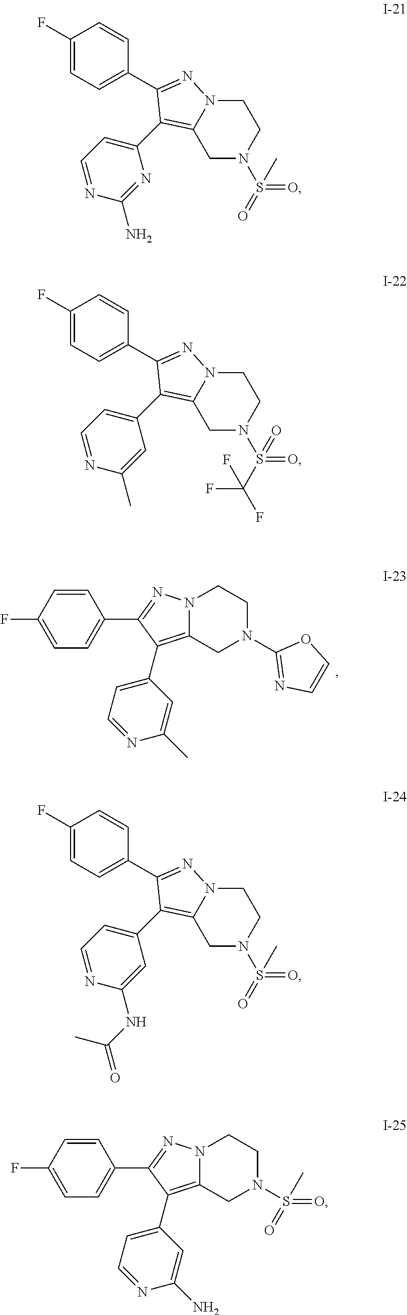 [SMILES: CS(=O)(=O)N1CCn2nc(-c3ccc(F)cc3)c(-c3ccnc(N)n3)c2C1 ; Cc1cc(-c2c(-c3ccc(F)cc3)nn3c2CN(S(=O)(=O)C(F)(F)F)CC3)ccn1 ; Cc1cc(-c2c(-c3ccc(F)cc3)nn3c2CN(c2ncco2)CC3)ccn1 ; CC(=O)Nc1cc(-c2c(-c3ccc(F)cc3)nn3c2CN(S(C)(=O)=O)CC3)ccn1 ; CS(=O)(=O)N1CCn2nc(-c3ccc(F)cc3)c(-c3ccnc(N)c3)c2C1]

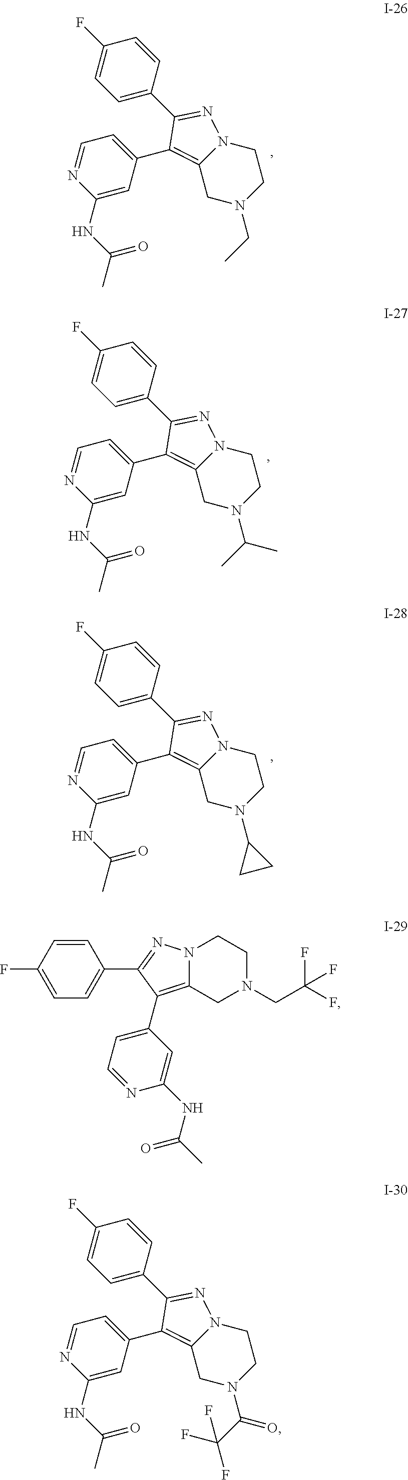

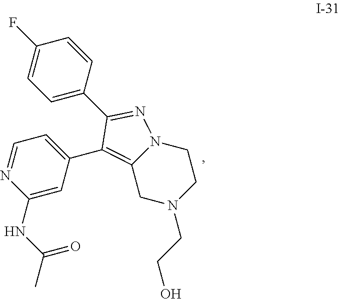 [SMILES: CC(=O)Nc1cc(-c2c(-c3ccc(F)cc3)nn3c2CN(CCO)CC3)ccn1]

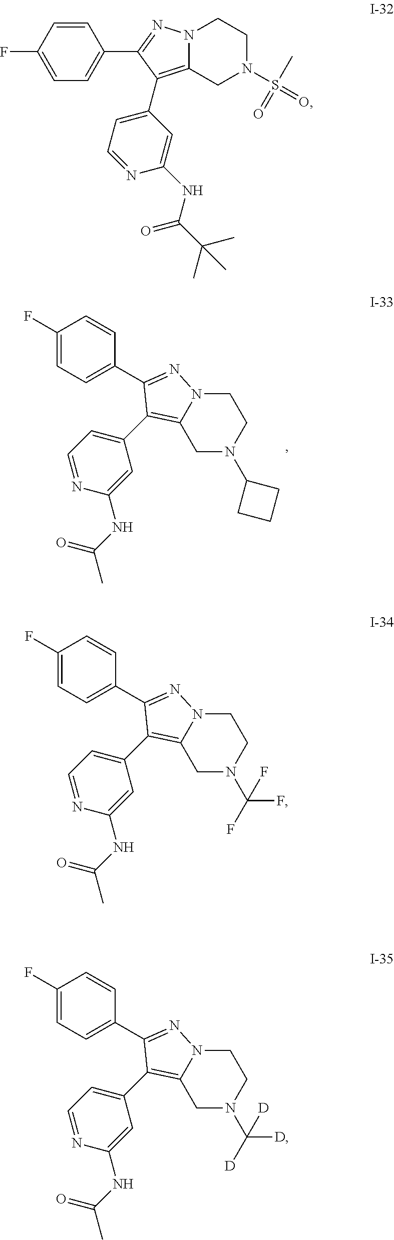

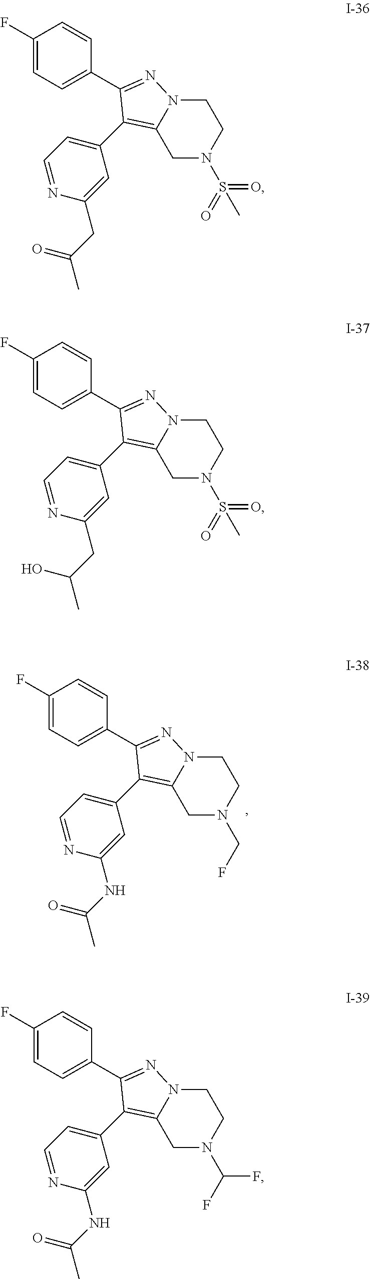 [SMILES: CC(=O)Cc1cc(-c2c(-c3ccc(F)cc3)nn3c2CN(S(C)(=O)=O)CC3)ccn1 ; CC(O)Cc1cc(-c2c(-c3ccc(F)cc3)nn3c2CN(S(C)(=O)=O)CC3)ccn1 ; CC(=O)Nc1cc(-c2c(-c3ccc(F)cc3)nn3c2CN(CF)CC3)ccn1 ; CC(=O)Nc1cc(-c2c(-c3ccc(F)cc3)nn3c2CN(C(F)F)CC3)ccn1]

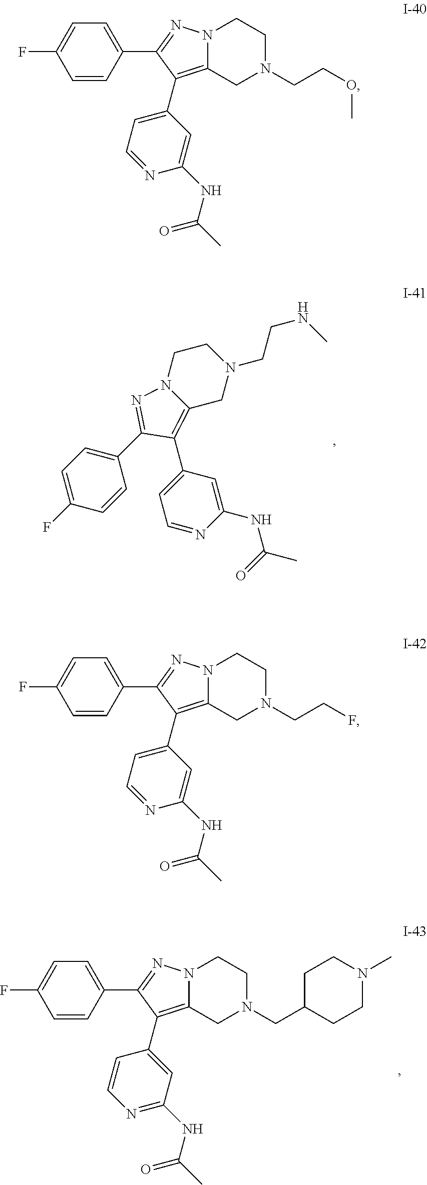 [SMILES: COCCN1CCn2nc(-c3ccc(F)cc3)c(-c3ccnc(NC(C)=O)c3)c2C1 ; CNCCN1CCn2nc(-c3ccc(F)cc3)c(-c3ccnc(NC(C)=O)c3)c2C1 ; CC(=O)Nc1cc(-c2c(-c3ccc(F)cc3)nn3c2CN(CCF)CC3)ccn1 ; CC(=O)Nc1cc(-c2c(-c3ccc(F)cc3)nn3c2CN(CC2CCN(C)CC2)CC3)ccn1]

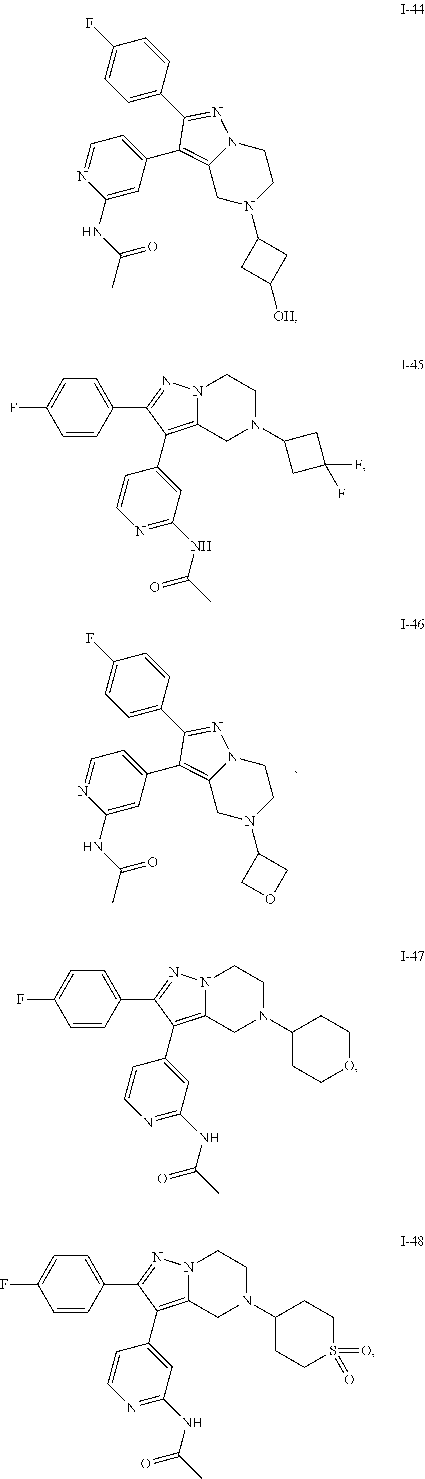 [SMILES: CC(=O)Nc1cc(-c2c(-c3ccc(F)cc3)nn3c2CN(C2CC(O)C2)CC3)ccn1 ; CC(=O)Nc1cc(-c2c(-c3ccc(F)cc3)nn3c2CN(C2CC(F)(F)C2)CC3)ccn1 ; CC(=O)Nc1cc(-c2c(-c3ccc(F)cc3)nn3c2CN(C2COC2)CC3)ccn1 ; CC(=O)Nc1cc(-c2c(-c3ccc(F)cc3)nn3c2CN(C2CCOCC2)CC3)ccn1 ; CC(=O)Nc1cc(-c2c(-c3ccc(F)cc3)nn3c2CN(C2CCS(=O)(=O)CC2)CC3)ccn1]

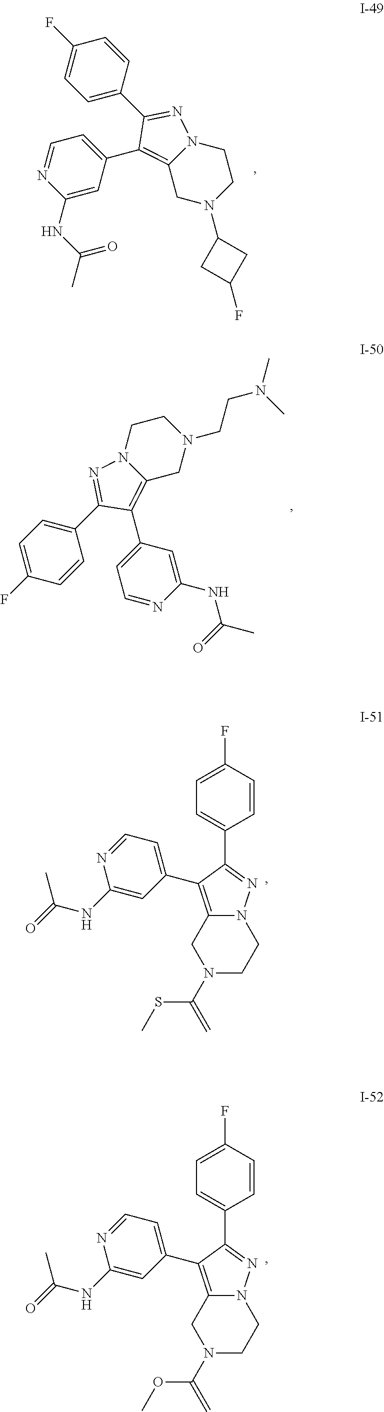 [SMILES: CC(=O)Nc1cc(-c2c(-c3ccc(F)cc3)nn3c2CN(C2CC(F)C2)CC3)ccn1 ; CC(=O)Nc1cc(-c2c(-c3ccc(F)cc3)nn3c2CN(CCN(C)C)CC3)ccn1 ; C=C(SC)N1CCn2nc(-c3ccc(F)cc3)c(-c3ccnc(NC(C)=O)c3)c2C1 ; C=C(OC)N1CCn2nc(-c3ccc(F)cc3)c(-c3ccnc(NC(C)=O)c3)c2C1]

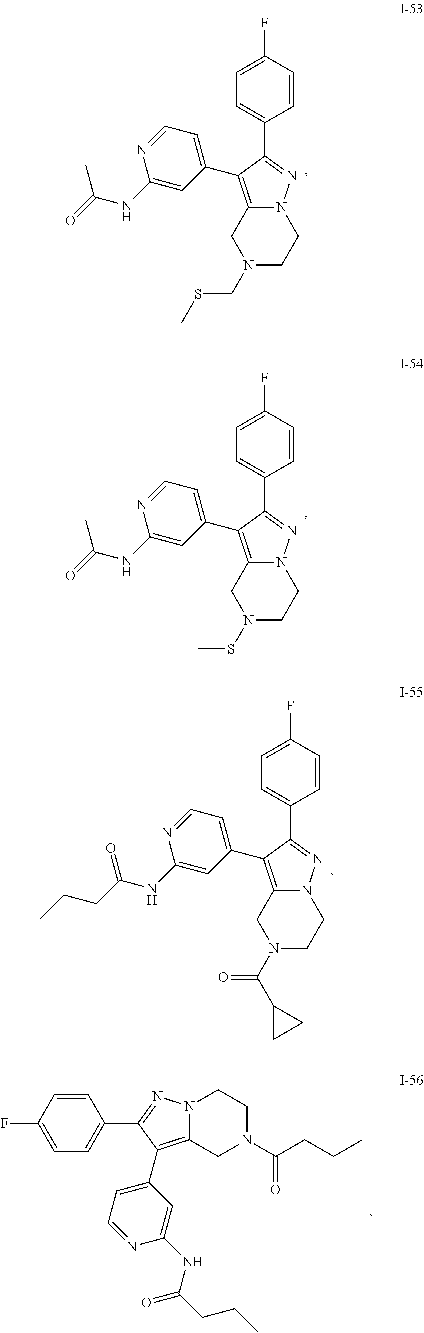 [SMILES: CSCN1CCn2nc(-c3ccc(F)cc3)c(-c3ccnc(NC(C)=O)c3)c2C1 ; CSN1CCn2nc(-c3ccc(F)cc3)c(-c3ccnc(NC(C)=O)c3)c2C1 ; CCCC(=O)Nc1cc(-c2c(-c3ccc(F)cc3)nn3c2CN(C(=O)C2CC2)CC3)ccn1 ; CCCC(=O)Nc1cc(-c2c(-c3ccc(F)cc3)nn3c2CN(C(=O)CCC)CC3)ccn1]

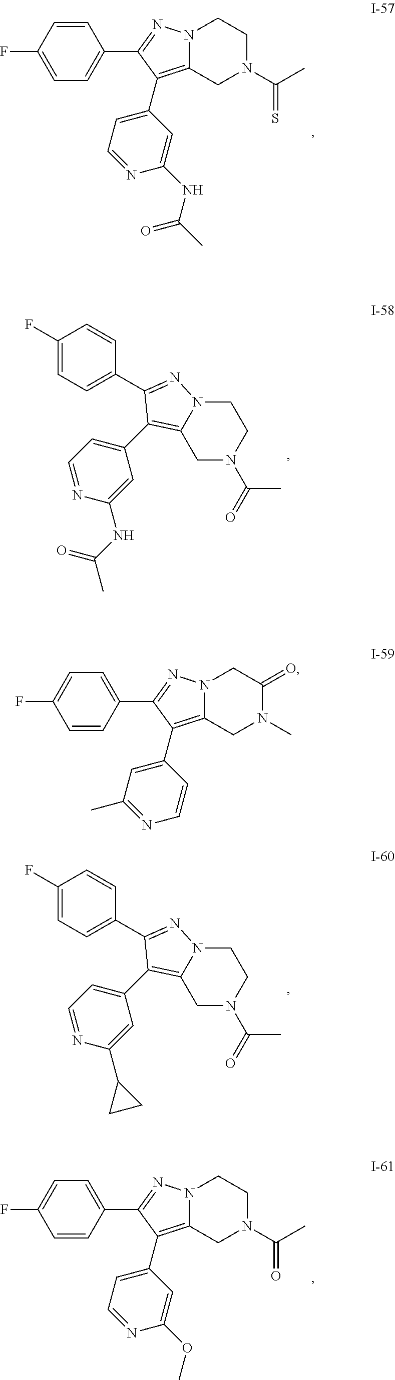 [SMILES: CC(=O)Nc1cc(-c2c(-c3ccc(F)cc3)nn3c2CN(C(C)=S)CC3)ccn1 ; CC(=O)Nc1cc(-c2c(-c3ccc(F)cc3)nn3c2CN(C(C)=O)CC3)ccn1 ; Cc1cc(-c2c(-c3ccc(F)cc3)nn3c2CN(C)C(=O)C3)ccn1 ; CC(=O)N1CCn2nc(-c3ccc(F)cc3)c(-c3ccnc(C4CC4)c3)c2C1 ; COc1cc(-c2c(-c3ccc(F)cc3)nn3c2CN(C(C)=O)CC3)ccn1]

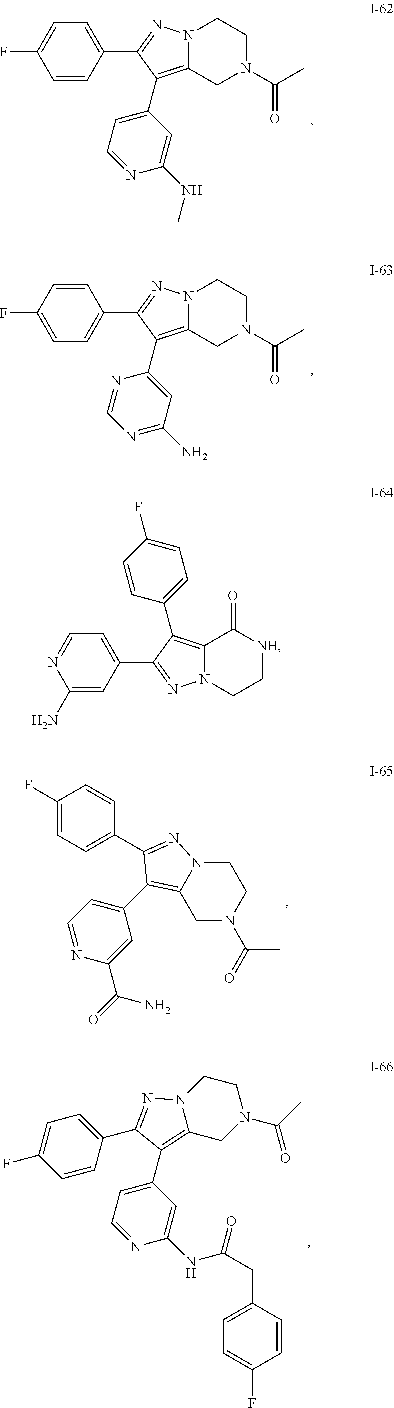 [SMILES: CNc1cc(-c2c(-c3ccc(F)cc3)nn3c2CN(C(C)=O)CC3)ccn1 ; CC(=O)N1CCn2nc(-c3ccc(F)cc3)c(-c3cc(N)ncn3)c2C1 ; Nc1cc(-c2nn3c(c2-c2ccc(F)cc2)C(=O)NCC3)ccn1 ; CC(=O)N1CCn2nc(-c3ccc(F)cc3)c(-c3ccnc(C(N)=O)c3)c2C1 ; CC(=O)N1CCn2nc(-c3ccc(F)cc3)c(-c3ccnc(NC(=O)Cc4ccc(F)cc4)c3)c2C1]

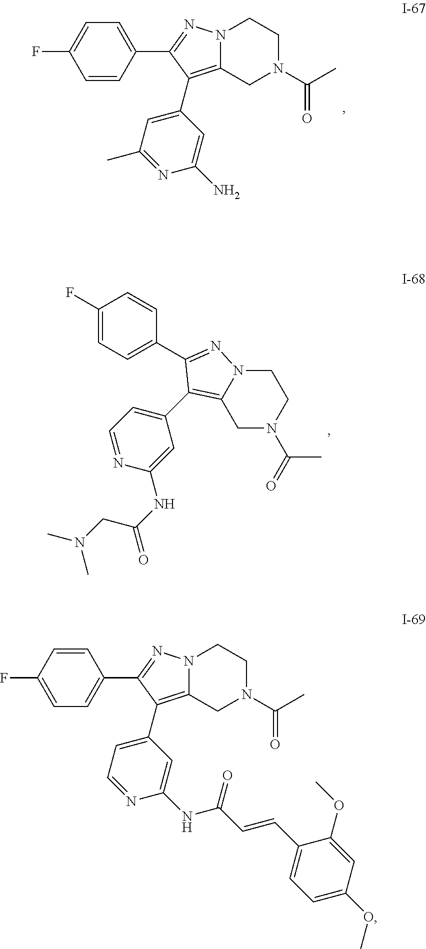

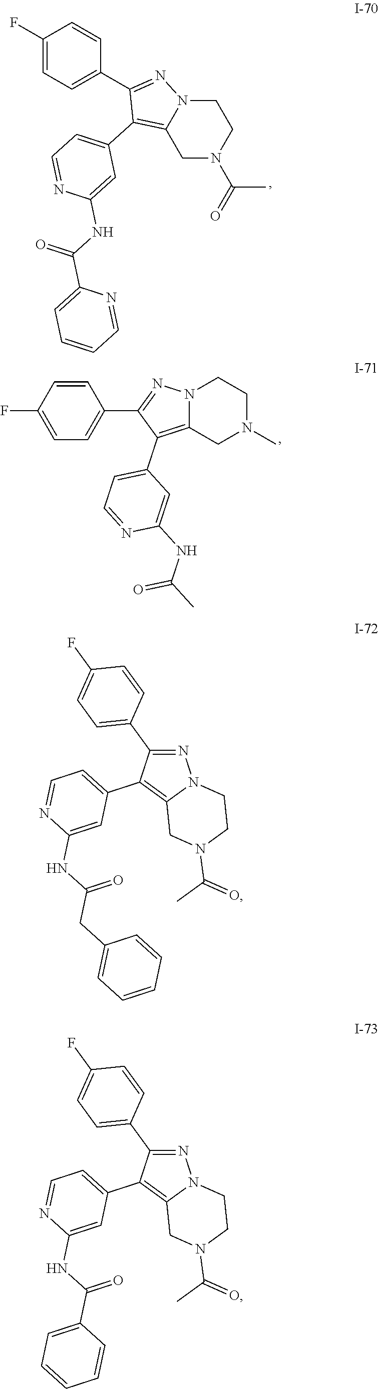

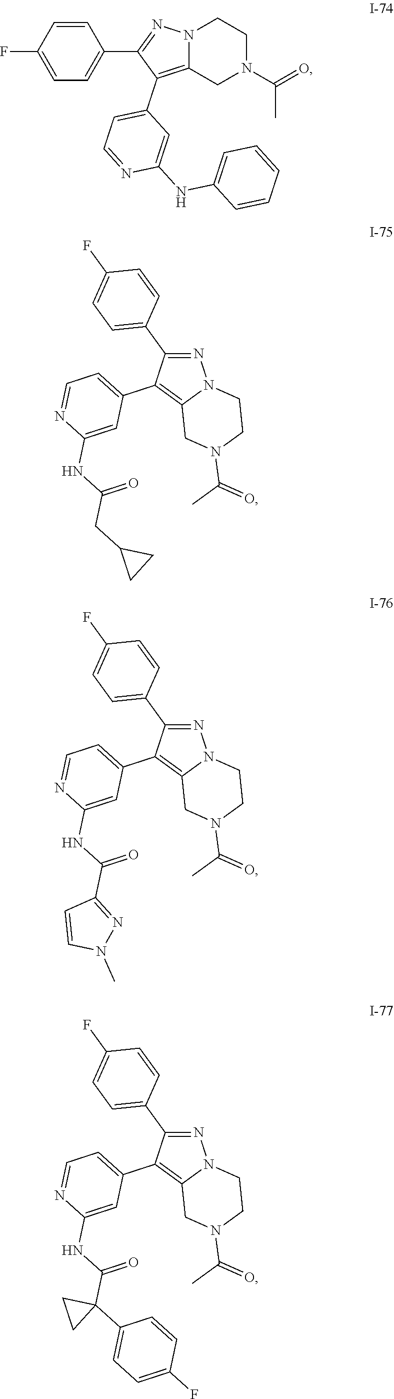 [SMILES: CC(=O)N1CCn2nc(-c3ccc(F)cc3)c(-c3ccnc(Nc4ccccc4)c3)c2C1 ; CC(=O)N1CCn2nc(-c3ccc(F)cc3)c(-c3ccnc(NC(=O)CC4CC4)c3)c2C1 ; CC(=O)N1CCn2nc(-c3ccc(F)cc3)c(-c3ccnc(NC(=O)c4ccn(C)n4)c3)c2C1 ; CC(=O)N1CCn2nc(-c3ccc(F)cc3)c(-c3ccnc(NC(=O)C4(c5ccc(F)cc5)CC4)c3)c2C1]

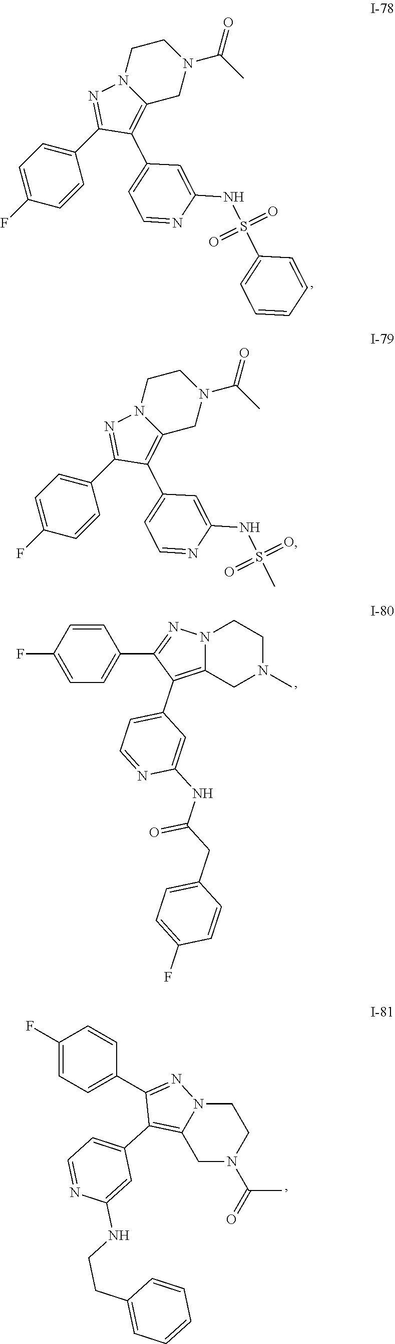

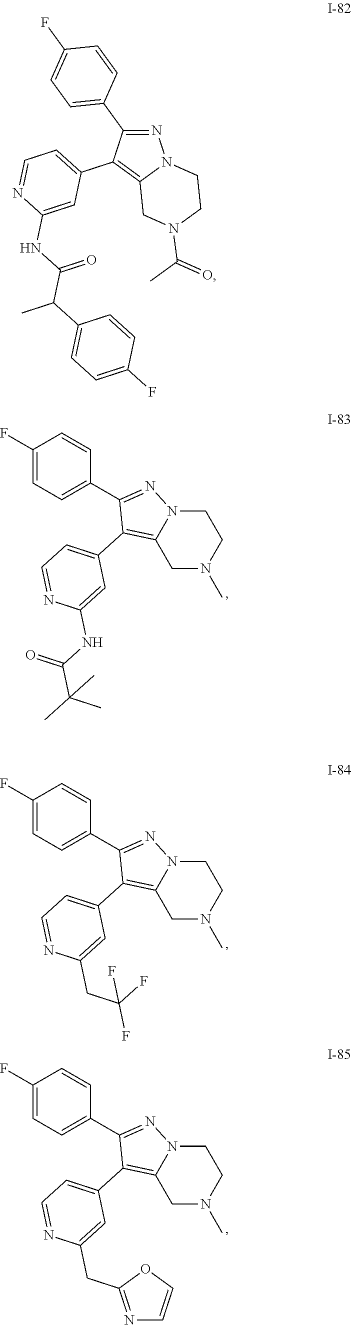 [SMILES: CC(=O)N1CCn2nc(-c3ccc(F)cc3)c(-c3ccnc(NC(=O)C(C)c4ccc(F)cc4)c3)c2C1 ; CN1CCn2nc(-c3ccc(F)cc3)c(-c3ccnc(NC(=O)C(C)(C)C)c3)c2C1 ; CN1CCn2nc(-c3ccc(F)cc3)c(-c3ccnc(CC(F)(F)F)c3)c2C1 ; CN1CCn2nc(-c3ccc(F)cc3)c(-c3ccnc(Cc4ncco4)c3)c2C1]

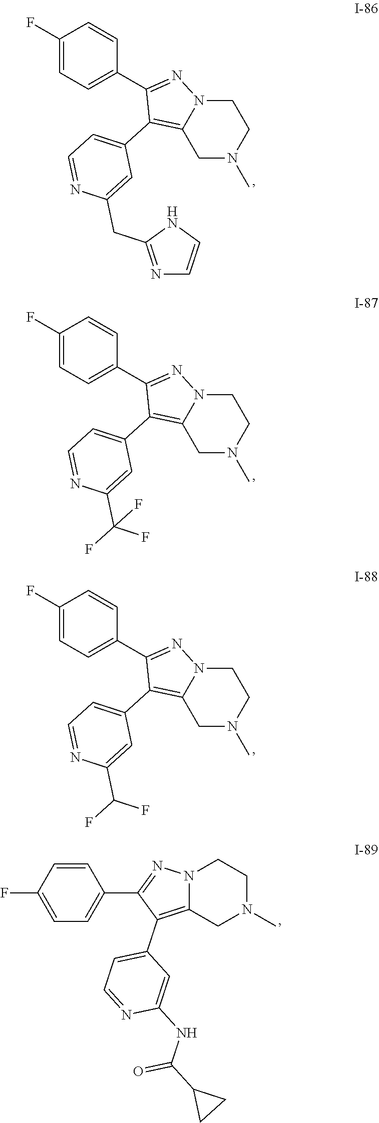

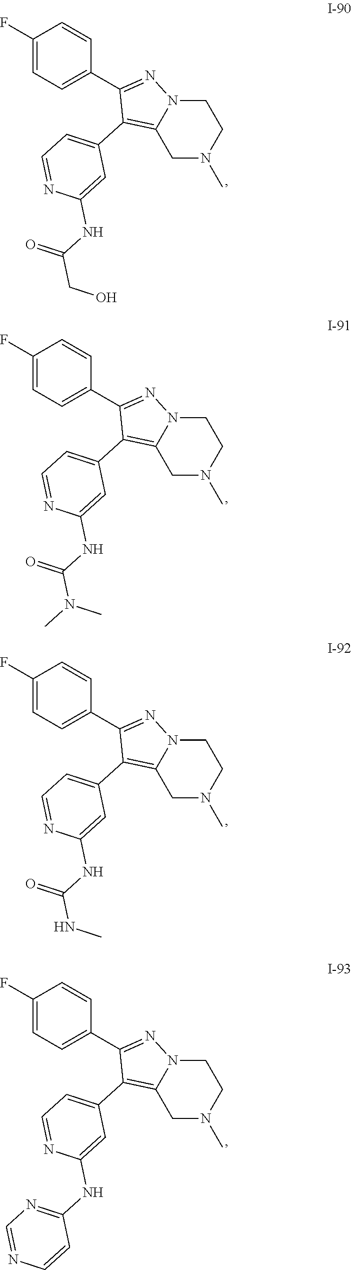

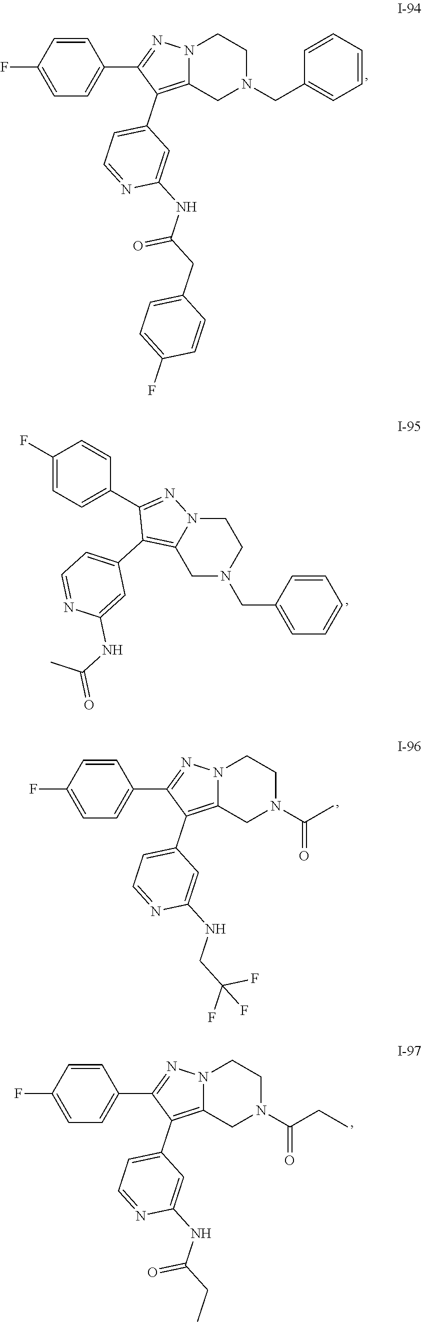 [SMILES: O=C(Cc1ccc(F)cc1)Nc1cc(-c2c(-c3ccc(F)cc3)nn3c2CN(Cc2ccccc2)CC3)ccn1 ; CC(=O)Nc1cc(-c2c(-c3ccc(F)cc3)nn3c2CN(Cc2ccccc2)CC3)ccn1 ; CC(=O)N1CCn2nc(-c3ccc(F)cc3)c(-c3ccnc(NCC(F)(F)F)c3)c2C1 ; CCC(=O)Nc1cc(-c2c(-c3ccc(F)cc3)nn3c2CN(C(=O)CC)CC3)ccn1]

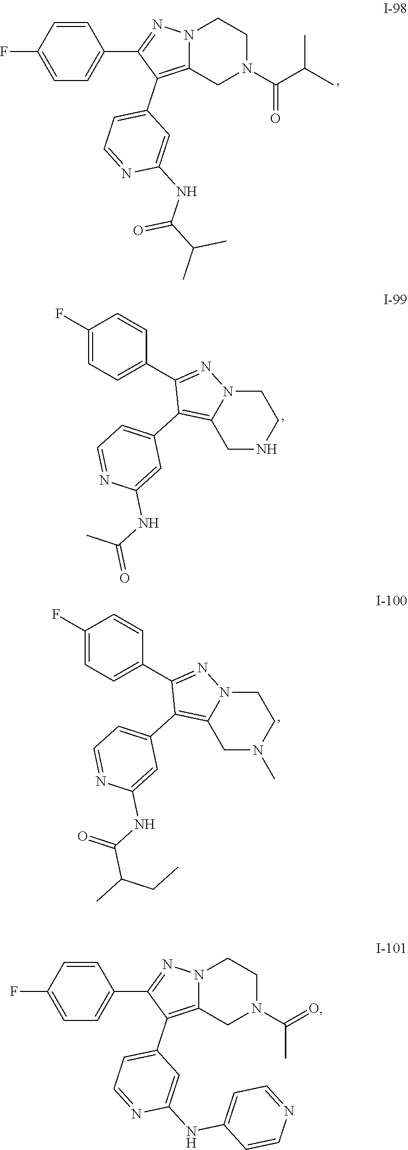

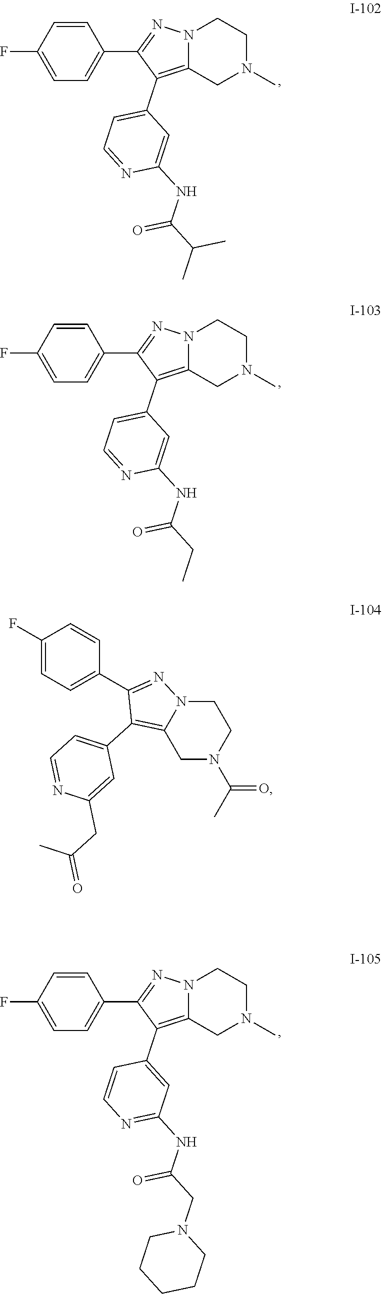 [SMILES: CC(C)C(=O)Nc1cc(-c2c(-c3ccc(F)cc3)nn3c2CN(C)CC3)ccn1 ; CCC(=O)Nc1cc(-c2c(-c3ccc(F)cc3)nn3c2CN(C)CC3)ccn1 ; CC(=O)Cc1cc(-c2c(-c3ccc(F)cc3)nn3c2CN(C(C)=O)CC3)ccn1 ; CN1CCn2nc(-c3ccc(F)cc3)c(-c3ccnc(NC(=O)CN4CCCCC4)c3)c2C1]

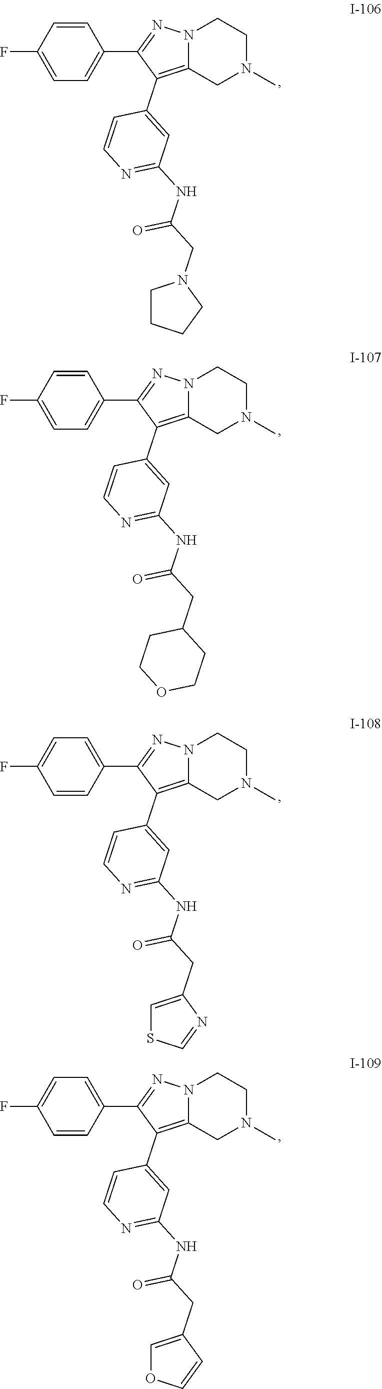

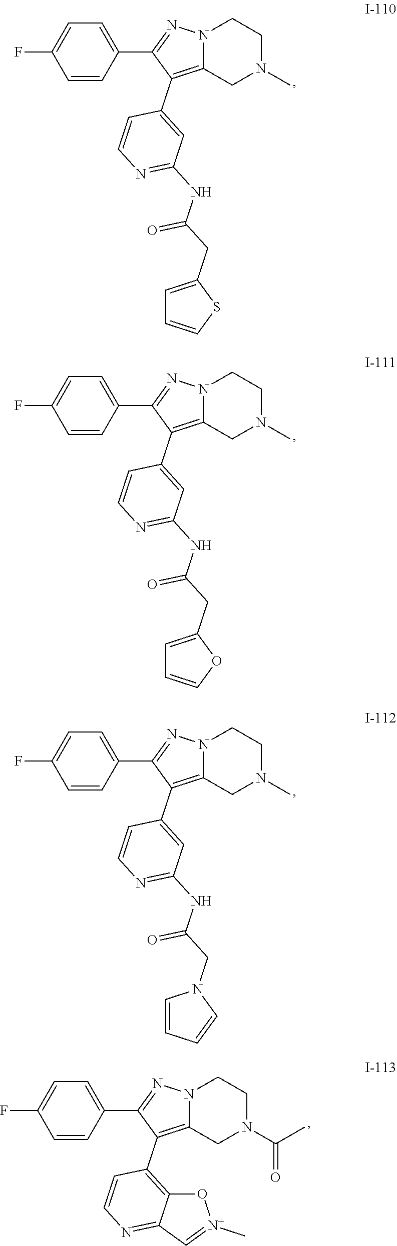 [SMILES: CN1CCn2nc(-c3ccc(F)cc3)c(-c3ccnc(NC(=O)Cc4cccs4)c3)c2C1 ; CN1CCn2nc(-c3ccc(F)cc3)c(-c3ccnc(NC(=O)Cc4ccco4)c3)c2C1 ; CN1CCn2nc(-c3ccc(F)cc3)c(-c3ccnc(NC(=O)Cn4cccc4)c3)c2C1 ; CC(=O)N1CCn2nc(-c3ccc(F)cc3)c(-c3ccnc4c[n+](C)oc34)c2C1]

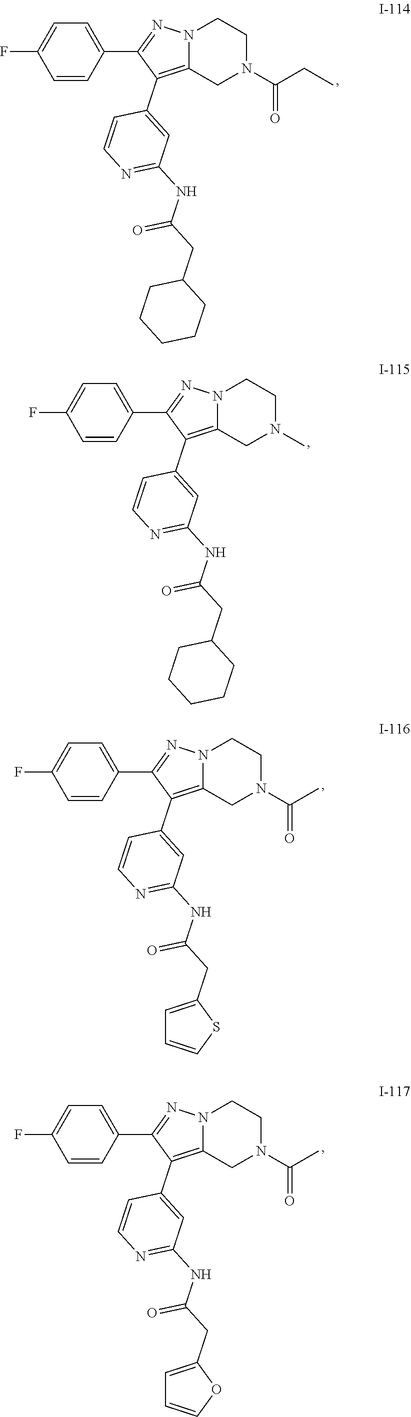

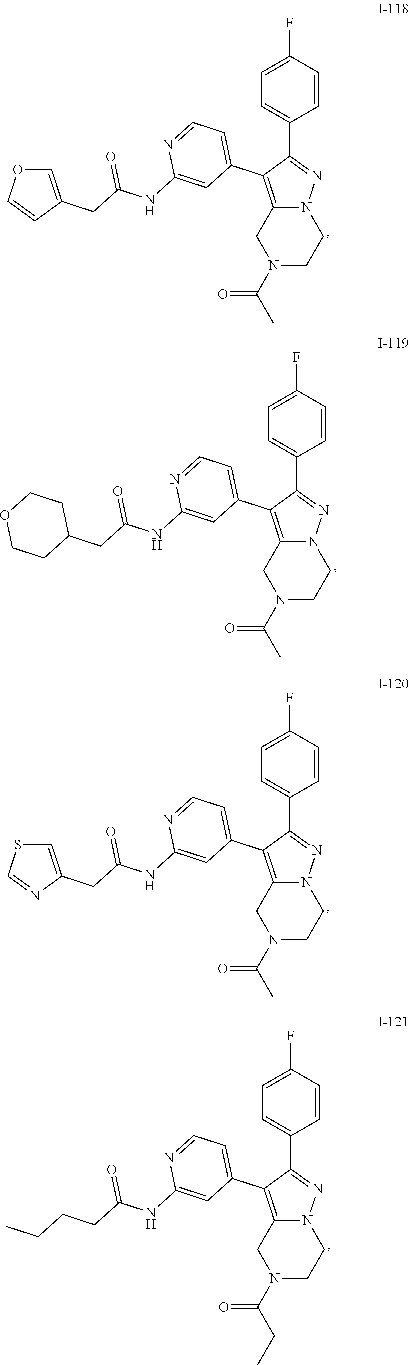

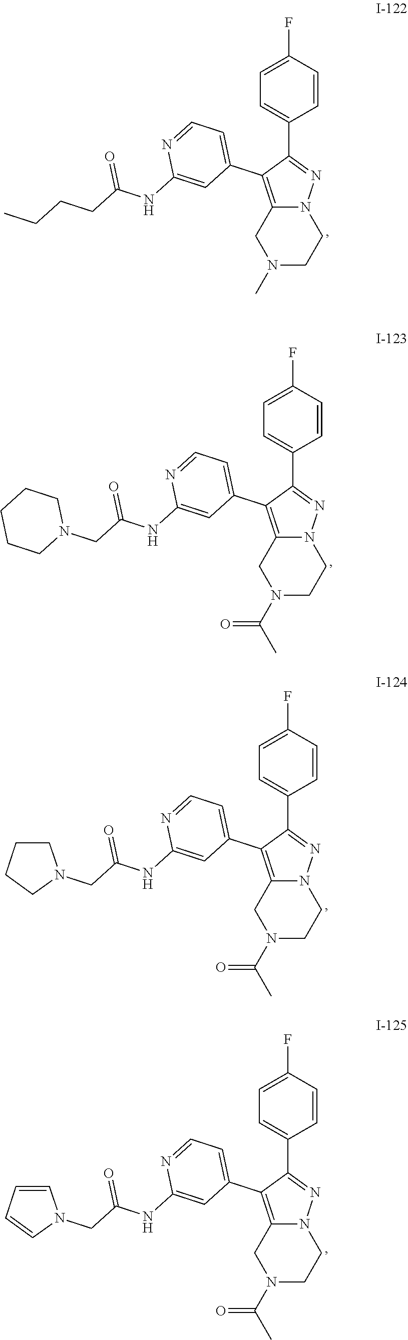

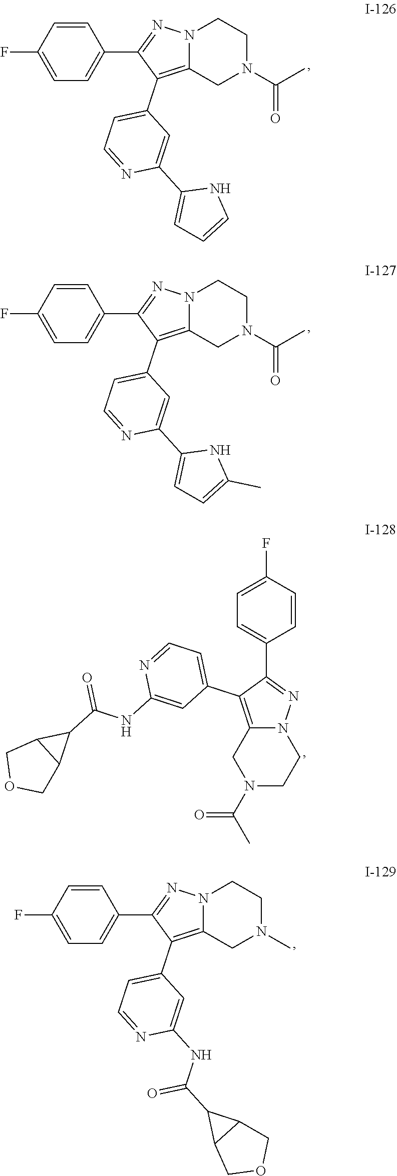

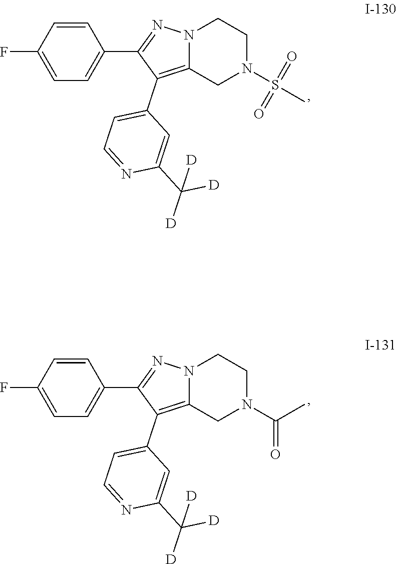 [SMILES: [2H]C([2H])([2H])c1cc(-c2c(-c3ccc(F)cc3)nn3c2CN(S(C)(=O)=O)CC3)ccn1 ; [2H]C([2H])([2H])c1cc(-c2c(-c3ccc(F)cc3)nn3c2CN(C(C)=O)CC3)ccn1]

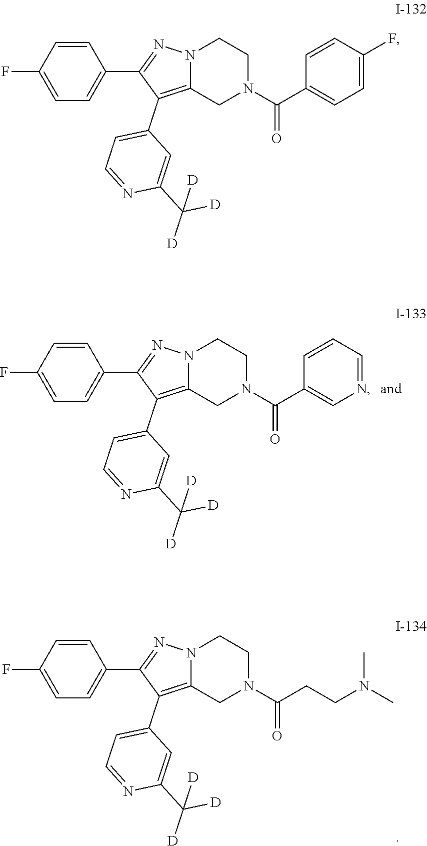 [SMILES: [2H]C([2H])([2H])c1cc(-c2c(-c3ccc(F)cc3)nn3c2CN(C(=O)c2ccc(F)cc2)CC3)ccn1 ; [2H]C([2H])([2H])c1cc(-c2c(-c3ccc(F)cc3)nn3c2CN(C(=O)c2cccnc2)CC3)ccn1 ; [2H]C([2H])([2H])c1cc(-c2c(-c3ccc(F)cc3)nn3c2CN(C(=O)CCN(C)C)CC3)ccn1]

In another aspect, the present application provides a composition comprising a compound of any one of the present application or of any of formula (I), or a pharmaceutically acceptable salt, prodrug, or metabolite thereof, or a solvate or hydrate of any of the foregoing, and optionally a pharmaceutically acceptable carrier.

In another aspect, the present application provides a method for inhibiting casein kinase (CK) activity, said method comprising administering to a subject in need thereof an effective amount of the compound of any of the present application or of any of formula (I), or a pharmaceutically acceptable salt, prodrug, or metabolite thereof, or a solvate or hydrate of any of the foregoing.

In some embodiments, wherein said casein kinase (CK) is selected from the group consisting of casein kinase 1 alpha (CK1α), casein kinase I delta (CK1δ) and casein kinase I epsilon (CK1ε).

In some embodiments, wherein said method is selected from the group consisting of an in vitro method, an ex vivo method, and an in vivo method.

In another aspect, the present application provides a method for preventing and/or treating a disease or disorder, said method comprising administering to a subject in need thereof an effective amount of the compound of the present application or of any of formula (I), or a pharmaceutically acceptable salt, prodrug, or metabolite thereof, or a solvate or hydrate of any of the foregoing.

In some embodiments, wherein said disease or disorder is selected from the group consisting of neurological disease and psychiatric disease.

In some embodiments, wherein said disease or disorder is selected from the group consisting of mood disorder, sleep disorder, and circadian disorder.

In some embodiments, wherein said disease or disorder is selected from the group consisting of depressive disorder and bipolar disorder.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1 to FIG. 134 illustrate the synthetic schemes of compound I-1 to compound I-134.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

As used herein, the term “alkyl”, either alone or within other terms, generally refers to a linear or branched-chain saturated hydrocarbyl substituent (i.e., a substituent obtained from a hydrocarbon by removal of a hydrogen) containing from one to twenty carbon atoms; for example, from one to twelve carbon atoms; in another example, from one to ten carbon atoms; in another embodiment, from one to six carbon atoms; and in another embodiment, from one to four carbon atoms (such as 1, 2, 3 or more carbon atoms). Examples of such substituents may include e.g., methyl, ethyl, propyl (including n-propyl and isopropyl), butyl (including n-butyl, isobutyl, sec-butyl and terf-butyl), pentyl, isoamyl, hexyl and the like. In some instances, the number of carbon atoms in a hydrocarbyl substituent (i.e., alkyl, alkenyl, cycloalkyl, aryl, etc.) may be indicated by the prefix “Cₐ-C_{b}” wherein a is the minimum and b is the maximum number of carbon atoms in the substituent. Thus, for example, “C₁-C₆ alkyl” may refer to an alkyl substituent containing from 1 to 6 carbon atoms. The “alkyl” groups may be optionally substituted with one or more substitutions.

As used herein, the term “alkenyl”, either alone or within other terms, generally refers to a linear or branched-carbon radicals having at least one carbon-carbon double bond. The term “alkenyl” may contain conjugated and non-conjugated carbon-carbon double bonds or combinations thereof. An alkenyl group, for example and without being limited thereto, may contain two to about twenty carbon atoms or, in a particular embodiment, two to about twelve carbon atoms. In embodiments, alkenyl groups may contain two to about four carbon atoms (such as 2, 3 or more carbon atoms). Examples of alkenyl groups include, but are not limited thereto, ethenyl, propenyl, allyl, propenyl, butenyl and 4-methylbutenyl. The terms “alkenyl” contain groups having “cis” and “trans” orientations, or alternatively, “E” and “Z” orientations. In some instances, the number of carbon atoms may be indicated by the prefix “Cₐ-C_{b}” wherein a is the minimum and b is the maximum number of carbon atoms in the substituent. The “alkenyl” groups may be optionally substituted with one or more substitutions.

As used herein, the term “alkynyl”, either alone or within other terms, generally refers to linear or branched carbon radicals having at least one carbon-carbon triple bond. The term “alkynyl” may contain conjugated and non-conjugated carbon-carbon triple bonds or combinations thereof. Alkynyl group, for example and without being limited thereto, may contain two to about twenty carbon atoms or, in a particular embodiment, two to about twelve carbon atoms. In embodiments, alkynyl groups may contain two to about ten carbon atoms. Some examples may be alkynyl having two to about four carbon atoms (such as 2, 3 or more carbon atoms). In some instances, the number of carbon atoms may be indicated by the prefix “Cₐ-C_{b}” wherein a is the minimum and b is the maximum number of carbon atoms in the substituent. Examples of such groups include propargyl, butynyl, and the like. The “alkynyl” groups may be optionally substituted with one or more substitutions.

As used herein, the term “amino”, either alone or within other terms, generally refers to formula —NH₂ group. The “amino” groups may be optionally substituted with one or more substitutions.

As used herein, the term “carbocycle”, either alone or within other terms, generally refers to a saturated or unsaturated non-aromatic monocylic, bicyclic, or polycyclic ring system having from 3 to 14 ring atoms (and all combinations and subcombinations of ranges and specific numbers of carbon atoms therein) wherein all of the ring atoms are carbon atoms. Monocylic carbocycles may have 3 to 6 ring atoms, or 5 to 6 ring atoms. Bicylic carbocycles may have 7 to 12 ring atoms, e.g., arranged as a bicyclo[4,5], [5,5], [5,6] or [6,6] system, or 9 or 10 ring atoms arranged as a bicyclo[5,6] or [6,6] system. The term “carbocycle” may contain, for example, a monocyclic carbocycle ring fused to an aryl ring (e.g., a monocyclic carbocycle ring fused to a benzene ring). Carbocycles may have 3 to 8 carbon ring atoms. In some instances, the number of carbon atoms may be indicated by the prefix “Cₐ-C_{b}” wherein a is the minimum and b is the maximum number of carbon atoms in the substituent. The “carbocycle” groups may be optionally substituted with one or more substitutions.

As used herein, the term “heterocycle”, either alone or within other terms, generally refers to a monocyclic, bicyclic, or polycyclic ring system having from 3 to 14 ring atoms (also referred to as ring members) wherein at least one ring atom in at least one ring may be a heteroatom selected from N, O, P, or S (and all combinations and subcombinations of ranges and specific numbers of carbon atoms and heteroatoms therein). The heterocycle may have from 1 to 4 ring heteroatoms independently selected from N, O, P, or S. One or more N, C, or S atoms in a heterocycle may be oxidized. A monocylic heterocycle may have 3 to 7 ring members (e.g., 2 to 6 carbon atoms and 1 to 3 heteroatoms independently selected from N, O, P, or S), and a bicyclic heterocycle may have 5 to 10 ring members (e.g., 4 to 9 carbon atoms and 1 to 3 heteroatoms independently selected from N, O, P, or S). The heterocycle that contains the heteroatom may be non-aromatic. Unless otherwise noted, the heterocycle is attached to its pendant group at any heteroatom or carbon atom that results in a stable structure. In some instances, the number of carbon atoms may be indicated by the prefix “Cₐ-C_{b}” wherein a is the minimum and b is the maximum number of carbon atoms in the substituent. The “heterocycle” groups may be optionally substituted with one or more substitutions.

As used herein, the term “aryl”, either alone or within other terms, generally refers to an aromatic substituent containing one ring or two or three fused rings. The aryl substituent may have six to eighteen carbon atoms. As an example, the aryl substituent may have six to fourteen carbon atoms. The term “aryl” may refer to substituents such as phenyl, naphthyl and anthracenyl. The term “aryl” may also contain substituents such as phenyl, naphthyl and anthracenyl that are fused to a C₄-C₁₀ carbocyclic ring, such as a C, or a C₆ carbocyclic ring, or to a 4- to 10-membered heterocyclic ring, wherein a group having such a fused aryl group as a substituent is bound to an aromatic carbon of the aryl group. When such a fused aryl group is substituted with one more substituent, the one or more substituents, unless otherwise specified, may be each bound to an aromatic carbon of the fused aryl group. The fused C₄-C₁₀ carbocyclic or 4- to 10-membered heterocyclic ring may optionally be optionally substituted. Examples of aryl groups may include accordingly phenyl, naphthalenyl, tetrahydronaphthalenyl (also known as “tetralinyl”), indenyl, isoindenyl, indanyl, anthracenyl, phenanthrenyl, benzonaphthenyl (also known as “phenalenyl”), and fluorenyl. In some instances, the number of carbon atoms may be indicated by the prefix “Cₐ-C_{b}” wherein a is the minimum and b is the maximum number of carbon atoms in the substituent. The “aryl” groups may be optionally substituted with one or more substitutions.

As used herein, the term “heteroaryl”, either alone or within other terms, generally refers to an aromatic ring structure containing from 5 to 14 ring atoms in which at least one of the ring atoms is a heteroatom (for example, oxygen, nitrogen, or sulfur), with the remaining ring atoms being independently selected from the group consisting of carbon, oxygen, nitrogen, and sulfur. A heteroaryl may be a single ring or 2 or 3 fused rings. Examples of heteroaryl substituents may include but not limited to: 6-membered ring substituents such as pyridyl, pyrazyl, pyrimidinyl, and pyridazinyl; 5-membered ring substituents such as triazolyl, imidazolyl, furanyl, thiophenyl, pyrazolyl, oxazolyl, isoxazolyl, thiazolyl, 1,2,3-, 1,2, 4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused ring substituents such as quinolinyl, isoquinolinyl, oinnolinyl, quinazolinyl, and 1,4-benzoxazinyl. In a group that has a heteroaryl substituent, the ring atom of the heteroaryl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heteroaryl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom. In some instances, the number of carbon atoms may be indicated by the prefix “Cₐ-C_{b}” wherein a is the minimum and b is the maximum number of carbon atoms in the substituent. The “heteroaryl” groups may be optionally substituted with one or more substitutions.

As used herein, the term “halogen”, either alone or within other terms, generally refers to fluorine (which may be depicted as —F), chlorine (which may be depicted as —Cl), bromine (which may be depicted as —Br), or iodine (which may be depicted as —I). In one embodiment, the halogen may be chlorine. In another embodiment, the halogen may be fluorine. In another embodiment, the halogen may be bromine.

As used herein, the term “cyano”, either alone or within other terms, generally refers to formula —CN group.

As used herein, the term “nitro”, either alone or within other terms, generally refers to formula —NO₂ group.

As used herein, the term “hydroxy”, either alone or within other terms, generally refers to formula —OH group. The “hydroxy” groups may be optionally substituted with one or more substitutions.

As used herein, the term “phosphorous-containing group”, either alone or within other terms, generally refers to functional group containing on or more phosphorous atoms. The phosphorous-containing group may refer to —O—P—(OH)₂, —O—PH—(OH), —O—PH₂, —P—(OH)₂, —PH—(OH), —PH₄, —PH₂═CH₂, —CH═PH₃, —O—P(═O)₂, —O—P(═O)—(OH)₂, —O—PH(═O)—OH, —P(═O)—(OH)₂, —O—PH₂(═O), —PH(═O)—OH, —PH₂(═O), —O—P(═O)(OH)—P(═O)(OH)₂, —O—P(═O)(OH)—O—P(═O)(OH)₂, —PH—PH₂, or —P═PH. The “phosphorous-containing group” may be optionally substituted with one or more substitutions.

As used herein, the term “silicon-containing group”, either alone or within other terms, generally refers to functional group containing on or more silicon atoms. The silicon-containing group may refer to —SiH₃. The “silicon-containing group” may be optionally substituted with one or more substitutions.

As used herein, the term “thio”, either alone or within other terms, generally refers to formula —SH group. The “thio” groups may be optionally substituted with one or more substitutions.

As used herein, the term “carboxyl”, either alone or within other terms, generally refers to formula —C(═O)OH group. The “carboxyl” groups may be optionally substituted with one or more substitutions.

As used herein, the term “sulfonyl”, either alone or within other terms, generally refers to formula —S(═O)₂—H, group. The “sulfonyl” groups may be optionally substituted with one or more substitutions.

As used herein, the term “sulfinyl”, either alone or within other terms, generally refers to formula —S(═O)—H group. The “sulfinyl” groups may be optionally substituted with one or more substitutions.

As used herein, the term “acyl”, either alone or within other terms, generally refers to a carboxylic acid ester of the formula —C(O)R in which the non-carbonyl moiety of the ester group (i.e., R) may be selected from straight, branched, or cyclic alkyl. The term acyl may include but not limited to acetyl, propionyl, butyryl and pentanoyl. In some instances, the number of carbon atoms may be indicated by the prefix “Cₐ-C_{b}” wherein a is the minimum and b is the maximum number of carbon atoms in the substituent. The “acyl” groups may be optionally substituted with one or more substitutions.

As used herein, the term “thioacyl”, either alone or within other terms, generally refers to the formula —C(S)R in which the moiety of the ester group (i.e., R) may be selected from straight, branched, or cyclic alkyl. In some instances, the number of carbon atoms may be indicated by the prefix “Cₐ-C_{b}” wherein a is the minimum and b is the maximum number of carbon atoms in the substituent. The “thioacyl” groups may be optionally substituted with one or more substitutions.

As used herein, the term “ring”, either alone or within other terms, generally refers to any covalently closed structure. Rings may include, for example, carbocycles, heterocycles, aryls and heteroaryls. Rings may be monocyclic or polycyclic. The “ring” groups may be optionally substituted with one or more substitutions.

As used herein, the term “treating”, unless otherwise indicated, generally refers to reversing, alleviating the progress of, or preventing the disorder or condition to which such term applies, or one or more symptoms of such disorder or condition. The term “treatment”, as used herein, unless otherwise indicated, generally refers to the act of treating as “treating” is defined immediately above. The term “treating” may also include adjuvant and neo-adjuvant treatment of a subject.

As used herein, the term “preventing” unless otherwise indicated, generally refers to precluding, averting, obviating, forestalling, stopping, or hindering something from happening, especially by advance action. It may be understood that where reduce, inhibit or prevent are used herein, unless specifically indicated otherwise, the use of the other two words may be also expressly disclosed.

As used herein, the term “pharmaceutically acceptable salt” generally refers to a salt that may be pharmaceutically acceptable and that may possess the desired pharmacological activity of the parent compound. Such salts may include: acid addition salts, formed with inorganic acids or formed with organic acids or basic addition salts formed with the conjugate bases of any of the inorganic acids wherein the conjugate bases comprise a cationic component.

As used herein, the term “pharmaceutically acceptable carrier” generally refers to aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles may include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity may be maintained, for example, by the use of coating materials such as lecithin, by the maintenance of the required particle size in the case of dispersions and by the use of surfactants. These compositions may also contain adjuvants such as preservatives, wetting agents, emulsifying agents and dispersing agents. Prevention of the action of microorganisms may be ensured by the inclusion of various antibacterial and antifungal agents such as paraben, chlorobutanol, phenol, sorbic acid and the like. It may also be desirable to include isotonic agents such as sugars, sodium chloride and the like. Prolonged absorption of the injectable pharmaceutical form may be brought about by the inclusion of agents, such as aluminum monostearate and gelatin, which delay absorption. Injectable depot forms may be made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release may be controlled. Depot injectable formulations may be also prepared by entrapping the drug in liposomes or microemulsions which are compatible with body tissues. The injectable formulations may be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers may include sugars such as lactose. Desirably, at least 95% by weight of the particles of the active ingredient may have an effective particle size in the range of 0.01 to 10 micrometers.

As used herein, the term “prodrug” generally refers to a compound that is metabolized, for example hydrolyzed or oxidized, in the host to form the compound of the present invention. Typical examples of prodrugs may include compounds that have biologically labile protecting groups on a functional moiety of the active compound. Prodrugs may include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound.

As used herein, the term “casein kinase” generally refers to a protein having an activity of catalyzing the serine/threonine-selective phosphorylation of proteins. This activity may be referred to as “casein kinase activity”. The Gene ID for gene encoding casein kinase may be 1453 or 1454.

As used herein, the term “subject” generally refers to an animal, which may include, but not limited to, cattle, pigs, sheep, chicken, turkey, buffalo, llama, ostrich, dogs, cats, and humans, and the subject may be a human. It may be contemplated that in the method of treating a subject thereof of the sixth embodiment can be any of the compounds either alone or in combination with another compound of the present invention.

As used herein, the term “effective amount” generally refers to an amount of an agent or a compound being administered which will treat a disease or disorder, or some or all of the symptom. The result can be reduction and/or alleviation of the signs, symptoms, or causes of a disease or disorder, or any other desired alteration of a biological system. For example, an “effective amount” for therapeutic uses is the amount of the composition including a compound as disclosed herein required to provide a clinically significant decrease in a disease or disorder symptoms without undue adverse side effects.

As used herein, the term “administering” generally refers to the compound may be administered by any appropriate route, for example, orally, parenterally, intravenously, intradermally, subcutaneously, or topically, in liquid or solid form.

As used herein, a substituent is “substitutable” or can be “substituted” if it comprises at least one atom that is bonded to one or more hydrogen atoms. If a substituent is described as being “substituted,” hydrogen or a non-hydrogen substituent is in the place of a hydrogen substituent on a atom of the substituent. Thus, for example, a substituted alkyl substituent is an alkyl substituent wherein at least one hydrogen or a non-hydrogen substituent is in the place of a hydrogen substituent on the alkyl substituent. To illustrate, monofluoroalkyl is alkyl substituted with a fluoro substituent, and difluoroalkyl is alkyl substituted with two fluoro substituents. It should be recognized that if there is more than one substitution on a substituent, each substituent may be identical or different (unless otherwise stated).

If substituents are described as being “independently selected” from a group, each substituent may be selected independent of the other(s). Each substituent therefore may be identical to or different from the other substituent(s).

As used herein, the term “optionally substituted” generally refers to a given moiety may consist of only hydrogen substituents through available valencies (unsubstituted) or may further comprise one or more non-hydrogen substituents through available valencies (substituted) that are not otherwise specified by the name of the given moiety. For example, “R^{x} is optionally substituted” or R is optionally substituted with R^{y}″ may mean that R^{x} may be substituted with 0, 1, 2, 3, 4, 5, 6, 7, 8, or 9 R^{y}, for example, R^{x} may be substituted with 0, 1, 2, 3, 4, or 5 R^{y}, for example, R^{x} may be substituted with 1, 2, or 3 R^{y}, for example, R^{x} may be substituted with one R^{y}, for example, R^{x} may be substituted with 2 R^{y}, for example, R^{x} may be substituted with 3 R^{y}, for example, R^{x} may be substituted with 4 R^{y}, for example, R^{x} may be substituted with 5 R^{y}, for example, R^{x} may be substituted with 6 R^{y}, for example, R^{x} may be substituted with 7 R^{y}, for example, R^{x} may be substituted with 8 R^{y}, for example, R^{x} may be substituted with 9 R^{y}. In general, a non-hydrogen substituent may be any substituent that may be bound to an atom of the given moiety that is specified to be substituted. Examples of substituents include, but are not limited to, hydrogen, protium, deuterium, tritium, halogen, cyano, nitro, ═S, ═O, N₃, trifluoromethyl, hydroxy, phosphorous-containing group, silicon-containing group, thio, amino, carboxyl, sulfonyl, sulfinyl, (C₁-C₆) acyl, (C₁-C₆)thioacyl, (C₁-C₆)alkyl, (C₂-C₆)alkenyl, (C₂-C₆)alkynyl, (C₃-C₁₀)carbocycle, (C₂-C₉)heterocycle, (C₆-C₁₀) aryl, (C₁-C₉)heteroaryl, trifluoromethyl(C₁-C₆)alkyl, cyano(C₁-C₆)alkyl, halo(C₁-C₆)alkyl, nitro(C₁-C₆)alkyl, hydroxy(C₁-C₆)alkyl, (C₁-C₆)alkoxy, (C₁-C₆)alkylthio, thio(C₁-C₆)alkyl, amino(C₁-C₆)alkyl, (C₁-C₆)alkylamino, ((C₁-C₆)alkyl)₂ amino, (C₁-C₆) acyl(C₁-C₆)alkyl, (C₁-C₆)alkylsulfonyl, (C₁-C₆ alkysulfonyl, hydroxysulfonyl, hydroxysulfinyl, (C₃-C₁₀)carbocycle(C₁-C₆)alkyl, (C₂-C₉)heterocycle(C₁-C₆)alkyl, (C₆-C₁₀) aryl(C₁-C₆)alkyl, and (C₁-C₉)heteroaryl(C₁-C₆)alkyl. In addition, the substituent is itself optionally substituted by a further substituent. In one particular embodiment, examples of the further substituent include, but are not limited to, hydrogen, protium, deuterium, tritium, halogen, cyano, nitro, ═S, ═O, N₃, trifluoromethyl, hydroxy, phosphorous-containing group, silicon-containing group, thio, amino, carboxyl, sulfonyl, sulfonyl, (C₁-C₆) acyl, (C₁-C₆)thioacyl, (C₁-C₆)alkyl, (C₂-C₆)alkenyl, (C₂-C₆)alkynyl, (C₃-C₁₀)carbocycle, (C₂-C₉)heterocyle, (C₆-C₁₀) aryl, (C₁-C₉)heteroaryl, trifluoromethyl(C₁-C₆)alkyl, cyano(C₁-C₆)alkyl, halo(C₁-C₆)alkyl, nitro(C₁-C₆)alkyl, hydroxy(C₁-C₆)alkyl, (C₁-C₆)alkoxy, (C₁-C₆)alkylthio, thio(C₁-C₆)alkyl, amino(C₁-C₆)alkyl, (C₁-C₆)alkylamino, ((C₁-C₆)alkyl)₂ amino, (C₁-C₆) acyl(C₁-C₆)alkyl, (C₁-C₆)alkylsulfonyl, (C₁-C₆)alkylsulfinyl, hydroxysulfonyl, hydroxysulfinyl, (C₃-C₁₀)carbocycle(C₁-C₆)alkyl, (C₂-C₉)heterocycle(C₁-C₆)alkyl, (C₆-C₁₀) aryl(C₁-C₆)alkyl, and (C₁-C₉)heteroaryl(C₁-C₆)alkyl.

As used herein, the term “formula” may be hereinafter referred to as a “compound(s) of the invention”. Such terms are also defined to include all forms of the compound of formula, including hydrates, solvates, isomers, crystalline and non-crystalline forms, isomorphs, polymorphs, and metabolites thereof. For example, the compounds of formula, or pharmaceutically acceptable salts thereof, may exist in unsolvated and solvated forms. When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

The compounds of “formula” may have asymmetric carbon atoms. The carbon-carbon bonds of the compounds of formula may be depicted herein using a solid line, a solid wedge, or a dotted wedge. The use of a solid line to depict bonds to asymmetric carbon atoms may be meant to indicate that all possible stereoisomers (e.g. specific enantiomers, racemic mixtures, etc.) at that carbon atom we included. The use of either a solid or dotted wedge to depict bonds to asymmetric carbon atoms may be meant to indicate that only the stereoisomer shown is meant to be included. It is possible that compounds of the present application may contain more than one asymmetric carbon atom. In those compounds, the use of a solid line to depict bonds to asymmetric carbon atoms may be meant to indicate that all possible stereoisomers we meant to be included. For example, unless stated otherwise, it may be intended that the compounds of formula can exist as enantiomers and diastereomers or as racemates and mixtures thereof. The use of a solid line to depict bonds to one or more asymmetric carbon atoms in a compound of formula and the use of a solid or dotted wedge to depict bonds to other asymmetric carbon atoms in the same compound may be meant to indicate that a mixture of diastereomers is present.

The compounds of the present application (e.g., the compounds of formula) may exist as clathrates or other complexes. Included within the scope of the invention we complexes such as clathrates, drug-host inclusion complexes wherein, in contrast to the aforementioned solvates, the drug and host may be present in stoichiometric or non-stoichiometric amounts. Also included may be complexes of formula containing two or more organic and/or inorganic components which may be in stoichiometric or non-stoichiometric amounts. The resulting complexes may be ionized, partially ionized, or non-ionized. For a review of such complexes, see J. Pharm. Sci., 64 (8), 1269-1288 by Haleblian (August 1975).

Stereoisomers of formula may include cis and trans isomers, optical isomers such as R and S enantiomers, diastereomers, geometric isomers, rotational isomers, conformational isomers, and tautomers of the compounds of formula, including compounds exhibiting more than one type of isomerism; and mixtures thereof (such as racemates and diastereomeric pairs). Also included may be acid addition or base addition salts wherein the counterion is optically active, for example, D-lactate or L-lysine, or racemic, for example, DL-tartrate or DL-arginine.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

The compounds of formula may exhibit the phenomena of tautomerism and structural isomerism. For example, the compounds of formula may exist in several tautomeric forms, including the enol and imine forms, and the keto and enamine forms, and geometric isomers and mixtures thereof. All such tautomeric forms may be included within the scope of compounds of formula. Tautomers may exist as mixtures of a tautomeric set in solution. In solid form, usually one tautomer predominates. Even though one tautomer may be described, the present invention includes all tautomers of the compounds of formula.

The present invention also includes isotopically-labeled compounds, which we identical to those recited in formula above, but for the fact that one or more atoms may be replaced by an atom having an atomic mass or mass number different from the atomic mass or mass number usually found in nature. Examples of isotopes that may be incorporated into compounds of formula include isotopes of hydrogen, carbon, nitrogen, oxygen, phosphorus, fluorine and chlorine, such as, but not limited to, ²H, ³H, ¹³C, ¹⁴C, ¹⁵N, ¹⁸O, ¹⁷O, ³¹P, ³²P, ³³S, ¹⁸F, and ³⁶Cl, Certain isotopically-labeled compounds of formula, for example those into which radioactive isotopes such as ³H and ¹⁴C are incorporated, may be useful in drug and/or substrate tissue distribution assays. Tritiated, i.e., H, and carbon-14. i.e., ¹⁴C, isotopes may be particularly used for their ease of preparation and detectability. Further, substitution with heavier isotopes such as deuterium, i.e., ²H, may afford certain therapeutic advantages resulting from greater metabolic stability, for example increased in vivo half-life or reduced dosage requirements and, hence, may be used in some circumstances. Isotopically-labeled compounds of formula may generally be prepared by carrying out the procedures disclosed in the Schemes and/or in the Examples below, by substituting an isotopically-labeled reagent for a non-isotopically-labeled reagent.

The compounds of the present application may be used in the form of salts derived from inorganic or organic acids. Depending on the particular compound, a salt of the compound may be advantageous due to one or more of the salt's physical properties, such as enhanced pharmaceutical stability in differing temperatures and humidity, or a desirable solubility in water or oil. In some instances, a salt of a compound also may be used as an aid in the isolation, purification, and/or resolution of the compound.

### Compounds

In one aspect, the present application provides a compound having the structure of formula (I),

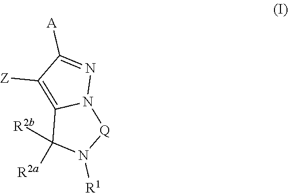

or a pharmaceutically acceptable salt, or prodrug thereof, or a solvate or hydrate of any of the forgoing,
each A and Z may be independently selected from the group consisting of optionally substituted C₆-C₁₄ aryl, and optionally substituted C₂-C₉ heteroaryl;
Q may be selected from the group consisting of optionally substituted-CH₂—, optionally substituted —CH₂—CH₂—, optionally substituted —CH═CH—, and optionally substituted —CH₂—CH₂—CH₂—;
R¹ may be selected from the group consisting of hydrogen, protium, deuterium, tritium, halogen, cyano, nitro, N₃, optionally substituted hydroxy, optionally substituted phosphorous-containing group, optionally substituted silicon-containing group, optionally substituted thio, optionally substituted amino, optionally substituted carboxyl, optionally substituted sulfonyl optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocyle, optionally substituted (C₆-C₁₀) aryl, and optionally substituted (C₁-C₉)heteroaryl;
each R^{2a} and R^{2b} may be independently absent or may be independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl and optionally substituted (C₁-C₉)heteroaryl,
or R^{2a} and R^{2b} combined with the atoms to which they are attached may form an optionally substituted ring.

In some embodiments,

wherein, when R¹ is H, Z may not be selected from the group consisting of:

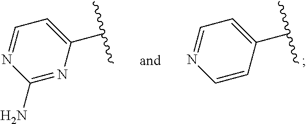 [SMILES: *c1ccnc(N)n1 ; *c1ccncc1]

when R¹ is —CH₃, Z may not be selected from the group consisting of:

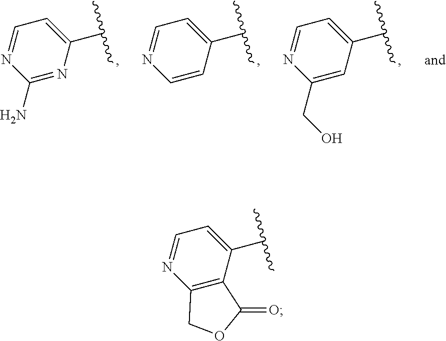 [SMILES: *c1ccnc(N)n1 ; *c1ccncc1 ; *c1ccnc(CO)c1 ; *c1ccnc2c1C(=O)OC2]

when R¹ is selected from the group consisting of: —C(═O)—C₂H₅, —C(═O)—CH—(CH₃)₂, —C(═O)—CH₂—CN, —C(═O)—NH—CH₃,

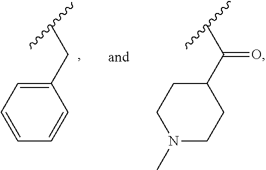

### Z may not be

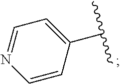 [SMILES: *c1ccncc1]

when R¹ is

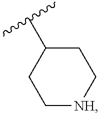 [SMILES: *C1CCNCC1]

### Z may not be

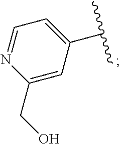 [SMILES: *c1ccnc(CO)c1]

when R¹ is

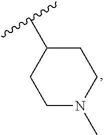 [SMILES: *C1CCN(C)CC1]

### Z may not be

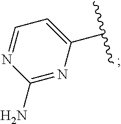 [SMILES: *c1ccnc(N)n1]

when R¹ is —C(═O)—CH₃, Z may not be selected from the group consisting of:

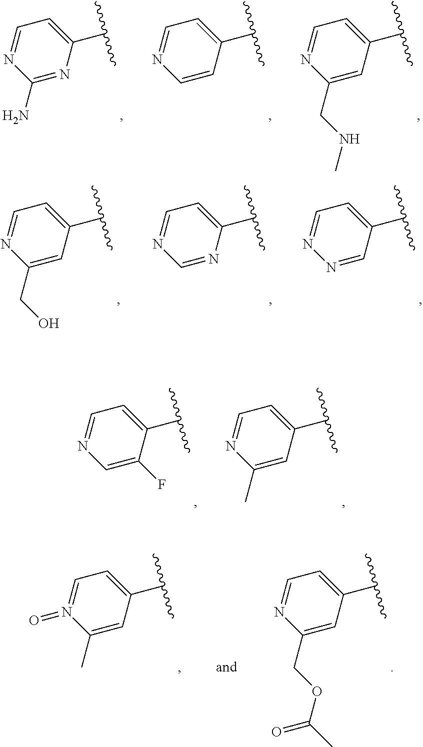

In some embodiments,
R¹ may be selected from the group consisting of deuterium, optionally substituted-methyl-(C₁-C₉)heteroaryl, optionally substituted-halomethyl, optionally substituted deuterated methyl, optionally substituted-methyl-(C₃-C₁₀)carbocycle, optionally substituted-methyl-thio, optionally substituted (C₂-C₆)alkyl, optionally substituted —(C₁) acyl-(C₁-C₉)heteroaryl, optionally substituted —(C₁) acyl-(C₆-C₁₀) aryl, optionally substituted —(C₁) acyl-tetrahydrofuran, optionally substituted —(C₁) acyl-(C₃-C₁₀)carbocycle, optionally substituted-halo(C₂) acyl, optionally substituted —(C₂) acyl-hydroxy, optionally substituted (C₂)thioacyl, optionally substituted —(C₃) acyl-amino, optionally substituted —C(═O)—(CH₂)₂—CH₃, optionally substituted —(C₅-C₆) acyl, optionally substituted oxetane, optionally substituted tetrahydropyran, optionally substituted tetrahydrothiopyran, optionally substituted sulfonyl, optionally substituted phosphorous-containing group, optionally substituted silicon-containing group, optionally substituted thio, optionally substituted (C₂-C₆)alkenyl, optionally substituted (C₃-C₁₀)carbocycle, and optionally substituted (C₁-C₉)heteroaryl.

In some embodiments,
R¹ may be selected from the group consisting of optionally substituted (C₂-C₆)alkyl, optionally substituted —(C₅-C₆) acyl, optionally substituted oxetane, optionally substituted tetrahydropyran, optionally substituted tetrahydrothiopyran, optionally substituted sulfonyl, optionally substituted thio, optionally substituted (C₂-C₆)alkenyl, optionally substituted (C₃-C₁₀)carbocycle, and optionally substituted (C₁-C₉)heteroaryl.

In some embodiments,
R¹ may be optionally substituted (C₂-C₆)alkyl.

In some embodiments,
R¹ may be selected from the group consisting of optionally substituted oxetane, optionally substituted tetrahydropyran, and optionally substituted tetrahydrothiopyran.

In some embodiments,
R¹ may be optionally substituted sulfonyl.

In some embodiments,
R¹ may be optionally substituted thio.

In some embodiments,
R¹ may be optionally substituted (C₂-C₆)alkenyl.

In some embodiments,
R¹ may be optionally substituted (C₃-C₁₀)carbocycle.

In some embodiments,
R¹ may be optionally substituted (C₁-C₉)heteroaryl.

In some embodiments,
said R¹ may be substituted with one or more R³, each R³ may be independently absent or may be independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl. For example, R¹ may be substituted with 1, 2, 3, 4, 5, 6, 7, 8, or 9 R³. For example, R¹ may be substituted with 1, 2, 3, 4, or 5 R³. For example, R¹ may be substituted with 1, 2, or 3 R³. For example, R¹ may be substituted with one R³. For example, R¹ may be substituted with 2 R³. For example, R¹ may be substituted with 3 R³.

In some embodiments,
each R³ may be independently absent or may be independently selected from the group consisting of hydrogen, deuterium, halogen, cyano, ═O, optionally substituted hydroxy, optionally substituted thio, optionally substituted amino, optionally substituted (C₁-C₆)alkyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl.

In some embodiments,
each R³ may be independently absent or may be independently selected from the group consisting of hydrogen, optionally substituted hydroxy, optionally substituted thio, optionally substituted amino, optionally substituted (C₁-C₆)alkyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl.

In some embodiments,
each R³ may be independently absent or may be independently selected from the group consisting of hydrogen, optionally substituted (C₁-C₆)alkyl, and optionally substituted (C₆-C₁₀) aryl.

In some embodiments,
each R³ may be independently absent or may be independently selected from the group consisting of hydrogen, optionally substituted methyl, and optionally substituted phenyl.

In some embodiments,
R¹ may be selected from the group consisting of optionally substituted (C₂-C₆)alkyl, optionally substituted —(C₅-C₆) acyl, optionally substituted oxetane, optionally substituted tetrahydropyran, optionally substituted tetrahydrothiopyran, optionally substituted sulfonyl, optionally substituted thio, optionally substituted (C₂-C₆)alkenyl, optionally substituted (C₃-C₁₀)carbocycle, and optionally substituted (C₁-C₉)heteroaryl, said R¹ may be substituted with one or more R³, each R³ may be independently absent or may be independently selected from the group consisting of hydrogen, optionally substituted hydroxy, optionally substituted thio, optionally substituted amino, optionally substituted (C₁-C₆)alkyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl.

In some embodiments,
R¹ may be optionally substituted (C₂-C₆)alkyl, said R¹ may be substituted with one or more R³, each R³ may be independently absent or may be independently selected from the group consisting of hydrogen, optionally substituted (C₁-C₆)alkyl, and optionally substituted (C₆-C₁₀) aryl.

In some embodiments,
said R³ may be substituted with one or more R⁴, each R⁴ may be independently absent or may be independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl. For example, R³ may be substituted with 1, 2, 3, 4, 5, 6, 7, 8, or 9 R⁴. For example, R³ may be substituted with 1, 2, 3, 4, or 5 R⁴. For example, R³ may be substituted with 1, 2, or 3 R⁴. For example, R³ may be substituted with one R⁴. For example, R³ may be substituted with 2 R⁴. For example, R³ may be substituted with 3 R⁴.

In some embodiments,
each R⁴ may be independently absent or may be independently selected from the group consisting of hydrogen, halogen, optionally substituted (C₁-C₆) acyl, and optionally substituted (C₁-C₆)alkyl.

In some embodiments,
each R⁴ may be independently absent or may be independently selected from the group consisting of hydrogen and halogen.

In some embodiments,
each R⁴ may be independently absent or may be independently selected from the group consisting of hydrogen and F.

In some embodiments,
R¹ may be selected from the group consisting of optionally substituted (C₂-C₆)alkyl, optionally substituted —(C₅-C₆) acyl, optionally substituted oxetane, optionally substituted tetrahydropyran, optionally substituted tetrahydrothiopyran, optionally substituted sulfonyl, optionally substituted thio, optionally substituted (C₂-C₆)alkenyl, optionally substituted (C₃-C₁₀)carbocycle, and optionally substituted (C₁-C₉)heteroaryl, said R¹ may be substituted with one or more R³, each R³ may be independently absent or may be independently selected from the group consisting of hydrogen, optionally substituted hydroxy, optionally substituted thio, optionally substituted amino, optionally substituted (C₁-C₆)alkyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl, said R³ may be substituted with one or more R⁴, each R⁴ may be independently absent or may be independently selected from the group consisting of hydrogen, halogen, optionally substituted (C₁-C₆) acyl, and optionally substituted (C₁-C₆)alkyl.

In some embodiments,
R¹ may be optionally substituted (C₁-C₆)alkyl, said R¹ may be substituted with one or more R³, each R³ may be independently absent or may be independently selected from the group consisting of hydrogen, optionally substituted (C₁-C₆)alkyl, and optionally substituted (C₆-C₁₀) aryl, said R³ may be substituted with one or more R⁴, each R⁴ may be independently absent or may be independently selected from the group consisting of hydrogen and halogen.

In some embodiments,
R¹ may be selected from the group consisting of optionally substituted oxetane, optionally substituted tetrahydropyran, and optionally substituted tetrahydrothiopyran, said R¹ may be substituted with one or more R³, each R³ may be independently absent or may be independently selected from the group consisting of hydrogen, optionally substituted (C₁-C₆)alkyl, and optionally substituted (C₆-C₁₀) aryl, said R³ may be substituted with one or more R⁴, each R⁴ may be independently absent or may be independently selected from the group consisting of hydrogen and halogen.

In some embodiments,
R¹ may be optionally substituted sulfonyl, said R¹ may be substituted with one or more R³, each R³ may be independently absent or may be independently selected from the group consisting of hydrogen, optionally substituted (C₁-C₆)alkyl, and optionally substituted (C₆-C₁₀) aryl, said R³ may be substituted with one or more R⁴, each R⁴ may be independently absent or may be independently selected from the group consisting of hydrogen and halogen.

In some embodiments,
R¹ may be optionally substituted thio, said R¹ may be substituted with one or more R³, each R³ may be independently absent or may be independently selected from the group consisting of hydrogen, optionally substituted (C₁-C₆)alkyl, and optionally substituted (C₄-C₁₀) aryl, said R³ may be substituted with one or more R⁴, each R⁴ may be independently absent or may be independently selected from the group consisting of hydrogen and halogen.

In some embodiments,
R¹ may be optionally substituted (C₂-C₆)alkenyl said R¹ may be substituted with one or more R³, each R³ may be independently absent or may be independently selected from the group consisting of hydrogen, optionally substituted (C₁-C₆)alkyl, and optionally substituted (C₆-C₁₀) aryl, said R³ may be substituted with one or more R⁴, each R⁴ may be independently absent or may be independently selected from the group consisting of hydrogen and halogen.

In some embodiments,
R¹ may be optionally substituted (C₃-C₁₀)carbocycle, said R¹ may be substituted with one or more R³, each R³ may be independently absent or may be independently selected from the group consisting of hydrogen, optionally substituted (C₁-C₆)alkyl, and optionally substituted (C₆-C₁₀) aryl, said R³ may be substituted with one or more R⁴, each R⁴ may be independently absent or may be independently selected from the group consisting of hydrogen and halogen.

In some embodiments,
R¹ may be optionally substituted (C₁-C₉)heteroaryl, said R¹ may be substituted with one or more R³, each R³ may be independently absent or may be independently selected from the group consisting of hydrogen, optionally substituted (C₁-C₆)alkyl, and optionally substituted (C₆-C₁₀) aryl, said R³ may be substituted with one or more R⁴, each R⁴ may be independently absent or may be independently selected from the group consisting of hydrogen and halogen.

In some embodiments, said R⁴ may be substituted with one or more R⁴⁰, each R⁴⁰ may be independently absent or may be independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl.

In some embodiments, said R⁴⁰ may be optionally substituted with one or more substitutions.

In some embodiments, said R^{2a} may be hydrogen and said Ra may be hydrogen.

In some embodiments, said R^{2a} may be ═O and said Ra may be absent.

In some embodiments, Q may be optionally substituted —CH₂—CH₂—.

In some embodiments, said Z may be optionally substituted C₆-C₁₄ heteroaryl.

In some embodiments, said Z may be selected from the group consisting of optionally substituted pyridine and optionally substituted pyrimidine.

In some embodiments,
said Z may be substituted with one or more R⁵, each R⁵ may be independently absent or may be independently selected from the group consisting of hydrogen, protium, deuterium, tritium, halogen, cyano, nitro, —S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl. For example, Z may be substituted with 1, 2, 3, 4, 5, 6, 7, 8, or 9 R⁵. For example, Z may be substituted with 1, 2, 3, 4, or 5 R⁵. For example, Z may be substituted with 1, 2, or 3 R⁵. For example, Z may be substituted with one R⁵. For example, Z may be substituted with 2 R⁵. For example, Z may be substituted with 3 R⁵.

In some embodiments,
each R⁵ may be independently absent or may be independently selected from the group consisting of hydrogen, optionally substituted amino, optionally substituted (C₁-C₆)alkyl, and optionally substituted (C₂-C₆)heterocycle.

In some embodiments,
each R⁵ may be independently absent or may be independently selected from the group consisting of hydrogen, optionally substituted amino, and optionally substituted (C₁-C₆)alkyl.

In some embodiments,
each R⁵ may be independently absent or may be independently selected from the group consisting of hydrogen, optionally substituted amino, and optionally substituted methyl.

In some embodiments,
said Z may be optionally substituted C₆-C₁₄ heteroaryl, said Z may be substituted with one or more R⁵, each R⁵ may be independently absent or may be independently selected from the group consisting of hydrogen, optionally substituted amino, optionally substituted (C₁-C₆)alkyl, and optionally substituted (C₂-C₉)heterocycle.

In some embodiments,
said Z may be selected from the group consisting of optionally substituted pyridine and optionally substituted pyrimidine, said Z may be substituted with one or more R⁵, each R⁵ may be independently absent or may be independently selected from the group consisting of hydrogen, optionally substituted amino, and optionally substituted (C₁-C₆)alkyl.

In some embodiments,
said R⁵ may be substituted with one or more R⁶, each R⁶ may be independently absent or may be independently selected from the group consisting of hydrogen, protium, deuterium, tritium, halogen, cyano, nitro, —S, —O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl. For example, R⁵ may be substituted with 1, 2, 3, 4, 5, 6, 7, 8, or 9 R⁶. For example, R⁵ may be substituted with 1, 2, 3, 4, or 5 R⁶. For example, R⁵ may be substituted with 1, 2, or 3 R⁶. For example, R⁵ may be substituted with one R⁶. For example, R⁵ may be substituted with 2 R⁶. For example, R⁵ may be substituted with 3 R⁶.

In some embodiments,
each R⁶ may be independently absent or may be independently selected from the group consisting of hydrogen, optionally substituted hydroxy, optionally substituted (C₁-C₆) acyl, and optionally substituted (C₁-C₆)heteroaryl.

In some embodiments,
each R⁶ may be independently absent or may be independently selected from the group consisting of hydrogen and optionally substituted (C₂-C₆) acyl.

In some embodiments,
said Z may be optionally substituted C₆-C₁₄ heteroaryl, said Z may be substituted with one or more R⁵, each R⁵ may be independently absent or may be independently selected from the group consisting of hydrogen, optionally substituted amino, optionally substituted (C₁-C₆)alkyl, and optionally substituted (C₂-C₆)heterocycle, said R⁵ may be substituted with one or more R⁶, each R⁶ may be independently absent or may be independently selected from the group consisting of hydrogen, optionally substituted hydroxy, optionally substituted (C₁-C₆) acyl, and optionally substituted (C₁-C₆)heteroaryl.

In some embodiments,
said Z may be selected from the group consisting of optionally substituted pyridine and optionally substituted pyrimidine, said Z may be substituted with one or more R⁵, each R⁵ may be independently absent or may be independently selected from the group consisting of hydrogen, optionally substituted amino, and optionally substituted (C₁-C₆)alkyl, said R⁵ may be substituted with one or more R⁶, each R⁶ may be independently absent or may be independently selected from the group consisting of hydrogen and optionally substituted (C₂-C₆) acyl.

In some embodiments,
said A may be optionally substituted C₆-C₁₄ aryl.

In some embodiments,
said A may be optionally substituted phenyl.

In some embodiments, said A may be substituted with one or more R⁷, each R⁷ may be independently absent or may be independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl. For example, A may be substituted with 1, 2, 3, 4, 5, 6, 7, 8, or 9 R⁷. For example, A may be substituted with 1, 2, 3, 4, or 5 R⁷. For example, A may be substituted with 1, 2, or 3 R⁷. For example, A may be substituted with one R⁷. For example, A may be substituted with 2 R⁷. For example, A may be substituted with 3 R⁷.

In some embodiments,
each R⁷ may be halogen.

In some embodiments,
each R⁷ may be F.

In some embodiments,
said A may be optionally substituted C₆-C₁₄ aryl, said A may be substituted with one or more R⁷, each R⁷ may be independently absent or may be independently selected from the group consisting of hydrogen and halogen.

In some embodiments,
said A may be optionally substituted C₆-C₁₄ aryl, said A may be substituted with one or more R⁷, each R⁷ may be independently absent or may be independently selected from the group consisting of hydrogen and F.

In some embodiments, the present application provides a compound having the structure of formula (I),

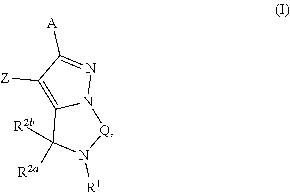

or a pharmaceutically acceptable salt, or prodrug thereof, or a solvate or hydrate of any of the forgoing,
R¹ may be selected from the group consisting of optionally substituted (C₂-C₆)alkyl, optionally substituted —(C₅-C₆) acyl, optionally substituted oxetane, optionally substituted tetrahydropyran, optionally substituted tetrahydrothiopyran, optionally substituted sulfonyl, optionally substituted thio, optionally substituted (C₂-C₆)alkenyl, optionally substituted (C₃-C₁₀)carbocycle, and optionally substituted (C₁-C₉)heteroaryl, said R¹ may be substituted with one or more R³, each R³ may be independently absent or may be independently selected from the group consisting of hydrogen, optionally substituted hydroxy, optionally substituted thio, optionally substituted amino, optionally substituted (C₁-C₆)alkyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl, said R³ may be substituted with one or more R⁴, each R⁴ may be independently absent or may be independently selected from the group consisting of hydrogen, halogen, optionally substituted (C₁-C₆) acyl, and optionally substituted (C₁-C₆)alkyl;
said R² may be hydrogen and said R^{2b} may be hydrogen;
Q may be optionally substituted —CH₂—CH₂—.
said Z may be optionally substituted C₆-C₁₄ heteroaryl, said Z may be substituted with one or more R⁵, each R⁵ may be independently absent or may be independently selected from the group consisting of hydrogen, optionally substituted amino, optionally substituted (C₁-C₆)alkyl, and optionally substituted (C₂-C₉)heterocycle, said R⁵ may be substituted with one or more R⁶, each R⁶ may be independently absent or may be independently selected from the group consisting of hydrogen, optionally substituted hydroxy, optionally substituted (C₁-C₆) acyl, and optionally substituted (C₁-C₉)heteroaryl;
said A may be optionally substituted C₆-C₁₄ aryl, said A may be substituted with one or more R⁷, each R⁷ may be independently absent or may be independently selected from the group consisting of hydrogen and halogen.

In some embodiments, the present application provides a compound having the structure of formula (I),

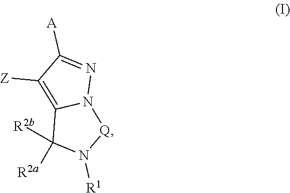

or a pharmaceutically acceptable salt, or prodrug thereof, or a solvate or hydrate of any of the forgoing,
R¹ may be optionally substituted (C₂-C₆)alkyl, said R¹ may be substituted with one or more R³, each R³ may be independently absent or may be independently selected from the group consisting of hydrogen, optionally substituted (C₁-C₆)alkyl, and optionally substituted (C₆-C₁₀) aryl, said R³ may be substituted with one or more R⁴, each R⁴ may be independently absent or may be independently selected from the group consisting of hydrogen and halogen;
said R^{2a} may be hydrogen and said R^{2b} may be hydrogen;
Q may be optionally substituted —CH₂—CH₂—;
said Z may be selected from the group consisting of optionally substituted pyridine and optionally substituted pyrimidine, said Z may be substituted with one or more R⁵, each R⁵ may be independently absent or may be independently selected from the group consisting of hydrogen, optionally substituted amino, and optionally substituted (C₁-C₆)alkyl said R⁵ may be substituted with one or more R⁶, each R⁶ may be independently absent or may be independently selected from the group consisting of hydrogen and optionally substituted (C₂-C₆) acyl;
said A may be optionally substituted C₆-C₁₄ aryl, said A may be substituted with one or more R⁷, each R⁷ may be independently absent or may be independently selected from the group consisting of hydrogen and F.

In some embodiments, the present application provides a compound having the structure of formula (I).

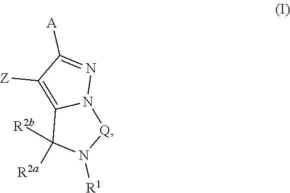

or a pharmaceutically acceptable salt, or prodrug thereof, or a solvate or hydrate of any of the forgoing,
R¹ may be selected from the group consisting of optionally substituted octane, optionally substituted tetrahydropyran, and optionally substituted tetrahydrothiopyran, said R¹ may be substituted with one or more R³, each R³ may be independently absent or may be independently selected from the group consisting of hydrogen, optionally substituted (C₁-C₆)alkyl, and optionally substituted (C₆-C₁₀) aryl, said R³ may be substituted with one or more Re, each R⁴ may be independently absent or may be independently selected from the group consisting of hydrogen and halogen;
said R² may be hydrogen and said R^{2b} may be hydrogen;
Q may be optionally substituted —CH₂—CH₂—;
said Z may be selected from the group consisting of optionally substituted pyridine and optionally substituted pyrimidine, said Z may be substituted with one or more R⁵, each R⁵ may be independently absent or may be independently selected from the group consisting of hydrogen, optionally substituted amino, and optionally substituted (C₁-C₆)alkyl, said R⁵ may be substituted with one or more R⁶, each R⁶ may be independently absent or may be independently selected from the group consisting of hydrogen and optionally substituted (C₂-C₆) acyl;
said A may be optionally substituted C₆-C₁₄ aryl, said A may be substituted with one or more R⁷, each R⁷ may be independently absent or may be independently selected from the group consisting of hydrogen and F.

In some embodiments, the present application provides a compound having the structure of formula (I),

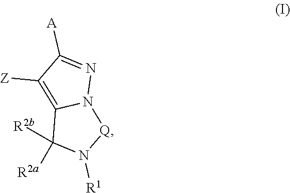

or a pharmaceutically acceptable salt, or prodrug thereof, or a solvate or hydrate of any of the forgoing.
R¹ may be optionally substituted sulfonyl, said R¹ may be substituted with one or more R³ each R³ may be independently absent or may be independently selected from the group consisting of hydrogen, optionally substituted (C₁-C₆)alkyl, and optionally substituted (C₆-C₁₀) aryl, said R³ may be substituted with one or more R⁴, each R⁴ may be independently absent or may be independently selected from the group consisting of hydrogen and halogen;
said R^{2a} may be hydrogen and said R^{2b} may be hydrogen;
Q may be optionally substituted —CH₂—C₂—;
said Z may be selected from the group consisting of optionally substituted pyridine and optionally substituted pyrimidine, said Z may be substituted with one or more R⁵, each R⁵ may be independently absent or may be independently selected from the group consisting of hydrogen, optionally substituted amino, and optionally substituted (C₁-C₆)alkyl, said R⁵ may be substituted with one or more R⁶, each R⁶ may be independently absent or may be independently selected from the group consisting of hydrogen and optionally substituted (C₂-C₆) acyl;
said A may be optionally substituted C₆-C₁₄ aryl, said A may be substituted with one or more R⁷, each R⁷ may be independently absent or may be independently selected from the group consisting of hydrogen and F.

In some embodiments, the present application provides a compound having the structure of formula (I),

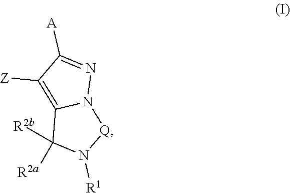

or a pharmaceutically acceptable salt, or prodrug thereof, or a solvate or hydrate of any of the forgoing,
R¹ may be optionally substituted thio, said R¹ may be substituted with one or more R³, each R³ may be independently absent or may be independently selected from the group consisting of hydrogen, optionally substituted (C₁-C₆)alkyl, and optionally substituted (C₆-C₁₀) aryl, said R³ may be substituted with one or more R⁴, each R⁴ may be independently absent or may be independently selected from the group consisting of hydrogen and halogen;
said R^{2a} may be hydrogen and said R^{2b} may be hydrogen;
Q may be optionally substituted —CH₂—CH₂—;
said Z may be selected from the group consisting of optionally substituted pyridine and optionally substituted pyrimidine, said Z may be substituted with one or more R⁵, each R⁵ may be independently absent or may be independently selected from the group consisting of hydrogen, optionally substituted amino, and optionally substituted (C₁-C₆)alkyl, said R⁵ may be substituted with one or more R⁶, each R⁶ may be independently absent or may be independently selected from the group consisting of hydrogen and optionally substituted (C₂-C₆) acyl;
said A may be optionally substituted C₆-C₁₄ aryl, said A may be substituted with one or more R⁷, each R⁷ may be independently absent or may be independently selected from the group consisting of hydrogen and F.

In some embodiments, the present application provides a compound having the structure of formula (I),

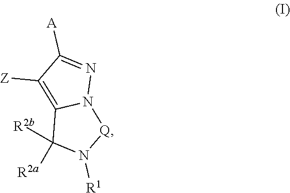

or a pharmaceutically acceptable salt, or prodrug thereof, or a solvate or hydrate of any of the forgoing,
R¹ may be optionally substituted (C₂-C₆)alkenyl, said R¹ may be substituted with one or more R³, each R³ may be independently absent or may be independently selected from the group consisting of hydrogen, optionally substituted (C₁-C₆)alkyl, and optionally substituted (C₆-C₁₀) aryl, said R³ may be substituted with one or more R⁴, each R⁴ may be independently absent or may be independently selected from the group consisting of hydrogen and halogen;
said R^{2a} may be hydrogen and said R^{2b} may be hydrogen;
Q may be optionally substituted —CH₂—CH₂—;
said Z may be selected from the group consisting of optionally substituted pyridine and optionally substituted pyrimidine, said Z may be substituted with one or more R⁵, each R⁵ may be independently absent or may be independently selected from the group consisting of hydrogen, optionally substituted amino, and optionally substituted (C₁-C₆)alkyl, said R⁵ may be substituted with one or more R⁶, each R⁶ may be independently absent or may be independently selected from the group consisting of hydrogen and optionally substituted (C₂-C₆) acyl;
said A may be optionally substituted C₆-C₁₄ aryl, said A may be substituted with one or more R⁷, each R⁷ may be independently absent or may be independently selected from the group consisting of hydrogen and F.

In some embodiments, the present application provides a compound having the structure of formula (I),

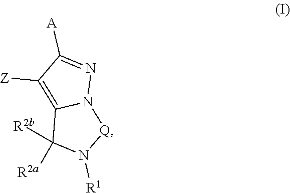

or a pharmaceutically acceptable salt, or prodrug thereof, or a solvate or hydrate of any of the forgoing,
R¹ may be optionally substituted (C₃-C₁₀)carbocycle, said R¹ may be substituted with one or more R³, each R³ may be independently absent or may be independently selected from the group consisting of hydrogen, optionally substituted (C₁-C₆)alkyl, and optionally substituted (C₆-C₁₀) aryl, said R³ may be substituted with one or more R⁴, each R⁴ may be independently absent or may be independently selected from the group consisting of hydrogen and halogen;
said R^{2a} may be hydrogen and said R^{2b} may be hydrogen;
Q may be optionally substituted —CH₂—CH₂—;
said Z may be selected from the group consisting of optionally substituted pyridine and optionally substituted pyrimidine, said Z may be substituted with one or more R⁵, each R⁵ may be independently absent or may be independently selected from the group consisting of hydrogen, optionally substituted amino, and optionally substituted (C₁-C₆)alkyl, said R⁵ may be substituted with one or more R⁶, each R⁶ may be independently absent or may be independently selected from the group consisting of hydrogen and optionally substituted (C₂-C₆) acyl;
said A may be optionally substituted C₆-C₁₄ aryl, said A may be substituted with one or more R⁷, each R⁷ may be independently absent or may be independently selected from the group consisting of hydrogen and F.

In some embodiments, the present application provides a compound having the structure of formula (I),

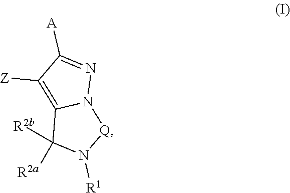

or a pharmaceutically acceptable salt, or prodrug thereof, or a solvate or hydrate of any of the forgoing,
R¹ may be optionally substituted (C₁-C₉)heteroaryl said R¹ may be substituted with one or more R³, each R³ may be independently absent or may be independently selected from the group consisting of hydrogen, optionally substituted (C₁-C₆)alkyl, and optionally substituted (C₆-C₁₀) aryl, said R³ may be substituted with one or more R⁴, each R⁴ may be independently absent or may be independently selected from the group consisting of hydrogen and halogen;
said R^{2a} may be hydrogen and said R^{2b} may be hydrogen;
Q may be optionally substituted —CH₂—CH₂—;
said Z may be selected from the group consisting of optionally substituted pyridine and optionally substituted pyrimidine, said Z may be substituted with one or more R⁵, each R⁵ may be independently absent or may be independently selected from the group consisting of hydrogen, optionally substituted amino, and optionally substituted (C₁-C₄)alkyl said R³ may be substituted with one or more R⁶, each R⁶ may be independently absent or may be independently selected from the group consisting of hydrogen and optionally substituted (C₂-C₆) acyl;
said A may be optionally substituted C₆-C₁₄ aryl, said A may be substituted with one or more R⁷, each R⁷ may be independently absent or may be independently selected from the group consisting of hydrogen and F.

In one aspect, the present application provides a compound having the structure of formula (I).

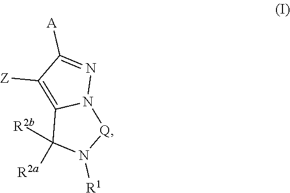

or a pharmaceutically acceptable salt, or prodrug thereof, or a solvate or hydrate of any of the forgoing,
Z may represent C₂-C₉ heteroaryl, which may be optionally substituted with one or more R¹⁰³,
A may represent C₆-C₁₄ aryl, or C₂-C₉ heteroaryl, which may be optionally substituted with one or more R¹⁰⁴,
Q may represent C, C—C, C═C, or C—C—C, which may be optionally substituted with one or more R¹⁰⁵,
R¹ may represent hydrogen, protium, deuterium, tritium, halogen, CN, NO₂, ═O, ═S, OR¹⁰⁶, SR¹⁰⁶, C(O)R¹⁰⁶, C(S)R¹⁰⁶, N(R^{106a})(R^{106b}), C(═NR^{106a})R^{106b}, SO(═NR^{106a})R^{106b}, or SO₂R¹⁰⁶,
or may represent C₁-C₆ alkyl, C₃-C₈ cycloalkyl, C₂-C₆ alkenyl, C₂-C₆ alkynyl, C₆-C₁₄ aryl, C₂-C₉ heterocyclyl, or C₂-C₉ heteroaryl, each of which may be mono- or polysubstituted by identical or different substituents from the group consisting of R¹⁰⁶,
each of R^{2a} and R^{2b} may be absent or may represent hydrogen, protium, deuterium, tritium, halogen, CN, NO₂, ═O, ═S, OR¹⁰⁷, SR¹⁰⁷, C(O)R¹⁰⁷, C(S)R¹⁰⁷, N(R^{107a})(R^{107b}), C(—NR^{107a})R^{107b}, SO(—NR^{107a})R^{107b}, or SO₂R¹⁰⁷,
or may represent C₁-C₆ alkyl, C₃-C₈ cycloalkyl, C₂-C₆ alkenyl, C₂-C₆ alkynyl, C₆-C₁₄ aryl, C₂-C₉ heterocyclyl, or C₂-C₉ heteroaryl, each of which may be mono- or polysubstituted by identical or different substituents from the group consisting of R¹⁰⁷,
or R¹ and R^{2a} or R¹ and R^{2b} combined with the atoms to which they are attached form a C₂-C₉ heterocyclyl or a C₂-C₉ heteroaryl, each of which may be mono- or polysubstituted by identical or different substituents from the group consisting of R¹⁰⁸,
each R¹⁰³ may be absent or independently selected from the group consisting of hydrogen, protium, deuterium, tritium, halogen, CN, NO₂, ═O, ═S, OR¹⁰³, SR¹⁰³, C(O)R¹⁰³, C(S)R¹⁰³, N(R^{103a})(R^{103b}), C(—NR^{103a})R^{103b}, SO(—NR^{103a})R^{103b}, and SO₂R¹⁰³,
or independently selected from the group consisting of C₁-C₆ alkyl, C₃-C₈ cycloalkyl, C₂-C₆ alkenyl, C₂-C₆ alkynyl, C₆-C₁₄ aryl, C₂-C₉ heterocyclyl, and C₂-C₉ heteroaryl, each of which may be mono- or polysubstituted by identical or different substituents from the group consisting of R¹⁰⁹,
or any two R¹⁰³ combined with the atoms to which they are attached form a C₃-C₈ cycloalkyl, a C₂-C₉ heterocyclyl, a C₆-C₁₄ aryl, or a C₂-C₉ heteroaryl, each of which may be mono- or polysubstituted by identical or different substituents from the group consisting of R¹⁰⁹,
each R¹⁰⁹, R^{109a}, and R^{109b} may be absent or may represent hydrogen, protium, deuterium, tritium, halogen, CN, NO₂, ═O, ═S, OR¹¹⁰, SR¹¹⁰, C(O)R¹¹⁰, C(S)R¹¹⁰, N(R^{110a})(R^{110b}), C(—NR^{110a})R^{110b}, SO(═NR^{110a})R^{110b}, or SO₂R¹¹⁰,
or may represent C₁-C₆ alkyl, C₃-C₈ cycloalkyl, C₂-C₆ alkenyl, C₂-C₆ alkynyl, C₆-C₁₄ aryl, C₂-C₉ heterocyclyl, or C₂-C₉ heteroaryl, each of which may be mono- or polysubstituted by identical or different substituents from the group consisting of R¹¹⁰,
each R¹¹⁰, R^{110a}, and R^{110b} may be absent or may represent hydrogen, protium, deuterium, tritium, halogen CN, NO₂, ═O, ═S, OR¹¹¹, SR¹¹¹, C(O)R¹¹¹, C(S)R¹¹¹, N(R¹¹¹)(R¹¹¹), C(—N^{111a})R^{111b}, SO(—NR^{111a})R^{111b}, or SO₂R¹¹¹,
or may represent C₁-C₆ alkyl, C₃-C₈ cycloalkyl, C₂-C₆ alkenyl, C₂-C₆ alkynyl, C₆-C₁₄ aryl, C₂-C₉ heterocyclyl, or C₂-C₉ heteroaryl, each of which may be mono- or polysubstituted by identical or different substituents from the group consisting of R¹¹¹,
each R¹¹¹, R^{111a}, and R^{111b} may be absent or may represent hydrogen, protium, deuterium, tritium, halogen, CN, NO₂, ═O, —S, OR¹¹², SR¹¹², C(O)R¹¹², C(S)R¹¹², N(R^{112a})(R^{112b}), C(—NR^{112a})R^{112b}, SO(═NR^{112a})R^{112b}, or SO₂R¹¹²,
or may represent C₁-C₆ alkyl, C₃-C₈ cycloalkyl, C₂-C₆ alkenyl, C₂-C₆ alkynyl, C₆-C₁₄ aryl, C₂-C₉ heterocyclyl, or C₂-C₉ heteroaryl, each of which may be mono- or polysubstituted by identical or different substituents from the group consisting of R¹¹²,
each R¹⁰⁴ and R¹⁰⁵ may be absent or may represent hydrogen, protium, deuterium, tritium, halogen, CN, NO₂, ═O, ═S, OR¹⁰⁰, SR¹⁰⁰, C(O)R¹⁰⁰, C(S)R, N(R^{10a})(R^{10b}), C(═NR^{10a})R^{10b}, SO(═NR^{10a})R^{10b}, or SO₂R¹⁰⁰,
or may represent C₁-C₆ alkyl, C₃-C₈ cycloalkyl, C₂-C₆ alkenyl, C₂-C₆ alkynyl, C₆-C₁₄ aryl, C₂-C₉ heterocyclyl, or C₂-C₉ heteroaryl, each of which may be mono- or polysubstituted by identical or different substituents from the group consisting of R¹⁰⁰,
or any two R¹⁰⁴ or any two R¹⁰⁵ combined with the atoms to which they are attached form C₃-C₈ cycloalkyl, C₂-C₉ heterocyclyl, C₆-C₁₄ aryl, or C₂-C₉ heteroaryl, each of which may be mono- or polysubstituted by identical or different substituents from the group consisting of R¹⁰⁰,
each R¹⁰⁶, R^{106a}, R^{106b}, R¹⁰⁷, R^{107a}, R^{107b}, R⁸, R¹¹², R^{112a}, and R^{112b} may be absent or may represent hydrogen, protium, deuterium, tritium, halogen, CN, NO₂, ═O, ═S, OR¹⁰⁰, SR¹⁰⁰, C(O)R¹⁰⁰, C(S)R¹⁰⁰, N(R^{10a})(R^{10b}), C(═NR^{10a})R^{10b}, SO(═NR^{10a})R^{10b}, or SO₂R¹⁰⁰,
or may represent C₁-C₆ alkyl, C₃-C₈ cycloalkyl, C₂-C₆ alkenyl, C₂-C₆ alkynyl, C₆-C₁₄ aryl, C₂-C₉ heterocyclyl, or C₂-C₉ heteroaryl, each of which may be mono- or polysubstituted by identical or different substituents from the group consisting of R¹⁰⁰,
each R¹⁰⁰, R^{10a}, and R^{10b} may be absent or may represent hydrogen, protium, deuterium, tritium, halogen, CN, NO₂, ═O, ═S, OH, SH, C(O)H, C(S)H, NH₂, C(═NH)H, SO(═NH)H, or SO₂H,
or may represent C₁-C₆ alkyl, C₃-C₈ cycloalkyl, C₂-C₆ alkenyl, C₂-C₆ alkynyl, C₆-C₁₄ amyl, C₂-C₉ heterocyclyl, or C₂-C₉ heteroaryl.

In one aspect, the present application provides a compound having the structure of formula (II),

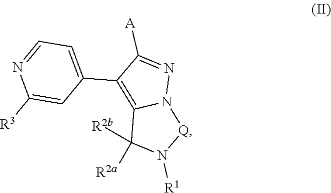

or a pharmaceutically acceptable salt, or prodrug thereof, or a solvate or hydrate of any of the forgoing, each A may be independently selected from the group consisting of optionally substituted C₆-C₁₄ aryl, and optionally substituted C₂-C₉ heteroaryl;
Q may be selected from the group consisting of optionally substituted —CH₂—, optionally substituted —CH₂—CH₂—, optionally substituted —CH═CH—, and optionally substituted —CH₂—CH₂—CH₂—;
R¹ may be selected from the group consisting of hydrogen, protium, deuterium, tritium, halogen, cyano, nitro, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl;
each R^{2a} and R^{2b} may be independently absent or may be independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl,
or R^{2a} and R^{2b} combined with the atoms to which they are attached may form an optionally substituted ring,
R³ may be optionally substituted amino.

In one aspect, the present application provides a compound having the structure of formula (II),

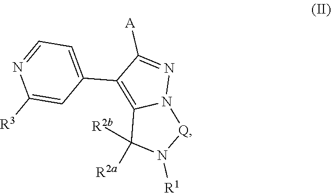

or a pharmaceutically acceptable salt, or prodrug thereof, or a solvate or hydrate of any of the forgoing,
each A may be independently selected from the group consisting of optionally substituted C₆-C₁₄ aryl, and optionally substituted C₂-C₉ heteraryl;
Q may be selected from the group consisting of optionally substituted —CH₂—, optionally substituted —CH₂—CH₂—, optionally substituted —CH═CH—, and optionally substituted —CH₂—CH₂—CH₂—;
R¹ may be selected from the group consisting of hydrogen, protium, deuterium, tritium, halogen, cyano, nitro, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₆)heterocycle, optionally substituted (C₆-C₁₀) aryl, and optionally substituted (C₂-C₉)heteroaryl;
each R^{2a} and R^{2b} may be independently absent or may be independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₂-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl,
or R^{2a} and R^{2b} combined with the atoms to which they are attached may form an optionally substituted ring,
R³ may be selected from the group consisting of protium, deuterium, tritium, cyano, nitro, N₃, optionally substituted —OH, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₂-C₆)haloalkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, optionally substituted (C₁-C₉)heteroaryl, optionally substituted —CH₂—(C₁-C₆) acyl, and optionally substituted —CH₂—(C₁-C₉)heteroaryl.

In some embodiments, R¹, R^{2a}, R^{2b}, R³, Q and A in the compound having the structure of formula (II) may be defined as R¹, R^{2a}, R^{2b}, R³, Q and A in any one of the compounds having the structure of formula (I).

In some embodiments, the present application provides a compound having the structure of formula (III),

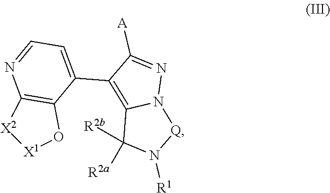

or a pharmaceutically acceptable salt, or prodrug thereof, or a solvate or hydrate of any of the forgoing,
each dashed line [symbol] represents a single or double bond, each X¹ and X² may be independently selected from the group consisting of S, O, N, optionally substituted NH, optionally substituted CH and optionally substituted CH₂; for example, each X¹ and X² may be independently optionally substituted with one or more R³ consisting of hydrogen, protium, deuterium, tritium, halogen, cyano, nitro, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₁₀)heterocycle, optionally substituted (C₆-C₁₀) aryl, and optionally substituted (C₁-C₉)heteroaryl;
each A may be independently selected from the group consisting of optionally substituted C₆-C₁₄ aryl, and optionally substituted C₂-C₉ heteroaryl;
Q may be selected from the group consisting of optionally substituted —CH₂—, optionally substituted —CH₂—CH₂—, optionally substituted —CH═CH—, and optionally substituted —CH₂—CH₂—CH₂—;
R¹ may be selected from the group consisting of hydrogen, protium, deuterium, tritium, halogen, cyano, nitro, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl;
each R^{2a} and R^{2b} may be independently absent or may be independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl,
or R^{2a} and R^{2b} combined with the atoms to which they are attached may form an optionally substituted ring.

For example, each X¹ and X² may be independently optionally substituted with one or more R³ consisting of hydrogen, protium, deuterium, tritium, halogen, cyano, nitro, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl. In some embodiments, R¹, R^{2a}, R^{2b}, R³, Q and A in the compound having the structure of formula (III) may be defined as R¹, R^{2a}, R^{2b}, R³, Q and A in any one of the compounds having the structure of formula (I).

In some oases, the compound may be one of the compounds in Table 1.

TABLE 1

| Compound | Structure | IUPAC NAME |
|---|---|---|
| I-1 | 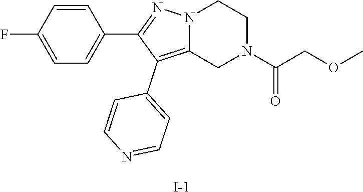 [SMILES: COCC(=O)N1CCn2nc(-c3ccc(F)cc3)c(-c3ccncc3)c2C1 ; [I-]] | 1-(2-(4-fluorophenyl)-3-(pyridin-4-yl)-6,7- dihydropyrazolo[1,5-a]pyrazin-5(4H)-yl)- 2-methoxyethan-1-one |
| I-2 | 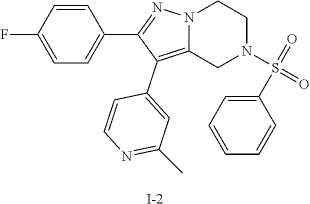 | 2-(4-fluorophenyl)-3-(2-methylpyridin-4- yl)-5-(phenylsulfonyl)-4,5,6,7- tetrahydropyrazolo[1,5-a]pyrazine |
| I-3 | 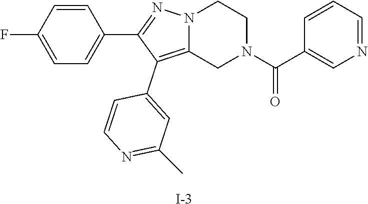 | (2-(4-fluorophenyl)-3-(2-methylpyridin-4- yl)-6,7-dihydropyrazolo[1,5-a]pyrazin-5 (4H)-yl)(pyridin-3-yl)methanone |
| I-4 | 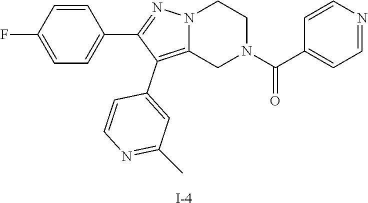 | (2-(4-fluorophenyl)-3-(2-methylpyridin-4- yl)-6,7-dihydropyrazolo[1,5-a]pyrazin-5 (4H)-yl)(pyridin-4 yl)methanone |
| I-5 | 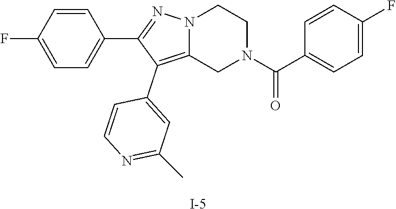 | (4-fluorophenyl)(2-(4-fluorophenyl)-3-(2- methylpyridin-4-yl)-6,7-dihydropyrazolo [1.5-a]pyrazin-5(4H)-yl)methanone |
| I-6 | 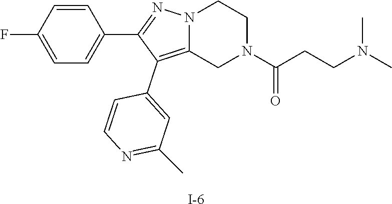 | 3-(dimethylamino)-1-(2-(4-fluorophenyl)- 3-(2-methylpyridin-4-yl)-6,7- dihydropyrazolo[1,5-a]pyrazin- 5(4H)-yl)propan-1-one |
| I-7 | 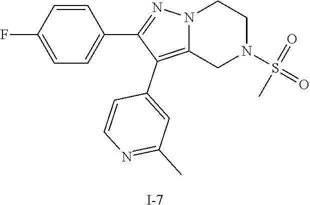 | 2-(4-fluorophenyl)-3-(2-methylpyridin-4- yl)-5-(methylsulfonyl)-4,5,6,7- tetrahydropyrazolo[1,5-a]pyrazine |
| I-8 | 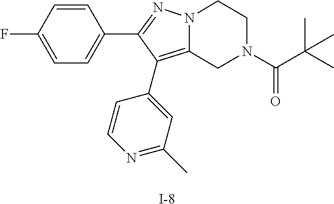 | 1-(2-(4-fluorophenyl)-3-(2-methylpyridin- 4-yl)-6,7-dihydropyrazolo[1,5-a]pyrazin-5 (4H)-yl)-2,2-dimethylpropan-1-one |
| I-9 | 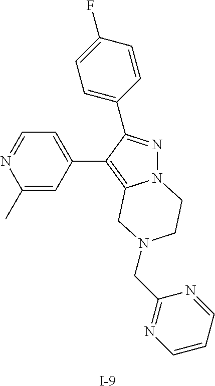 | 2-(4-fluorophenyl)-3-(2-methylpyridin-4-y)- 5-(pyrimidin-2-ylmethyl)-4,5,6,7- tetrahydropyrazolo[1,5-a]pyrazine |
| I-10 | 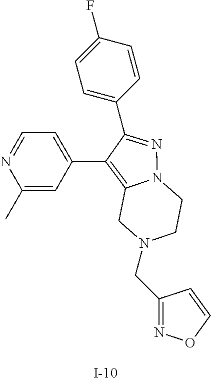 | 3-((2-(4-fluorophenyl)-3-(2-methylpyridin- 4-yl)-6,7-dihydropyrazolo[1,5-a]pyrazin- 5(4H)-yl)methyl)isoxazole |
| I-11 | 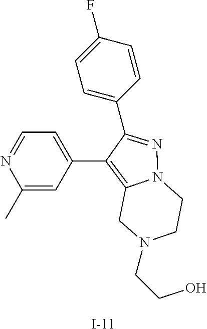 | 2-(2-(4-fluorophenyl)-3-(2-methylpyridin- 4-yl)-6,7-dihydropyrazolo[1,5-a]pyrazin-5. (4H)-yl)ethan-1-ol |
| I-12 | 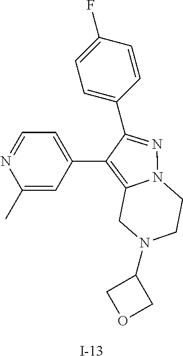 | 2-(4-fluorophenyl)-3-(2-methylpyridin-4- yl)-5-(oxetan-3-yl)-4,5,6,7- tetrahydropyrazolo[1,5-a]pyrazine |
| I-13 | 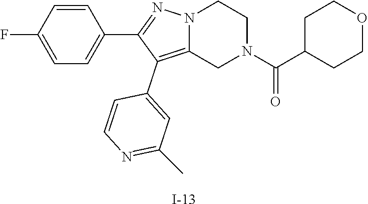 | (2-(4-fluorophonyl)-3-(2-methylpyridin-4- yl)-6,7-dihydropyrazolo[1,5-a]pyrazin-5(4H)- yl)(tetrahydro-2H-pyran-4-yl)methanone |
| I-14 | 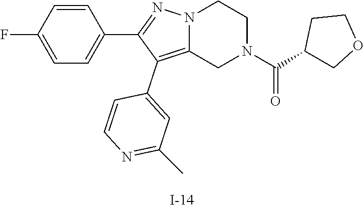 | (R)-(2-(4-fluorophenyl)-3-(2-methylpyridin- 4-yl)-6,7-dihydropyrazolo[1,5-a]pyrazin- 5(4H)-yl)(tetrahydrofuran-3-yl)methanone |
| I-15 | 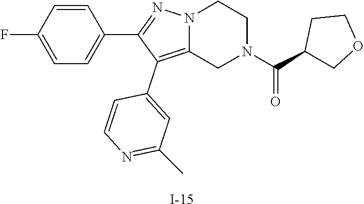 | (S)-(2-(4-fluorophenyl)-3-(2-methylpyridin- 4-yl)-6,7-dihydropyrazolo[1,5-a]pyrazin- 5(4H)-yl)(tetrahydrofuran-3-yl)methanone |
| I-16 | 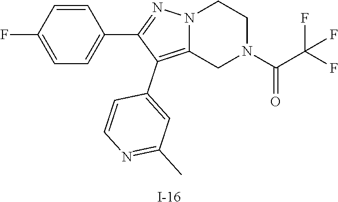 | 2,2,2-trifluoro-1-(2-(4-fluorophenyl)-3-(2- methylpyridin-4-yl)-6,7-dihydropyrazolo [1,5-a]pyrazin-5(4H)-yl)ethan-1-one |
| I-17 | 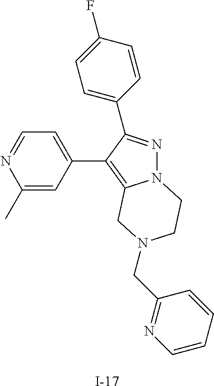 | 2-(4-fluorophenyl)-3-(2-methylpyridin-4- yl)-5-(pyridin-2-ylmethyl)-4,5,6,7- tetrahydropyrazolo[1,5-a]pyrazine |
| I-18 | 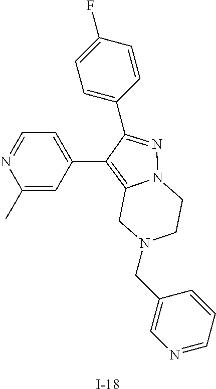 | 2-(4-fluorophenyl)-3-(2-methylpyridin-4- yl)-5-(pyridin-3-ylmethyl)-4,5,6,7- tetrahydropyrazolo[1,5-a]pyrazine |
| I-19 | 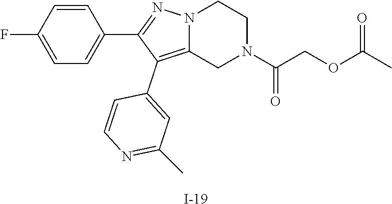 | 2-(2-(4-fluorophenyl)-3-(2-methylpyridin- 4-yl)-6,7-dihydropyrazolo[1,5-a]pyrazin-5 (4H)-yl)-2-oxoethyl acetate |
| I-20 | 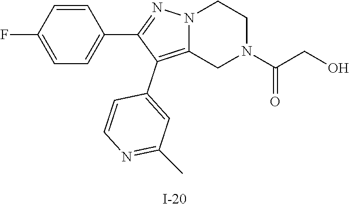 | 1-(2-(4-fluorophenyl)-3-(2-methylpyridin- 4-yl)-6,7-dihydropyrazolo[1,5-a]pyrazin-5 (4H)-yl)-2-hydroxyethan-1-one |
| I-21 | 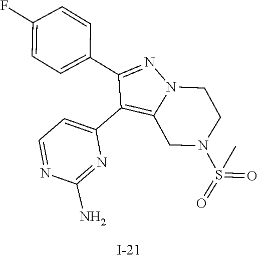 | 4-(2-(4-fluorophenyl)-5-(methylsulfonyl)- 4,5,6,7-tetrahydropyrazolo[1,5-a]pyrazin- 3-yl)pyrimidin-2-amine. |
| I-22 | 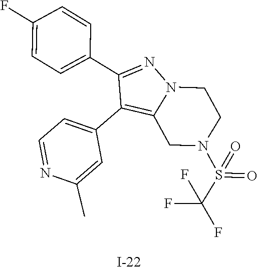 | 2-(4-fluorophenyl)-3-(2-methylpyridin-4- yl)-5-((trifluoromethyl)sulfonyl)-4,5,6,7- tetrahydropyrazolo[1,5-a]pyrazine |
| I-23 | 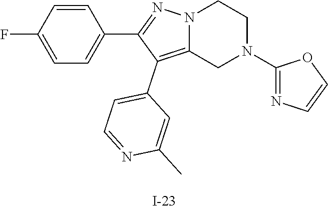 | 2-(2-(4-fluorophenyl)-3-(2-methylpyridin- 4-yl)-6,7-dihydropyrazolo[1,5-a]pyrazin-5 (4H)-yl)oxazole |
| I-24 | 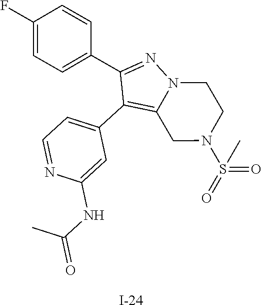 | N-(4-(2-(4-fluorophenyl)-5-(methylsulfonyl)- 4,5,6,7-tetrahydropyrazolo[1,5-a]pyrazin- 3-yl)pyridin-2-yl)acetamide |
| I-25 | 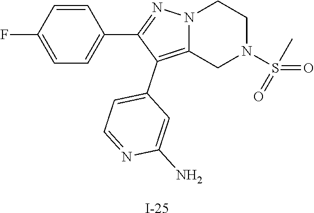 | 4-(2-(4-fluorophenyl)-5-(methylsulfonyl)- 4,5,6,7-tetrahydropyrazolo[1,5-a]pyrazin- 3-yl)pyridin-2-amine |
| I-26 | 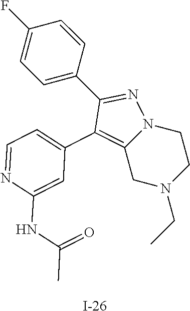 | N-(4-(5-ethyl-2-(4-fluorophenyl)-4,5,6,7- tetrahydropyrazolo[1,5-a]pyrazin-3-yl) pyridin-2-yl)acetamide |
| I-27 | 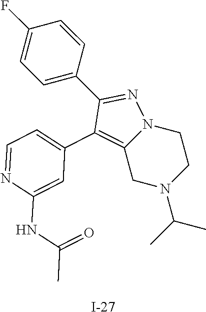 | N-(4-(2-(4-fluorophenyl)-5-isopropyl-4,5, 6,7-tetrahydropyrazolo[1,5-a]pyrazin-3-yl) pyridin-2-yl)acetamide |
| I-28 | 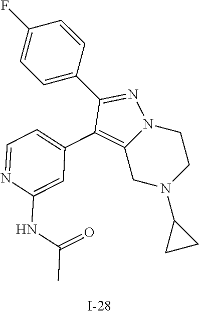 | N-(4-(5-cyclopropyl-2-(4-fluorophenyl)-4, 5,6,7-tetrahydropyrazolo[1,5-a]pyrazin-3- yl)pyridin-2-ylacetamide |
| I-29 | 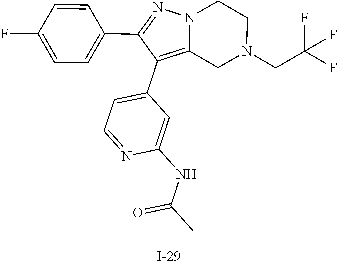 | N-(4-(2-(4-fluorophenyl)-5-(2,2,2-trifluoroethyl)- 4,5,6,7-tetrahydropyrazolo[1,5-a] pyrazin-3-yl)pyridin-2-yl)acetamide |
| I-30 | 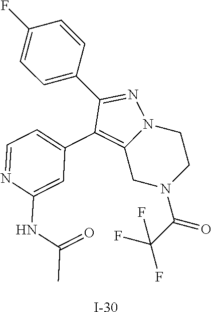 | N-(4-(2-(4-fluorophenyl)-5-(2,2,2-trifluoroacetyl)- 4,5,6,7-tetrahydropyrazolo[1,5-a] pyrazin-3-yl)pyridin-2-yl)acetamide |
| I-31 | 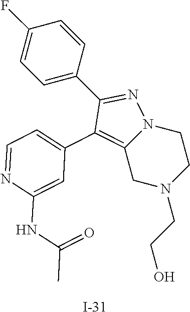 | N-(4-(2-(4-fluorophenyl)-5-(2-hydroxyethyl)- 4,5,6,7-tetrahydropyrazolo[1,5-a]pyrazin- 3-yl)pyridin-2-yl)acetamide |
| I-32 | 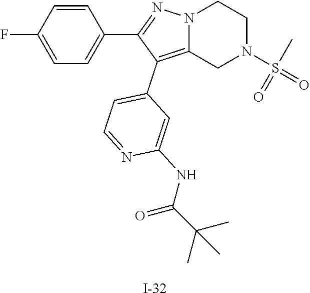 | N-(4-(2-(4-fluorophenyl)-5-(methylsulfonyl)- 4,5,6,7-tetrahydropyrazolo[1,5-a]pyrazin- 3-yl)pyridin-2-yl)pivalamide |
| I-33 | 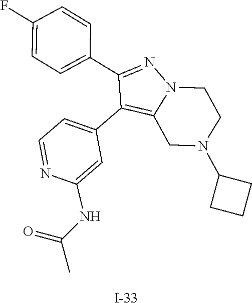 | N-(4-(5-cyclobutyl-2-(4-fluorophenyl)-4,5, 6,7-tetrahydropyrazolo[1,5-a]pyrazin-3-yl) pyridin-2-yl)acetamide |
| I-34 | 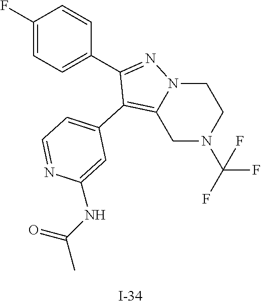 | N-(4-(2-(4-fluorophenyl)-5-(trifluoromethyl)- 4,5,6,7-tetrahydropyrazolo[1,5-a]pyrazin- 3-yl)pyridin-2-yl)acetamide |
| I-35 | 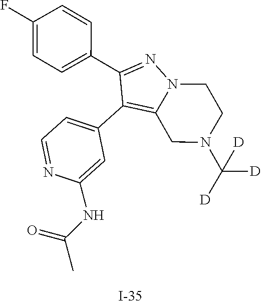 | N-(4-(2-(4-fluorophenyl)-5-(methyl-d3)-4,5, 6,7-tetrahydropyrazolo[1,5-a]pyrazin-3- yl)pyridin-2-yl)acetamide |
| I-36 | 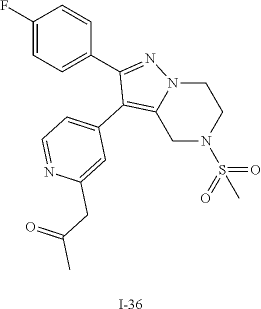 | 1-(4-(2-(4-fluorophenyl)-5-(methylsulfonyl)- 4,5,6,7-tetrahydropyrazolo[1,5-a]pyrazin- 3-yl)pyridin-2-yl)propan-2-one |
| I-37 | 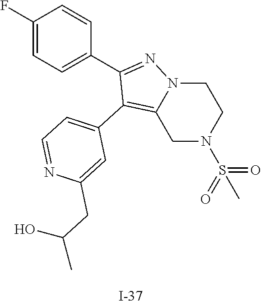 | 1-(4-(2-(4-fluorophenyl)-5-(methylsulfonyl)- 4,5,6,7-tetrahydropyrazolo[1,5-a]pyrazin-3- yl)pyridin-2-yl)propan-2-ol |
| I-38 | 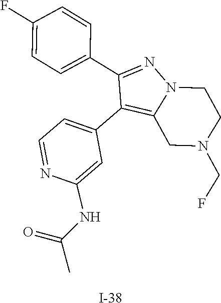 | N-(4-(5-(fluoromethyl)-2-(4-fluorophenyl)- 4,5,6,7-tetrahydropyrazolo[1,5-a]pyrazin- 3-yl)pyridin-2-yl)acetamide |
| I-39 | 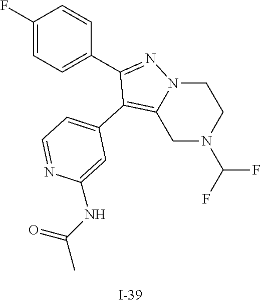 | N-(4-(5-(difluoromethyl)-2-(4-fluorophenyl)- 4,5,6,7-tetrahydropyrazolo[1,5-a]pyrazin- 3-yl)pyridin-2-yl)acetamide |
| I-40 | 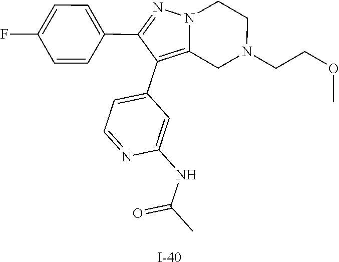 | N-(4-(2-(4-fluorophenyl)-5-(2-methoxyethyl)- 4,5,6.7-tetrahydropyrazolo[1,5-a] pyrazin-3-yl)pyridin-2-yl)acetamide |
| I-41 | 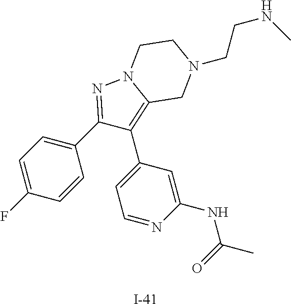 | N-(4-(2-(4-fluorophenyl)-5-(2-(methylamino) ethyl)-4,5,6,7-tetrahydropyrazolo[1,5- a]pyrazin-3-yl)pyridin-2-yl)acetamide |
| I-42 | 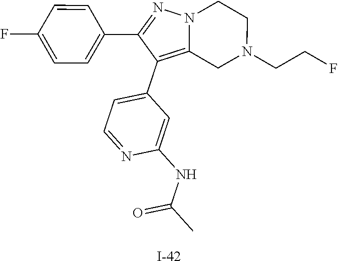 | N-(4-(5-(2-fluoroethyl)-2-(4-fluorophenyl)- 4,5,6,7-tetrahydropyrazolo[1,5-a]pyrazin- 3-yl)pyridin-2-yl)acetamide |
| I-43 | 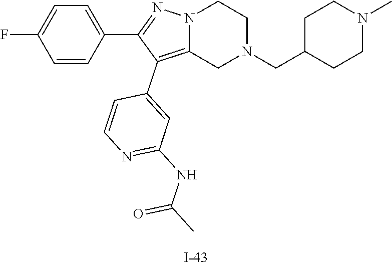 | N-(4-(2-(4-fluorophenyl)-5-((1-methylpiperidin- 4-yl)methyl)-4,5,6,7-tetrahydropyrazalo [1,5-a]pyrazin-3-yl)pyridin-2-yl)acetamide |
| I-44 | 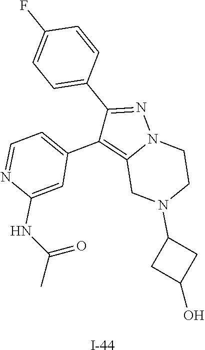 | N-(4-(2-(4-fluorophenyl)- 5-(3-hydroxycyclobutyl)- 4,5,6,7-tetrahydropyrazolo[1,5-a] pyrazin-3-yl)pyridin-2-yl)acetamide |
| I-45 | 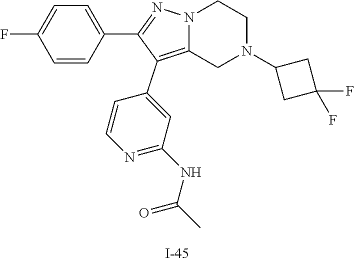 | N-(4-(5-(3,3-difluorocyclobutyl)-2-(4- fluorophenyl)-4,5,6,7-tetrahydropyrazolo[1,5- a]pyrazin-3-yl)pyridin-2-yl)acetamide |
| I-46 | 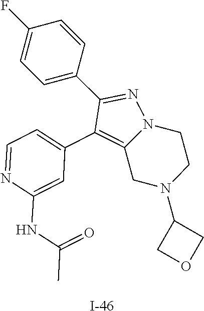 | N-(4-(2-(4-fluorophenyl)-5-(oxctan-3-yl)- 4,5,6,7-tetrahydropyrazolo[1,5-a]pyrazin- 3-yl)pyridin-2-yl)acetamide |
| I-47 | 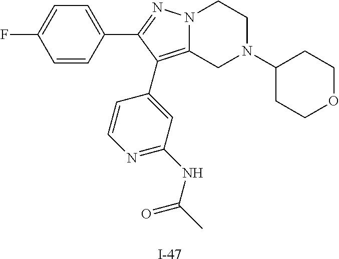 | N-(4-(2-(4-fluorophenyl)-5-(tetrahydro-2H- pyran-4-yl)-4,5,6,7-tetrahydropyrazolo [1,5-a]pyrazin-3-yl)pyridin-2-yl)acetamide |
| I-48 | 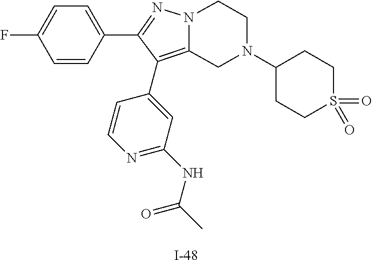 | N-(4-(5-(1,1-dioxidotetrahydro-2H]-thiopyran- 4-yl)-2-(4-fluorophenyl)-4,5,6,7- tetrahydropyrazolo[1,5-a]pyrazin-3-yl)pyridin- 2-yl)acetamide |
| I-49 | 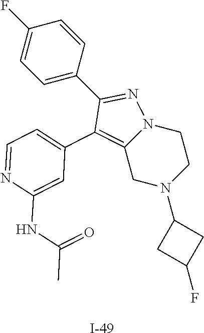 | N-(4-(5-(3-fluorocyclobutyl)- 2-(4-fluorophenyl)- 4,5,6,7-tetrahydropyrazolo[1,5-a] pyrazin-3-yl)pyridin-2-yl)acetamide |
| I-50 | 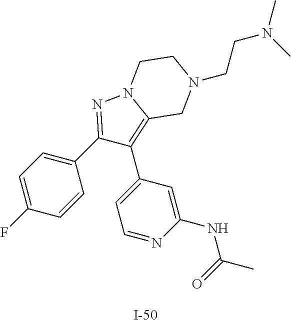 | N-(4-(5-(2-(dimethylamino)ethyl)-2-(4- fluorophenyl)-4,5,6,7-tetrahydropyrazolo [1,5-a]pyrazin-3-yl)pyridin-2-yl)acetamide |
| I-51 | 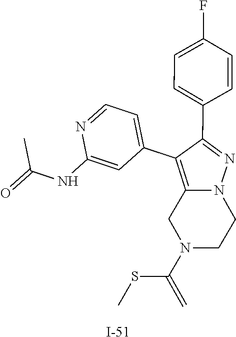 | N-(4-(2-(4-fluorophenyl)-5-(1-(methylthio) vinyl)-4,5,6,7-tetrahydropyrazolo[1,5-a] pyrazin-3-yl)pyridin-2-yl)acetamide |
| I-52 | 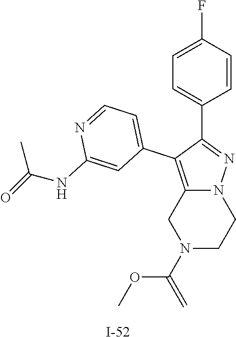 | N-(4-(2-(4-fluorophenyl)-5-(1-methoxyvinyl)- 4,5,6,7-tetrahydropyrazolo[1,5-a] pyrazin-3-yl)pyridin-2-yl)acetamide |
| I-53 | 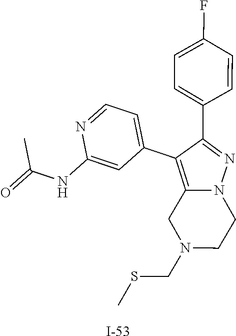 | N-(4-(2-(4-fluorophenyl)-5-((methylthio) methyl)-4,5,6,7-tetrahydropyrazolo[1,5-a] pyrazin-3-yl)pyridin-2-yl)acetamide |
| I-54 | 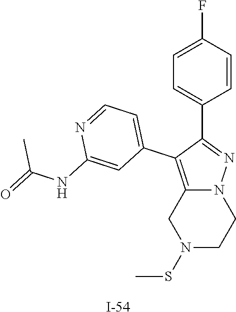 | N-(4-(2-(4-fluorophenyl)-5-(methylthio)- 4,5,6,7-tetrahydropyrazolo[1,5-a]pyrazin- 3-yl)pyridin-2-yl)acetamide |
| I-55 | 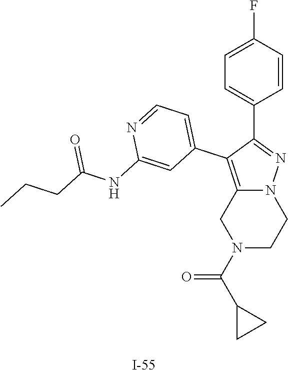 | N-(4-(5-(cyclopropanecarbonyl)-2-(4- fluorophenyl)-4,5,6,7-tetrahydropyrazolo[1,5- a]pyrazin-3-yl)pyridin-2-yl)butyramide |
| I-56 | 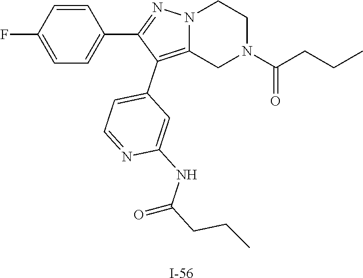 | N-(4-(5-butyryl-2-(4-fluorophenyl)-4,5,6, 7-tetrahydropyrazolo[1,5-a]pyrazin-3-yl) pyridin-2-yl)butyramide |
| I-57 | 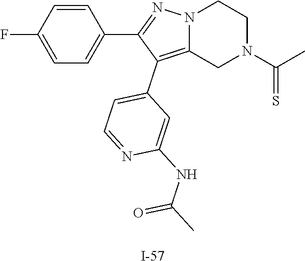 | N-(4-(5-ethanethioyl-2-(4-fluorophenyl)-4, 5,6,7-tetrahydropyrazolo[1,5-a]pyrazin-3- yl)pyridin-2-ylacetamide |
| I-58 | 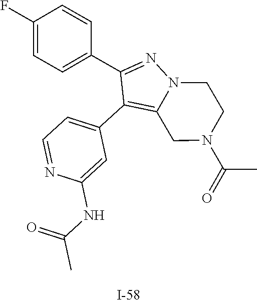 | N-(4-(5-acetyl-2-(4-fluorophenyl)-4,5,6,7- tetrahydropyrazolo[1,5-a]pyrazin-3-yl) pyridin-2-yl)acetamide |
| I-59 | 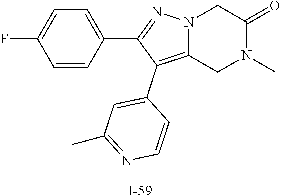 | 2-(4-fluorophenyl)-5-methyl-3-(2- methylpyridin-4-yl)-4,5- dihydropyrazolo[1,5-a] pyrazin-6(7H)-one |
| I-60 | 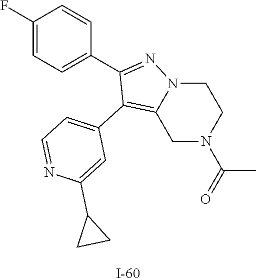 | 1-(3-(2-cyclopropylpyridin-4-yl)-2-(4- fluorophenyl)-6,7-dihydropyrazolo[1.5-a] pyrazin-5(4H)-yl)ethan-1-one |
| I-61 | 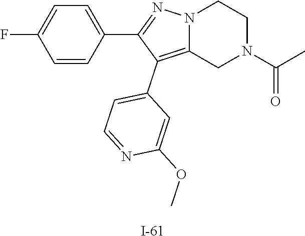 | 1-(2-(4-fluorophenyl)-3-(2-methoxypyridin- 4-yl)-6,7-dihydropyrazolo[1,5-a]pyrazin- 5(4H)-yl)ethan-1-one |
| I-62 | 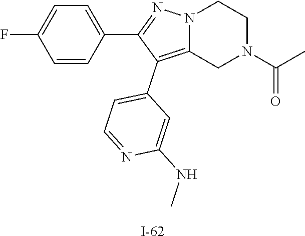 | 1-(2-(4-fluorophenyl)-3-(2-(methylamino) pyridin-4-yl)-6,7-dihydropyrazolo[1,5-a] pyrazin-5(4H)-yl)ethan-1-one |
| I-63 | 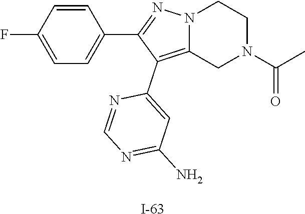 | 1-(3-(6-aminopyrimidin-4-yl)-2-(4-fluoro phenyl)-6,7-dihydropyrazolo[1,5-a] pyrazin-5(4H)-yl)ethan-1-one |
| I-64 | 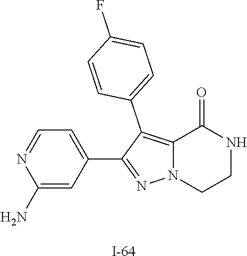 | 2-(2-aminopyridin-4-yl)-3-(4-fluorophenyl)- 6,7-dihydropyrazolo[1,5-a]pyrazin-4(5H)- one |
| I-65 | 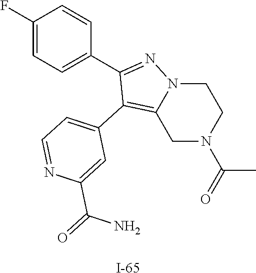 | 4-(5-acetyl-2-(4-fluorophenyl)-4,5,6,7- tetrahydropyrazolo[1,5-a]pyrazin-3-yl) picolinamide |
| I-66 | 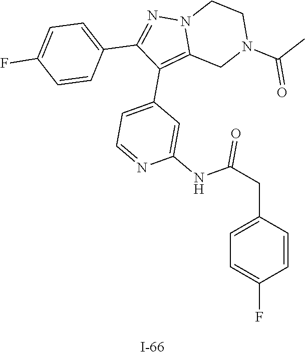 | N-(4-(5-acetyl-2-(4-fluorophenyl)-4,5,6,7- tetrahydropyrazolo[1,5-a]pyrazin-3-yl) pyridin-2-yl)-2-(4-fluorophenyl)acetamide |
| I-67 | 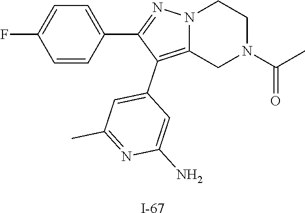 | 1-(3-(2-amino-6-methylpyridin-4-yl)-2-(4- fluorophenyl)-6,7-dihydropyrazolo[1,5-a] pyrazin-5(4H)-yl)ethan-1-one |
| I-68 | 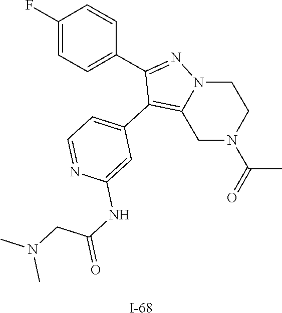 | N-(4-(5-acetyl-2-(4-fluorophenyl)-4,5,6,7- tetrahydropyrazolo[1,5-a]pyrazin-3-yl)pyridin- 2-yl)-2-(dimethylamino)acetamide |
| I-69 | 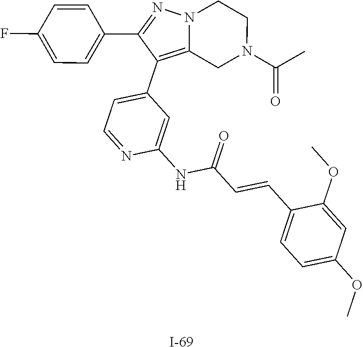 | (E)-N-(4-(5-acetyl-2-(4-fluorophonyl)-4,5, 6,7-tetrahydropyrazolo[1,5-a]pyrazin-3-yl) pyridin-2-yl)-3-(2,4-dimethoxyphenyl) acrylamide |
| I-70 | 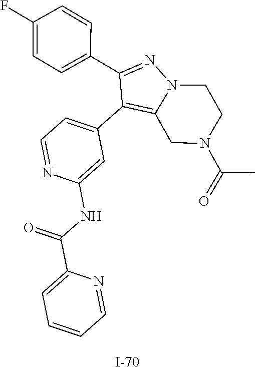 | N-(4-(5-acetyl-2-(4-fluorophenyl)-4,5,6,7- tetrahydropyrazolo[1,5-a]pyrazin-3-yl) pyridin-2-yl)picolinamide |
| I-71 | 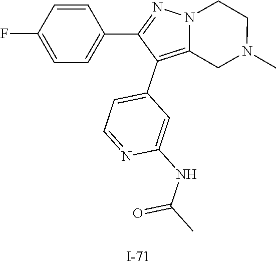 | N-(4-(2-(4-fluorophenyl)-5-methyl-4,5,6, 7-tetrahydropyrazolo[1,5-a]pyrazin-3-yl) pyridin-2-yl)acetamide |
| I-72 | 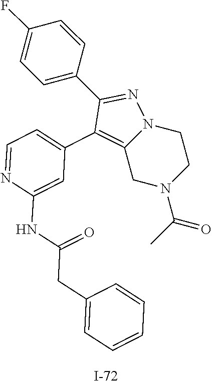 | N-(4-(5-acetyl-2-(4-fluorophenyl)-4,5,6,7- tetrahydropyrazolo[1,5-a]pyrazin-3-yl) pyridin-2-yl)-2-phenylacetamide |
| I-73 | 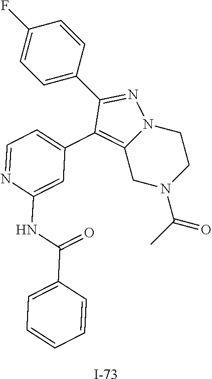 | N-(4-(5-acetyl-2-(4-fluorophenyl)-4,5,6,7- tetrahydropyrazolo[1,5-a]pyrazin-3-yl) pyridin-2-yl)benzamide |
| I-74 | 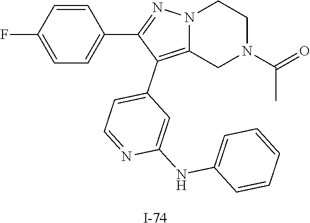 | 1-(2-(4-fluorophenyl)-3-(2-(phenylamino) pyridin-4-yl)-6,7-dihydropyrazolo[1,5-a] pyrazin-5(4H)-yl)ethan-1-one |
| I-75 | 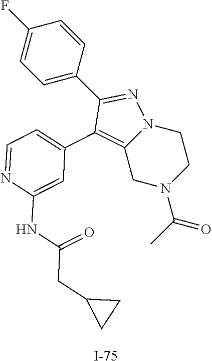 | N-(4-(5-acetyl-2-(4-fluorophenyl)-4,5,6,7- tetrahydropyrazolo[1,5-a]pyrazin-3-yl) pyridin-2-yl)-2-cyclopropylacetamide |
| I-76 | 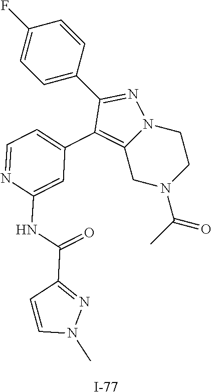 | N-(4-(5-acetyl-2-(4-fluorophenyl)-4,5,6,7- tetrahydropyrazolo[1,5-a]pyrazin-3-yl) pyridin-2-yl)-1-methyl-1H-pyrazole-3- carboxamide |
| I-77 | 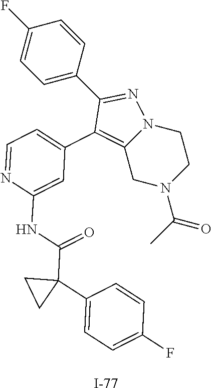 | N-(4-(5-acetyl-2-(4-fluorophenyl)-4,5,6,7- tetrahydropyrazolo[1,5-a]pyrazin-3-yl) pyridin-2-yl)-1-(4-fluorophenyl)cyclopropane- 1-carboxamide |
| I-78 | 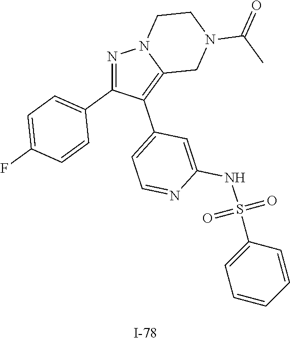 | N-(4-(5-acetyl-2-(4-fluorophenyl)-4,5,6,7- tetrahydropyrazolo[1,5-a]pyrazin-3-yl) pyridin-2-yl)benzenesulfonamide |
| I-79 | 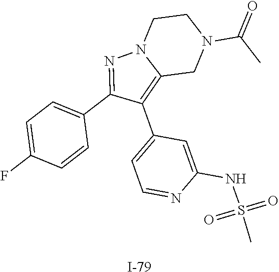 | N-(4-(5-acetyl-2-(4-fluorophenyl)-4,5,6,7- tetrahydropyrazolo[1,5-a]pyrazin-3-yl) pyridin-2-yl)methanesulfonamide |
| I-80 | 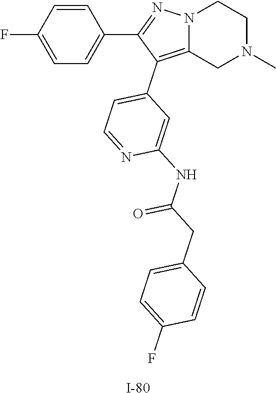 | 2-(4-fluorophenyl)-N-(4-(2-(4-fluorophenyl)- 5-methyl-4,5,6,7-tetrahydropyrazolo [1,5-a]pyrazin-3-yl)pyridin-2-yl)acetamide |
| I-81 | 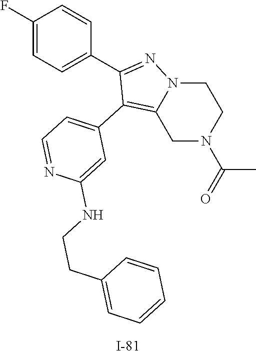 | 1-(2-(4-fluorophenyl)-3-(2-(phenethylamino) pyridin-4-yl)-6,7-dihydropyrazolo[1,5- a]pyrazin-5(4H)-yl)ethan-1-one |
| I-82 | 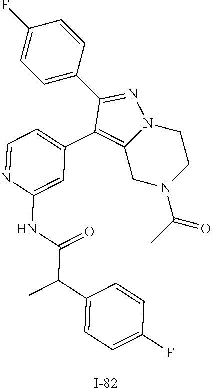 | N-(4-(5-acetyl-2-(4-fluorophenyl)-4,5,6,7- tetrahydropyrazolo[1,5-a]pyrazin-3-yl)pyridin- 2-yl)-2-(4-fluorophenyl)propanamide |
| I-83 | 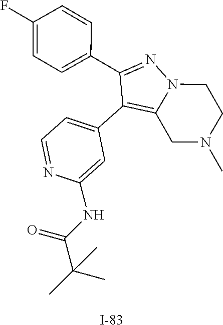 | N-(4-(2-(4-fluorophenyl)-5-methyl-4.5,6, 7-tetrahydropyrazolo[1,5-a]pyrazin-3-yl) pyridin-2-yl)pivalamide |
| I-84 | 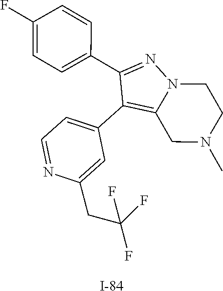 | 2-(4-fluorophenyl)-5-methyl-3-(2-(2,2,2- trifluoroethyl)pyridin-4-yl)-4,5,6,7- tetrahydropyrazolo[1,5-a]pyrazine |
| I-85 | 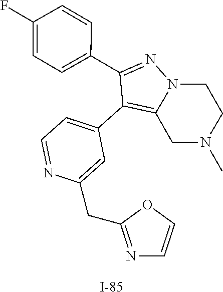 | 2-((4-(2-(4-fluorophenyl)-5-methyl-4,5,6, 7-tetrahydropyrazolo[1,5-a]pyrazin-3-yl) pyridin-2-yl)methyl)oxazole |
| I-86 | 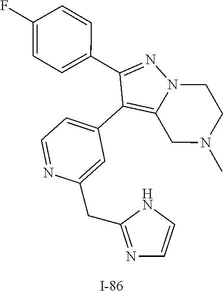 | 3-(2-((1H-imidazol-2-yl)methyl)pyridin-4- yl)-2-(4-fluorophenyl)-5-methyl-4,5,6,7- tetrahydropyrazolo[1,5-a]pyrazine |
| I-87 | 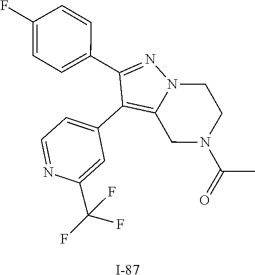 | 1-(2-(4-fluorophenyl)-3-(2-(trifluoromethyl) pyridin-4-yl)-6,7-dihydropyrazolo[1,5- a]pyrazin-5(4H)-yl)ethan-1-one |
| I-88 | 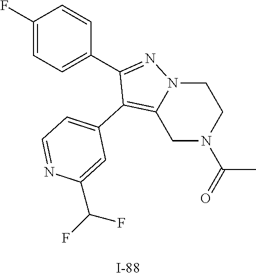 | 1-(3-(2-(difluoromethyl)pyridin-4-yl)-2-(4- fluorophenyl)-6,7-dihydropyrazolo[1,5-a] pyrazin-5(4H)-yl)ethan-1-one |
| I-89 | 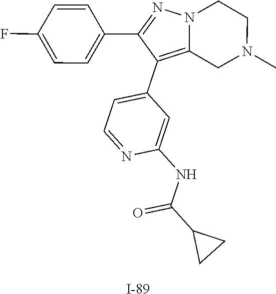 | N-(4-(2-(4-fluorophenyl)-5-methyl-4,5,6, 7-tetrahydropyrazolo[1,5-a]pyrazin-3-yl) pyridin-2-yl)cyclopropanecarboxamide |
| I-90 | 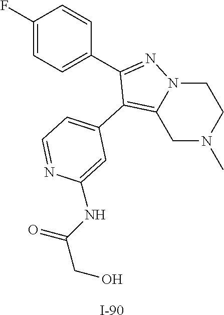 | N-(4-(2-(4-fluorophenyl)-5-methyl-4,5,6, 7-tetrahydropyrazolo[1,5-a]pyrazin-3-yl) pyridin-2-yl)-2-hydroxyacetamide |
| I-91 | 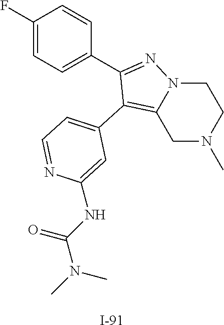 | 3-(4-(2-(4-fluorophenyl)-5-methyl-4,5,6,7- tetrahydropyrazolo[1,5-a]pyrazin-3-yl) pyridin-2-yl)-1,1-dimethylurea |
| I-92 | 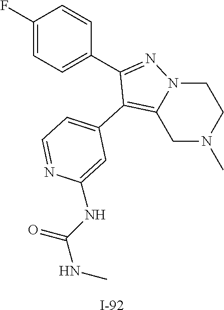 | 1-(4-(2-(4-fluorophenyl)-5-methyl-4,5,6,7- tetrahydropyrazolo[1,5-a]pyrazin-3-yl) pyridin-2-yl)-3-methylurea |
| I-93 | 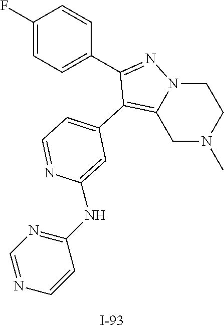 | N-(4-(2-(4-fluorophenyl)-5-methyl-4.5,6, 7-tetrahydropyrazolo[1,5-a]pyrazin-3-yl) pyridin-2-yl)pyrimidin-4-amine |
| I-94 | 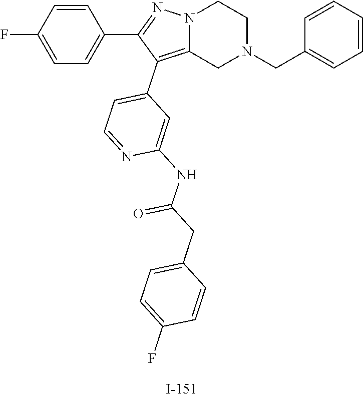 [SMILES: O=C(Cc1ccc(F)cc1)Nc1cc(-c2c(-c3ccc(F)cc3)nn3c2CN(Cc2ccccc2)CC3)ccn1] | N-(4-(5-benzyl-2-(4-fluorophenyl)-4,5,6,7- tetrahydropyrazolo[1,5-a]pyrazin-3-yl) pyridin-2-yl)-2-(4-fluorophenyl)acetamide |
| I-95 | 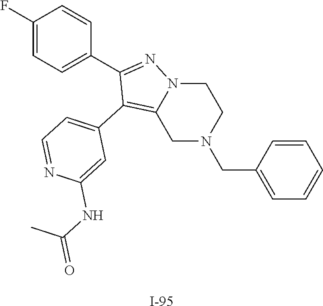 | N-(4-(5-benzyl-2-(4-fluorophenyl)-4,5,6,7- tetrahydropyrazolo[1,5-a]pyrazin-3-yl) pyridin-2-yl)acetamide |
| I-96 | 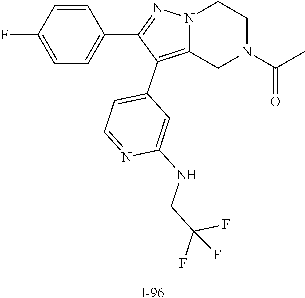 | 1-(2-(4-fluorophenyl)-3- (2-((2,2,2-trifluoroethyl) amino)pyridin-4-yl)-6,7-dihydropyrazolo [1,5-a]pyrazin-5(4H)-yl)ethan-1-one |
| I-97 | 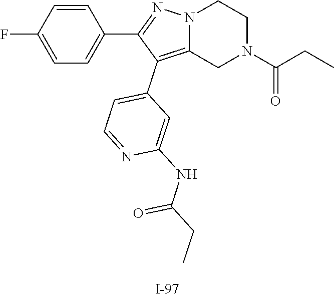 | N-(4-(2-(4-fluorophenyl)-5-propionyl-4,5, 6,7-tetrahydropyrazolo[1,5-a]pyrazin-3-yl) pyridin-2-yl)propionamide |
| I-98 | 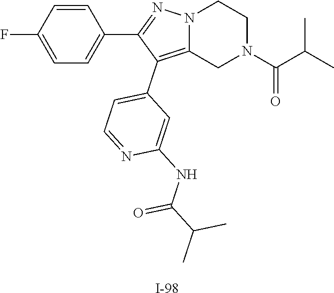 | N-(4-(2-(4-fluorophenyl)-5-isobutyryl-4,5, 6,7-tetrahydropyrazolo[1,5-a]pyrazin-3-yl) pyridin-2-yl)isobutyramide |
| I-99 | 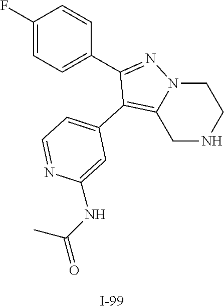 | N-(4-(2-(4-fluorophenyl)-4,5,6,7- tetrahydropyrazolo[1,5-a]pyrazin- 3-yl)pyridin-2-yl)acetamide |
| I-100 | 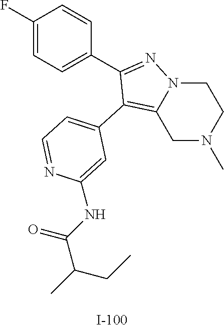 [SMILES: CCC(C)C(=O)Nc1cc(-c2c(-c3ccc(F)cc3)nn3c2CN(C)CC3)ccn1] | N-(4-(2-(4-fluorophenyl)-5-methyl-4,5,6, 7-tetrahydropyrazolo[1,5-a]pyrazin-3-yl) pyridin-2-yl)-2-methylbutanamide |
| I-101 | 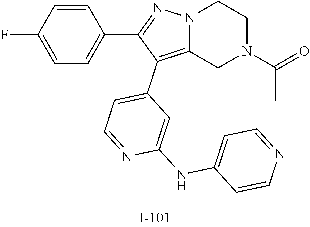 [SMILES: CC(=O)N1CCn2nc(-c3ccc(F)cc3)c(-c3ccnc(Nc4ccncc4)c3)c2C1] | 1-(2-(4-fluorophenyl)-3-(2-(pyridin-4-yl amino)pyridin-4-yl)-6,7-dihydropyrazolo[1, 5-a]pyrazin-5(4H)-yl)ethan-1-one |
| I-102 | 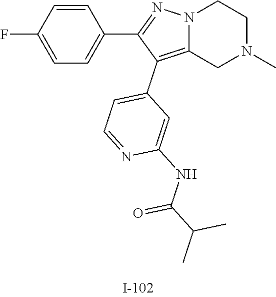 [SMILES: CC(C)C(=O)Nc1cc(-c2c(-c3ccc(F)cc3)nn3c2CN(C)CC3)ccn1] | N-(4-(2-(4-fluorophenyl)-5-methyl-4,5,6, 7-tetrahydropyrazolo[1,5-a]pyrazin-3-yl) pyridin-2-yl)isobutyramide |
| I-103 | 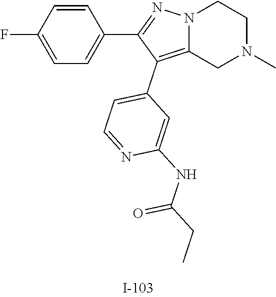 [SMILES: CCC(=O)Nc1cc(-c2c(-c3ccc(F)cc3)nn3c2CN(C)CC3)ccn1] | N-(4-(2-(4-fluorophenyl)-5-methyl-4,5,6, 7-tetrahydropyrazolo[1,5-a]pyrazin-3-yl) pyridin-2-yl)propionamide |
| I-104 | 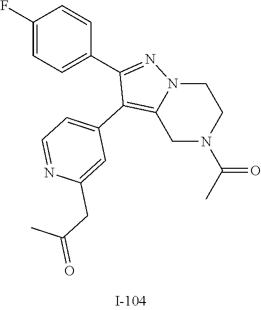 [SMILES: CC(=O)Cc1cc(-c2c(-c3ccc(F)cc3)nn3c2CN(C(C)=O)CC3)ccn1] | 1-(4-(5-acetyl-2-(4-fluorophenyl)-4,5,6,7- tetrahydropyrazolo[1,5-a]pyrazin-3-yl) pyridin-2-yl)propan-2-one |
| I-105 | 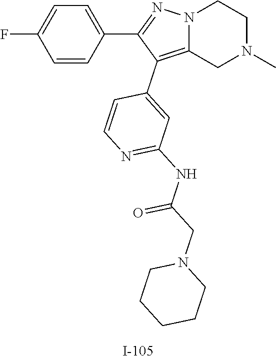 [SMILES: CN1CCn2nc(-c3ccc(F)cc3)c(-c3ccnc(NC(=O)CN4CCCCC4)c3)c2C1] | N-(4-(2-(4-fluorophenyl)-5-methyl-4,5,6, 7-tetrahydropyrazolo[1,5-a]pyrazin-3-yl) pyridin-2-yl)-2-(piperidin-1-yl)acetamide |
| I-106 | 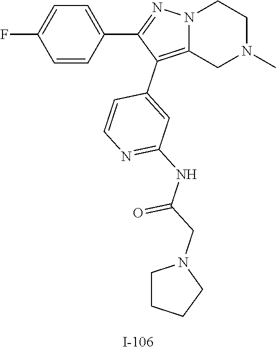 [SMILES: CN1CCn2nc(-c3ccc(F)cc3)c(-c3ccnc(NC(=O)CN4CCCC4)c3)c2C1] | N-(4-(2-(4-fluorophenyl)-5-methyl-4,5,6, 7-tetrahydropyrazolo[1,5-a]pyrazin-3-yl) pyridin-2-yl)-2-(pyrrolidin-1-yl)acetamide |
| I-107 | 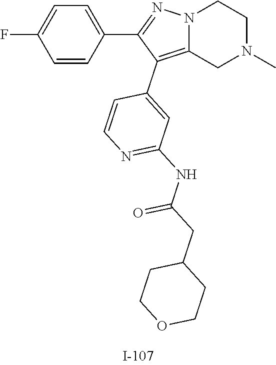 [SMILES: CN1CCn2nc(-c3ccc(F)cc3)c(-c3ccnc(NC(=O)CC4CCOCC4)c3)c2C1] | N-(4-(2-(4-fluorophenyl)-5-methyl-4,5,6. 7-tetrahydropyrazolo[1,5-a]pyrazin-3-yl) pyridin-2-yl)-2-(tetrahydro-2H-pyran-4-yl) acetamide |
| I-108 | 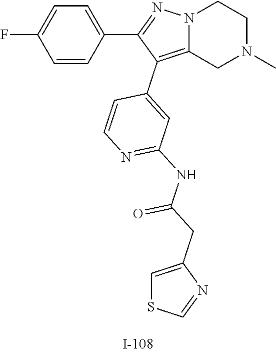 [SMILES: CN1CCn2nc(-c3ccc(F)cc3)c(-c3ccnc(NC(=O)Cc4cscn4)c3)c2C1] | N-(4-(2-(4-fluorophenyl)-5-methyl-4,5,6, 7-tetrahydropyrazolo[1,5-a]pyrazin-3-yl) pyridin-2-yl)-2-(thiazol-4-yl)acetamide |
| I-109 | 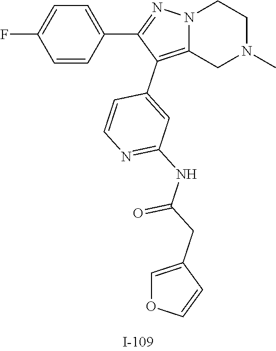 [SMILES: CN1CCn2nc(-c3ccc(F)cc3)c(-c3ccnc(NC(=O)Cc4ccoc4)c3)c2C1] | N-(4-(2-(4-fluorophenyl)-5-methyl-4,5,6, 7-tetrahydropyrazolo[1,5-a]pyrazin-3-yl) pyridin-2-yl)-2-(furan-3-yl)acetamide |
| I-110 | 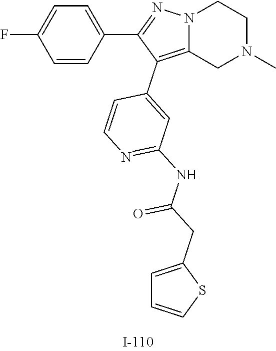 [SMILES: CN1CCn2nc(-c3ccc(F)cc3)c(-c3ccnc(NC(=O)Cc4cccs4)c3)c2C1] | N-(4-(2-(4-fluorophenyl)-5-methyl-4,5,6, 7-tetrahydropyrazolo[1,5-a]pyrazin-3-yl) pyridin-2-yl)-2-(thiophen-2-yl)acetamide |
| I-111 | 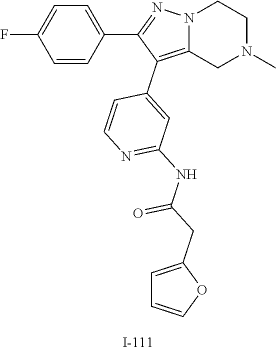 [SMILES: CN1CCn2nc(-c3ccc(F)cc3)c(-c3ccnc(NC(=O)Cc4ccco4)c3)c2C1] | N-(4-(2-(4-fluorophenyl)-5-methyl-4,5,6, 7-tetrahydropyrazolo[1,5-a]pyrazin-3-yl) pyridin-2-yl)-2-(furan-2-yl)acetamide |
| I-112 | 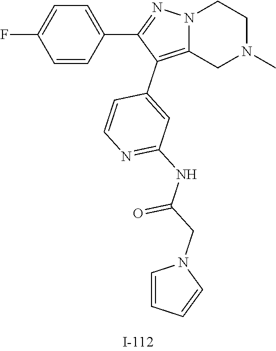 [SMILES: CN1CCn2nc(-c3ccc(F)cc3)c(-c3ccnc(NC(=O)Cn4cccc4)c3)c2C1] | N-(4-(2-(4-fluorophenyl)-5-methyl-4,5,6, 7-tetrahydropyrazolo[1,5-a]pyrazin-3-yl) pyridin-2-yl)-2-(1H-pyrrol-1-yl)acetamide |
| I-113 | 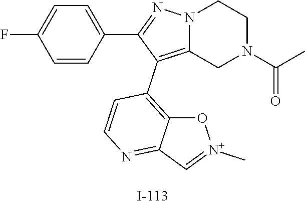 [SMILES: CC(=O)N1CCn2nc(-c3ccc(F)cc3)c(-c3ccnc4c[n+](C)oc34)c2C1] | 7-(5-acetyl-2-(4-fluorophenyl)-4,5,6,7- tetrahydropyrazolo[1,5-a]pyrazin-3-yl)-2- methylisoxazolo[4,5-b]pyridin-2-ium |
| I-114 | 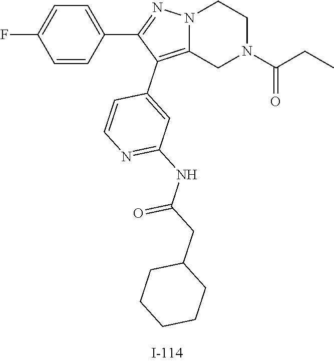 [SMILES: CCC(=O)N1CCn2nc(-c3ccc(F)cc3)c(-c3ccnc(NC(=O)CC4CCCCC4)c3)c2C1] | 2-cyclohexyl-N-(4-(2-(4-fluorophenyl)-5- propionyl-4,5,6,7-tetrahydropyrazolo[1,5- a]pyrazin-3-yl)pyridin-2-yl)acetamide |
| I-115 | 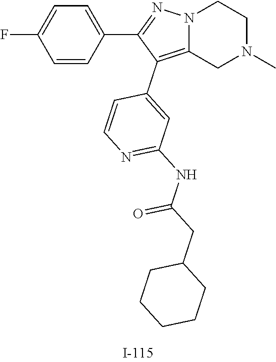 [SMILES: CN1CCn2nc(-c3ccc(F)cc3)c(-c3ccnc(NC(=O)CC4CCCCC4)c3)c2C1] | 2-cyclohexyl-N-(4-(2-(4-fluorophenyl)-5- methyl-4,5,6,7-tetrahydropyrazolo [1,5-a]pyrazin-3-yl)pyridin-2-yl)acetamide |
| I-116 | 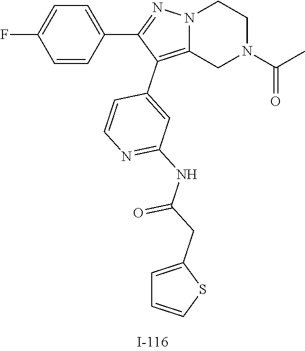 [SMILES: CC(=O)N1CCn2nc(-c3ccc(F)cc3)c(-c3ccnc(NC(=O)Cc4cccs4)c3)c2C1] | N-(4-(5-acetyl-2-(4-fluorophenyl)-4,5,6,7- tetrahydropyrazolo[1,5-a]pyrazin-3-yl) pyridin-2-yl)-2-(thiopben-2-yl)acetamide |
| I-117 | 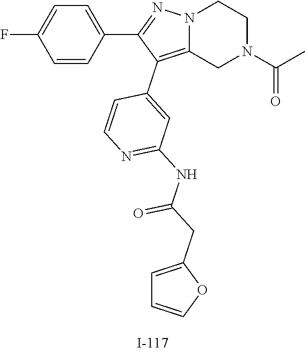 [SMILES: CC(=O)N1CCn2nc(-c3ccc(F)cc3)c(-c3ccnc(NC(=O)Cc4ccco4)c3)c2C1] | N-(4-(5-acetyl-2-(4-fluorophenyl)-4,5,6,7- tetrahydropyrazolo[1,5-a]pyrazin-3-yl) pyridin-2-yl)-2-(furan-2-yl)acetamide |
| I-118 | 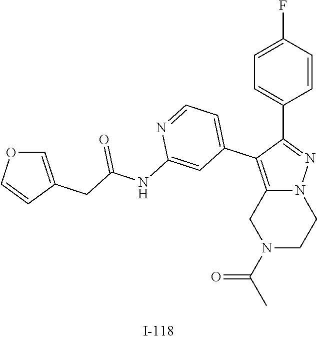 [SMILES: CC(=O)N1CCn2nc(-c3ccc(F)cc3)c(-c3ccnc(NC(=O)Cc4ccoc4)c3)c2C1] | N-(4-(5-acetyl-2-(4-fluorophenyl)-4,5,6,7- tetrahydropyrazolo[1,5-a]pyrazin-3-yl) pyridin-2-yl)-2-(furan-3-yl)acetamide |
| I-119 | 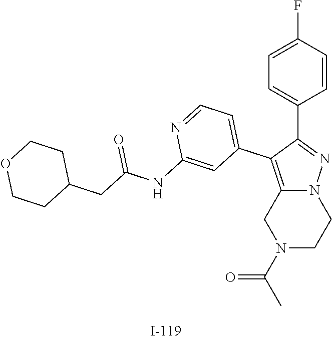 [SMILES: CC(=O)N1CCn2nc(-c3ccc(F)cc3)c(-c3ccnc(NC(=O)CC4CCOCC4)c3)c2C1] | N-(4-(5-acetyl-2-(4-fluorophenyl)-4,5,6,7- tetrahydropyrazolo[1,5-a]pyrazin-3-yl) pyridin-2-yl)-2-(tetrahydro-2H-pyran-4-yl) acetamide |
| I-120 | 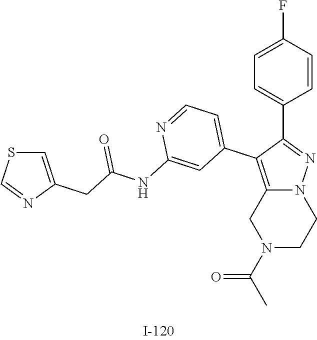 [SMILES: CC(=O)N1CCn2nc(-c3ccc(F)cc3)c(-c3ccnc(NC(=O)Cc4cscn4)c3)c2C1] | N-(4-(5-acetyl-2-(4-fluorophenyl)-4,5,6,7- tetrahydropyrazolo[1,5-a]pyrazin-3-yl) pyridin-2-yl)-2-(thiazol-4-yl)acetamide |
| I-121 | 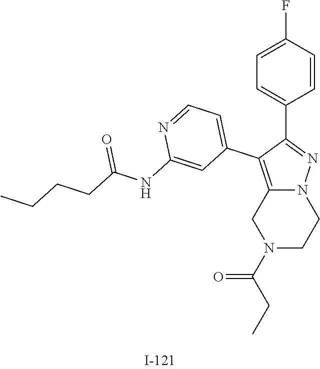 [SMILES: CCCCC(=O)Nc1cc(-c2c(-c3ccc(F)cc3)nn3c2CN(C(=O)CC)CC3)ccn1] | N-(4-(2-(4-fluorophenyl)-5-propionyl-4,5, 6,7-tetrahydropyrazolo[1,5-a]pyrazin-3-yl) pyridin-2-yl)pentanamide |
| I-122 | 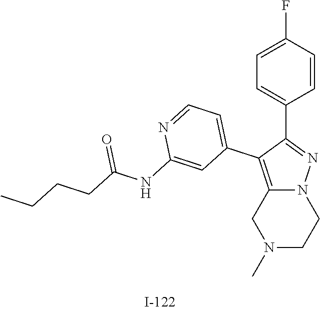 [SMILES: CCCCC(=O)Nc1cc(-c2c(-c3ccc(F)cc3)nn3c2CN(C)CC3)ccn1] | N-(4-(2-(4-fluorophenyl)-5-methyl-4,5,6, 7-tetrahydropyrazolo[1,5-a]pyrazin-3-yl) pyridin-2-yl)pentanamide |
| I-123 | 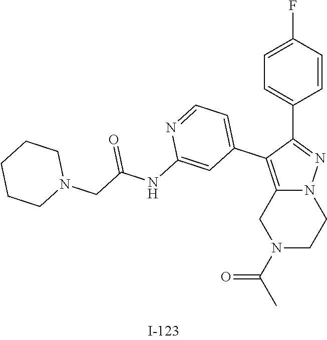 [SMILES: CC(=O)N1CCn2nc(-c3ccc(F)cc3)c(-c3ccnc(NC(=O)CN4CCCCC4)c3)c2C1] | N-(4-(5-acetyl-2-(4-fluorophenyl)-4,5,6,7- tetrahydropyrazolo[1,5-a]pyrazin-3-yl) pyridin-2-yl)-2-(piperidin-1-yl)acetamide |
| I-124 | 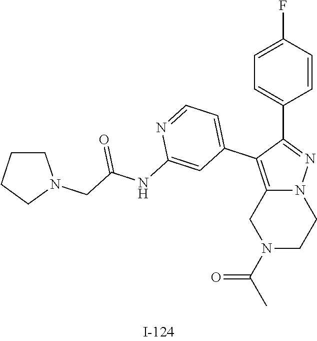 [SMILES: CC(=O)N1CCn2nc(-c3ccc(F)cc3)c(-c3ccnc(NC(=O)CN4CCCC4)c3)c2C1] | N-(4-(5-acetyl-2-(4-fluorophenyl)-4,5,6,7- tetrahydropyrazolo[1,5-a]pyrazin-3-yl) pyridin-2-yl)-2-(pyrrolidin-1-yl)acetamide |
| I-125 | 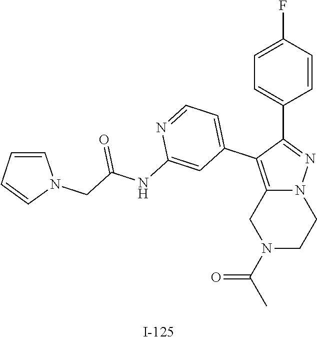 [SMILES: CC(=O)N1CCn2nc(-c3ccc(F)cc3)c(-c3ccnc(NC(=O)Cn4cccc4)c3)c2C1] | N-(4-(5-acetyl-2-(4-fluorophenyl)-4,5,6,7- tetrahydropyrazolo[1,5-a]pyrazin-3-yl) pyridin-2-yl)-2-(1H-pyrrol-1-yl)acetamide |
| I-126 | 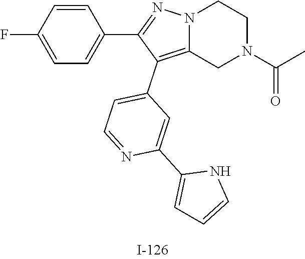 [SMILES: CC(=O)N1CCn2nc(-c3ccc(F)cc3)c(-c3ccnc(-c4ccc[nH]4)c3)c2C1] | 1-(3-(2-(1H-pyrrol-2-yl)pyridin-4-yl)-2-(4- fluorophenyl)-6,7-dihydropyrazolo[1,5-a] pyrazin-5(4H)-yl)ethan-1-one |
| I-127 | 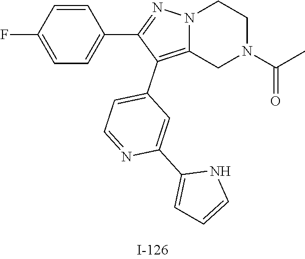 [SMILES: CC(=O)N1CCn2nc(-c3ccc(F)cc3)c(-c3ccnc(-c4ccc[nH]4)c3)c2C1] | 1-(2-(4-fluorophenyl)-3-(2-(5-methyl-1H- pyrrol-2-yl)pyridin-4-yl)-6,7-dihydropyrazolo [1,5-a]pyrazin-5(4H)-yl)ethan-1-one |
| I-128 | 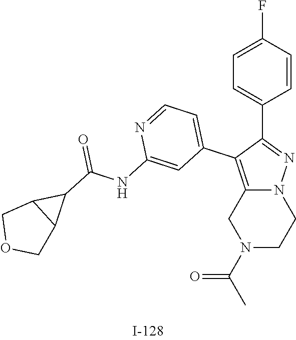 [SMILES: CC(=O)N1CCn2nc(-c3ccc(F)cc3)c(-c3ccnc(NC(=O)C4C5COCC54)c3)c2C1] | N-(4-(5-acetyl-2-(4-fluorophenyl)-4,5,6,7- tetrahydropyrazolo[1,5-a]pyrazin-3-yl) pyridin-2-yl)-3-oxabicyclo[3.1.0]hexane- 6-carboxamide |
| I-129 | 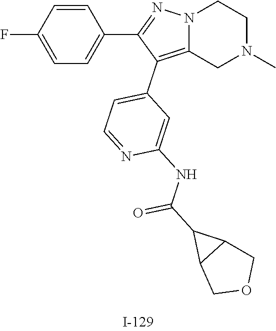 [SMILES: CN1CCn2nc(-c3ccc(F)cc3)c(-c3ccnc(NC(=O)C4C5COCC54)c3)c2C1] | N-(4-(2-(4-fluorophenyl)-5-methyl-4,5,6, 7-tetrahydropyrazolo[1,5-a]pyrazin-3-yl) pyridin-2-yl)-3-oxabicyclo[3.1.0]hexane-6- carboxamide |
| I-130 | 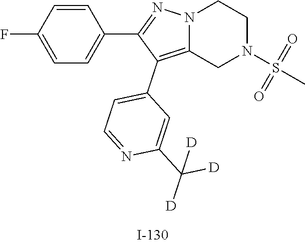 [SMILES: [2H]C([2H])([2H])c1cc(-c2c(-c3ccc(F)cc3)nn3c2CN(S(C)(=O)=O)CC3)ccn1] | 2-(4-fluorophenyl)-3-(2-(methyl-d3)pyridin- 4-yl)-5-(methylsulfonyl)-4,5,6,7- tetrahydropyrazolo[1,5-a]pyrazine |
| I-131 | 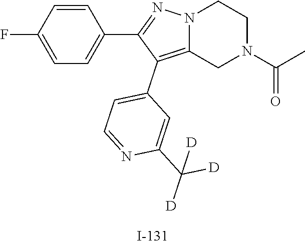 [SMILES: [2H]C([2H])([2H])c1cc(-c2c(-c3ccc(F)cc3)nn3c2CN(C(C)=O)CC3)ccn1] | 1-(2-(4-fluorophenyl)-3-(2-(methyl-d3) pyridin-4-yl)-6,7-dihydropyrazolo[1,5-a] pyrazin-5(4H)-yl)ethan-1-one |
| I-132 | 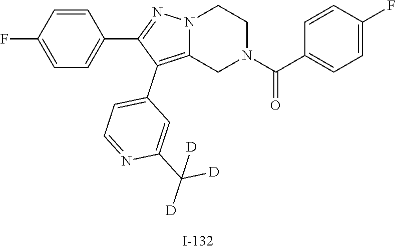 [SMILES: [2H]C([2H])([2H])c1cc(-c2c(-c3ccc(F)cc3)nn3c2CN(C(=O)c2ccc(F)cc2)CC3)ccn1] | (4-fluorophenyl)(2-(4-fluorophenyl)-3-(2- (methyl-d3)pyridin-4-yl)-6,7-dihydropyrazolo [1,5-a]pyrazin-5(4H)-yl)methanone |
| I-133 | 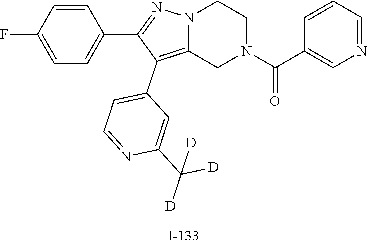 [SMILES: [2H]C([2H])([2H])c1cc(-c2c(-c3ccc(F)cc3)nn3c2CN(C(=O)c2cccnc2)CC3)ccn1] | (2-(4-fluorophenyl)-3-(2-(methyl-d3)pyridin- 4-yl)-6,7-dihydropyrazolo[1,5-a]pyrazin- 5(4H)-yl)(pyridin-3-yl)methanone |
| I-134 | 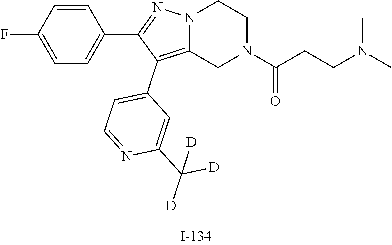 [SMILES: [2H]C([2H])([2H])c1cc(-c2c(-c3ccc(F)cc3)nn3c2CN(C(=O)CCN(C)C)CC3)ccn1] | 3-(dimethylamino)-1-(2-(4-fluorophenyl)- 3-(2-(methyl-d3)pyridin-4-yl)-6,7- dihydropyrazolo[1,5-a] pyrazin-5(4H)-yl)propan-1- one |

### Medical Use

In one aspect, the present application provides a method for inhibiting casein kinase (CK) activity, said method comprising administering to a subject in need thereof an effective amount of the compound of the present application, or a pharmaceutically acceptable salt, prodrug, or metabolite thereof, or a solvate or hydrate of any of the foregoing. For example, the casein kinase (CK) may be selected from the group consisting of casein kinase I alpha (CK1α), casein kinase I delta (CK1δ) and casein kinase I epsilon (CK1ε). For example, the method may be selected from the group consisting of an in vitro method, an ex vivo method, and an in vivo method.

In another embodiment, the present application provides use the compound of the present application, or a pharmaceutically acceptable salt, prodrug, or metabolite thereof, or a solvate or hydrate of any of the foregoing of the present application in the preparation of a drug and/or a kit for use in inhibiting casein kinase (CK) activity. For example, the casein kinase (CK) may be selected from the group consisting of casein kinase I alpha (CK1α), casein kinase I delta (CK1δ) and casein kinase I epsilon (CK1ε). For example, the method may be selected from the group consisting of an in vitro method, an ex vivo method, and an in vivo method.

In another embodiment, the present application provides the compound of the present application, or a pharmaceutically acceptable salt, prodrug, or metabolite thereof, or a solvate or hydrate of any of the foregoing of the present application for use in inhibiting casein kinase (CK) activity. For example, the casein kinase (CK) may be selected from the group consisting of casein kinase I alpha (CK1α), casein kinase I delta (CK1δ) and casein kinase I epsilon (CK1ε). For example, the method may be selected from the group consisting of an in vitro method, an ex vivo method, and an in vivo method.

In another aspect, the present application provides a method for preventing and/or treating a disease or disorder, said method comprising administering to a subject in need thereof an effective amount of the compound of the present application, or a pharmaceutically acceptable salt, prodrug, or metabolite thereof, or a solvate or hydrate of any of the foregoing. For example, the disease or disorder may be selected from the group consisting of neurological disease and psychiatric disease. For example, the disease or disorder may be selected from the group consisting of mood disorder, sleep disorder, and circadian disorder. For example, the disease or disorder may be selected from the group consisting of depressive disorder and bipolar disorder.

In another embodiment, the present application provides use the compound of the present application, or a pharmaceutically acceptable salt, prodrug, or metabolite thereof, or a solvate or hydrate of any of the foregoing of the present application in the preparation of a drug and/or a kit for use in preventing and/or treating a disease or disorder. For example, the disease or disorder may be selected from the group consisting of neurological disease and psychiatric disease. For example, the disease or disorder may be selected from the group consisting of mood disorder, sleep disorder, and circadian disorder. For example, the disease or disorder may be selected from the group consisting of depressive disorder and bipolar disorder.

In another embodiment, the present application provides the compound of the present application, or a pharmaceutically acceptable salt, prodrug, or metabolite thereof, or a solvate or hydrate of any of the foregoing of the present application for use in preventing and/or treating a disease or disorder. For example, the disease or disorder may be selected from the group consisting of neurological disease and psychiatric disease. For example, the disease or disorder may be selected from the group consisting of mood disorder, sleep disorder, and circadian disorder. For example, the disease or disorder may be selected from the group consisting of depressive disorder and bipolar disorder.

In another embodiment, the present application provides compositions comprising a compound of the present application or a pharmaceutically acceptable salt, prodrug, or metabolite thereof, or a solvate or hydrate of any of the foregoing, and optionally a pharmaceutically acceptable carrier.

The compounds of the application may be administered orally. Oral administration may involve swallowing, so that the compound enters the gastrointestinal tract, or buccal or sublingual administration may be employed by which the compound enters the blood stream directly from the mouth.

In some cases, the compounds of the present application may also be administered directly into the blood stream, into muscle, or into an internal organ. Suitable means for parenteral administration may include intravenous, intraarterial, intraperitoneal, intrathecal, intraventricular, intraurethral, intrasternal, intracranial, intramuscular and subcutaneous. Suitable devices for parenteral administration may include needle (including microneedle) injectors, needle-free injectors and infusion techniques.

The compounds of the present application may also be administered topically to the skin or mucosa, that is, dermally or transdermally. In some cases, the compounds of the present application may also be administered intranasally or by inhalation. In some cases, the compounds of the present application may be administered rectally or vaginally. In another embodiment, the compounds of the present application may also be administered directly to the eye or ear.

The dosage regimen for the compounds and/or compositions containing the compounds is based on a variety of factors, including the type, age, weight, sex and medical condition of the patient; the severity of the condition; the route of administration; and the activity of the particular compound employed. Thus, the dosage regimen may vary widely. Dosage levels of the order from about 0.01 mg to about 100 mg per kilogram of body weight per day may be useful in the treatment of the above-indicated conditions.

Suitable subjects according to the present invention include mammalian subjects. Mammals according to the present invention may include, but are not limited to, canine, feline, bovine, caprine, equine, ovine, porcine, rodents, lagomorphs, primates, and the like, and encompass mammals in utero. In one embodiment, humans are suitable subjects. Human subjects may be of either gender and at any stage of development.

In another embodiment, the present application provides use of one or more compounds of the present application for the preparation of a medicament for the treatment of the conditions recited herein.

For the treatment of the conditions referred to above, the compounds of the present application may be administered as compound per se. Alternatively, pharmaceutically acceptable salts may be suitable for medical applications because of their greater aqueous solubility relative to the parent compound.

In another embodiment, the present application provides compositions. Such compositions may comprise a compound of the present application presented with a pharmaceutically acceptable carrier. The carrier may be a solid product, a liquid, or both, and may be formulated with the compound as a unit-dose composition, for example, a tablet, which can contain from 0.05% to 95% by weight of the active compounds. A compound of the present application may be coupled with suitable polymers as targetable drug carriers. Other pharmacologically active substances may also be present.

The compounds of the present invention may be administered by any suitable route, may be in the form of a pharmaceutical composition adapted to such a route, and in a dose effective for the treatment intended. The active compounds and compositions, for example, may be administered orally, rectally, parenterally, or topically.

The compounds of the present application may be used, alone or in combination with other therapeutic agents, in the treatment of various conditions or disease states. The compound(s) of the present application and other therapeutic agent(s) may be administered simultaneously (either in the same dosage form or in separate dosage forms) or sequentially.

The administration of two or more compounds “in combination” may mean that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” may mean that the compounds are administered in combination.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below am all or the only experiments performed. Efforts have been made to ensure accuracy with respect to numbers used (e.g. amounts, temperature, etc.) but some experimental errors and deviations should be accounted for. Unless indicated otherwise, pats are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Example 1 Preparation of Compounds

### Example 1-1 Preparation of Compound I-1

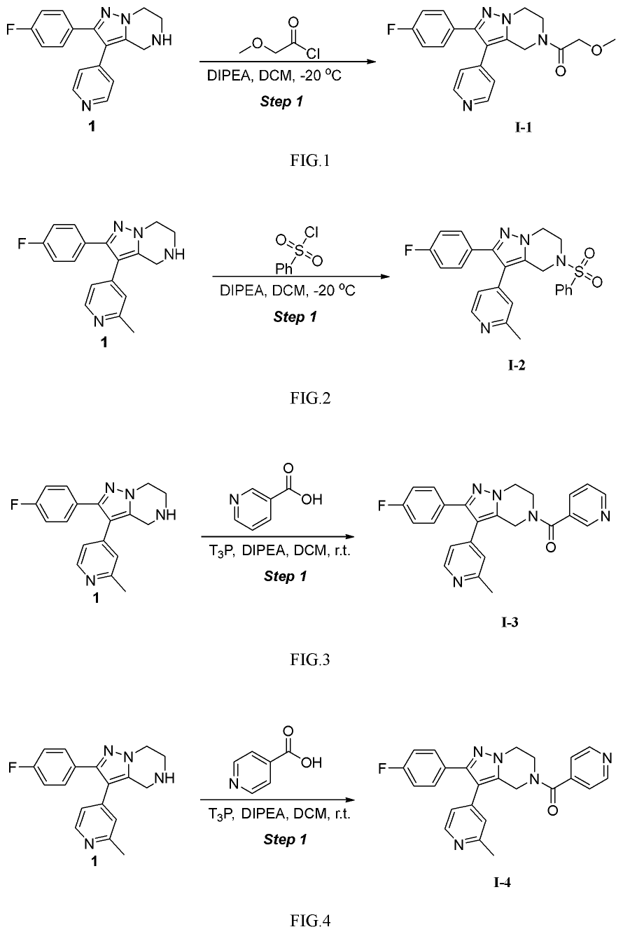

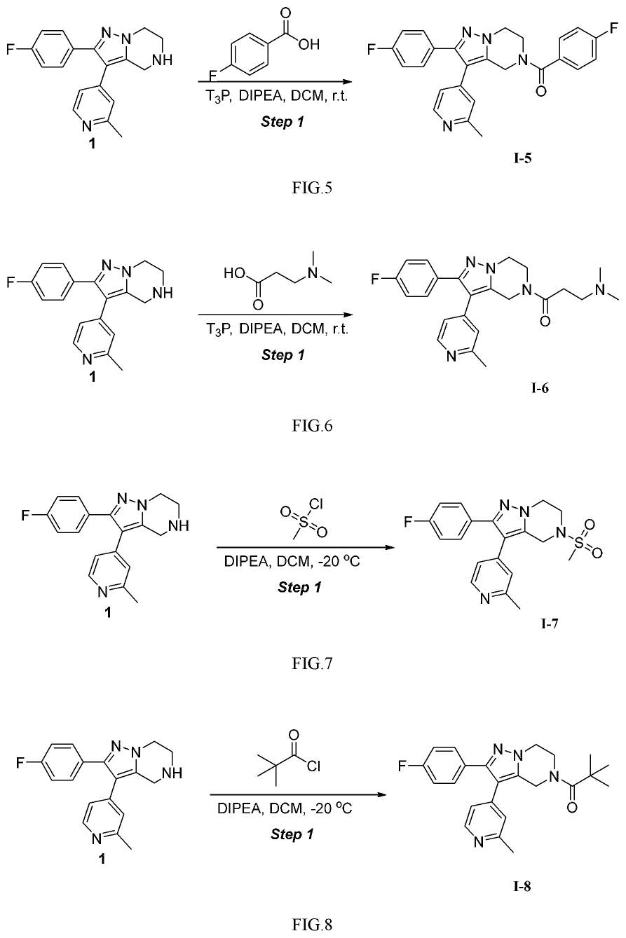

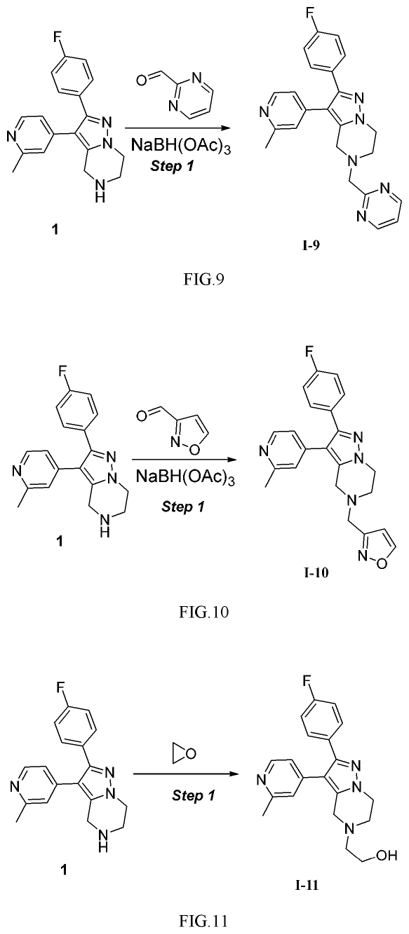

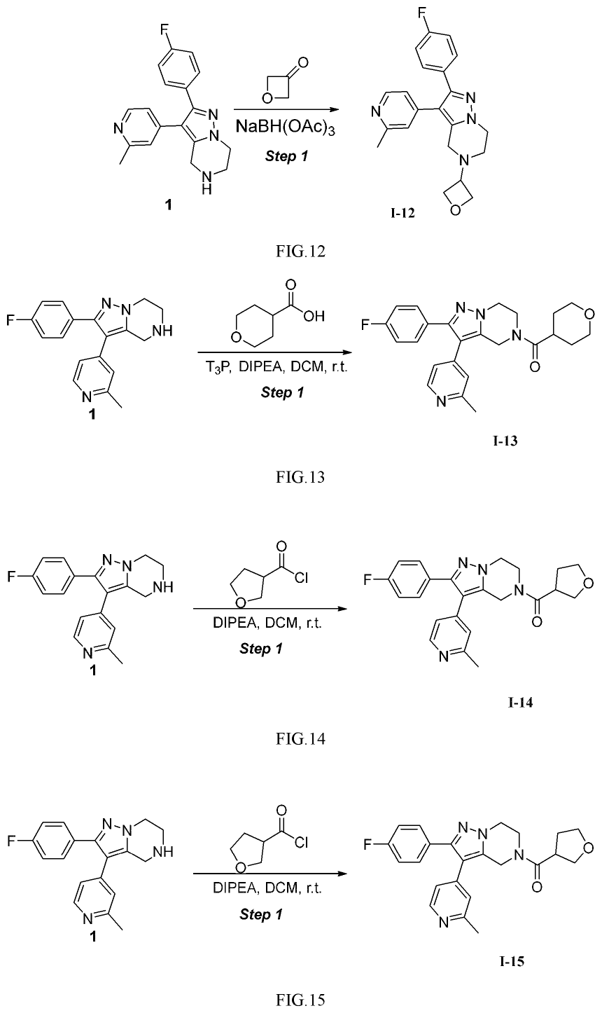

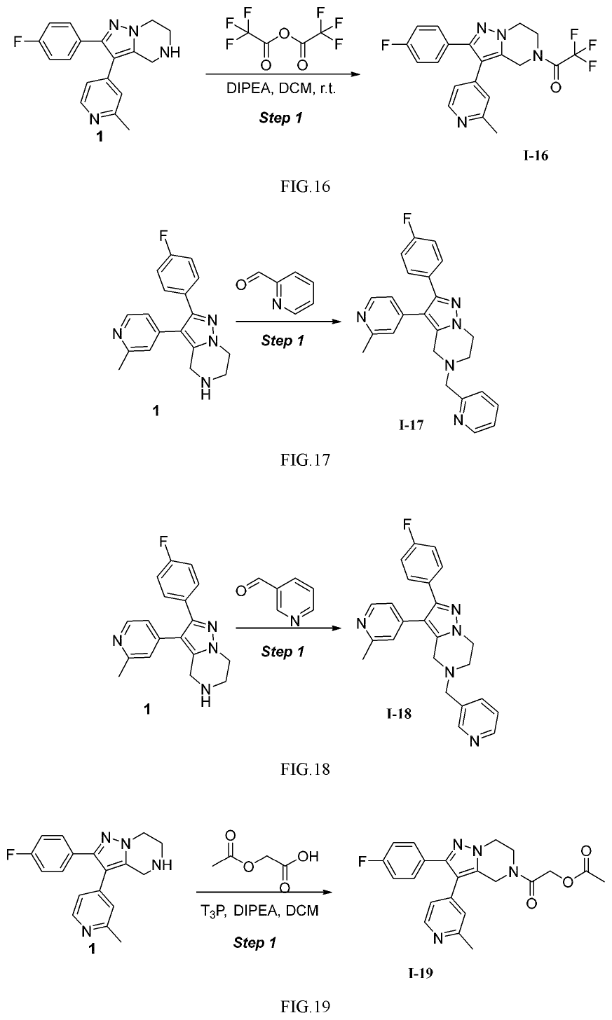

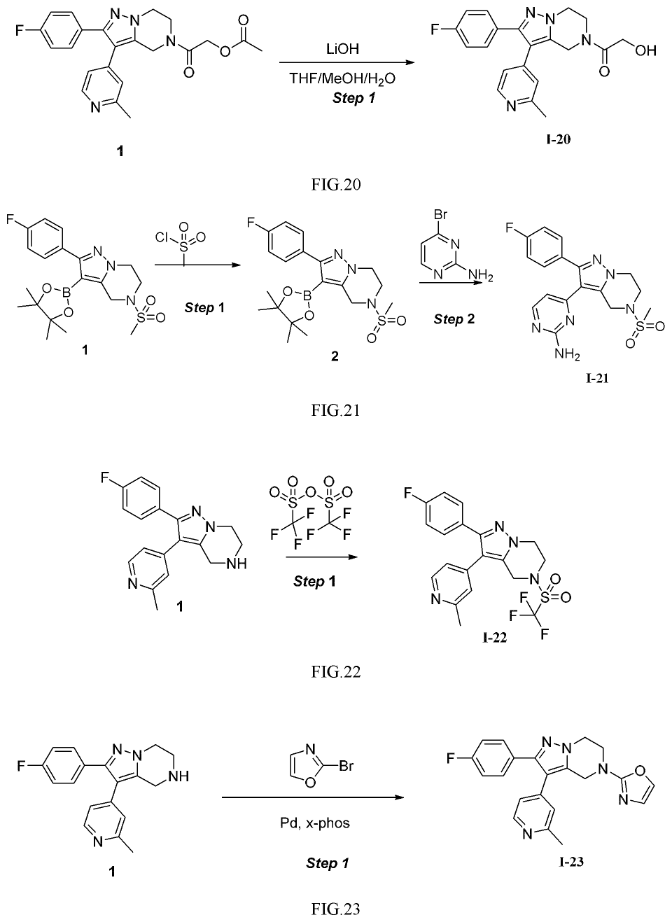

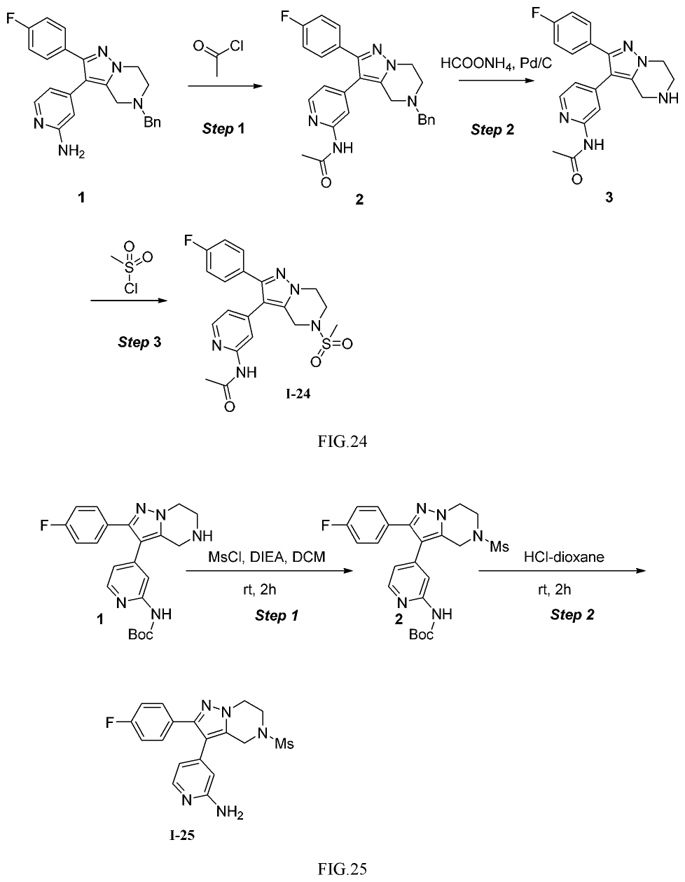

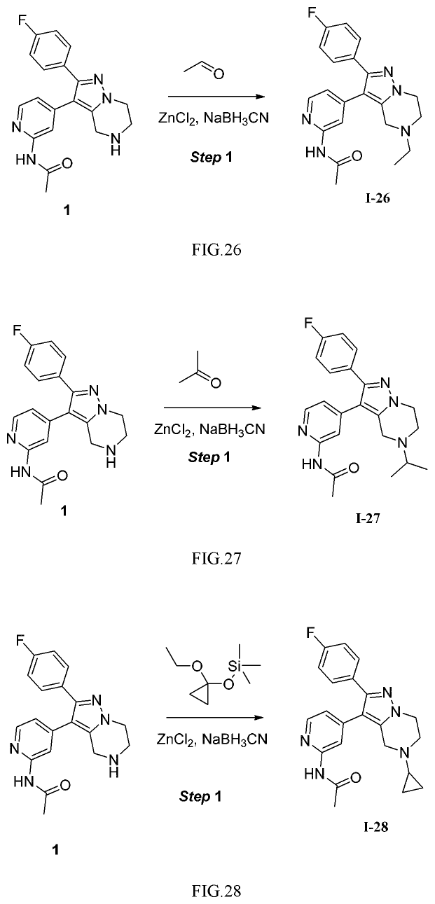

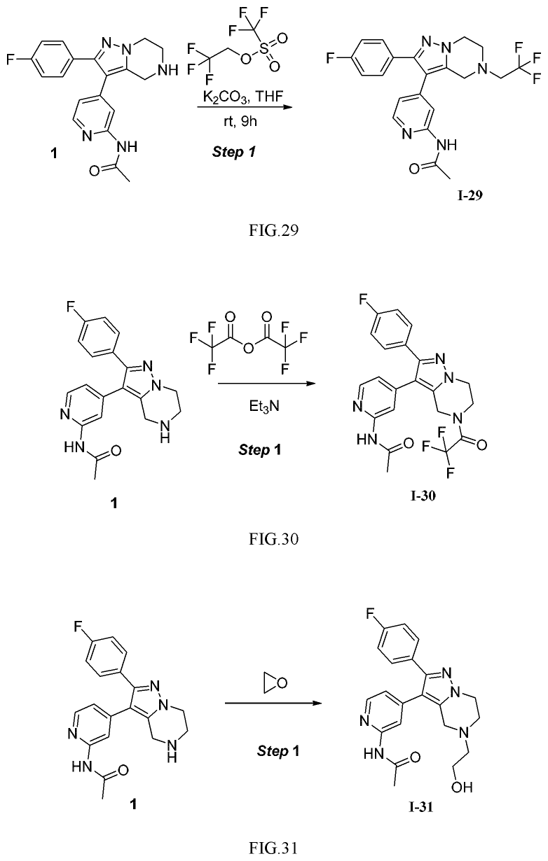

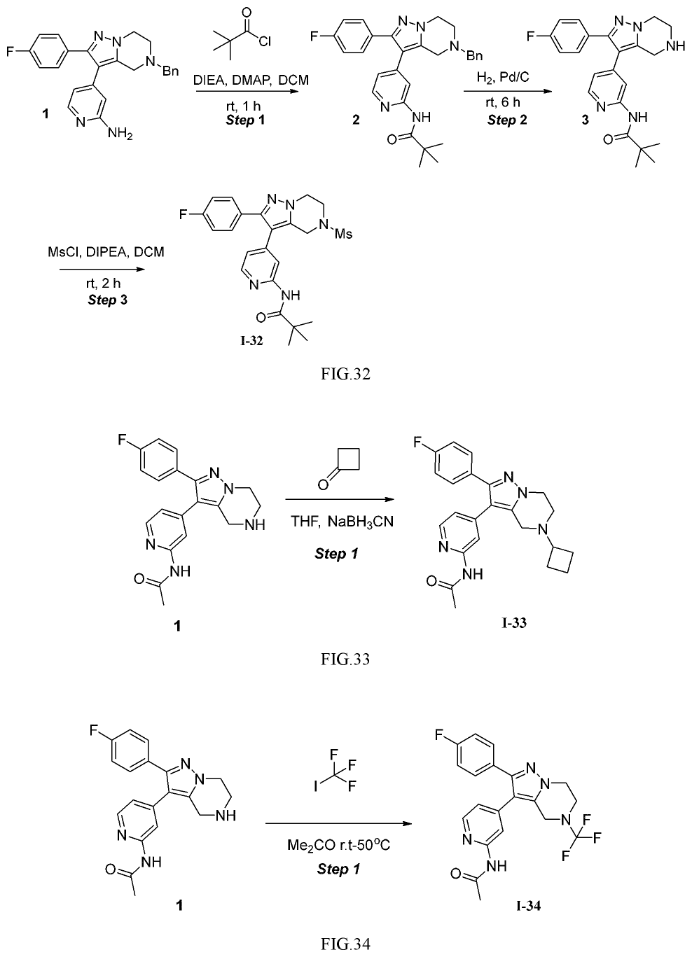

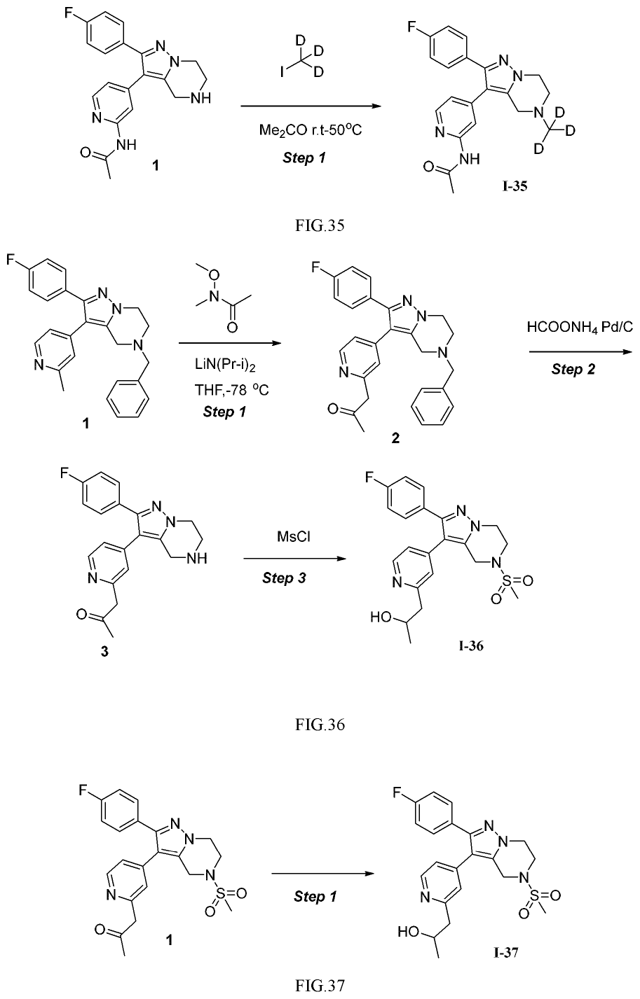

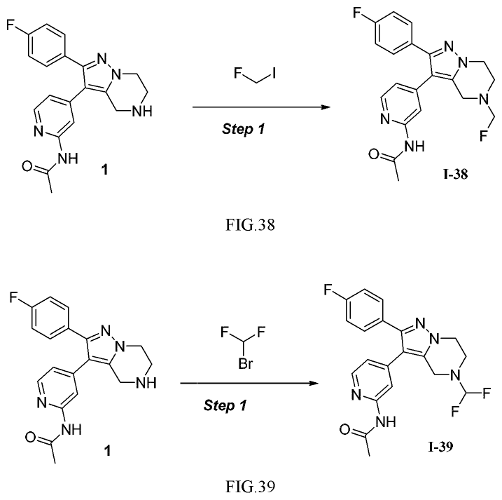

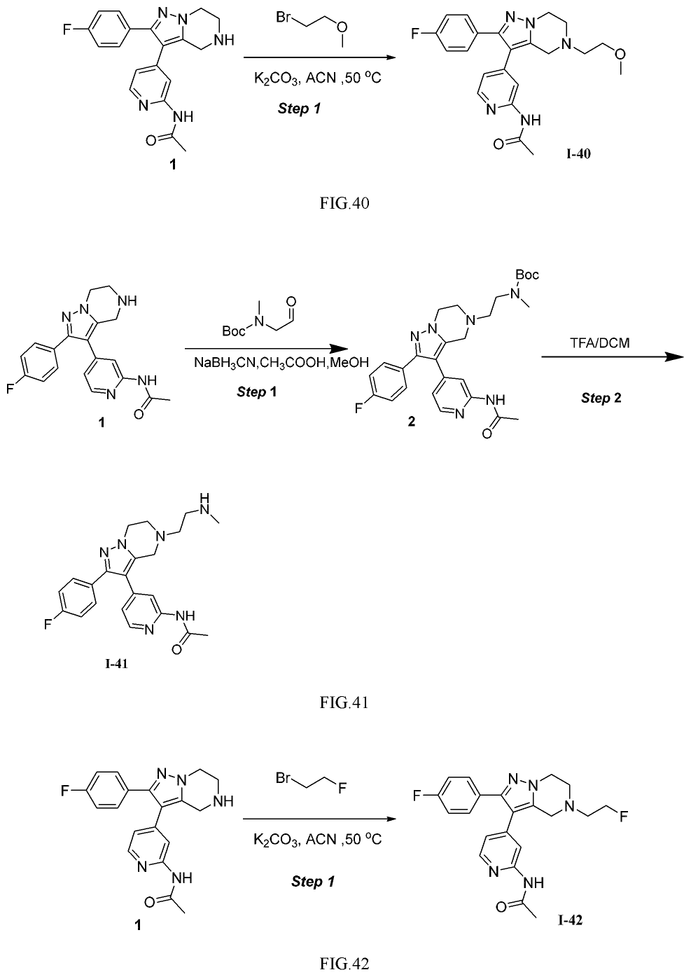

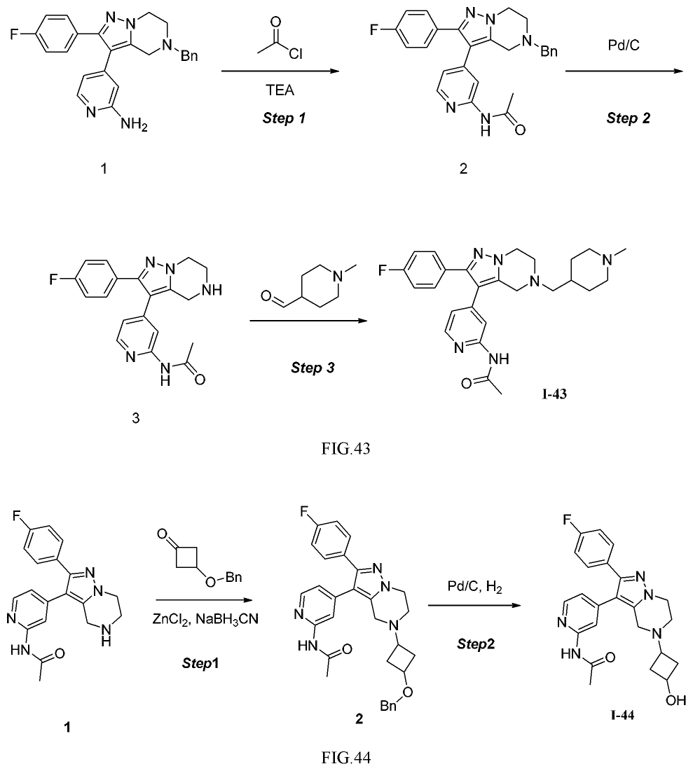

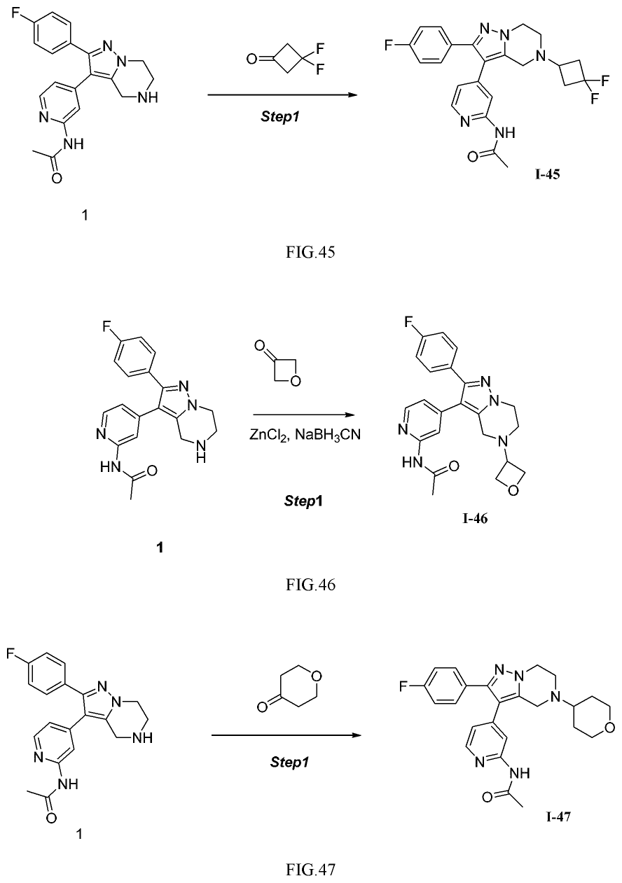

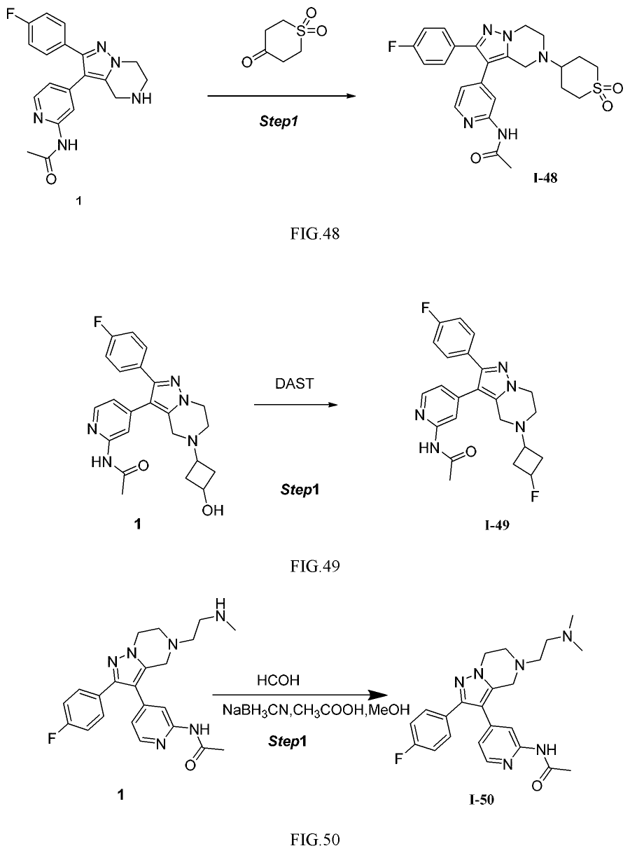

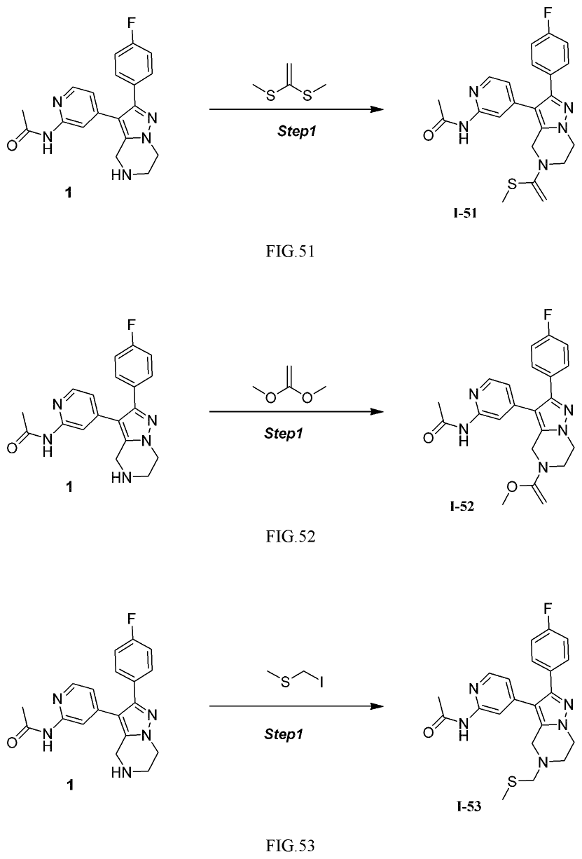

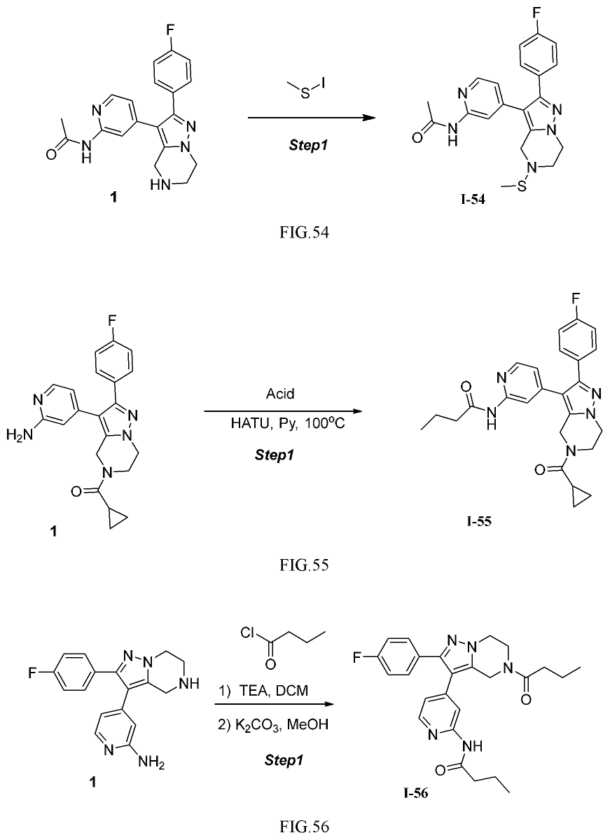

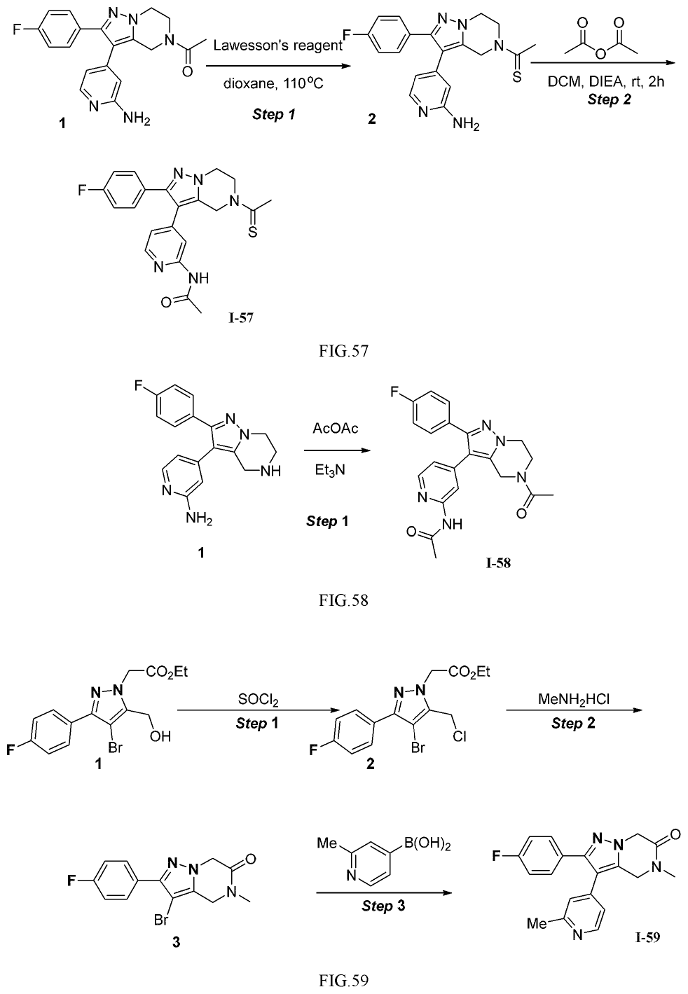

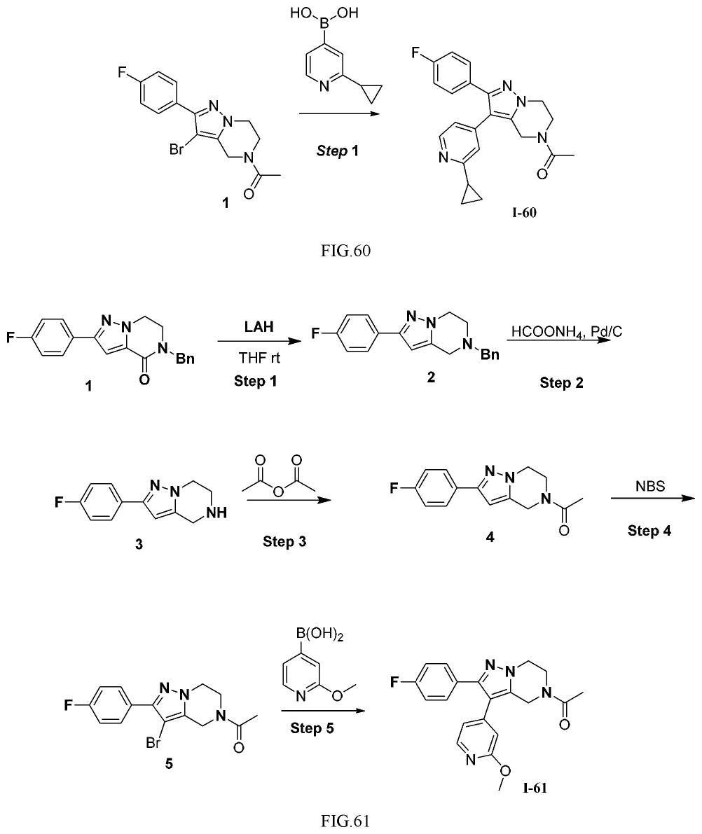

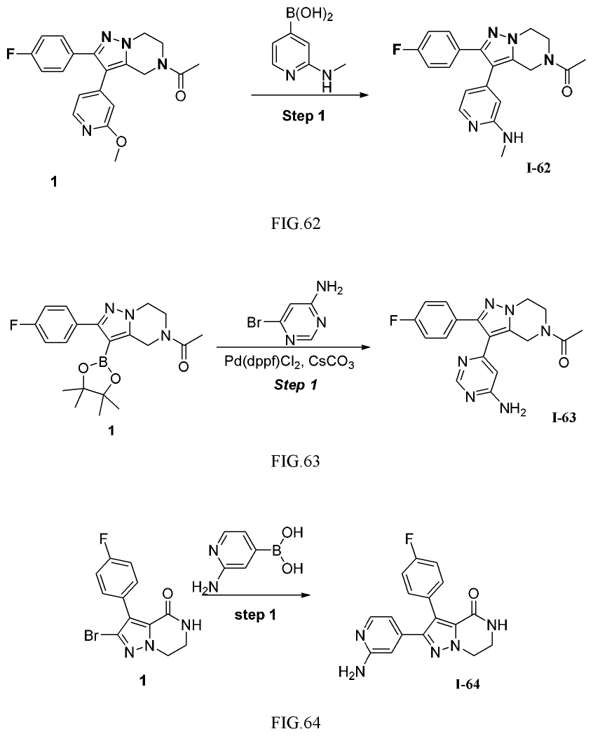

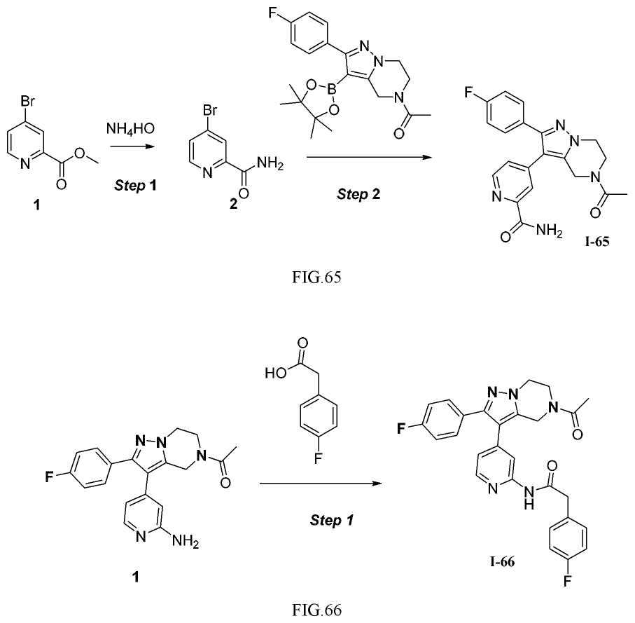

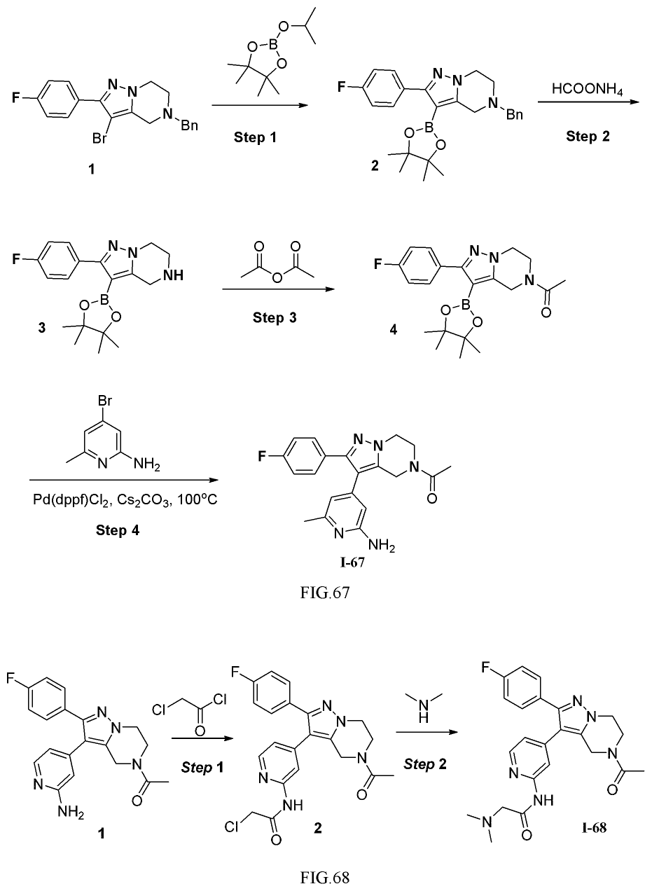

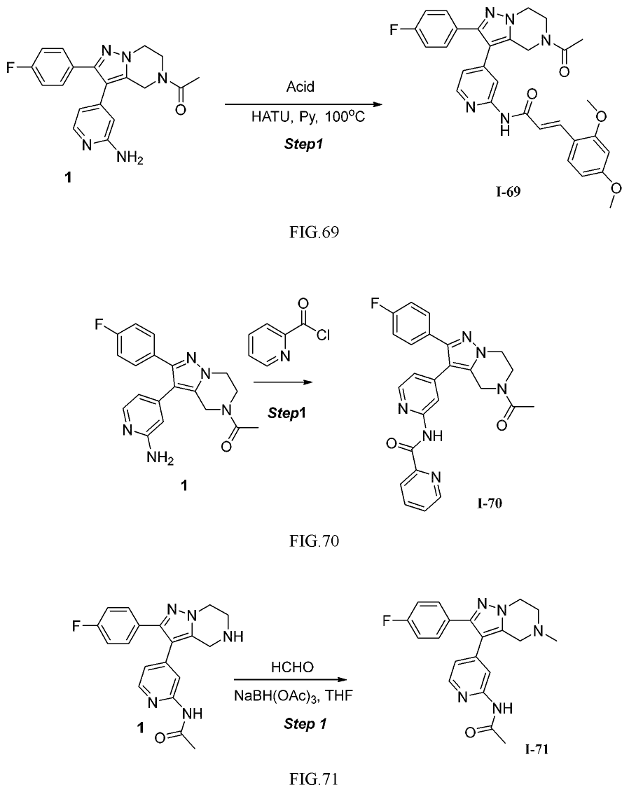

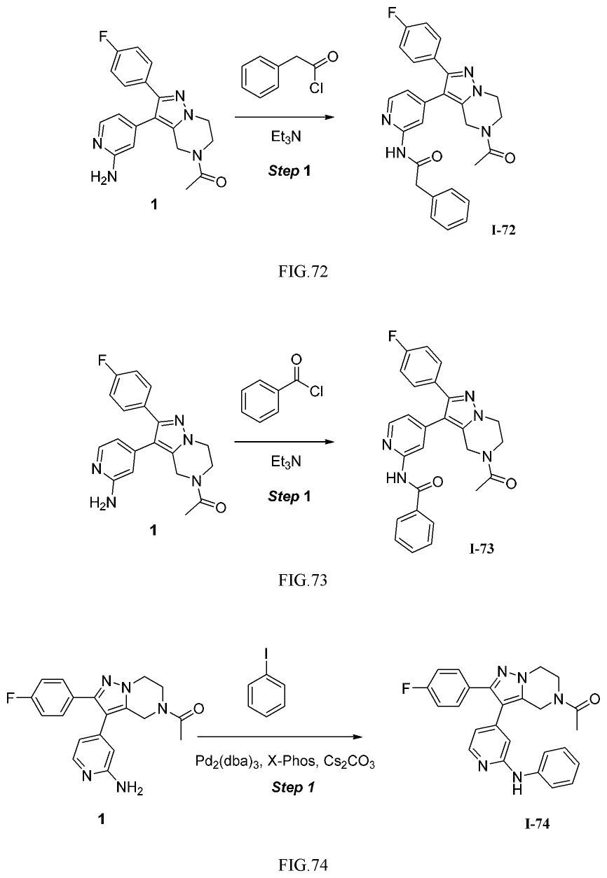

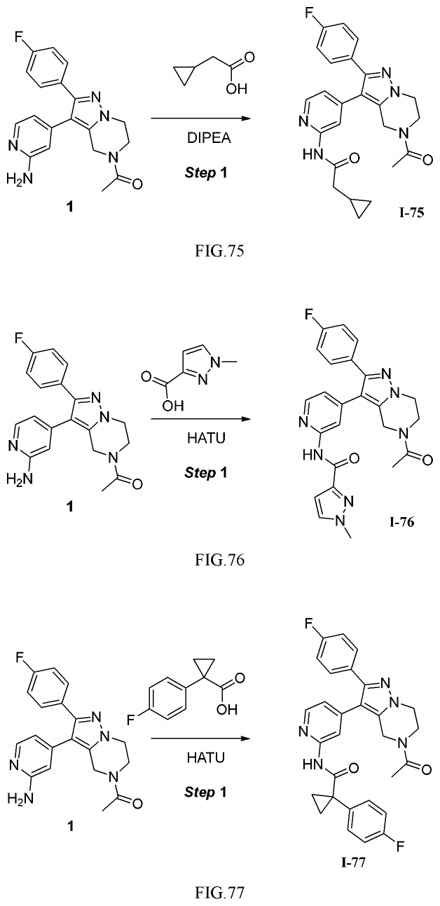

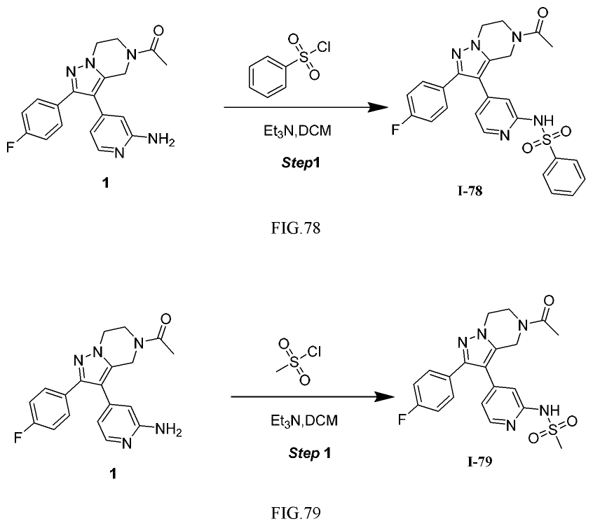

FIG. 1 illustrates the synthetic scheme of compound I-1. As shown in FIG. 1, the specific synthesis step is as follows:

### Step 1: 1-(2-(4-fluorophenyl)-3-(pyridin-4-yl-6,7-dihydropyrazolo[1,5-a]pyrazin-5(4H)-yl)-2-methoxyethan-1-one

To a mixture of 2-(4-fluorophenyl)-3-(pyridin-4-yl)-4,5,6,7-tetrahydropyrazolo[1,5-a]pyrazine (30 mg, 0.1 mmol) in DCM (10 mL) was added DIPEA (38.7 mg, 0.3 mmol) at 25° C., then 2-methoxyacetyl chloride (11.94 mg, 0.11 mmol) in DCM (2 mL) was dropwise in the mixture at −20° C. The reaction was stirred at −20° C. for 0.5 h, LCMS showed desired MS was detected as main peak. The mixture was quenched with 2 drops MeOH and extracted with DCM (10 mL*2) and H₂O (20 mL). The organic layer was washed with brine (20 mL), dried over anhydrous Na₂SO₄, filtered and concentrated, then purified by column chromatography (DCM/MeOH=20/1) to give a white solid (4.4 mg, yield: 11.8%). Chemical Formula: calculated for (M+H⁺) C₂₀H₁₉FN₄O₂: 366.15, Found: 367. ¹H NMR (400 MHz, MeOD) δ 8.69 (d, J=4.9 Hz, 2H), 7.73 (d, J=5.6 Hz, 2H), 7.52-7.44 (m, 2H), 7.18 (t, J=8.7 Hz, 2H), 5.04 (s, 2H), 4.40 (d, J=4.9 Hz, 1H), 4.34 (s, 2H), 4.28 (s, 1H), 4.24-4.09 (m, 2H), 3.42 (d, J=29.7 Hz, 3H).

### Example 1-2 Preparation of Compound 1-2

FIG. 2 illustrates the synthetic scheme of compound I-2. As shown in FIG. 2, the specific synthesis step is as follows:

### Step 1: 2-(4-fluorophenyl)-3-(2-methylpyridin-4-yl)-5-(phenylsulfonyl)-4,5,6,7-tetrahydropyrazolo[1,5-a]pyrazine

To a mixture of 2-(4-fluorophenyl)-3-(2-methylpyridin-4-yl)-4,5,6,7-tetrahydropyrazolo[1,5-a]pyrazine (85 mg, 0.28 mmol) in DCM (10 mL) was added DIPEA (108.5 mg, 0.84 mmol) at 25° C., then benzenesulfonyl chloride (59.3 mg, 0.34 mmol) in DCM (2 mL) was dropwised in the mixture at −20° C. The reaction was stirred at −20° C. for 0.5 h. LCMS showed desired MS was detected as main peak. The mixture was quenched with 2 drops MeOH and extracted with DCM (10 mL*2) and H₂O (20 mL). The organic layer was washed with brine (20 mL), dried over anhydrous Na₂SO₄, filtered and concentrated, then purified by column chromatography (DCM/MeOH=15/1) to give a white solid (12.4 mg, yield: 9.8%). Chemical Formula: calculated for (M+H⁺) C₂₄H₂₁FN₄O₂S: 448.14, Found: 449 ¹H NMR (400 MHz, CDCl₃) δ 8.49 (d, J=5.1 Hz, 1H), 7.88-7.83 (m, 2H), 7.69 (dd, J=8.4, 6.4 Hz, 1H), 7.61 (t, J=7.5 Hz, 2H), 7.39-7.32 (m, 2H), 7.04-6.97 (m, 2H), 6.94-6.85 (m, 2H), 4.41-4.33 (m, 4H), 3.74-3.68 (m, 2H), 2.57 (s, 3H).

### Example 1-3 Preparation of Compound I-3

FIG. 3 illustrates the synthetic scheme of compound I-3. As shown in FIG. 3, the specific synthesis step is as follows:

### Step 1: (2-(4-fluorophenyl)-3-(2-methylpyridin-4-yl)-6,7-dihydropyrazolo[1,5-a]pyrazin-5(4H)-yl)(pyridin-3-yl)methanone

To a mixture of 2-(4-fluorophenyl)-3-(2-methylpyridin-4-yl)-4,5,6,7-tetrahydropyrazolo[1,5-a]pyrazine (80 mg, 0.26 mmol) in DCM (20 mL) was added DIPEA (167.7 mg, 1.3 mmol), T₃P (513 mg, 0.65 mmol) and pyridine-3-carboxylic acid (38.5 mg, 0.31 mmol) at 25° C. The reaction was stirred at 25° C. for 1 h. LCMS showed desired MS was detected as main peak. The mixture was extracted with DCM (10 mL*2) and H₂O (20 mL). The organic layer was washed with brine (20 mL), dried over anhydrous Na₂SO₄, filtered and concentrated, then purified by column chromatography (DCM/MeOH=25/1) to give a white solid (11.1 mg, yield: 10.8%). Chemical Formula: calculated for (M+H⁺) C₂₄H₂₀FN₅O: 413.17, Found: 414 ¹H NMR (400 MHz, CDCl₃) δ 8.78 (dd, J=6.7, 3.2 Hz, 2H), 8.51 (s, 1H), 7.92-7.85 (m, 1H), 7.51-7.35 (m, 3H), 7.04 (dd, J=20.1, 11.4 Hz, 4H), 4.98 (s, 2H), 4.41 (s, 2H), 4.10 (s, 2H), 2.59 (s, 3H).

### Example 1-4 Preparation of Compound I-4

FIG. 4 illustrates the synthetic scheme of compound I-4. As shown in FIG. 4, the specific synthesis step is as follows:

### Step 1: (2-(4-fluorophenyl)-3-(2-methylpyridin-4-yl)-6,7-dihydropyrazolo[1,5-a]pyrazin-5(4H)-yl)(pyridin-4-yl)methanone

To a mixture of 2-(4-fluorophenyl)-3-(2-methylpyridin-4-yl)-4,5,6,7-tetrahydropyrazolo[1,5-a]pyrazine (80 mg, 0.26 mmol) in DCM (20 mL) was added DIPEA (167.7 mg, 1.3 mmol), T₃P (513 mg, 0.65 mmol) and pyridine-4-carboxylic acid (38.5 mg, 0.31 mmol) at 25° C. The reaction was stirred at 25° C. for 1 h. LCMS showed desired MS was detected as main peak. The mixture was extracted with DCM (10 mL*2) and H₂O (20 mL). The organic layer was washed with brine (20 mL), dried over anhydrous Na₂SO₄, filtered and concentrated, then purified by column chromatography (DCM/MeOH=25/1) to give a white solid (11.1 mg, yield: 10.8%). Chemical Formula: calculated for (M+H⁺) C₂₄H₂₀FN₅O: 413.17, Found: 41411H NMR (400 MHz, CDCl₃) δ 8.80 (s, 2H), 8.48 (s, 1H), 7.38 (d, J=4.6 Hz, 4H), 7.04 (t, J=8.6 Hz, 4H), 5.04 (s, 2H), 4.35 (s, 2H), 3.95 (s, 2H), 2.60 (s, 3H).

### Example 1-5 Preparation of Compound I-5

FIG. 5 illustrates the synthetic scheme of compound I-5. As shown in FIG. 5, the specific synthesis step is as follows:

### Step 1:(4-fluorophenyl)(2-(4-fluorophenyl)-3-(2-methylpyridin-4-yl)-6,7-dihydropyrazolo[1,5-a]pyrazin-5(4H)-yl)methanone

To a mixture of 2-(4-fluorophenyl)-3-(2-methylpyridin-4-yl)-4,5,6,7-tetrahydropyrazolo[1,5-a]pyrazine (80 mg, 0.26 mmol) in DCM (20 mL) was added DIPEA (167.7 mg, 1.3 mmol), T₃P (513 mg, 0.65 mmol) and 4-fluorobenzoic acid (43.7 mg, 0.31 mmol) at 25° C. The reaction was stirred at 25° C. for 1 h. LCMS showed desired MS was detected as main peak. The mixture was extracted with DCM (10 mL*2) and H₂O (20 mL). The organic layer was washed with brine (20 mL), dried over anhydrous Na₂SO₄, filtered and concentrated, then purified by column chromatography (DCM/MeOH=25/1) to give a white solid (12.2 mg, yield: 10.8%). Chemical Formula: calculated for (M+H⁺) C₂₃H₂₀F₂N₄O: 430.16, Found: 431 ¹H NMR (400 MHz, DMSO) δ 8.40 (s, 1H), 7.65-7.56 (m, 2H), 7.43-7.36 (m, 2H), 7.32 (t, J=8.8 Hz, 2H), 7.20 (t, J=8.9 Hz, 2H), 7.00 (d, J=52.5 Hz, 2H), 4.86 (s, 2H), 4.31 (t, J=5.4 Hz, 2H), 3.93 (a, 2H), 2.43 (s, 3H).

### Example 1-6 Preparation of Compound I-6

FIG. 6 illustrates the synthetic scheme of compound I-6. As shown in FIG. 6, the specific synthesis step is as follows:

### Step 1:3-(dimethylamino)-1-(2-(4-fluorophenyl)-3-(2-methylpyridin-4-yl)-6,7-dihydropyrazolo[1,5-a]pyrazin-5(4H)-yl)propan-1-one

To a mixture of 2-(4-fluorophenyl)-3-(2-methylpyridin-4-yl)-4,5,6,7-tetrahydropyrazolo[1,5-a]pyrazine (80 mg, 0.26 mmol) in DCM (20 mL) was added DIPEA (167.7 mg, 1.3 mmol), T₃P (513 mg, 0.65 mmol) and 3-(dimethylamino)propanoic acid (36.5 mg, 0.31 mmol) at 25° C. The reaction was stirred at 25° C. for 1 h. LCMS showed desired MS was detected as main peak. The mixture was extracted with DCM (10 mL*2) and H₂O (20 mL). The organic layer was washed with brine (20 mL), dried over anhydrous Na₂SO₄, filtered and concentrated, then purified by column chromatography (DCM/MeOH=20/1) to give a white solid (3.8 mg, yield: 3.6%). Chemical Formula: calculated for (M+H⁺) C₂₃H₂₆FN₅O: 407.21, Found: 408 ¹H NMR (400 MHz, CDCl₃) δ 8.55-8.32 (m, 2H), 7.45-7.34 (m, 2H), 7.02 (t, J=8.6 Hz, 2H), 6.91 (dd, J=13.8, 4.7 Hz, 1H), 4.83 (d, J=21.2 Hz, 2H), 4.23 (dd, J=87.9, 36.6 Hz, 4H), 3.27-2.87 (m, 4H), 2.69-2.52 (m, 9H).

### Example 1-7 Preparation of Compound I-7

FIG. 7 illustrates the synthetic scheme of compound I-7. As shown in FIG. 7, the specific synthesis step is as follows:

### Step 1: 2-(4-fluorophenyl)-3-(2-methylpyridin-4-yl-5-(methylsulfonyl)-4,5,6,7-tetrahydropyrazolo[1,5-a]pyrazine

To a mixture of 2-(4-fluorophenyl)-3-(2-methylpyridin-4-yl)-4,5,6,7-tetrahydropyrazolo[1,5-a]pyrazine (85 mg, 0.28 mmol) in DCM (10 mL) was added DIPEA (108.5 mg, 0.84 mmol) at 25° C., then methanesulfonyl chloride (0.027 ml, 0.34 mmol) in DCM (2 mL) was dropwised in the mixture at −20° C. The reaction was stirred at −20° C. for 0.5 h. LCMS showed desired MS was detected as main peak. The mixture was quenched with 2 drops MeOH and extracted with DCM (10 mL*2) and H₂O (20 mL). The organic layer was washed with brine (20 mL), dried over anhydrous Na₂SO₄, filtered and concentrated, then purified by column chromatography (DCM/MeOH=20/1) to give a white solid (10.1 mg, yield: 9.3%). Chemical Formula: calculated for (M+H⁺) C₁₉H₁₉FN₄O₂S: 386.12, Found: 387. ¹H NMR (400 MHz, CDCl₃) δ 8.46 (d, J=5.2 Hz, 1H), 7.40-7.35 (m, 2H), 7.04-6.98 (m, 2H), 6.97-6.88 (m, 2H), 4.57 (s, 2H), 4.43-4.38 (m, 2H), 3.90-3.85 (m, 2H), 2.96 (s, 3H), 2.56 (s, 3H).

### Example 1-8 Preparation of Compound I-8

FIG. 8 illustrates the synthetic scheme of compound I-8. As shown in FIG. 8, the specific synthesis step is as follows:

### Step 1:1-(2-(4-fluorophenyl)-3-(2-methylpyridin-4-yl)-6,7-dihydropyrazolo[1,5-a]pyrazin-5(4H)-yl)-2,2-dimethylpropan-1-one

To a mixture of 2-(4-fluorophenyl)-3-(2-methylpyridin-4-yl)-4,5,6,7-tetrahydropyrazolo[1,5-a]pyrazine (8 mg, 0.2 mmol) in DCM (10 mL) was added DIPEA (100.4 mg, 0.78 mmol) at 25° C., then 2,2-dimethylpropanoyl chloride (37.5 mg, 0.31 mmol) in DCM (2 mL) was dropwised in the mixture at −20° C. The reaction was stirred at −20° C. for 0.5 h. LCMS showed desired MS was detected as main peak. The mixture was quenched with 2 drops MeOH and extracted with DCM (10 mL*2) and H₂O (20 mL). The organic layer was washed with brine (20 mL), dried over anhydrous Na₂SO₄, filtered and concentrated, then purified by column chromatography (DCM/MeOH=15/1) to give a white solid (12.8 mg, yield: 12.5%). Chemical Formula: calculated for (M+H⁺) C₂₃H₂₅FN₄O: 392.20, Found: 393. ¹H NMR (400 MHz, CDCl3) δ 8.53 (d, J=4.9 Hz, 1H), 7.39 (dd, J=8.6, 5.4 Hz, 2H), 7.10-7.02 (m, 4H), 4.91 (s, 2H), 4.35 (t, J=5.4 Hz, 2H), 4.21 (t, J=5.4 Hz, 2H), 2.67 (s, 3H), 1.34 (s, 9H).

### Example 1-9 Preparation of Compound I-9

FIG. 9 illustrates the synthetic scheme of compound I-9. As shown in FIG. 9, the specific synthesis step is as follows:

### Step 1: 2-(4-fluorophenyl)-3-(2-methylpyridin-4-yl)-5-(pyrimidin-2-ylmethyl)-4,5,6,7-tetrahydropyrazolo[1,5-a]pyrazine

NaBH(OAc)₃ (101 mg, 0.48 mmol) was added to a solution of 2-(4-fluorophenyl)-3-(2-methylpyridin-4-yl)-4,5,6,7-tetrahydropyrazolo[1,5-a]pyrazine (50 mg, 0.16 mmol) and 2-pyrimidinecarbaldehyde (26 mg, 0.24 mmol) in THF (3 mL). The reaction mixture was stirred at 25° C. for 2 h. The solution was filtered, filtrate was collected. The reaction mixture was concentrated under pressure. The residue was purified via Genal-Prep-HPLC and give the product.

¹H NMR (400 MHz, MeOD) δ 8.81 (d, J=5.0 Hz, 2H), 8.31 (d, J=5.3 Hz, 1H), 7.47-7.35 (m, 3H), 7.10 (dd, J=14.8, 5.9 Hz, 3H), 7.02-6.96 (m, 1H), 4.31 (t, J=5.5 Hz, 2H), 4.11 (s, 2H), 4.01 (s, 2H), 3.26 (t, J=5.5 Hz, 2H), 2.47 (s, 3H).

### Example 1-16 Preparation of Compound I-10

FIG. 10 illustrates the synthetic scheme of compound I-10. As shown in FIG. 10, the specific synthesis step is as follows:

### Step 1: 3-((2-(4-fluorophenyl)-3-(2-methylpyridin-4-yl)-6,7-dihydropyrazolo[1,5-a]pyrazin-5(4H)-yl)methyl)isoxazole

NaBH(OAc)₃ (101 mg, 0.48 mmol) was added to a solution of 2-(4-fluorophenyl)-3-(2-methylpyridin-4-yl)-4,5,6,7-tetrahydropyrazolo[1,5-a]pyrazine (50 mg, 0.16 mmol) and isoxazole-3-carbaldehyde (23.3 mg, 0.24 mmol) in THF (3 mL). The reaction mixture was stirred at 25° C. for 2 h. The solution was filtered, filtrate was collected. The reaction mixture was concentrated under pressure. The residue was purified via Genal-Prep-HPLC and give the product.

¹H NMR (400 MHz, MeOD) δ 8.66 (d, J=1.6 Hz, 1H), 8.32 (d, J=5.3 Hz, 1H), 7.44-7.34 (m, 2H), 7.09 (dd, J=15.1, 6.3 Hz, 3H), 6.98 (dd, J=5.3, 1.2 Hz, 1H), 6.56 (d, J=1.7 Hz, 1H), 4.28 (t, J=5.5 Hz, 2H), 3.97 (s, 2H), 3.88 (s, 2H), 3.14 (t, J=5.6 Hz, 2H), 2.48 (s, 3H).

### Example 1-11 Preparation of Compound I-11

FIG. 11 illustrates the synthetic scheme of compound I-11. As shown in FIG. 11, the specific synthesis step is as follows:

### Step 1: 2-(2-(4-fluorophenyl)-3-(2-methylpyridin-4-yl-6,7-dihydropyrazolo[1,5-a]pyrazin-5(4H)-yl)ethan-1-ol

To a solution of 2-(4-fluorophenyl)-3-(2-methylpyridin-4-yl)-4,5,6,7-tetrahydropyrazolo[1,5-a]pyrazine (50 mg, 0.16 mmol) in EtOH (5 mL), a solution of oxirane in THF (0.02 mL, 0.032 mmol, 2 mmol/L) was added. The reaction mixture was stirred at 50° C. for 2 h. The reaction mixture was concentrated under pressure. The residue was purified via Genal-Prep-HPLC and give the product.

¹H NMR (400 MHz, MeOD) δ 8.31 (d, J=5.1 Hz, 1H), 7.43-7.34 (m, 2H), 7.12-7.04 (m, 3H), 6.98 (d, J=5.3 Hz, 1H), 4.27 (t, J=5.5 Hz, 2H), 3.87 (s, 2H), 3.74 (t, J=5.5 Hz, 2H), 3.15 (t, J=5.4 Hz, 2H), 2.79 (t, J=5.4 Hz, 2H), 2.46 (s, 3H).

### Example 1-12 Preparation of Compound I-12

FIG. 12 illustrates the synthetic scheme of compound I-12. As shown in FIG. 12, the specific synthesis step is as follows:

### Step 1: 2-(4-fluorophenyl)-3-(2-methylpyridin-4-yl)-5-(oxetan-3-yl)-4,5,6,7-tetrahydropyrazolo[1,5-a]pyrazine

NaBH(OAc)₃ (101 mg, 0.48 mmol) was added to a solution of 2-(4-fluorophenyl)-3-(2-methylpyridin-4-yl)-4,5,6,7-tetrahydropyrazolo[1,5-a]pyrazine (5 mg, 0.0162 mmol) and 3-oxetanone (17.3 mg, 0.24 mmol) in THF (3 mL). The reaction mixture was stirred at 25° C. for 2 h. The solution was filtered, filtrate was collected. The reaction mixture was concentrated under pressure. The residue was purified via Genal-Prep-HPLC and give the product.

¹H NMR (400 MHz, MeOD) δ 8.30 (d, J=5.3 Hz, 1H), 7.43-7.35 (m, 2H), 7.09 (dd, J=12.3, 5.5 Hz, 3H), 6.98 (d, J=5.3 Hz, 1H), 4.76 (t, J=6.7 Hz, 2H), 4.64 (t, J=6.2 Hz, 2H), 4.28 (t, J=5.5 Hz, 2H), 3.86 (dd, J=12.5, 6.2 Hz, 1H), 3.70 (s, 2H), 3.03-2.93 (m, 2H), 2.46 (s, 3H).

### Example 1-13 Preparation of Compound I-13

FIG. 13 illustrates the synthetic scheme of compound I-13. As shown in FIG. 13, the specific synthesis step is as follows:

### Step 1:(2-(4-fluorophenyl-3-(2-methylpyridin-4-yl)-6,7-dihydropyrazolo[1,5-a]pyrazin-5(4H)-yl)(tetrahydro-2H-pyran-4-yl)methanone

To a mixture of 2-(4-fluorophenyl)-3-(2-methylpyridin-4-yl)-4,5,6,7-tetrahydropyrazolo[1,5-a]pyrazine (80 mg, 0.26 mmol) in DCM (20 mL) was added DIPEA (167.7 mg, 1.3 mmol), T₃P (513 mg, 0.65 mmol) and oxane-4-carboxylic acid (40.6 mg, 0.31 mmol) at 25° C. The reaction was stirred at 25° C. for 1 h, LCMS showed desired MS was detected as main peak. The mixture was extracted with DCM (10 mL*2) and H₂O (20 mL). The organic layer was washed with brine (20 mL), dried over anhydrous Na₂SO₄, filtered and concentrated, then purified by column chromatography (DCM/MeOH=25/1) to give a white solid (10.7 mg, yield: 9.7%). Chemical Formula: calculated for (M+H⁺) C₂₄H₂₅FN₄O₂: 420.49, Found: 421. ¹H NMR (400 MHz, CDCl3) δ 8.47 (s, 1H), 7.43-7.31 (m, 2H), 7.00 (dd, J=23.3, 14.6 Hz, 4H), 4.83 (d, J=39.4 Hz, 2H), 4.41-3.95 (m, 6H), 3.47 (d, J=10.0 Hz, 2H), 2.79 (d, J=47.1 Hz, 1H), 2.57 (s, 3H), 1.95 (s, 2H), 1.66 (d, J=12.4 Hz, 2H).

### Example 1-14 Preparation of Compound I-14

FIG. 14 illustrates the synthetic scheme of compound I-14. As shown in FIG. 14, the specific synthesis step is as follows:

### Step 1:(2-(4-fluorophenyl-3-(2-methylpyridin-4-yl)-6,7-dihydropyrazolo[1,5-a]pyrazin-5(4H)-yl)(tetrahydrofuran-3-yl)methanone

To a mixture of 2-(4-fluorophenyl)-3-(2-methylpyridin-4-yl)-4,5,6,7-tetrahydropyrazolo[1,5-a]pyrazine (80 mg, 0.26 mmol) in DCM (10 mL) was added DIPEA (100.6 mg, 0.78 mmol) at 25° C., then oxolane-3-carbonyl chloride (42 mg, 0.312 mmol) in DCM (2 mL) was dropwised in the mixture at −20° C. The reaction was stirred at −20° C. for 0.5 h, LCMS showed desired MS was detected as main peak. The mixture was quenched with 2 drops MeOH and extracted with DCM (10 mL*2) and H₂O (20 mL). The organic layer was washed with brine (20 mL), dried over anhydrous Na₂SO₄, filtered and concentrated, then purified by column chromatography (DCM/MeOH=20/1) to give crude product, then purified by SFC (chiralpak-OJ, CO2-MEOH (DEA) to give Compound I-14 (P1=12.8 mg, yield: 11.9%) and Compound I-15 (P2=10.6 mg, yield: 10%), Chemical Formula: calculated for (M+H⁺) C₂₃H₂₃FN₄O₂: 406.18, Found: 407.

Compound I-14: ¹H NMR (400 MHz, CDCl₃) δ 8.52-8.41 (m, 1H), 7.38 (dd, J=8.7, 5.4 Hz, 2H), 6.99 (dd, J=29.5, 20.7 Hz, 4H), 4.89 (d, J=8.4 Hz, 1H), 4.76 (s, 1H), 4.33 (d, J=26.5 Hz, 2H), 4.19 (s, 1H), 4.08 (s, 2H), 3.91 (d, J=7.6 Hz, 3H), 3.28 (d, J=54.8 Hz, 1H), 2.56 (s, 3H), 2.27-2.07 (m, 2H).

### Example 1-15 Preparation of Compound I-15

FIG. 15 illustrates the synthetic scheme of compound I-15. As shown in FIG. 15, the specific synthesis step is as follows:

### Step 1:(2-(4-fluorophenyl-3-(2-methylpyridin-4-yl)-6,7-dihydropyrazolo[1,5-a]pyrazin-5(4H)-yl)(tetrahydrofuran-3-yl)methanone

To a mixture of 2-(4-fluorophenyl)-3-(2-methylpyridin-4-yl)-4,5,6,7-tetrahydropyrazolo[1,5-a]pyrazine (80 mg, 0.26 mmol) in DCM (10 mL) was added DIPEA (100.6 mg, 0.78 mmol) at 25° C., then oxolane-3-carbonyl chloride (42 mg, 0.312 mmol) in DCM (2 mL) was dropwised in the mixture at −20° C. The reaction was stirred at −20° C. for 0.5 h, LCMS showed desired MS was detected as main peak. The mixture was quenched with 2 drops MeOH and extracted with DCM (10 mL*2) and H₂O (20 mL). The organic layer was washed with brine (20 mL), dried over anhydrous Na₂SO₄, filtered and concentrated, then purified by column chromatography (DCM/MeOH=20/1) to give crude product, then purified by SFC (chiralpak-OJ, CO2-MEOH (DEA) to give Compound I-14 (P1=12.8 mg, yield: 11.9%) and Compound I-15 (P2=10.6 mg, yield: 10%), Chemical Formula: calculated for (M+H⁺) C₂₃H₂₃FN₄O₂: 406.18, Found: 407.

Compound I-15: ¹H NMR (400 MHz, CDCl₃) δ 8.43 (d, J=4.6 Hz, 1H), 7.41-7.35 (m, 2H), 7.05-6.88 (m, 4H), 4.91-4.74 (m, 2H), 4.33 (d, J=25.2 Hz, 2H), 4.14 (d, J=43.9 Hz, 3H), 3.93 (dd, J=18.2, 7.2 Hz, 3H), 3.36-3.18 (m, 1H), 2.55 (s, 3H), 2.18 (d, J=7.5 Hz, 2H).

### Example 1-16 Preparation of Compound I-16

FIG. 16 illustrates the synthetic scheme of compound I-16. As shown in FIG. 16, the specific synthesis step is as follows:

### Step 1:2,2,2-trifluoro-1-(2-(4-fluorophenyl-3-(2-methylpyridin-4-yl)-6,7-dihydropyrazolo[1,5-a]pyrazin-5(4H)-yl)ethan-1-one

To a mixture of 2-(4-fluorophenyl)-3-(2-methylpyridin-4-yl)-4,5,6,7-tetrahydropyrazolo[1,5-a]pyrazine (80 mg, 0.26 mmol) in DCM (20 mL) was added DIPEA (67 mg, 0.52 mmol) and 2,2,2-trifluoroacetyl 2,2,2-trifluoroacetate (109 mg, 0.52 mmol) at 25° C. The reaction was stirred at 25° C. for 1 h. LCMS showed desired MS was detected as main peak. The mixture was extracted with DCM (10 mL*2) and H₂O (20 mL). The organic layer was washed with brine (20 mL), dried over anhydrous Na₂SO₄, filtered and concentrated, then purified by column chromatography (DCM/MeOH=20/1) to give a white solid (10.7 mg, yield: 10.1%). Chemical Formula: calculated for (M+H⁺) C₂₀H₁₆F₄N₄O: 404.13, Found: 405 ¹H NMR (400 MHz, CDCl₃) δ 8.50 (dd, J=11.7, 5.1 Hz, 1H), 7.37 (ddd, J=8.2, 5.3, 2.6 Hz, 2H), 7.06-6.88 (m, 4H), 4.89 (d, J=25.9 Hz, 2H), 4.44-4.37 (m, 2H), 4.21 (dt, J=10.6, 5.5 Hz, 2H), 2.57 (s, 3H).

### Example 1-17 Preparation of Compound I-17

FIG. 17 illustrates the synthetic scheme of compound I-17. As shown in FIG. 17, the specific synthesis step is as follows:

### Step 1: 2-(4-fluorophenyl)-3-(2-methylpyridin-4-yl)-5-(pyridin-2-ylmethyl)-4,5,6,7-tetrahydropyrazolo[1,5-a]pyrazine

To a solution of 2-(4-fluorophenyl)-3-(2-methylpyridin-4-yl)-4,5,6,7-tetrahydropyrazolo[1,5-a]pyrazine (50 mg, 0.16 mmol) and picolinaldehyde (25.7 mg, 0.24 mmol) in THF (5 mL), was added NaBH(OAc)₃ (101 mg, 0.48 mmol) and molecular sieve (100 mg). The reaction mixture was stirred at 50° C. for 2 h. The solution was filtered, filtrate was collected. The reaction mixture was concentrated under pressure. The residue was purified via Genal-Prep-HPLC and give the product.

¹H NMR (400 MHz, MeOD) δ 8.50 (d, J=4.2 Hz, 1H), 8.28 (d, J=5.3 Hz, 1H), 7.82 (dd, J=7.7, 1.7 Hz, 1H), 7.58 (d, J=7.8 Hz, 11H), 7.41-7.31 (m, 3H), 7.12-7.02 (m, 3H), 6.95 (dd, J=5.2, 1.2 Hz, 11H), 4.26 (t, J=5.5 Hz, 2H), 3.94 (a, 2H), 3.86 (s, 2H), 3.12 (t, J=5.6 Hz, 2H), 2.44 (s, 3H).

### Example 1-18 Preparation of Compound I-18

FIG. 18 illustrates the synthetic scheme of compound I-18. As shown in FIG. 18, the specific synthesis step is as follows:

### Step 1: 2-(4-fluorophenyl)-3-(2-methylpyridin-4-yl)-5-(pyridin-3-ylmethyl)-4,5,6,7-tetrahydropyrazolo[1,5-a]pyrazine

NaBH(OAc)₃ (165 mg, 0.78 mmol) and molecular sieve (100 mg) was added to a solution of 2-(4-fluorophenyl)-3-(2-methylpyridin-4-yl)-4,5,6,7-tetrahydropyrazolo[1,5-a]pyrazine (80 mg, 0.26 mmol) and nicotinaldehyde (41.7 mg, 0.39 mmol) in THF (10 mL). The reaction mixture was stirred at 50° C. for 15 h. The solution was filtered, filtrate was collected. The reaction mixture was concentrated under pressure. The residue was purified via Genal-Prep-HPLC and give the product.

¹H NMR (400 MHz, MeOD) δ 8.52 (d, J=40.2 Hz, 2H), 8.28 (d, J=5.2 Hz, 1H), 7.90 (d, J=7.8 Hz, 1H), 7.47-7.41 (m, 1H), 7.41-7.35 (m, 2H), 7.13-7.01 (m, 31H), 6.97-6.93 (m, 1H), 4.24 (t, J=5.5 Hz, 2H), 3.84 (d, J=11.9 Hz, 1H), 3.07 (t, J=5.6 Hz, 2H), 2.44 (s, 3H).

### Example 1-19 Preparation of Compound I-19

FIG. 19 illustrates the synthetic scheme of compound I-19. As shown in FIG. 19, the specific synthesis step is as follows:

### Step 1: 2-(2-(4-fluorophenyl)-3-(2-methylpyridin-4-yl)-6,7-dihydropyrazolo[1,5-a]pyrazin-5(4H)-yl)-2-oxoethyl Acetate

To a mixture of 2-(4-fluorophenyl)-3-(2-methylpyridin-4-yl)-4,5,6,7-tetrahydropyrazolo[1,5-a]pyrazine (80 mg, 0.26 mmol) in DCM (20 mL) was added DIPEA (167.7 mg, 1.3 mmol), T₃P (513 mg, 0.65 mmol) and (acetyloxy) acetic acid (36.85 mg, 0.31 mmol) at 25° C. The reaction was stirred at 25° C. for 1 h, LCMS showed desired MS was detected as main peak. The mixture was extracted with DCM (10 mL*2) and H₂O (20 mL). The organic layer was washed with brine (20 mL), dried over anhydrous Na₂SO₄, filtered and concentrated, then purified by column chromatography (DCM/MeOH=30/1) to give a white solid (46 mg, yield: 39.5%). Chemical Formula: calculated for (M+H⁺) C₂₂H₂₁FN₄O₃: 408.16, Found: 409 ¹H NMR (400 MHz, CDCl₃) δ 8.45 (s, 1H), 7.41-7.35 (m, 2H), 7.05-6.88 (m, 4H), 4.78 (dd, J=57.1, 16.8 Hz, 4H), 4.35 (d, J=28.5 Hz, 2H), 4.18 (s, 1H), 3.97 (s, 1H), 2.56 (s, 3H), 2.19 (s, 3H).

### Example 1-26 Preparation of Compound I-20

FIG. 20 illustrates the synthetic scheme of compound I-20. As shown in FIG. 20, the specific synthesis step is as follows:

### Step 1: 1-(2-(4-fluorophenyl)-3-(2-methylpyridin-4-yl)-6,7-dihydropyrazolo[1,5-a]pyrazin-5(4H)-yl)-2-hydroxyethan-1-one

To a solution of 2-(2-(4-fluorophenyl)-3-(2-methylpyridin-4-yl)-6,7-dihydropyrazolo[1,5-a]pyrazin-5(4H)-yl)-2-oxoethyl acetate (40 mg, 0.1 mmol) in THF/MeOH/H₂O (6:6:2 mL) was added LiOH (8.4 mg, 0.2 mmol) at 25° C., the mixture was stirred at 25° C. for 2 h, LCMS showed desired MS was detected as main peak. The mixture was concentrated, then purified by column chromatography (DCM/MeOH=10/1) to give a white solid (14.4 mg, yield: 38.5%). Chemical Formula: calculated for (M+H⁺) C₂₀H₁₉FN₄O₂: 366.15, Found: 367 ¹H NMR (400 MHz, DMSO) δ 8.41 (d, J=5.1 Hz, 1H), 7.37 (d, J=5.3 Hz, 2H), 7.19 (t, J=8.9 Hz, 2H), 7.01 (d, J=50.1 Hz, 2H), 4.94 (s, 1H), 4.77 (s, 2H), 4.24 (t, J=20.7 Hz, 4H), 3.99 (d, J=20.2 Hz, 2H), 2.44 (s, 3H).

### Example 1-21 Preparation of Compound I-21

FIG. 21 illustrates the synthetic scheme of compound I-21. As shown in FIG. 21, the specific synthesis step is as follows:

### Step 1: 2-(4-fluorophenyl)-5-(methylsulfonyl)-3-(4,4,5,5-tetramethyl-1,3,2-dioxaborolan-2-yl)-4,5,6,7-tetrahydropyrazolo[1,5-a]pyrazine (2)

A solution of methanesulfonyl chloride (0.169 mL, 2.19 mmol) and Et₃N (221.6 mg, 2.19 mmol) in DCM (5 mL) were added to a solution of 2-(4-fluorophenyl)-5-(methylsulfonyl)-3-(4,4,5,5-tetramethyl-1,3,2-dioxaborolan-2-yl)-4,5,6,7-tetrahydropyrazolo[1,5-a]pyrazine (500 mg, 1.46 mmol) in DCM (5 mL). The mixture was stirred at 0° C. for 30 min. The reaction mixture was concentrated under pressure. The residue was purified via Prep-TLC PE/FA (5:1) and give the product. Chemical Formula: calculated for (M+H⁺) C₁₉H₂₅BFN₃O₄S: 421.29, Found: 421.8.

### Step 2: 4-(2-(4-fluorophenyl)-5-(methylsulfonyl)-4,5,6,7-tetrahydropyrazolo[1,5-a]pyrazin-3-yl)pyrimidin-2-amine

4-bromopyrimidin-2-amine (413 mg, 2.37 mmol), 2 M Na₂CO₃ aqueous solutions (0.5 mL) and Pd(dppf)Cl2 (69 mg, 0.095 mmol) were added to a solution of 2-(4-fluorophenyl)-5-(methylsulfonyl)-3-(4,4,5,5-tetramethyl-1,3,2-dioxaborolan-2-yl)-4,5,6,7-tetrahydropyrazolo[1,5-a]pyrazine (200 mg, 0.47 mmol) in EtOH/toluene=8:2 (5 mL). The mixture was stirred at 100° C. for 1 h under nitrogen atmosphere on microwave. The reaction mixture was concentrated under pressure. The residue was purified via Prep-TLC PE/EA (1:50) and give the product. Chemical Formula: calculated for (M+H⁺) C₁₇H₁₇FN₆O₂S388.42, Found: 388.8. ¹H NMR (400 MHz, CDCl₃) δ 7.97 (s, 11H), 7.46 (dd, J=8.7, 5.4 Hz, 2H), 7.12 (t, J=8.7 Hz, 2H), 6.31 (d, J=5.5 Hz, 1H), 5.64 (s, 2H), 4.91 (s, 2H), 4.39 (t, J=5.4 Hz, 2H), 3.95-3.79 (m, 2H), 3.00 (s, 3H).

### Example 1-22 Preparation of Compound I-22

FIG. 22 illustrates the synthetic scheme of compound I-22. As shown in FIG. 22, the specific synthesis step is as follows:

### Step 1: 2-(4-fluorophenyl)-3-(2-methylpyridin-4-yl)-5-((trifluoromethyl)sulfonyl)-4,5,6,7-tetrahydropyrazolo[1,5-a]pyrazine

Trifluoromethanesulfonic anhydride (95 mg, 0.34 mmol) and Et₃N (34 mg, 0.34 mmol) were added to a solution of 2-(4-fluorophenyl)-3-(2-methylpyridin-4-yl)-4,5,6,7-tetrahydropyrazolo[1,5-a]pyrazine (70 mg, 0.23 mmol) in DCM (10 mL). The mixture was stirred at 25° C. for 1 h. The reaction mixture was concentrated under pressure. The residue was purified via Prep-TLC PE/EA (1:1) and give the product. Chemical Formula: calculated for (M+H⁺) C₁₉H₁₆F₄N₄O₂S: 400.42, Found: 400.8. ¹H NMR (400 MHz, CDCl₃) δ 8.48 (d, J=5.0 Hz, 1H), 7.44-7.35 (m, 2H), 7.11-6.96 (m, 2H), 6.92 (s, 1H), 6.86 (d, J=4.9 Hz, 1H), 4.76 (s, 2H), 4.42 (t, J=5.5 Hz, 2H), 4.08 (s, 2H), 2.55 (s, 3H).

### Example 1-23 Preparation of Compound I-23

FIG. 23 illustrates the synthetic scheme of compound I-23. As shown in FIG. 23, the specific synthesis step is as follows:

### Step 1: 2-(2-(4-fluorophenyl)-3-(2-methylpyridin-4-yl)-6,7-dihydropyrazolo[1,5-a]pyrazin-5(4H)-yl)oxazole

To a solution of 4-[2-(4-fluorophenyl)-4H,5H,6H,7H-pyrazolo[1,5-a]pyrazin-3-yl]-2-methylpyridine (70 mg, 0.23 mmol) in PhMe (10 mL) was added 2-bromo-1,3-oxazole (68.0607 mg, 0.46 mmol), Cs₂CO₃ (149.9600 mg, 0.46 mmol), Pd₂(dba)₃ (26.5650 mg, 0.0230 mmol) and x-Phos (26.5650 mg, 0.0460 mmol). The reaction mixture was stirred at 100° C. for 16 h. The reaction was complete detected by LCMS. The solution was filtered, filtrate was collected. The reaction mixture was concentrated under pressure. The residue was purified via Genal-Prep-HPLC and give the product.

### Example 1-24 Preparation of Compound I-24

FIG. 24 illustrates the synthetic scheme of compound I-24. As shown in FIG. 24, the specific synthesis step is as follows:

### Step 1: N-(4-(5-benzyl-2-(4-fluorophenyl)-4,5,6,7-tetrahydropyrazolo[1,5-a]pyrazin-3-yl)pyridin-2-yl) acetamide (2)

To a solution of 4-(5-benzyl-2-(4-fluorophenyl)-4,5,6,7-tetrahydropyrazolo[1,5-a]pyrazin-3-yl)pyridin-2-amine (200 mg, 0.50 mmol) in DCM (6 mL) at 0° C., Et₃N (15.8 mg, 0.75 mmol) was added and acetyl chloride (58 mg, 0.75 mmol) in DCM (2 mL) was added drop by drop. The mixture was stirred at 0° C. for 1 h. The reaction mixture was concentrated under pressure. The residue was purified via Prep-TLC PE/EA (5:1) and give the product. Chemical Formula: calculated for (M+H⁺) C₂₆H₂₄FN₅O: 441.51, Found: 441.8.

### Step 2: N-(4-(2-(4-fluorophenyl)-4,5,6,7-tetrahydropyrazolo[1,5-a]pyrazin-3-yl)pyridin-2-yl) acetamide (3)

HCOONH₄ (598 mg, 9.49 mmol) and Pd/C (210 mg) were added to a solution of N-(4-(5-benzyl-2-(4-fluorophenyl)-4,5,6,7-tetrahydropyrazolo[1,5-a]pyrazin-3-yl)pyridin-2-yl) acetamide (210 mg, 0.47 mmol) in MeOH (10 mL). The mixture was stirred at 60° C. for 3 h. The solution was filtered, filtrate was collected and the solution was concentrated under pressure. Chemical Formula: calculated for (M+H⁺) C₁₉H₁₈FN₅: 351.39, Found: 351.8.

### Step 3: N-(4-(2-(4-fluorophenyl)-5-(methylsulfonyl)-4,5,6,7-tetrahydropyrazolo[1,5-a]pyrazin-3-yl)pyridin-2-yl) acetamide

Methanesulfonyl chloride (0.067 mL, 0.86 mmol) and Et₃N (87 mg, 0.86 mmol) were added to a solution of N-(4-(2-(4-fluorophenyl)-4,5,6,7-tetrahydropyrazolo[1,5-a]pyrazin-3-yl)pyridin-2-yl) acetamide (200 mg, 0.57 mmol) in DCM (10 mL). The mixture was stirred at 25° C. for 1 h. The solution was concentrated under pressure. Chemical Formula: calculated for (M+H⁺) C₂₀H₂₀FN₅O₃S: 429.47, Found: 429.8. ¹H NMR (400 MHz, CDCl₃) δ 8.13 (dd, J=19.9, 14.7 Hz, 3H), 7.57-7.34 (m, 2H), 7.02 (t, J=8.7 Hz, 2H), 6.72 (dd, J=5.2, 1.5 Hz, 1H), 4.63 (s, 2H), 4.40 (t, J=5.4 Hz, 2H), 4.02-3.71 (m, 2H), 2.98 (s, 3H), 2.22 (s, 3H).

### Example 1-25 Preparation of Compound I-25

FIG. 25 illustrates the synthetic scheme of compound I-25. As shown in FIG. 25, the specific synthesis step is as follows:

### Step 1: tert-butyl (4-(2-(4-fluorophenyl)-5-methylsulfonyl)-4,5,6,7-tetrahydropyrazolo[1,5-a]pyrazin-3-yl)pyridin-2-yl) carbamate (2)

To a solution of tert-butyl {4-[2-(4-fluorophenyl)-4H,5H,6H,7H-pyrazolo[1,5-a]pyrazin-3-yl]pyridin-2-yl}aminoformate (250 mg, 0.61 mmol) in DCM (10 mL), MsCl (83.4480 mg, 0.732 mmol), DIPEA (157.3800 mg, 1.22 mmol) was added. The reaction mixture was stirred at 25° C. for 2 h. The reaction was complete detected by lcms. The reaction mixture was concentrated under pressure. The crude material was added to a silica gel column and was eluted with PE/EtOAc (1:1). Chemical Formula: calculated for (M+H⁺) C₂H₆FN₅O₄S: 487.2, Found: 488.0

### Step 2: 4-(2-(4-fluorophenyl)-5-(methylsulfonyl)-4,5,6,7-tetrahydropyrazolo[1,5-a]pyrazin-3-yl)pyridin-2-amine

To a solution of tert-butyl (4-(2-(4-fluorophenyl)-5-(methylsulfonyl)-4,5,6,7-tetrahydropyrazolo[1,5-a]pyrazin-3-yl)pyridin-2-yl)carbamate (130 mg, 0.27 mmol) in DCM (4 mL), HCl-dioxane (4 mL) was added. The reaction mixture was stirred at 25° C. for 2 h. The reaction was complete detected by lcms. The reaction mixture was concentrated under pressure. The organic phase was washed with 2 M sodium carbonate solution (5 mL). The residue was extracted with EA (5 mL*3). The combined organic layers were washed with brine (10 mL), dried over sodium sulfate, filtered and concentrated to give the product. The residue was purified via Genal-Prep-HPLC and give the product.

¹H NMR (400 MHz, DMSO) δ 7.88 (d, J=5.2 Hz, 1H), 7.46-7.41 (m, 2H), 7.20 (t, J=8.8 Hz, 2H), 6.27 (dd, J=10.8, 6.8 Hz, 2H), 6.00 (s, 2H), 4.47 (a, 2H), 4.30 (t, J=5.6 Hz, 2H), 3.77 (t, J=5.6 Hz, 2H), 3.09 (s, 3H).

### Example 1-26 Preparation of Compound I-26

FIG. 26 illustrates the synthetic scheme of compound I-26. As shown in FIG. 26, the specific synthesis step is as follows:

### Step 1:N-(4-(5-ethyl-2-(4-fluorophenyl)-4,5,6,7-tetrahydropyazolo[1,5-a]pyrazin-3-yl)pyridin-2-yl) acetamide

To a solution of N-(4-(2-(4-fluorophenyl)-4,5,6,7-tetrahydropyrazolo[1,5-a]pyrazin-3-yl)pyridin-2-yl) acetamide (150 mg, 0.43 mmol) in MeOH (10 mL), acetaldehyde (38 mg, 0.85 mmol), ZnCl₂ (54 mg, 0.085 mmol) and NaBH₃CN (116.4 mg, 0.85 mmol) were added. The mixture was stirred at 25° C. for 3 h. The reaction mixture was concentrated under pressure. Chemical Formula: calculated for (M+H⁺) C₂₁H₂₂FN₅O: 379.44, Found: 379.8. ¹H NMR (400 MHz, DMSO) δ 8.25 (d, J=5.1 Hz, 1H), 7.89 (s, 1H), 7.53-7.31 (m, 2H), 7.20 (t, J=8.9 Hz, 2H), 6.82 (dd, J=5.2, 1.5 Hz, 1H), 4.36 (s, 2H), 4.15 (s, 2H), 3.43 (s, 2H), 3.04 (d, J=35.2 Hz, 2H), 2.07 (s, 3H), 1.19 (t, J=6.9 Hz, 3H).

### Example 1-27 Preparation of Compound I-27

FIG. 27 illustrates the synthetic scheme of compound I-27. As shown in FIG. 27, the specific synthesis step is as follows:

### Step 1:N-(4-(2-(4-fluorophenyl)-5-isopropyl-4,5,6,7-tetrahydropyrazolo[1,5-a]pyrazin-3-yl)pyridin-2-yl) acetamide

To a solution of N-(4-(2-(4-fluorophenyl)-4,5,6,7-tetrahydropyrazolo[1,5-a]pyrazin-3-yl)pyridin-2-yl) acetamide (120 mg, 0.34 mmol) in MeOH (10 mL), acetone (40 mg, 0.68 mmol), NaBH₃CN (43 mg, 68 mmol) and ZnCl₂ (93 mg, 0.68 mmol) were added. The mixture was stirred at 25° C. for 3 h. The reaction mixture was concentrated under pressure. Chemical Formula: calculated for (M+H⁺) C₂₂H₂₄FN₅O: 393.47, Found: 393.8. ¹H NMR (400 MHz, DMSO) δ 10.49 (s, 1H), 8.20 (d, J=5.1 Hz, 1H), 7.93 (s, 1H), 7.39 (dd, J=8.8, 5.6 Hz, 2H), 7.17 (t, J=8.9 Hz, 2H), 6.76 (dd, J=5.2, 1.5 Hz, 1H), 4.15 (s, 2H), 3.75 (s, 2H), 2.99 (s, 2H), 2.95-2.85 (m, 1H), 2.06 (s, 3H), 1.06 (d, J=6.5 Hz, 6H).

### Example 1-28 Preparation of Compound I-28

FIG. 28 illustrates the synthetic scheme of compound I-28. As shown in FIG. 28, the specific synthesis step is as follows:

### Step 1:N-(4-(5-cyclopropyl-2-(4-fluorophenyl)-4,5,6,7-tetrahydropyrazolo[1,5-a]pyrazin-3-yl)pyridin-2-yl) acetamide

To a solution of N-(4-(2-(4-fluorophenyl)-4,5,6,7-tetrahydropyrazolo[1,5-a]pyrazin-3-yl)pyridin-2-yl) acetamide (300 mg, 0.85 mmol) in THF (20 mL), (1-ethoxycyclopropoxy)trimethylsilane (298 mg, 1.71 mmol), NaBH₃CN (107 mg, 1.71 mmol) and ZnCl₂ (233 mg, 1.71 mmol) were added, then 1 drop 1 M HCl was added. The mixture was stirred at 60° C. for 3 h. The reaction mixture was concentrated under pressure. Chemical Formula: calculated for (M+H⁺) C₂₂H₂₂FN₅O: 391.45, Found: 391.8. ¹H NMR (400 MHz, DMSO) δ 10.50 (s, 1H), 8.21 (d, J=5.2 Hz, 1H), 7.93 (s, 1H), 7.48-7.32 (m, 2H), 7.17 (t, J=8.9 Hz, 2H), 6.77 (dd, J=5.2, 1.5 Hz, 1H), 4.16 (t, J=5.4 Hz, 2H), 3.81 (s, 2H), 3.15 (t, J=5.5 Hz, 2H), 2.06 (s, 3H), 2.01-1.89 (m, 1H), 0.54-0.44 (m, 2H), 0.41 (d, J=3.0 Hz, 2H).

### Example 1-29 Preparation of Compound I-29

FIG. 29 illustrates the synthetic scheme of compound I-29. As shown in FIG. 29, the specific synthesis step is as follows:

### Step 1: N-(4-(2-(4-fluorphenyl)-5-(2,2,2-trifluoroethyl)-4,5,6,7-tetrahydropyrazolo[1,5-a]pyrazin-3-yl)pyridin-2-yl) acetamide

To a solution of N-{4-[2-(4-fluorophenyl)₄H,5H,6H,7H-pyrazolo[1,5-a]pyrazin-3-yl]pyridin-2-yl}acetamide (100 mg, 0.2846 mmol) in THF (5 mL), 2,2,2-trifluoroethyl trifluoromethanesulfonate (99.08 mg, 0.4269 mmol), DIPEA (73.43 mg, 0.5692 mmol) was added. The reaction mixture was stirred at 25° C. for 2 h. The reaction mixture was concentrated under pressure. The crude material was added to a silica gel column and was eluted with PE/EA (1:1).

¹H NMR (400 MHz, DMSO) δ 10.50 (s, 1H), 8.22 (d, J=5.2 Hz, 1H), 7.90 (s, 1H), 7.44-7.34 (m, 2H), 7.22-7.11 (m, 2H), 6.77 (dd, J=5.2, 1.6 Hz, 1H), 4.21 (t, J=5.2 Hz, 2H), 3.96 (s, 2H), 3.50 (d, J=10.0 Hz, 2H), 3.25 (t, J=5.2 Hz, 2H), 2.06 (s, 3H).

### Example 1-30 Preparation of Compound I-30

FIG. 30 illustrates the synthetic scheme of compound I-30. As shown in FIG. 30, the specific synthesis step is as follows:

### Step 1:N-(4-(2-(4-fluorophenyl)-5-(2,2,2-trifluoroacetyl)-4,5,6,7-tetrahydropyrazolo[1,5-a]pyrazin-3-yl)pyridin-2-yl) acetamide

To a solution of N-(4-(2-(4-fluorophenyl)-4,5,6,7-tetrahydropyrazolo[1,5-a]pyrazin-3-yl)pyridin-2-yl) acetamide (200 mg, 0.57 mmol) in DCM (20 mL) at 0° C., 2,2,2-trifluoroacetic anhydride (179 mg, 0.86 mmol) and Et₃N (173 mg, 1.7 mmol) were added. The mixture was stirred at 25° C. for 2 h. The reaction mixture was concentrated under pressure. Chemical Formula: calculated for (M+H⁺) C₂₁H₁₇F₄N₅O₂: 477.39, Found: 477.8. ¹H NMR (400 MHz, DMSO) δ 10.56 (s, 1H), 8.27 (d, J=5.2 Hz, 1H), 7.97 (s, 1H), 7.40 (ddd, J=8.2, 5.4, 2.5 Hz, 2H), 7.20 (t, J=8.9 Hz, 2H), 6.80 (ddd, J=14.6, 5.1, 1.5 Hz, 1H), 4.89 (d, J=21.6 Hz, 2H), 4.46-4.24 (m, 2H), 4.24-4.07 (m, 2H), 2.07 (s, 3H).

### Example 1-31 Preparation of Compound I-31

FIG. 31 illustrates the synthetic scheme of compound I-31. As shown in FIG. 31, the specific synthesis step is as follows:

### Step 1:N-(4-(2-(4-fluorophenyl)-5-(2-hydroxyethyl)-4,5,6,7-tetrahydropyrazolo[1,5-a]pyrazin-3-yl)pyridin-2-yl) acetamide

To a solution of N-(4-(2-(4-fluorophenyl)-4,5,6,7-tetrahydropyrazolo[1,5-a]pyrazin-3-yl)pyridin-2-yl) acetamide (200 mg, 0.57 mmol) in EtOH (20 mL), oxirane (0.4 mL, 1.14 mmol) was added. The mixture was stirred at 50° C. for 16 h. The reaction mixture was concentrated under pressure. Chemical Formula: calculated for (M+H⁺) C₂H₂₂FN₅O₂: 395.44, Found: 395.8. ¹H NMR (400 MHz, DMSO) δ 10.50 (s, 1H), 8.20 (t, J=4.6 Hz, 1H), 7.92 (s, 1H), 7.46-7.28 (m, 2H), 7.26-7.08 (m, 2H), 6.76 (dd, J=5.2, 1.5 Hz, 1H), 4.56 (s, 1H), 4.18 (t, J=5.3 Hz, 2H), 3.74 (s, 2H), 3.56 (t, J=5.8 Hz, 2H), 3.05 (t, J=5.4 Hz, 2H), 2.65 (t, J=5.9 Hz, 2H), 2.07 (s, 3H).

### Example 1-32 Preparation of Compound I-32

FIG. 32 illustrates the synthetic scheme of compound I-32. As shown in FIG. 32, the specific synthesis step is as follows:

### Step 1: N-(4-(5-benzyl-2-(4-fluorophenyl)-4,5,6,7-tetrahydropyrazolo[1,5-a]pyrazin-3-yl)pyridin-2-yl)pivalamide (2)

To a solution of 4-[2-(4-fluorophenyl)-5-(1-methylphenyl)-4H,6H,7H-pyrazolo[1,5-a]pyrazin-3-yl]pyridin-2-amine (500 mg, 1.2485 mmol) in DCM (10 mL), 2,2-dimethylpropanoyl chloride (165.59 mg, 0.1.3733 mmol), DIPEA (322.11 mg, 2.497 mmol) was added. The reaction mixture was stirred at 25° C. for 2 h. The reaction was complete detected by lcms. The reaction mixture was concentrated under pressure. The crude material was added to a silica gel column and was eluted with PE/EtOAc (1:1). Chemical Formula: calculated for (M+H⁺) C₂₉H₃₀FN₅O: 483.2, Found: 483.7.

### Step 2: N-(4-(2-(4-fluorophenyl)-4,5,6,7-tetrahydropyrazolo[1,5-a]pyrazin-3-yl)pyridin-2-yl)pivalamide (3)

To a solution of N-(4-(5-benzyl-2-(4-fluorophenyl)-4,5,6,7-tetrahydropyrazolo[1,5-a]pyrazin-3-yl)pyridin-2-yl)pivalamide (700 mg, 1.4445 mmol) in EA (5 mL), Pd/C (400 mg), HCl (1 mL) was added. The reaction mixture was stirred at 25° C. under H₂ for 2 h. The reaction was complete detected by lcms. The solution was filtered, filtrate was collected. The reaction mixture was concentrated under pressure. The organic phase was washed with 2 M sodium carbonate solution (5 mL). The residue was extracted with EA (10 mL*3). The combined organic layers were washed with brine (10 mL), dried over sodium sulfate, filtered and concentrated to give the product. Chemical Formula: calculated for (M+H⁺) C₂₂H₇FN₅O: 393.2, Found: 393.8.

### Step 3: N-(4-(2-(4-fluorophenyl-5-(methylsulfonyl)-4,5,6,7-tetrahydropyrazolo[1,5-a]pyrazin-3-yl)pyridin-2-yl)pivalamide

To a solution of N-(4-(2-(4-fluorophenyl)-4,5,6,7-tetrahydropyrazolo[1,5-a]pyrazin-3-yl)pyridin-2-yl)pivalamide (150 mg, 0.3812 mmol) in DCM (10 mL), MsCl (65.19 mg, 0.5718 mmol), DIPEA (98.35 mg, 0.7624 mmol) was added. The reaction mixture was stirred at 25° C. for 2 h. The reaction was complete detected by lcms. The reaction mixture was concentrated under pressure. The residue was purified via Flash Chromatography and was eluted with Hex/EtOAc and give the product. The residue was purified via Genal-Prep-HPLC and give the product.

¹H NMR (400 MHz, DMSO) δ 9.89 (s, 1H), 8.29 (d, J=5.2 Hz, 1H), 7.92 (s, 1H), 7.44-7.37 (m, 2H), 7.23-7.15 (m, 2H), 6.86 (dd, J=5.2, 1.6 Hz, 11H), 4.51 (s, 2H), 4.33 (t, J=5.2 Hz, 2H), 3.80 (t, J=5.2 Hz, 2H), 3.08 (s, 3H), 1.22 (5, 9H).

### Example 1-33 Preparation of Compound I-33

FIG. 33 illustrates the synthetic scheme of compound I-33. As shown in FIG. 33, the specific synthesis step is as follows:

### Step 1: N-(4-(5-cyclobutyl-2-(4-fluorophenyl)-4,5,6,7-tetrahydropyrazolo[1,5-a]pyrazin-3-yl)pyridin-2-yl) acetamide

To a solution of N-{4-[2-(4-fluorophenyl)-4H,5H,6H,7H-pyrazolo[1,5-a]pyrazin-3-yl}pyridin-2-yl) acetamide (200 mg, 0.57 mmol) in THF (20 mL), NaBH₃CN (71.5 mg, 1.14 mmol) and cyclobutanone (80 mg, 1.14 mmol) was added. The reaction mixture was stirred at 25° C. for 20 h. The reaction mixture was concentrated under pressure. The residue was purified via Genal-Prep-HPLC and give the product.

¹H NMR (400 MHz, DMSO) δ 10.50 (s, 1H), 8.20 (d, J=5.0 Hz, 1H), 7.91 (s, 1H), 7.39 (dd, J=8.6, 5.6 Hz, 2H), 7.17 (t, J=8.9 Hz, 2H), 6.75 (dd, J=5.1, 1.2 Hz, 1H), 4.17 (t, J=5.1 Hz, 2H), 3.12-2.96 (m, 1H), 2.85 (t, J=5.1 Hz, 2H), 2.06 (s, 4H), 1.93-1.76 (m, 2H), 1.71-1.55 (m, 2H).

### Example 1-34 Preparation of Compound I-34

FIG. 34 illustrates the synthetic scheme of compound I-34. As shown in FIG. 34, the specific synthesis step is as follows:

### Step 1: N-(4-(2-(4-fluorophenyl)-5-(trifluoromethyl)-4,5,6,7-tetrahydropyrazolo[1,5-a]pyrazin-3-yl)pyridin-2-yl) acetamide

To a solution of N-(4-(2-(4-fluorophenyl)-4,5,6,7-tetrahydropyrazolo[1,5-a]pyrazin-3-yl)pyridin-2-yl) acetamide (100 mg, 0.285 mmol) in acetone (20 ml), K₂CO₃ (60 mg, 0.427 mmol), trifluoromethyl iodide (61.4 mg, 0.313 mmol) was added at r.t. The reaction mixture was stirred at 50° C. for 15 h. The reaction mixture was concentrated under pressure. The residue was purified via Prep-TLC (PE/EtOAc)=(2:1). The residue was purified via Genal-Prep-HPLC and obtained the product.

Chemical Formula: calculated for (M+H⁺) C₂₀H₁₇F₄N₅O: 419.14, Found: 420.24.

### Example 1-35 Preparation of Compound I-35

FIG. 35 illustrates the synthetic scheme of compound I-35. As shown in FIG. 35, the specific synthesis step is as follows:

### Step 1: N-(4-(2-(4-fluorophenyl)-5-(methyl-d3)-4,5,6,7-tetrahydropyrazolo[1,5-a]pyrazin-3-yl)pyridin-2-yl) acetamide

To a solution of N-(4-(2-(4-fluorophenyl)-4,5,6,7-tetrahydropyrazolo[1,5-a]pyrazin-3-yl)pyridin-2-yl) acetamide (100 mg, 0.285 mmol) in acetone (20 ml), K₂CO₃ (60 mg, 0.427 mmol), iodomethane-d3 (45.4 mg, 0.313 mmol) was added at r.t. The reaction mixture was stirred at 50° C. for 15 h. The reaction mixture was concentrated under pressure. The residue was purified via Prep-TLC (PE/EtOAc)=(2:1). The residue was purified via Genal-Prep-HPLC and obtained the product.

Chemical Formula: calculated for (M+H⁺) C₂₀H₁₇D₃FN₅O: 368.18, Found: 369.20.

### Example 1-36 Preparation of Compound I-36

FIG. 36 illustrates the synthetic scheme of compound I-36. As shown in FIG. 36, the specific synthesis step is as follows:

### Step 1: 1-(4-(5-benzyl-2-(4-fluorophenyl)-4,5,6,7-tetrahydropyrazolo[1,5-a]pyrazin-3-yl)pyridin-2-yl)propan-2-one

To a solution of 5-benzyl-2-(4-fluorophenyl)-3-(2-methylpyridin-4-yl)-4,5,6,7-tetrahydropyrazolo[1,5-a]pyrazine (200 mg, 0.50 mmol) in THF (6 mL) at −78° C., LiN(Pr-i)₂ (80.3 mg, 0.75 mmol) was added and mixture was stirred 30 min N-methoxy-N-methylacetamide (58 mg, 0.75 mmol) in THF (2 mL) was added drop by drop. The mixture was stirred at −78° C. for 1 h. The reaction mixture The reaction mixture was concentrated under pressure. The residue was purified via Prep-TLC PE/EA (5:1) and give the product.

Chemical Formula: calculated for (M+H⁺) C₂₇H₂₅FN₄O: 440.2, Found: 441.2.

### Step 2: 1-(4-(2-(4-fluorophenyl)-4,5,6,7-tetrahydropyrazolo[1,5-a]pyrazin-3-yl)pyridin-2-yl)propan-2-one

HCOONH₄ (286.5 mg, 4.54 mmol) and Pd/C (20 mg) were added to a solution of 1-(4-(5-benzyl-2-(4-fluorophenyl)-4,5,6,7-tetrahydropyrazolo[1,5-a]pyrazin-3-yl)pyridin-2-yl)propan-2-one (200 mg, 0.454 mmol) in MeOH (10 ml). The mixture was stirred at 60° C. for 3 h. The solution was filtered, filtrate was collected and the solution was concentrated under pressure and used without further purification.

Chemical Formula: calculated for (M+H⁺) C₂₀H₁FN₄O: 350.15, Found: 351.8.

### Step 3: 1-(4-(2-(4-fluorophenyl)-5-(methylsulfonyl)-4,5,6,7-tetrahydropyrazolo[1,5-a]pyrazin-3-yl)pyridin-2-yl) propan-2-one

To a solution of 1-(4-(2-(4-fluorophenyl)-4,5,6,7-tetrahydropyrazolo[1,5-a]pyrazin-3-yl)pyridin-2-yl)propan-2-one (150 mg, 0.428 mmol) in DCM (10 ml) at 0° C., MsCl (54.0 mg, 0.471 mmol) in DCM (2 ml) was added drop by drop and the mixture was stirred 3 h. SM was disappear on TLC. The reaction mixture was added the saturated aqueous solution of NaHCO₃ (10 ml) and extracted with DCM (20 ml). Then the organic layer was concentrated under pressure. The residue was purified via Genal-Prep-HPLC and obtained the product.

Chemical Formula: calculated for (M+H⁺) C₂₁H₂₁FN₄O₃S: 428.13, Found: 429.21.

### Example 1-37 Preparation of Compound I-37

FIG. 37 illustrates the synthetic scheme of compound I-37. As shown in FIG. 37, the specific synthesis step is as follows:

### Step 1: 1-(4-(2-(4-fluorophenyl)-5-(methylsulfonyl)-4,5,6,7-tetrahydropyrazolo[1,5-a]pyrazin-3-yl)pyridin-2-yl)propan-2-ol

To a solution of 1-(4-(2-(4-fluorophenyl)-5-(methylsulfonyl)-4,5,6,7-tetrahydropyrazolo[1,5-a]pyrazin-3-yl) pyridin-2-yl) propan-2-one (150 mg, 0.35 mmol) in THF (20 ml) at 0° C., and NaBH₃CN (44 mg, 0.7 mmol) was added at 0° C. The reaction mixture was stirred at 0° C. for 0.5 h and warmed to r.t. SM disappeared was monitored by TLC. The reaction mixture was quenched with H₂O and extracted with DCM (20 ml). Then the organic layer was concentrated under pressure. The residue was purified via Prep-TLC (PE/EtOAc)=(2:1). The residue was purified via Genal-Prep-HPLC and obtained the product.

Chemical Formula: calculated for (M+H⁺) C₂₁H₂₃FN₄O₃S: 430.15, Found: 431.20.

### Example 1-38 Preparation of Compound I-38

FIG. 38 illustrates the synthetic scheme of compound I-38. As shown in FIG. 38, the specific synthesis step is as follows:

### Step 1: N-(4-(5-(fluoromethyl)-2-(4-fluorophenyl)-4,5,6,7-tetrahydropyrazolo[1,5-a]pyrazin-3-yl)pyridin-2-yl) acetamide

To a solution of N-(4-(2-(4-fluorophenyl)-4,5,6,7-tetrahydropyrazolo[1,5-a]pyrazin-3-yl)pyridin-2-yl) acetamide (150 mg, 0.427 mmol) in acetone (20 ml), K₂CO₃ (117.9 mg, 0.854 mmol) at r.t. Then fluoroiodomethane (61.4 mg, 0.47 mmol) was added. The reaction mixture was stirred at 30° C. for 3 h. SM disappeared was monitored by TLC. The reaction mixture was quenched with H₂O and extracted with DCM (20 ml). The organic layer was concentrated under pressure. The residue was purified via Genal-Prep-HPLC and obtained the product.

Chemical Formula: calculated for (M+H⁺) C₂₀H₁₉F₂N₅O: 383.16, Found: 384.18.

### Example 1-39 Preparation of Compound I-39

FIG. 39 illustrates the synthetic scheme of compound I-39. As shown in FIG. 39, the specific synthesis step is as follows:

### Step 1: N-(4-(5-(difluoromethyl)-2-(4-fluorophenyl)-4,5,6,7-tetrahydropyrazolo[1,5-a]pyrazin-3-yl)pyridin-2-yl) acetamide

To a solution of N-(4-(2-(4-fluorophenyl)-4,5,6,7-tetrahydropyrazolo[1,5-a]pyrazin-3-yl)pyridin-2-yl) acetamide (150 mg, 0.427 mmol) in acetone (20 ml), K₂CO₃ (117.9 mg, 0.854 mmol) at r.t. Then bromodifluoromethane (61.1 mg, 0.47 mmol) was added. The reaction mixture was stirred at 30° C. for 3 h. SM disappeared was monitored by TLC. The reaction mixture was quenched with H₂O and extracted with DCM (20 ml). The organic layer was concentrated under pressure. The residue was purified via Genal-Prep-HPLC and obtained the product.

Chemical Formula: calculated for (M+H⁺) C₂₀H₁₈F₃N₅O: 401.15, Found: 402.22.

### Example 1-40 Preparation of Compound I-40

FIG. 40 illustrates the synthetic scheme of compound I-40. As shown in FIG. 40, the specific synthesis step is as follows:

### Step 1: N-(4-(2-(4-fluorophenyl)-5-(2-methoxyethyl)-4,5,6,7-tetrahydropyrazolo[1,5-a]pyrazin-3-yl)pyridin-2-yl) acetamide

To a solution of N-{4-[2-(4-fluorophenyl)-4H,5H,6H,7H-pyrazolo[1,5-a]pyrazin-3-yl]pyridin-2-yl}acetamide (150 mg, 0.43 mmol) in ACN (5 mL), 1-bromo-2-methoxyethane (71.2 mg, 0.51 mmol) and potassium carbonate (70.8 mg, 0.51 mmol) was added. The reaction mixture was stirred at 50° C. for 20 h. The reaction mixture was concentrated under pressure. The residue was extracted with EA (10 mL*2). The reaction mixture was concentrated under pressure. The residue was purified via Genal-Prep-HPLC and give the product.

¹H NMR (400 MHz, DMSO) δ 10.49 (s, 1H), 8.21 (d, J=5.1 Hz, 1H), 7.92 (s, 1H), 7.48-7.32 (m, 2H), 7.17 (t, J=8.9 Hz, 2H), 6.76 (dd, J=5.2, 1.5 Hz, 1H), 4.17 (t, J=5.3 Hz, 2H), 3.73 (s, 2H), 3.50 (t, J=5.5 Hz, 2H), 3.23 (s, 3H), 3.05 (t, J=5.4 Hz, 2H), 2.74 (t, J=5.6 Hz, 2H), 2.06 (s, 3H).

### Example 1-41 Preparation of Compound I-41

FIG. 41 illustrates the synthetic scheme of compound I-41. As shown in FIG. 41, the specific synthesis step is as follows:

### Step 1: N-(4-(2-(4-fluorophenyl)-5-(2-(methylamino)ethyl-4,5,6,7-tetrahydropyrazolo[1,5-a]pyrazin-3-yl)pyridin-2-yl) acetamide (2)

To a solution of N-(4-(2-(4-fluorophenyl)-4,5,6,7-tetrahydropyrazolo[1,5-a]pyrazin-3-yl)pyridin-2-yl) acetamide (200 mg, 0.5692 mmol) in MeOH (10 mL), tert-butyl methyl(2-oxoethyl)carbamate (148.75 mg, 0.8538 mmol) and CH₃COOH (0.3 mL) was added. After stirring for 1 h, NaBH₃CN (107.31 mg, 1.7076 mmol) was added. The mixture was stirred for 4 h at room temperature. LCMS showed product peak. The reaction was quenched with saturated aqueous solution of NH₄Cl and extracted with EA. The reaction was washed with water, dried over Na₂SO₄. The crude product was purified by silica gel column (200-300 mesh, DCM:MeOH=10:1) to give the product. Chemical Formula: calculated for (M+H⁺), C₂₇H₃₃FN₆O₃:508.60, Found: 509.0

### Step 2:N-(4-(2-(4-fluorophenyl)-5-(2-(methylamino)ethyl)-4,5,6,7-tetrahydropyrazolo[1,5-a]pyrazin-3-yl) pyridin-2-yl) acetamide

To a solution of N-(4-(2-(4-fluorophenyl)-5-(2-(methylamino)ethyl)-4,5,6,7-tetrahydropyrazolo[1,5-a]pyrazin-3-yl)pyridin-2-yl) acetamide (210 mg, 0.4121 mmol) in DCM (5 mL) was added TFA (3 mL) at room temperature. The reaction mixture was stirred at room temperature for 3 h. The reaction was complete detected by LCMS. The solution was concentrated in vacuo. The reaction mixture was concentrated under pressure. The residue was purified via Genal-Prep-HPLC and give the product. Chemical Formula: calculated for (M+H⁺) C₂₂H₂₃FN₆O 408.48, Found: 409.1.

¹H NMR (400 MHz, DMSO) δ 10.51 (s, 1H), 8.22 (d, J=5.2 Hz, 1H), 7.92 (s, 1H), 7.43-7.31 (m, 2H), 7.21-7.15 (m, 2H), 6.76 (dd, J=5.2, 1.5 Hz, 1H), 4.23 (t, J=5.2 Hz, 2H), 3.77 (s, 2H), 3.15-2.84 (m, 4H), 2.81-2.76 (m, 2H), 2.53-2.50 (m, 3H), 2.06 (s, 3H).

### Example 1-42 Preparation of Compound I-42

FIG. 42 illustrates the synthetic scheme of compound I-42. As shown in FIG. 42, the specific synthesis step is as follows:

### Step 1: N-(4-(5-(2-fluoroethyl)-2-(4-fluorophenyl)-4,5,6,7-tetrahydropyrazolo[1,5-a]pyrazin-3-yl)pyridin-2-yl) acetamide

To a solution of N-{4-[2-(4-fluorophenyl)-4H,5H,6H,7H-pyrazolo[1,5-a]pyrazin-3-yl]pyridin-2-yl}acetamide (150 mg, 0.43 mmol) in ACN (5 mL), 1-bromo-2-fluoroethane (65 mg, 0.51 mmol) and potassium carbonate (70.8 mg, 0.51 mmol) was added. The reaction mixture was stirred at 50° C. for 25 h. The reaction mixture was concentrated under pressure. The residue was extracted with EA (10 mL*2). The reaction mixture was concentrated under pressure. The residue was purified via Genal-Prep-HPLC and give the product.

¹H NMR (400 MHz, DMSO) δ 10.49 (s, 1H), 8.21 (d, J=5.1 Hz, 1H), 7.91 (s, 1H), 7.48-7.29 (m, 2H), 7.17 (t, J=8.9 Hz, 2H), 6.77 (dd, J=5.1, 1.5 Hz, 1H), 4.65 (d, J=4.8 Hz, 1H), 4.53 (d, J=4.7 Hz, 1H), 4.19 (t, J=5.3 Hz, 2H), 3.77 (s, 2H), 3.09 (t, J=5.4 Hz, 2H), 2.97-2.80 (m, 2H), 2.06 (s, 3H).

### Example 1-43 Preparation of Compound I-43

FIG. 43 illustrates the synthetic scheme of compound I-43. As shown in FIG. 43, the specific synthesis step is as follows:

### Step 1: N-(4-(5-benzyl-2-(4-fluorophenyl)-4,5,6,7-tetrahydropyrazolo[1,5-a]pyrazin-3-yl)pyridin-2-yl) acetamide (2)

To a solution of 4-(5-benzyl-2-(4-fluorophenyl)-4,5,6,7-tetrahydropyrazolo[1,5-a]pyrazin-3-yl)pyridin-2-amine (500 mg, 1.25 mmol) in DCM (10 mL), TEA (252.98 mg, 2.50 mmol) was added. Then acetyl chloride (98.13 mg, 1.25 mmol) was dropped in at 0° C. The reaction mixture was stirred at 25° C. for 2 h. The reaction was diluted with water (10 ml), and the water phase was extracted with DCM (5 ml) twice time, the organic phase was combined and concentrated under pressure. The residue was purified via Prep-TLC (PE/EtOAc)=(2:1). Light yellow solid was obtained (469.73 mg, y=85.7%), Chemical Formula: calculated for (M+H⁺) C₂H₂₄FN₅O: 441.51, Found: 442.

### Step 2: N-(4-(2-(4-fluorophenyl)-4,5,6,7-tetrahydropyrazolo[1,5-a]pyrazin-3-yl)pyridin-2-yl) acetamide (3)

To a solution of N-(4-(5-benzyl-2-(4-fluorophenyl)-4,5,6,7-tetrahydropyrazolo[1,5-a]pyrazin-3-yl)pyridin-2-yl) acetamide (450 mg, 1.02 mmol) in MeOH (10 mL), Pd/C (45 mg, 10% w/w) was added in. The mixture was stirred at 25° C. for 6 h under N₂ atmosphere. The reaction mixture was filtrated, The filtrate liquid was concentrated under pressure. Brown solid was obtained (315.52 mg, y=88.1%), Chemical Formula: calculated for (M+H⁺) C₁₉H₁₈FN₅O: 351.39, Found: 352.

### Step 3: N-(4-(2-(4-fluorophenyl)-5-((1-methylpiperidin-4-yl)methyl)-4,5,6,7-tetrahydropyrazolo[1,5-a]pyrazin-3-yl)pyridin-2-yl) acetamide

To a solution of N-(4-(2-(4-fluorophenyl)-4,5,6,7-tetrahydropyrazolo[1,5-a]pyrazin-3-yl)pyridin-2-yl) acetamide (50 mg, 0.14 mmol) in THF (5 mL), 1-methylpiperidine-4-carbaldehyde (19.08 mg, 0.15 mmol) was added in. The mixture was stirred at 25° C. for 30 min. Then sodium cyanoborohydride (18.85 mg, 0.30 mmol) was added in at 0° C. The reaction was stirred at 25° C. for 2 h. The reaction was quenched with water and was extracted with EA twice time, the organic phase was combined and concentrated under pressure. The residue was purified via Genal-Prep-HPLC and give the product as white solid (32.98 mg, 50.1%)

Chemical Formula: calculated for (M+H+)C₂₆H₃₁FN₆O: 462.57, Found: 463.

### Example 1-44 Preparation of Compound I-44

FIG. 44 illustrates the synthetic scheme of compound I-44. As shown in FIG. 44, the specific synthesis step is as follows:

### Step 1:N-(4-(5-(3-benzyloxy)cyclobutyl)-2-(4-fluorophenyl)-4,5,6,7-tetrahydropyrazolo[1,5-a]pyrazin-3-yl)pyridin-2-yl) acetamide (2)

To a solution of N-(4-(2-(4-fluorophenyl)-4,5,6,7-tetrahydropyrazolo[1,5-a]pyrazin-3-yl)pyridin-2-yl) acetamide (1 g, 2.8 mmol) in MeOH (50 mL), 3-(benzyloxy)cyclobutan-1-one (0.99 g, 5.6 mmol), ZnCl₂ (0.76 g, 5.6 mmol) and NaBH₃CN (0.35 g, 5.6 mmol) were added. The mixture was stirred at 25° C. for 3 h. The reaction mixture was concentrated under pressure. Chemical Formula: calculated for (M+H⁺) C₃₀H₃₀FN₅O₂: 511.62, Found: 511.8.

### Step 2:N-(4-(2-(4-fluorophenyl)-5-(3-hydroxycyclobutyl)-4,5,6,7-tetrahydropyrazolo[1,5-a]pyrazin-3-yl) pyridin-2-yl) acetamide

To a solution of N-(4-(5-(3-(benzyloxy)cyclobutyl)-2-(4-fluorophenyl)-4,5,6,7-tetrahydropyrazolo[1,5-a]pyrazin-3-yl )pyridin-2-yl) acetamide (1 g, 2 mmol) in MeOH (50 mL), Pd/C (100 mg) and 1 M HCl (1 mL) were added. The mixture was stirred at 60° C. for 16 h under hydrogen atmosphere. The solution was filtered, filtrate was collected and concentrated under pressure. Chemical Formula: calculated for (M+H⁺) C₂₃H₂₄FN₅O₂: 421.48, Found: 421.8. ¹H NMR (400 MHz, DMSO) δ 11.73 (s, 1H), 8.28 (d, J=5.8 Hz, 1H), 7.77 (d, J=8.3 Hz, 1H), 7.45 (dd, J=8.7, 5.5 Hz, 2H), 7.24 (t, J=8.9 Hz, 2H), 6.96 (d, J=5.8 Hz, 1H), 4.77-4.42 (m, 4H), 3.94-3.85 (m, 1H), 3.76 (s, 2H), 3.52-3.35 (m, 1H), 2.61 (d, J=6.4 Hz, 2H), 2.44-2.22 (m, 2H), 2.18 (s, 3H).

### Example 1-45 Preparation of Compound I-45

FIG. 45 illustrates the synthetic scheme of compound I-45. As shown in FIG. 45, the specific synthesis step is as follows:

### Step 1: N-(4-(5-(3,3-difluorocyclobutyl)-2-(4-fluorphenyl)-4,5,6,7-tetrahydropyrazolo[1,5-a]pyrazin-3-yl)pyridin-2-yl) acetamide

To a solution of N-(4-(2-(4-fluorophenyl)-4,5,6,7-tetrahydropyrazolo[1,5-a]pyrazin-3-yl)pyridin-2-yl) acetamide (50 mg, 0.14 mmol) in THF (6 mL), 3,3-difluorocyclobutan-1-one (15.91 mg, 0.15 mmol) was added in. The mixture was stirred at 25° C. for 30 min. Then sodium cyanoborohydride (18.85 mg, 0.30 mmol) was added in at 0° C. The reaction was stirred at 25° C. for 2 h. The reaction was quenched with water and was extracted with EA twice time, the organic phase was combined and concentrated under pressure. The residue was purified via Genal-Prep-HPLC and give the product as white solid (28.77 mg, 45.8%)

Chemical Formula: calculated for (M+H+)C₂₃H₂₂F₃N₅O: 441.46, Found:442.

### Example 1-46 Preparation of Compound I-46

FIG. 46 illustrates the synthetic scheme of compound I-46. As shown in FIG. 46, the specific synthesis step is as follows:

### Step 1:N-(4-(2-(4-fluorophenyl)-5-(oxetan-3-yl)-4,5,6,7-tetrahydropyrazolo[1,5-a]pyrazin-3-yl)pyridin-2-yl) acetamide

To a solution of N-(4-(2-(4-fluorophenyl)-4,5,6,7-tetrahydropyrazolo[1,5-a]pyrazin-3-yl)pyridin-2-yl) acetamide (200 mg, 0.57 mmol) in MeOH (5 mL), oxetan-3-one (82 mg, 1.13 mmol), NaBH₃CN (71.5 mg, 1.13 mmol) and ZnCl₂ (155.16 mg, 1.13 mmol) were added. The mixture was stirred at 25° C. for 2 h. The reaction mixture was concentrated under pressure. Chemical Formula: calculated for (M+H⁺) C₂₂H₂₂FN₅O₂: 407.45, Found: 407.8. ¹H NMR (400 MHz, DMSO) δ 10.49 (s, 1H), 8.21 (d, J=5.1 Hz, 1H), 7.89 (s, 1H), 7.46-7.29 (m, 2H), 7.17 (t, J=8.9 Hz, 2H), 6.79 (dd, J=5.2, 1.4 Hz, 1H), 4.60 (t, J=6.6 Hz, 2H), 4.50 (t, J=6.1 Hz, 2H), 4.21 (t, J=5.3 Hz, 2H), 3.87-3.69 (m, 1H), 3.61 (s, 2H), 2.91 (t, J=5.3 Hz, 2H), 2.05 (s, 3H).

### Example 1-47 Preparation of Compound I-47

FIG. 47 illustrates the synthetic scheme of compound I-47. As shown in FIG. 47, the specific synthesis step is as follows:

### Step 1: N-(4-(2-(4-fluorophenyl)-5-(tetrahydro-2H-pyran-4-yl)-4,5,6,7-tetrahydropyrazolo[1,5-a]pyrazin-3-yl)pyridin-2-yl) acetamide

To a solution of N-(4-(2-(4-fluorophenyl)-4,5,6,7-tetrahydropyrazolo[1,5-a]pyrazin-3-yl)pyridin-2-yl) acetamide (50 mg, 0.14 mmol) in THF (5 mL), tetrahydro-4H-pyran-4-one (15.02 mg, 0.15 mmol) was added in. The mixture was stirred at 25° C. for 30 min. Then sodium cyanoborohydride (18.85 mg, 0.30 mmol) was added in at 0° C. The reaction was stirred at 25° C. for 2 h. The reaction was quenched with water and was extracted with EA twice time, the organic phase was combined and concentrated under pressure. The residue was purified via Genal-Prep-HPLC and give the product as white solid (32.41 mg, 52.3%)

Chemical Formula: calculated for (M+H+)C₂₄H₂₆FN₅O₂: 435.50, Found:436.

### Example 1-48 Preparation of Compound I-48

FIG. 48 illustrates the synthetic scheme of compound I-48. As shown in FIG. 48, the specific synthesis step is as follows:

### Step 1: N-(4-(5-(1,1-dioxidotetrahydro-2H-thiopyran-4-yl)-2-(4-fluorophenyl)-4,5,6,7-tetrahydropyrazolo[1,5-a]pyrazin-3-yl)pyridin-2-yl) acetamide

To a solution of N-(4-(2-(4-fluorophenyl)-4,5,6,7-tetrahydropyrazolo[1,5-a]pyrazin-3-yl)pyridin-2-yl) acetamide (50 mg, 0.15 mmol) in THF (5 mL), tetrahydro-4H-thiopyran-4-one 1,1-dioxide (22.23 mg, 0.15 mmol) was added in. The mixture was stirred at 25° C. for 30 min. Then sodium cyanoborohydride (18.85 mg, 0.30 mmol) was added in at 0° C. The reaction was stirred at 25° C. for 2 h. The reaction was quenched with water and was extracted with EA twice time, the organic phase was combined and concentrated under pressure. The residue was purified via Genal-Prep-HPLC and give the product as white solid (31.44 mg, 45.7%)

Chemical Formula: calculated for (M+H+)C₂₄H₂₆FN₅O₃S: 483.56, Found:484.

### Example 1-49 Preparation of Compound I-49

FIG. 49 illustrates the synthetic scheme of compound I-49. As shown in FIG. 49, the specific synthesis step is as follows:

### Step 1:N-(4-(2-(4-fluorophenyl)-5-(3-hydroxycyclobutyl)-4,5,6,7-tetrahydropyrazolo[1,5-a]pyrazin-3-yl) pyridin-2-yl) acetamide

To a solution of N-(4-(2-(4-fluorophenyl)-5-(3-hydroxycyclobutyl)-4,5,6,7-tetrahydropyrazolo[1,5-a]pyrazin-3-yl)pyridin-2-yl) acetamide (240 mg, 0.57 mmol) in THF (10 mL) at 0° C., DAST (275 mg, 1.7 mmol) in THF (5 mL) was added. The mixture was stirred at 0° C. for 8 h. The reaction mixture was concentrated under pressure. Chemical Formula: calculated for (M+H⁺) C₂₃H₂₃F₂N₅O: 423.47, Found: 423.8. ¹H NMR (400 MHz, DMSO) δ 10.50 (s, 1H), 8.21 (d, J=5.1 Hz, 1H), 7.91 (s, 1H), 7.52-7.27 (m, 2H), 7.17 (dd, J=12.4, 5.5 Hz, 2H), 6.76 (dd, J=5.2, 1.5 Hz, 1H), 5.40-4.89 (m, 11H), 4.18 (t, J=5.3 Hz, 2H), 3.61 (s, 2H), 3.29-3.18 (m, 2H), 2.90 (t, J=5.3 Hz, 2H), 2.43-2.14 (m, 4H), 2.06 (s, 3H).

### Example 1-50 Preparation of Compound I-50

FIG. 50 illustrates the synthetic scheme of compound I-50. As shown in FIG. 50, the specific synthesis step is as follows:

### Step 1: N-(4-(5-(2-(dimethylamino)ethyl)-2-(4-fluorophenyl)-4,5,6,7-tetrahydropyrazolo[1,5-a]pyrazin-3-yl) pyridin-2-yl) acetamide

To a solution N-(4-(2-(4-fluorophenyl)-5-(2-(methylamino)ethyl)-4,5,6,7-tetrahydropyrazolo[1,5-a]pyrazin-3-yl)pyridin-2-yl) acetamide (232 mg, 0.5686 mmol) in MeOH (5 mL), formaldehyde (26 mg, 0.8529 mmol) and CH₃COOH (0.3 mL) was added. After stirring for 1 h, NaBH₃CN (71.4 mg, 1.137 mmol) was added. The mixture was stirred for 4 h at room temperature. LCMS showed product peak. The reaction was quenched with saturated aqueous solution of NH₄Cl and extracted with EA. The reaction was washed with water, dried over Na₂SO₄. The crude product was purified by prep-HPLC to give the product. Chemical Formula: calculated for (M+H⁺) C₂₇H₃₃FN₆O₃:422.51, Found: 423.1.

¹H NMR (400 MHz, DMSO) δ 10.50 (s, 1H), 8.37-8.09 (m, 2H), 7.92 (a, 1H), 7.58-7.29 (m, 2H), 7.23-7.10 (m, 2H), 6.76 (dd, J=5.2, 1.5 Hz, 1H), 4.18 (t, J=4.0 Hz, 2H), 3.74 (s, 2H), 3.04 (t, J=4.0 Hz, 2H), 2.74-2.66 (m, 2H), 2.64-2.55 (m, 2H), 2.32-2.20 (m, 6H), 2.06 (s, 3H).

### Example 1-51 Preparation of Compound I-51

FIG. 51 illustrates the synthetic scheme of compound I-51. As shown in FIG. 51, the specific synthesis step is as follows:

### Step 1: N-(4-(2-(4-fluorophenyl)-5-(1-(methylthio)vinyl)-4,5,6,7-tetrahydropyrazolo[1,5-a]pyrazin-3-yl)pyridin-2-yl) acetamide

To a solution of N-(4-(2-(4-fluorophenyl)-4,5,6,7-tetrahydropyrazolo[1,5-a]pyrazin-3-yl)pyridin-2-yl) acetamide [100 mg, 0.2849 mmol] in pyridine [5 mL], then was added ethene-1,1-diylbis(methylsulfane) [41 mg, 0.3415 mmol], The reaction mixture was stirred at room temperature for 16 h. The reaction was complete detected by lcms. The solution was filtered, filtrate was collected. The reaction was quenched by H₂O (50 mL), The mixture was concentrated. The mixture was extracted with EA (50 mL*3). The combined organic layers were washed with brine (50 mL), dried over sodium sulfate, filtered and concentrated to give the crude product. The reaction mixture was concentrated under pressure. The residue was purified via Genal-Prep-HPLC and give the product

### Example 1-52 Preparation of Compound I-52

FIG. 52 illustrates the synthetic scheme of compound I-52. As shown in FIG. 52, the specific synthesis step is as follows:

### Step 1: N-(4-(2-(4-fluorophenyl)-5-(1-methoxyvinyl)-4,5,6,7-tetrahydropyrazolo[1,5-a]pyrazin-3-yl)pyridin-2-yl) acetamide

To a solution of N-(4-(2-(4-fluorophenyl)-4,5,6,7-tetrahydropyrazolo[1,5-a]pyrazin-3-yl)pyridin-2-yl) acetamide [100 mg, 0.2849 mmol] in pyridine [5 mL], then was added 1,1-dimethoxyethene [30 mg, 0.3415 mmol], The reaction mixture was stirred at room temperature for 16 h. The reaction was complete detected by lcms. The solution was filtered, filtrate was collected. The reaction was quenched by H₂O (50 mL), The mixture was concentrated. The mixture was extracted with EA (50 mL*3). The combined organic layers were washed with brine (50 mL), dried over sodium sulfate, filtered and concentrated to give the crude product. The reaction mixture was concentrated under pressure. The residue was purified via Genal-Prep-HPLC and give the product.

### Example 1-53 Preparation of Compound I-53

FIG. 53 illustrates the synthetic scheme of compound I-53. As shown in FIG. 53, the specific synthesis step is as follows:

### Step 1: N-(4-(2-(4-fluorophenyl)-5-(1-methylthio)methyl)-4,5,6,7-tetrahydropyrazolo[1,5-a]pyrazin-3-yl)pyridin-2-yl) acetamide

To a solution of N-(4-(2-(4-fluorophenyl)-4,5,6,7-tetrahydropyrazolo[1,5-a]pyrazin-3-yl)pyridin-2-yl) acetamide [100 mg, 0.2849 mmol] in DMF [5 mL], then was added (iodomethyl)(methyl)sulfane [64 mg, 0.3415 mmol], and K₂CO₃[79 mg, 0.5698 mmol]. The reaction mixture was stirred at room temperature for 16 h. The reaction was complete detected by lcms. The solution was filtered, filtrate was collected. The reaction was quenched by H₂O (50 mL), The mixture was concentrated. The mixture was extracted with EA (50 mL*3). The combined organic layers were washed with brine (50 mL), dried over sodium sulfate, filtered and concentrated to give the crude product. The reaction mixture was concentrated under pressure. The residue was purified via Genal-Prep-HPLC and give the product.

### Example 1-54 Preparation of Compound I-54

FIG. 54 illustrates the synthetic scheme of compound I-54. As shown in FIG. 54, the specific synthesis step is as follows:

### Step 1: N-(4-(2-(4-fluorophenyl)-5-(methylthio)-4,5,6,7-tetrahydropyrazolo[1,5-a]pyrazin-3-yl)pyridin-2-yl) acetamide

To a solution of N-(4-(2-(4-fluorophenyl)-4,5,6,7-tetrahydropyrazolo[1,5-a]pyrazin-3-yl)pyridin-2-yl) acetamide [100 mg, 0.2849 mmol] in DMF [5 mL], then was added methyl hypoiodothioite [59 mg, 0.3415 mmol], and K₂CO₃[79 mg, 0.5698 mmol]. The reaction mixture was stirred at room temperature for 16 h. The reaction was complete detected by lcms. The solution was filtered, filtrate was collected. The reaction was quenched by H₂O (50 mL), The mixture was concentrated. The mixture was extracted with EA (50 mL*3). The combined organic layers were washed with brine (50 mL), dried over sodium sulfate, filtered and concentrated to give the crude product. The reaction mixture was concentrated under pressure. The residue was purified via Genal-Prep-HPLC and give the product.

### Example 1-55 Preparation of Compound I-55

FIG. 55 illustrates the synthetic scheme of compound I-55. As shown in FIG. 55, the specific synthesis step is as follows:

### Step 1: N-(4-(5-(cyclopropanecarbonyl)-2-(4-fluorophenyl)-4,5,6,7-tetrahydropyrazolo[1,5-a]pyrazin-3-yl)pyridin-2-yl)butyramide

To a solution of (3-(2-aminopyridin-4-yl)-2-(4-fluorophenyl)-6,7-dihydropyrazolo[1,5-a]pyrazin-5(4H)-yl)(cyclopropyl)methanone [100 mg, 0.265 mmol] in pyridine [5 mL], then was added butyric acid [30 mg, 0.3415 mmol], HATU [118.963 mg, 0.3130 mmol], The reaction mixture was stirred at 100° C. for 16 h. The reaction was complete detected by lams. The solution was filtered, filtrate was collected. The reaction was quenched by H₂O (50 mL), The mixture was concentrated. The mixture was extracted with EA (50 mL*3). The combined organic layers were washed with brine (50 mL), dried over sodium sulfate, filtered and concentrated to give the crude product. The reaction mixture was concentrated under pressure. The residue was purified via Genal-Prep-HPLC and give the product.

### Example 1-56 Preparation of Compound 1-56

FIG. 56 illustrates the synthetic scheme of compound I-56. As shown in FIG. 56, the specific synthesis step is as follows:

### Step 1: N-(4-(5-butyryl-2-(4-fluorophenyl)-4,5,6,7-tetrahydropyrazolo[1,5-a]pyrazin-3-yl)pyridin-2-yl)butyramide

To a solution of 4-[2-(4-fluorophenyl)-4H,5H,6H,7H-pyrazolo[1,5-a]pyrazin-3-yl]pyridin-2-amine (200 mg, 0.6465 mmol) in DCM (10 mL), then butanoyl chloride (172.21 mg, 1.6162 mmol) and DIPEA (250.2 mg, 1.9395 mmol) was added. The reaction mixture was stirred at 25° C. for 2 h. The reaction was complete detected by lcms. The reaction mixture was concentrated under pressure. Then MeOH (5 mL) was added, K₂CO₃ (106.23 mg, 0.7698 mmol) was added. The reaction mixture was stirred at 25° C. for 2 h. The reaction was complete detected by lcms. The reaction was complete detected by lams. The reaction mixture was filtered and concentrated under pressure. The crude material was added to a silica gel column and was eluted with PE/EtOAc (1:1).

¹H NMR (400 MHz, DMSO) δ 10.49 (s, 1H), 8.24 (d, J=5.2 Hz, 1H), 8.00 (s, 1H), 7.40 (dd, J=8.8, 5.6 Hz, 2H), 7.18 (t, J=8.8 Hz, 2H), 6.80 (dd, J=23.4, 4.4 Hz, 1H), 4.78 (d, J=32.6 Hz, 2H), 4.23 (d, J=38.8 Hz, 2H), 4.05 (s, 2H), 2.44-2.30 (m, 4H), 1.62-1.48 (m, 4H), 0.89 (dt, J=12.2, 7.4 Hz, 6H).

### Example 1-57 Preparation of Compound I-57

FIG. 57 illustrates the synthetic scheme of compound I-57. As shown in FIG. 57, the specific synthesis step is as follows:

### Step 1: 1-(3-(2-aminopyridin-4-yl)-2-(4-fluorophenyl)-6,7-dihydropyrazolo[1,5-a]pyrazin-5(4H)-yl)ethane-1-thione (2)

To a solution of 1-[3-(2-aminopyridin-4-yl)-2-(4-fluorophenyl)-4H,6H,7H-pyrazolo[1,5-a]pyrazin-5-yl]ethenone (500 mg, 1.4230 mmol) in PhMe (10 mL), then Lawesson's Reagent (316.19 mg, 0.7826 mmol) was added. The reaction mixture was stirred at 110° C. for 5 h. The reaction was complete detected by lcms. The organic phase was washed with water (5 mL). The solution was filtered, filter cake was collected. Chemical Formula: calculated for (M+H⁺) C₁₉H₁₈FN₅S: 367.1, Found: 367.7.

### Step 2: N-(4-(5-ethanethioyl-2-(4-fluorophenyl)-4,5,6,7-tetrahydropyrazolo[1,5-a]pyrazin-3-yl pyridin-2-yl) acetamide

To a solution of 1-[3-(2-aminopyridin-4-yl)-2-(4-fluorophenyl)-4H,6H,7H-pyrazolo[1,5-a]pyrazin-5-yl]ethanethione (300 mg, 0.8165 mmol) in DCM (10 mL), then acetyl acetate (125.03 mg, 1.2247 mmol), DIEA (210.66 mg, 1.6330 mmol) was added. The reaction mixture was stirred at 25° C. for 5 h. The reaction was complete detected by lcms. The reaction mixture was concentrated under pressure. The crude material was added to a silica gel column and was eluted with PE/EtOAc (1:1).

¹HNMR (400 MHz, DMSO) δ 10.50 (d, J=4.2 Hz, 1H), 8.27-8.20 (m, 1H), 7.91 (s, 1H), 7.39-7.31 (m, 2H), 7.19-7.11 (m, 2H), 6.82 (ddd, J=14.2, 5.2, 1.6 Hz, 1H), 5.23 (d, J=123.2 Hz, 2H), 4.44 (dtd, J=22.0, 10.8, 5.2 Hz, 4H), 2.66 (d, J=22.8 Hz, 3H), 2.02 (d, J=1.3 Hz, 3H).

### Example 1-58 Preparation of Compound I-58

FIG. 58 illustrates the synthetic scheme of compound I-58. As shown in FIG. 58, the specific synthesis step is as follows:

### Step 1: N-(4-(5-acetyl-2-(4-fluorophenyl)-4,5,6,7-tetrahydropyrazolo[1,5-a]pyrazin-3-yl)pyridin-2-yl) acetamide

AcOAc (60 mg, 0.6 mmol) and Et₃N (88 mg, 0.87 mmol) were added to a solution of 4-(2-(4-fluorophenyl)-4,5,6,7-tetrahydropyrazolo[1,5-a]pyrazin-3-yl)pyridin-2-amine (90 mg, 0.29 mmol) in DCM (10 mL). The mixture was stirred at room temperature for 16 h. The reaction mixture was concentrated under pressure. The residue was purified via Prep-TLC DCM/MeOH (20:1) and give the product. Chemical Formula: calculated for (M+H⁺) C₂₁H₂₀FN₅O₂: 393.42, Found: 393.8. ¹H NMR (400 MHz, CDCl₃) δ 8.23-8.13 (m, 1H), 8.10 (d, J=5.3 Hz, 1H), 7.41 (dt, J=9.3, 4.8 Hz, 2H), 7.02 (q, J=8.5 Hz, 2H), 6.73 (t, J=4.8 Hz, 1H), 4.88 (d, J=17.0 Hz, 2H), 4.41-4.24 (m, 2H), 4.20-3.94 (m, 2H), 2.23 (t, J=5.0 Hz, 3H), 2.21 (s, 3H).

### Example 1-59 Preparation of Compound I-59

FIG. 59 illustrates the synthetic scheme of compound I-59. As shown in FIG. 59, the specific synthesis step is as follows:

### Step 1: 1-(azidomethyl)-4-methoxybenzeneethyl 2-(4-bromo-5-(chloromethyl)-3-(4-fluorophenyl)-1H-pyrazol-1-yl)acetate (2)

To a solution of ethyl 2-[4-bromo-3-(4-fluorophenyl)-5-(hydroxymethyl)pyrazol-1-yl]acetate (500 mg, 1.4 mmol) in DCM (10 mL) was added sulfoaroyl dichloride (249.9 mg, 2.1 mmol). The reaction mixture was stirred at 25° C. for 1 h. The reaction was complete detected by LCMS. The mixture was concentrated to give the crude product. Chemical Formula: calculated for (M+H⁺) C₁₄H₁₃BrClFN₂O₂: 374.0, Found: 374.9.

### Step 2: 3-bromo-2-(4-fluorophenyl)-5-methyl-4,5-dihydropyrazolo[1,5-a]pyrazin-6(7H)-one (3)

To a solution of ethyl 2-[4-bromo-5-(chloromethyl)-3-(4-fluorophenyl)pyrazol-1-yl]acetate (500 mg, 1.33 mmol) in NMP (10 mL) was added methanamine hydrochloride (269.398 mg, 3.99 mmol) and K₂CO₃ (550.6200 mg, 3.99 mmol). The reaction mixture was stirred at 150° C. for 1 h. The reaction was complete detected by LCMS. The mixture was filtered and concentrated to give the product, the crude material was added to a silica gel column and was eluted with EtOAc. ¹H NMR (400 MHz, CDCl₃) δ 7.90-7.83 (m, 2H), 7.13 (t, J=8.8 Hz, 2H), 4.87 (d, J=7.2 Hz, 2H), 4.54 (dd, J=7.2, 5.6 Hz, 2H), 3.21 (s, 3H).

### Step 3: 2-(4-fluorophenyl)-5-methyl-3-(2-methylpyridin-4-yl)-4,5-dihydropyrazolo[1,5-a]pyrazin-6(7H)-one

To a solution of 3-bromo-2-(4-fluorophenyl)-5-methyl-4H,7H-pyrazolo[1,5-a]pyrazin-6-one (100 mg, 0.15 mmol) in EtOH/PhMe/H₂O (5 mL) was added (2-methylpyridin-4-yl)bornanediol (127.36 mg, 0.93 mmol), Na₂CO₃ (98.58 mg, 0.9299 mmol) and Pd(dppf)Cl₂ (45.32 mg, 0.062 mmol). The reaction mixture was stirred at 100° C. for 1 h. The reaction was complete detected by LCMS. The mixture was filtered and concentrated to give the crude product. The crude material was added to a silica gel column and was eluted with DCM/MeOH (10:1).

¹H NMR (301 MHz, CD₃OD) δ 8.34 (d, J=5.4 Hz, 2H), 7.45-7.36 (m, 2H), 7.14-7.05 (m, 3H), 4.97 (s, 2H), 4.77 (s, 2H), 3.14 (s, 3H), 2.48 (s, 3H).

### Example 1-60 Preparation of Compound I-60

FIG. 60 illustrates the synthetic scheme of compound I-60. As shown in FIG. 60, the specific synthesis step is as follows:

### Step 1: 1-(3-(2-cyclopropylpyridin-4-yl)-2-(4-fluorophenyl)-6,7-dihydropyrazolo[1,5-a]pyrazin-5(4H)-yl)ethan-1-one

(2-cyclopropylpyridin-4-yl)boronic acid (43 mg, 0.27 mmol), 2 M Na₂CO₃ aqueous solutions (0.5 mL) and Pd(dppf)Cl₂ (43 mg, 0.059 mmol) were added to a solution of 1-(3-bromo-2-(4-fluorophenyl)-6,7-dihydropyrazolo[1,5-a]pyrazin-5(4H)-yl)ethan-1-one (100 mg, 0.30 mmol) in EtOH/toluene-8:2 (5 mL). The mixture was stirred at 100° C. for 1 h under nitrogen atmosphere on microwave. The reaction mixture was concentrated under pressure. The residue was purified via Prep-TLC PE/EA (1:50) and give the product. Chemical Formula: calculated for (M+H⁺) C₂₂H₂₁FN₄O: 376.44, Found: 376.8. ¹H NMR (400 MHz, CDCl₃) δ 8.40 (dd, J=17.9, 4.7 Hz, 1H), 7.39 (dd, J=8.5, 5.5 Hz, 2H), 7.02 (t, J=8.7 Hz, 2H), 6.95-6.78 (m, 2H), 4.80 (d, J=53.6 Hz, 2H), 4.32 (dt, J=27.7, 5.4 Hz, 2H), 4.24-3.93 (m, 2H), 2.21 (d, J=35.7 Hz, 3H), 2.01 (s, 1H), 1.10-0.89 (m, 4H).

### Example 1-61 Preparation of Compound I-61

FIG. 61 illustrates the synthetic scheme of compound I-61. As shown in FIG. 61, the specific synthesis step is as follows:

### Step 1: 5-benzyl-2-(4-fluorophenyl)-4,5,6,7-tetrahydropyrazolo[1,5-a]pyrazine (2)

To a solution of LiAlH₄ (2356 mg, 62 mmol) in THF (30 mL), was added slowly a solution of 5-benzyl-2-(4-fluorophenyl)-6,7-dihydropyrazolo[1,5-a]pyrazin-4(5H)-one (10,000 mg, 31 mmol) at 0° C. The reaction mixture was stirred at 25° C. for 15 h. 3 M NaOH aqueous (20 mL) was added. The solution was filtered, filtrate was collected. The reaction mixture was concentrated under pressure to give product.

Chemical Formula: calculated for (M+H⁺): C₁₉H₁₈FN₃, Molecular Weight: 307.37, Found: 307.8.

### Step 2: 5-benzyl-2-(4-fluorophenyl)-4,5,6,7-tetrahydropyrazolo[1,5-a]pyrazine (3)

To a solution of 5-benzyl-2-(4-fluorophenyl)-4,5,6,7-tetrahydropyrazolo[1,5-a]pyrazine (8000 mg, 25.9 mmol) in MeOH (80 mL), was added HCOONH₄ (16317 mg, 259 mmol) and Pd/C (1378 mg, 12.9 mmol). The reaction mixture was stirred at 50° C. for 15 h. The solution was filtered, filtrate was collected. The reaction mixture was concentrated under pressure to give product.

Chemical Formula: calculated for (M+H⁺): C₁₂H₁₂FN₃, Molecular Weight: 217.25, Found: 217.8.

### Step 3: 1-(2-(4-fluorophenyl)-6,7-dihydropyrazolo[1,5-a]pyrazin-5(4H)-yl)ethan-1-one (4)

To a solution of 5-benzyl-2-(4-fluorophenyl)-4,5,6,7-tetrahydropyrazolo[1,5-a]pyrazine (8 g, 36.8 mmol) in DCM (100 mL), was added acetic anhydride (7.5 g, 73.6 mmol) and TEA (7.45 g, 73.6 mmol). The reaction mixture was stirred at 25° C. for 15 h. The reaction mixture was concentrated under pressure. The crude material was added to a silica gel column and was eluted with PE/EtOAc (1:1) to give product.

Chemical Formula: calculated for (M+H⁺): C₁₄H₁₄FN₃O, Molecular Weight: 259.28, Found: 259.8.

### Step 4: 1-(3-bromo-2-(4-fluorophenyl)-6,7-dihydropyrazolo[1,5-a]pyrazin-5(4H)-yl)ethan-1-one (5)

NBS (3.78 g, 21.2 mmol) was added to a solution of 1-(2-(4-fluorophenyl)-6,7-dihydropyrazolo[1,5-a]pyrazin-5(4H)-yl)ethan-1-one (5 g, 19.3 mmol) in ACN (50 mL). The reaction mixture was stirred at 25° C. for 15 h. The reaction mixture was concentrated under pressure. The crude material was added to a silica gel column and was eluted with PE/EtOAc (2:1) to give product.

Chemical Formula: calculated for (M+H⁺): C₁₄H₁₃BrFN₃O, Molecular Weight: 338.18, Found: 337.8.

### Step 5: 1-(2-(4-fluorophenyl-3-(2-methoxypyridin-4-yl)-6,7-dihydropyrazolo[1,5-a]pyrazin-5(4H)-yl)ethan-1-one

To a solution of 1-(3-bromo-2-(4-fluorophenyl)-6,7-dihydropyrazolo[1,5-a]pyrazin-5(4H)-yl)ethan-1-one (200 mg, 0.59 mmol) in EtOH (6 mL) and PhMe (1 mL), was added (2-methoxypyridin-4-yl)boronic acid (135 mg, 0.885 mmol), Pd(dppf)Cl₂ (43 mg, 0.059 mmol) and Na₂CO₃ (125 mg, 1.18 mmol). The reaction mixture was stirred at microwave at 100° C. for 1 h.

The solution was filtered, filtrate was collected. The residue was purified via Genal-Prep-HPLC and give the product.

¹H NMR (400 MHz, CDCl₃) δ 8.10 (dd, J=11.6, 5.4 Hz, 1H), 7.40 (dd, J=8.8, 5.4 Hz, 2H), 7.01 (t, J=8.3 Hz, 2H), 6.63 (d, J=5.3 Hz, 1H), 6.55 (s, 11H), 4.84 (s, 1H), 4.72 (s, 1H), 4.34 (d, J=5.7 Hz, 1H), 4.27 (d, J=5.8 Hz, 1H), 4.18-4.11 (m, 1H), 4.00 (t, J=5.5 Hz, 1H), 3.95 (d, J=7.5 Hz, 3H), 2.20 (d, J=34.0 Hz, 3H).

### Example 1-62 Preparation of Compound I-62

FIG. 62 illustrates the synthetic scheme of compound I-62. As shown in FIG. 62, the specific synthesis step is as follows:

### Step 1: 1-(2-(4-fluorophenyl)-3-(2-(methylamino)pyridin-4-yl)-6,7-dihydropyrazolo[1,5-a]pyrazin-5(4H)-yl) ethan-1-one

To a solution of 1-(3-bromo-2-(4-fluorophenyl)-6,7-dihydropyrazolo[1,5-a]pyrazin-5(4H)-yl)ethan-1-one (100 mg, 0.3 mmol) in EtOH (3 mL) and PhMe (0.5 mL), was added (2-(methylamino)pyridin-4-yl)boronic acid (105 mg, 0.45 mmol), Pd(dppf)Cl₂ (22 mg, 0.03 mmol) and Na₂CO₃ (63.6 mg, 0.6 mmol). The reaction mixture was stirred at microwave at 100° C. for 1 h. The solution was filtered, filtrate was collected. The residue was purified via Genal-Prep-HPLC and give the product.

¹H NMR (400 MHz, CDCl₃) δ 7.80 (s, 1H), 7.41 (dd, J=8.5, 5.1 Hz, 2H), 7.06 (t, J=8.6 Hz, 2H), 6.43 (s, 1H), 6.31 (s, 1H), 5.40 (s, 1H), 4.83 (d, J=56.3 Hz, 2H), 4.34 (dd, J=20.2, 14.7 Hz, 2H), 4.22-3.97 (m, 2H), 2.81 (s, 3H), 2.22 (d, J=32.6 Hz, 3H).

### Example 1-63 Preparation of Compound I-63

FIG. 63 illustrates the synthetic scheme of compound I-63. As shown in FIG. 63, the specific synthesis step is as follows:

### Step 1: 1-(3-(6-aminopyrimidin-4-yl)-2-(4-fluorophenyl)-6,7-dihydropyrazolo[1,5-a]pyrazin-5(4H)-yl)ethan-1-one

Pd(dppf)Cl₂ (28.5 mg, 0.039 mmol), 6-bromopyrimidin-4-amine (101.8 mg, 0.585 mmol), Cs₂CO₃ (381 mg, 1.17 mmol) was added to a solution of 1-[2-(4-fluorophenyl)-3-(4,4,5,5-tetramethyl-1,3,2-dioxaborolan-2-yl)-4H,6H,7H-pyrazolo[1,5-a]pyrazin-5-yl]ethanone (150 mg, 0.39 mmol) in dioxane/H₂O (20:4 mL). The mixture was allowed to react at 90° C. under N₂ for 16 h. The reaction mixture was cooled to room temperature and concentrated under pressure. The crude material was added to a silica gel column and was eluted with MeOH/DCM (25:1) to give product as yellow solid (10.3 mg, yield: 7.4%). Chemical Formula: calculated for (M+H⁺) Chemical Formula: C₁₈H₁₇FN₆O: 352.14, Found:352.9. ¹H NMR (400 MHz, CDCl₃) δ 8.55 (d, J=4.7 Hz, 1H), 7.48 (dd, J=8.6, 5.3 Hz, 2H), 7.11 (q, J=8.2 Hz, 2H), 6.12 (s, 1H), 5.19-4.98 (m, 4H), 4.35-4.23 (m, 2H), 4.16-3.97 (m, 2H), 2.24 (s, 3H).

### Example 1-64 Preparation of Compound I-64

FIG. 64 illustrates the synthetic scheme of compound I-64. As shown in FIG. 64, the specific synthesis step is as follows:

### Step 1: 2-(2-aminopyridin-4-yl)-3-(4-fluorophenyl)-6,7-dihydropyrazolo[1,5-a]pyrazin-4(5H)-one

To a solution of 2-bromo-3-(4-fluorophenyl)-5H,6H,7H-pyrazolo[1,5-a]pyrazin-4-one (200 mg, 0.64 mmol) in 1,4-Dioxane (5 mL) was added (6-aminopyridin-3-yl)bornanediol (176.553 mg, 1.28 mmol), Na₂CO₃ (203.52 mg, 1.92 mmol) and Pd(dppf)Cl₂ (93.568 mg, 0.128 mmol). The reaction mixture was stirred at 100° C. for 1 h. The reaction was complete detected by LCMS. The mixture was filtered and concentrated to give the product. The crude material was added to a silica gel column and was eluted with CH₂Cl₂/MeOH (10:1). The residue was purified via Genal-Prep-HPLC and give the product.

¹H NMR (301 MHz, CD₃OD) δ 8.40 (s, 1H), 7.74 (d, J=5.6 Hz, 1H), 7.39-7.27 (m, 2H), 7.09 (t, J=8.8 Hz, 2H), 6.68 (s, 1H), 6.59 (d, J=5.6 Hz, 1H), 4.51-4.44 (m, 2H), 3.82-3.74 (m, 2H).

### Example 1-65 Preparation of Compound I-65

FIG. 65 illustrates the synthetic scheme of compound I-65. As shown in FIG. 65, the specific synthesis step is as follows:

### Step 1: 4-bromopicolinamide (2)

A solution of methyl 4-bromopicolinate (500 mg, 2.3 mmol) in ammonia hydrate (100 mL) was stirred at 35° C. for 4 h. The residue was extracted with EA. The reaction mixture was concentrated under pressure and give the product. Chemical Formula: calculated for (M+H⁺) C₆H₅BrN₂O:201.02, Found: 200.8.

### Step 2: 4-(5-acetyl-2-(4-fluorophenyl)-4,5,6,7-tetrahydropyrazolo[1,5-a]pyrazin-3-yl)picolinamide

4-bromopicolinamide (156 mg, 0.78 mmol), Na₂CO₃ (338 mg, 1 mmol) and Pd(dppf)Cl₂ (40 mg, 0.05 mmol) were added to a solution of 1-(2-(4-fluorophenyl)-3-(4,4,5,5-tetramethyl-1,3,2-dioxaborolan-2-yl)-6,7-dihydropyrazolo[1,5-a]pyrazin-5(4H)-yl)ethan-1-one (200 mg, 0.52 mmol) in 1,4-dioxane:H₂O=5:1 (12 mL). The mixture was stirred at 90° C. for 3 h under nitrogen atmosphere. The reaction mixture was concentrated under pressure. The residue was purified via Prep-TLC PF/EA (1:50) and give the product. Chemical Formula: calculated for (M+H⁺) C₂₀H₁₈FN₅O₂: 379.40, Found: 379.8.1H NMR (400 MHz, CDCl₃) δ 8.48 (d, J=4.8 Hz, 1H), 8.04 (d, J=17.9 Hz, 1H), 7.87 (a, 1H), 7.47-7.30 (m, 2H), 7.15 (dd, J=12.8, 4.3 Hz, 1H), 7.01 (dd, J=14.3, 8.6 Hz, 2H), 5.62 (d, J=13.1 Hz, 1H), 4.83 (d, J=36.8 Hz, 2H), 4.41-4.26 (m, 2H), 4.23-3.96 (m, 2H), 2.20 (d, J=36.6 Hz, 3H).

### Example 1-66 Preparation of Compound I-66

FIG. 66 illustrates the synthetic scheme of compound I-66. As shown in FIG. 66, the specific synthesis step is as follows:

### Step 1: N-(4-(5-acetyl-2-(4-fluorophenyl)-4,5,6,7-tetrahydropyrazolo[1,5-a]pyrazin-3-yl) pyridin-2-yl)-2-(4-fluorophenyl) acetamide

To a solution of 1-[3-(2-aminopyridin-4-yl)-2-(4-fluorophenyl)-4H,6H,7H-pyrazolo[1,5-a]pyrazin-5-yl]ethanone (50 mg, 0.14 mmol) in pyridine (5 mL) was added (4-fluorophenyl) acetic acid (43.1588 mg, 0.28 mmol) and HATU (53.2 mg, 0.14 mmol). The reaction mixture was stirred at 100° C. for 16 h. The reaction was complete detected by LCMS. The solution was filtered, filtrate was collected. The reaction was quenched by H₂O (50 mL). The mixture was concentrated. The mixture was extracted with EA (50 mL*3). The combined organic layers were washed with brine (50 mL), dried over sodium sulfate, filtered and concentrated to give the crude product. The reaction mixture was concentrated under pressure. The residue was purified via Genal-Prep-HPLC and give the product.

¹H NMR (301 MHz, DMSO) δ 10.77 (s, 1H), 8.25 (d, J=4.8 Hz, 1H), 7.94 (s, 1H), 7.36 (dt, J=11.7, 8.7 Hz, 4H), 7.15 (dd, J=18.6, 9.0 Hz, 1H), 6.77 (d, J=5.1 Hz, 1H), 4.74 (d, J=23.7 Hz, 2H), 4.28 (a, 2H), 4.01 (s, 2H), 3.70 (s, 2H), 2.09 (d, J=23.4 Hz, 3H).

### Example 1-67 Preparation of Compound I-67

FIG. 67 illustrates the synthetic scheme of compound I-67. As shown in FIG. 67, the specific synthesis step is as follows:

### Step 1: 5-benzyl-2-(4-fluorophenyl)-3-(4,4,5,5-tetramethyl-1,3,2-dioxaborolan-2-yl-4,5,6,7-tetrahydropyrazolo[1,5-a]pyrazine (2)

To a solution of 5-benzyl-3-bromo-2-(4-fluorphenyl)-4,5,6,7-tetrahydropyrazolo[1,5-a]pyrazine (10 g, 25.9 mmol) in THF (100 mL), was added n-BuLi (22 mL, 51.8 mmol, 2.4N) at −78° C. The reaction mixture was stirred at −78° C. for 1 h. 2-isopropoxy-4,4,5,5-tetramethyl-1,3,2-dioxaborolane (48.2 g, 0.259 mol) was added. The reaction mixture was stirred at 25° C. for 15 h. The reaction mixture was concentrated under pressure. The crude material was added to a silica gel column and was eluted with PE/EtOAc (3:1) to give product.

Chemical Formula: calculated for (M+H⁺): C₂₅H₂₉BFN₃O₂, Molecular Weight: 433.33, Found: 433.8.

### Step 2: 2-(4-fluorophenyl)-3-(4,4,5,5-tetramethyl-1,3,2-dioxaborolan-2-yl)-4,5,6,7-tetrahydropyrazolo[1,5-a]pyrazine (3)

To a solution of 5-benzyl-2-(4-fluorophenyl)-3-(4,4,5,5-tetramethyl-1,3,2-dioxaborolan-2-yl)-4,5,6,7-tetrahydropyrazolo[1,5-a]pyrazine (8000 mg, 18.46 mmol) in MeOH (100 mL), was added HCOONH₄ (11640 mg, 184.6 mmol) and Pd/C (1964 mg, 18.46 mmol). The reaction mixture was stirred at 50° C. for 5 h. The solution was filtered, filtrate was collected. The reaction mixture was concentrated under pressure to give product.

Chemical Formula: calculated for (M+H⁺): C₁₈H₂₃BFN₃O₂, Molecular Weight: 343.21, Found: 343.8.

### Step 3: 1-(2-(4-fluorophenyl)-3-(4,4,5,5-tetramethyl-1,3,2-dioxaborolan-2-yl)-6,7-dihydropyrazolo[1,5-a]pyrazin-5(4H)-yl)ethan-1-one (4)

To a solution of 2-(4-fluorophenyl)-3-(4,4,5,5-tetramethyl-1,3,2-dioxaborolan-2-yl)-4,5,6,7-tetrahydropyrazolo[1,5-a]pyrazine (10 g, 29.1 mmol) in DCM (100 mL), was added acetic anhydride (5.94 g, 58.2 mmol) and TEA (5.89 g, 58.2 mmol). The reaction mixture was stirred at 25° C. for 15 h. The reaction mixture was concentrated under pressure. The crude material was added to a silica gel column and was eluted with PE/EtOAc (1:1) to give product.

Chemical Formula: calculated for (M+H⁺): C₂₀H₂₅BFN₃O₃, Molecular Weight: 385.25, Found: 385.8.

### Step 4: 1-(3-(2-amino-6-methylpyridin-4-yl)-2-(4-fluorophenyl)-6,7-dihydropyrazolo[1,5-a]pyrazin-5(4H)-yl)ethan-1-one

To a solution of 1-(2-(4-fluorophenyl)-3-(4,4,5,5-tetramethyl-1,3,2-dioxaborolan-2-yl)-6,7-dihydropyrazolo[1,5-a]pyrazin-5(4H)-yl)ethan-1-one (200 mg, 0.52 mmol) in dioxane (10 mL) and water (2 mL), was added 4-bromo-6-methylpyridin-2-amine (146 mg, 0.78 mmol), Pd(dppf)Cl₂ (38 mg, 0.052 mmol) and Cs₂CO₃ (339 mg, 1.04 mmol). The reaction mixture was stirred at 100° C. for 15 h. The reaction mixture was concentrated under pressure. The crude material was added to a silica gel column and was eluted with DCM/EtOAc (1:2). The residue was purified via Genal-Prep-HPLC and give the product.

¹H NMR (400 MHz, CDCl₃) δ 8.48 (s, 1H), 7.40 (dd, J=8.6, 5.2 Hz, 2H), 7.06 (t, J=8.6 Hz, 3H), 6.32-6.15 (m, 2H), 4.87 (s, 1H), 4.75 (s, 1H), 4.40-4.24 (m, 2H), 4.22-3.94 (m, 2H), 2.41 (s, 3H), 2.22 (d, J=25.0 Hz, 3H).

### Example 1-68 Preparation of Compound I-68

FIG. 68 illustrates the synthetic scheme of compound I-68. As shown in FIG. 68, the specific synthesis step is as follows:

### Step 1: N-(4-(5-acetyl-2-(4-fluorophenyl)-4,5,6,7-tetrahydropyrazolo[1,5-a]pyrazin-3-yl)pyridin-2-yl)-2-chloroacetamide (2)

2-chloroacetyl chloride (65 mg, 0.57 mmol) and Et₃N (58 mg, 0.57 mmol) were added to a solution of 1-(3-(2-aminopyridin-4-yl)-2-(4-fluorophenyl)-6,7-dihydropyrazolo[1,5-a]pyrazin-5(4H)-yl)ethan-1-one (100 mg, 0.28 mmol) in DCM (10 mL) at 0° C. The mixture was stirred 0° C. for 1 h. The reaction mixture was concentrated under pressure. The residue was purified via Prep-TLC PE/EA (2:3) and give the product. Chemical Formula: calculated for (M+H⁺) C₂₁H₁ClFN₃O₂: 427.86, Found: 427.8.

### Step 2: N-(4-(5-acetyl-2-(4-fluorophenyl)-4,5,6,7-tetrahydropyrazolo[1,5-a]pyrazin-3-yl)pyridin-2-yl)-2-(dimethylamino) acetamide

Dimethylamine (1 mL, 2.3 mmol) and Et₃N (47 mg, 0.46 mmol) were added to a solution of N-(4-(S-acetyl-2-(4-fluorophenyl)-4,5,6,7-tetrahydropyrazolo[1,5-a]pyrazin-3-yl)pyridin-2-yl)-2-chloroacetamide (100 mg, 0.23 mmol) in DCM (10 mL) at 0° C. The mixture was stirred 0° C. for 1 h. The solution was concentrated under pressure. The residue was purified via Prep-TLC PE/EA (4:1) and give the product. Chemical Formula: calculated for (M+H⁺) C₂₃H₂₅FN₆O₂: 436.49, Found: 436.8.

¹H NMR (400 MHz, CDCl₃) δ 8.28-8.02 (m, 2H), 7.40 (dt, J=8.5, 5.7 Hz, 2H), 7.01 (q, J=8.6 Hz, 2H), 6.73 (d, J=4.8 Hz, 1H), 4.88 (d, J=25.4 Hz, 2H), 4.39-4.24 (m, 2H), 4.23-3.94 (m, 2H), 3.36 (d, J=32.6 Hz, 2H), 2.55 (d, J=20.6 Hz, 6H), 2.22 (d, J=15.0 Hz, 3H).

### Example 1-69 Preparation of Compound I-69

FIG. 69 illustrates the synthetic scheme of compound I-69. As shown in FIG. 69, the specific synthesis step is as follows:

### Step 1: (E)-N-(4-(5-acetyl-2-(4-fluorophenyl)-4,5,6,7-tetrahydropyrazolo[1,5-a]pyrazin-3-yl)pyridin-2-yl)-3-(2,4-dimethoxyphenyl) acrylamide

To a solution of 1-[3-(2-aminopyridin-4-yl)-2-(4-fluorophenyl)-4H,6H,7H-pyrazolo[1,5-a]pyrazin-5-yl]ethanone (100 mg, 0.2846 mmol) in pyridine (5 mL) was added (2E)-3-(2,4-dimethoxyphenyl)prop-2-enoic acid (71.1081 mg, 0.3415 mmol), and HATU (118.963 mg, 0.3130 mmol). The reaction mixture was stirred at 100° C. for 16 h. The reaction was complete detected by LCMS. The solution was filtered, filtrate was collected. The reaction was quenched by H₂O (50 mL). The mixture was concentrated. The mixture was extracted with EA (50 mL*3). The combined organic layers were washed with brine (50 mL), dried over sodium sulfate, filtered and concentrated to give the crude product. The reaction mixture was concentrated under pressure. The residue was purified via Genal-Prep-HPLC and give the product.

¹H NMR (400 MHz, CDCl₃) δ 8.35 (s, 1H), 8.08-7.89 (m, 2H), 7.53-7.38 (m, 3H), 7.09-6.99 (m, 2H), 6.74 (d, J=4.4 Hz, 2H), 6.56-6.44 (m, 2H), 4.96 (d, J=11.6 Hz, 2H), 4.32 (d, J=22.4 Hz, 2H), 4.17 (s, 1H), 4.03 (s, 1H), 3.91 (d, J=6.0 Hz, 3H), 3.84 (d, J=13.6 Hz, 3H), 2.24 (s, 3H).

### Example 1-79 Preparation of Compound I-70

FIG. 70 illustrates the synthetic scheme of compound I-70. As shown in FIG. 70, the specific synthesis step is as follows:

### Step 1: N-(4-(5-acetyl-2-(4-fluorophenyl)-4,5,6,7-tetrahydropyrazolo[1,5-a]pyrazin-3-yl)pyridin-2-yl)picolinamide

Picolinoyl chloride (73 mg, 0.512 mmol) and Et₃N (65 mg, 0.64 mmol) was added to a solution of 1-(3-(2-aminopyridin-4-yl)-2-(4-fluorophenyl)-6,7-dihydropyrazolo[1,5-a]pyrazin-5(4H)-yl)ethan-1-one (150 mg, 0.43 mmol) in DCM (20 mL). The mixture was stirred at 25° C. for 1 h. The reaction mixture was concentrated under pressure. The residue was purified via Prep-TLC DCM/MeOH (10:1) and give the product. Chemical Formula: calculated for (M+H⁺) C₂₅H₂₁FN₆O₂: 456.48, Found: 456.8. NMR (400 MHz, CDCl₃) δ 8.68 (d, J=4.4 Hz, 1H), 8.41 (d, J=7.2 Hz, 1H), 8.26 (dd, J=12.7, 6.6 Hz, 2H), 8.03-7.83 (m, 11H), 7.59-7.47 (m, 1H), 7.49-7.37 (m, 2H), 7.03 (dd, J=16.6, 8.4 Hz, 2H), 6.78 (t, J=6.2 Hz, 1H), 4.94 (d, J=18.9 Hz, 2H), 4.42-4.26 (m, 2H), 4.24-3.97 (m, 2H), 2.23 (d, J=9.2 Hz, 3H).

### Example 1-71 Preparation of Compound I-71

FIG. 71 illustrates the synthetic scheme of compound I-71. As shown in FIG. 71, the specific synthesis step is as follows:

### Step 1: N-(4-(2-(4-fluorophenyl)-5-methyl-4,5,6,7-tetrahydropyrazolo[1,5-a]pyrazin-3-yl)pyridin-2-yl) acetamide

To a solution of N-{4-[2-(4-fluorophenyl)-4H,5H,6H,7H-pyrazolo[1,5-a]pyrazin-3-yl]pyridin-2-yl}acetamide (200 mg, 0.57 mmol) in THF (5 mL), formaldehyde (25.6500 mg, 0.855 mmol) was added. The reaction mixture was stirred at 60° C. for 16 h. Sodium triacetoxyborohydride (181.2600 mg, 0.855 mmol) was added. The reaction mixture was stirred at 25° C. for 2 h. The reaction was complete detected by lcms. The reaction mixture was concentrated under pressure. The organic phase was washed with water (5 mL). The residue was extracted with EA (50 mL*3). The combined organic layers were washed with brine (50 mL), dried over sodium sulfate, filtered and concentrated to give the crude product. The residue was purified via Genal-Prep-HPLC and give the product.

¹H NMR (400 MHz, DMSO) δ 10.49 (s, 1H), 8.20 (dd, J=5.2, 0.5 Hz, 1H), 7.92 (s, 1H), 7.42-7.35 (m, 2H), 7.21-7.12 (m, 2H), 6.76 (dd, J=5.2, 1.6 Hz, 1H), 4.19 (t, J=5.6 Hz, 2H), 3.61 (s, 2H), 2.92 (t, J=5.6 Hz, 2H), 2.41 (s, 3H), 2.06 (s, 3H).

### Example 1-72 Preparation of Compound I-72

FIG. 72 illustrates the synthetic scheme of compound I-72. As shown in FIG. 72, the specific synthesis step is as follows:

### Step 1:N-(4-(5-acetyl-2-(4-fluorophenyl)-4,5,6,7-tetrahydropyrazolo[1,5-a]pyrazin-3-yl)pyridin-2-yl)-2-phenylacetamide

To a solution of 1-(3-(2-aminopyridin-4-yl)-2-(4-fluorophenyl)-6,7-dihydropyrazolo[1,5-a]pyrazin-5(4H)-yl)ethan-1-one (100 mg, 0.28 mmol) in DCM (10 mL), 2-phenylacetyl chloride (52 mg, 0.336 mmol) and Et₃N (267 mg, 0.84 mmol) were added. Then the mixture was stirred at 25° C. for 1 h. The reaction mixture was concentrated under pressure. Chemical Formula: calculated for (M+H⁺) C₂₇H₂₄FN₅O₂: 469.52, Found: 469.8. ¹H NMR (400 MHz, CDCl₃) δ 8.20 (s, 1H), 8.06 (d, J=5.2 Hz, 1H), 7.39 (ddt, J=15.6, 11.0, 5.4 Hz, 7H), 7.15-6.90 (m, 2H), 6.70 (d, J=5.1 Hz, 1H), 4.87 (d, J=19.6 Hz, 2H), 4.45-4.22 (m, 2H), 4.15 (t, J=5.5 Hz, 1H), 4.00 (t, J=5.4 Hz, 1H), 3.78 (d, J=8.8 Hz, 2H), 2.22 (d, J=13.5 Hz, 3H).

### Example 1-73 Preparation of Compound I-73

FIG. 73 illustrates the synthetic scheme of compound I-73. As shown in FIG. 73, the specific synthesis step is as follows:

### Step 1:N-(4-(5-acetyl-2-(4-fluorophenyl)-4,5,6,7-tetrahydropyrazolo[1,5-a]pyrazin-3-yl)pyridin-2-yl)benzamide

To a solution of 1-(3-(2-aminopyridin-4-yl)-2-(4-fluorophenyl)-6,7-dihydropyrazolo[1,5-a]pyrazin-5(4H)-yl)ethan-1-one (100 mg, 0.28 mmol) in DCM (20 mL), benzoyl chloride (48 mg, 0.34 mmol) and Et₃N (86.4 mg, 0.85 mmol) were added. The mixture was stirred at 25° C. for 1 h. The reaction mixture was concentrated under pressure. Chemical Formula: calculated for (M+H⁺) C₂₆H₂₂FN₅O₂: 455.49, Found: 455.8. ¹H NMR (400 MHz, CDCl₃) δ 8.82 (s, 11H), 8.37 (d, J=6.1 Hz, 1H), 8.15 (d, J=5.2 Hz, 1H), 7.94 (t, J=7.2 Hz, 2H), 7.55 (ddd, J=23.8, 12.9, 7.4 Hz, 3H), 7.44 (dt, J=5.3, 4.7 Hz, 2H), 7.03 (q, J=8.6 Hz, 2H), 6.76 (t, J=3.6 Hz, 1H), 4.94 (d, J=12.1 Hz, 2H), 4.44-4.21 (m, 2H), 4.18 (t, J=5.5 Hz, 1H), 4.03 (t, J=5.4 Hz, 1H), 2.24 (d, J=4.1 Hz, 3H).

### Example 1-74 Preparation of Compound I-74

FIG. 74 illustrates the synthetic scheme of compound I-74. As shown in FIG. 74, the specific synthesis step is as follows:

### Step 1: 1-(2-(4-fluorophenyl)-3-(2-(phenylamino)pyridin-4-yl)-6,7-dihydropyrazolo[1,5-a]pyrazin-5(4H)-yl) ethan-1-one

To a solution of 1-[3-(2-aminopyridin-4-yl)-2-(4-fluorophenyl)-4H,6H,7H-pyrazolo[1,5-a]pyrazin-5-yl]ethanone (100 mg, 0.28 mmol) in dioxane (20 mL), Cs₂CO₃ (182 mg, 0.56 mmol), iodobenzene (114 mg, 0.56 mmol), Pd₂(dba)₃ (25.6 mg, 0.028 mmol) and x-Phos (1.34 mg, 0.028 mmol) was added. The reaction mixture was stirred at 100° C. for 15 h. The reaction mixture was concentrated under pressure. The residue was purified via Prep-TLC (PE/EtOAc)=(2:1). The residue was purified via Genal-Prep-HPLC and give the product.

¹H NMR (400 MHz, DMSO) δ 9.02 (d, J=17.8 Hz, 1H), 8.13 (t, J=6.5 Hz, 1H), 7.57 (t, J=6.2 Hz, 2H), 7.45 (dd, J=8.7, 5.5 Hz, 2H), 7.29-7.14 (m, 4H), 6.87 (t, J=7.3 Hz, 1H), 6.62 (s, 1H), 6.51 (dd, J=5.2, 1.2 Hz, 1H), 4.78 (d, J=20.6 Hz, 2H), 4.27 (dd, J=30.2, 24.8 Hz, 2H), 4.02 (t, J=5.5 Hz, 2H), 2.13 (d, J=21.6 Hz, 3H).

### Example 1-75 Preparation of Compound I-75

FIG. 75 illustrates the synthetic scheme of compound I-75. As shown in FIG. 75, the specific synthesis step is as follows:

### Step 1:N-(4-(5-acetyl-2-(4-fluorophenyl)-4,5,6,7-tetrahydropyrazolo[1,5-a]pyrazin-3-yl)pyridin-2-yl)-2-cyclopropylacetamide

To a solution of 1-(3-(2-aminopyridin-4-yl)-2-(4-fluorophenyl)-6,7-dihydropyrazolo[1,5-a]pyrazin-5(4H)-yl)ethan-1-one (100 mg, 0.29 mmol) in DCM (10 mL), 2-cyclopropylacetic acid (34 mg, 0.34 mmol), T₃P (181 mg, 0.57 mmol) and DIPEA (220 mg, 1.7 mmol) were added. The mixture was stirred at 25° C. for 24 h. The reaction mixture was concentrated under pressure. Chemical Formula: calculated for (M+H⁺) C₂₄H₂₄FN₃O₂: 433.49, found: 433.8. ¹H NMR (400 MHz, DMSO) δ 10.41 (d, J=7.0 Hz, 1H), 8.24 (t, J=5.2 Hz, 1H), 8.01 (s, 1H), 7.59-7.29 (m, 2H), 7.19 (t, J=8.9 Hz, 2H), 7.00-6.70 (m, 1H), 4.77 (d, J=31.4 Hz, 2H), 4.27 (dd, J=29.5, 24.2 Hz, 2H), 4.11-3.91 (m, 2H), 2.39-2.20 (m, 2H), 2.12 (d, J=24.7 Hz, 3H), 1.11-0.81 (m, 1H), 0.45 (dd, J=5.2, 2.9 Hz, 2H), 0.29-0.09 (m, 2H).

### Example 1-76 Preparation of Compound I-76

FIG. 76 illustrates the synthetic scheme of compound I-76. As shown in FIG. 76, the specific synthesis step is as follows:

### Step 1:N-(4-(5-acetyl-2-(4-fluorophenyl)-4,5,6,7-tetrahydropyrazolo[1,5-a]pyrazin-3-yl)pyridin-2-yl)-1-methyl-1H-pyrazole-3-carboxamide

To a solution of 1-(3-(2-aminopyridin-4-yl)-2-(4-fluorophenyl)-6,7-dihydropyrazolo[1,5-a]pyrazin-5(4H)-yl)ethan-1-one (100 mg, 0.28 mmol) in pyridine (10 mL), 1-methyl-1H-pyrazole-3-carboxylic acid (53.84 mg, 0.43 mmol) and HATU (162.32 mg, 0.43 mmol) were added. The mixture was stirred at 100° C. for 16 h. The reaction mixture was concentrated under pressure. Chemical Formula: calculated for (M+H⁺) C₂₄H₂₂FN₇O₂: 459.49, found: 459.8. ¹H NMR (400 MHz, DMSO) δ 9.58 (s, 1H), 8.30 (d, J=5.1 Hz, 1H), 8.05 (s, 1H), 7.89 (d, J=2.1 Hz, 1H), 7.43 (dd, J=8.7, 5.5 Hz, 2H), 7.20 (t, J=8.9 Hz, 2H), 6.98-6.85 (m, 1H), 6.83 (d, J=2.3 Hz, 1H), 4.82 (d, J=31.2 Hz, 2H), 4.44-4.14 (m, 2H), 4.14-3.99 (m, 2H), 3.96 (s, 3H), 2.13 (d, J=20.7 Hz, 3H).

### Example 1-77 Preparation of Compound I-77

FIG. 77 illustrates the synthetic scheme of compound I-77. As shown in FIG. 77, the specific synthesis step is as follows:

### Step 1:N-(4-(5-acetyl-2-(4-fluorophenyl)-4,5,6,7-tetrahydropyrazolo[1,5-a]pyrazin-3-yl)pyridin-2-yl)-1-(4-fluorophenyl)cyclopropane-1-carboxamide

To a solution of 1-(3-(2-aminopyridin-4-yl)-2-(4-fluorophenyl)-6,7-dihydropyrazolo[1,5-a]pyrazin-5(4H)-yl)ethan-1-one (200 mg, 0.57 mmol) in pyridine (10 mL), 1-(4-fluorophenyl)cyclopropane-1-carboxylic acid (154 mg, 0.85 mmol) and HATU (325 mg, 0.85 mmol) were added. The mixture was stirred at 100° C. for 16 h. The reaction mixture was concentrated under pressure. Chemical Formula: calculated for (M+H⁺) C₂₉H₂₅F₂N₅O₂: 513.55, Found: 513.8. ¹H NMR (400 MHz, DMSO) δ 8.48 (d, J=10.2 Hz, 1H), 8.15 (t, J=6.2 Hz, 1H), 7.92 (d, J=4.2 Hz, 1H), 7.56 (dt, J=12.5, 6.3 Hz, 2H), 7.43-7.31 (m, 2H), 7.26 (t, J=8.9 Hz, 2H), 7.18 (t, J=8.9 Hz, 2H), 6.97-6.64 (m, 1H), 4.74 (d, J=29.0 Hz, 2H), 4.39-4.11 (m, 2H), 4.11-3.87 (m, 2H), 2.11 (d, J=29.7 Hz, 3H), 1.48 (dd, J=6.9, 4.1 Hz, 2H), 1.15 (dd, J=6.9, 4.2 Hz, 2H).

### Example 1-78 Preparation of Compound I-78

FIG. 78 illustrates the synthetic scheme of compound I-78. As shown in FIG. 78, the specific synthesis step is as follows:

### Step 1: N-(4-(5-acetyl-2-(4-fluorophenyl)-4,5,6,7-tetrahydropyrazolo[1,5-a]pyrazin-3-yl)pyridin-2-yl)benzenesulfonamide

To a solution 1-(3-(2-aminopyridin-4-yl)-2-(4-fluorophenyl)-6,7-dihydropyrazolo[1,5-a]pyrazin-5(4H)-yl)ethan-1-one (130 mg, 0.37 mmol) in DCM (10 mL), triethylamine (112.32 mg, 1.11 mmol) and benzenesulfonyl chloride (261.4 mg, 1.48 mmol) was added at 0° C. The mixture was stirred at room temperature for 16 h. LCMS showed product peak. The reaction mixture was washed with water and extracted with DCM. The organic layer was dried over Na₂SO₄. The solution was concentrated in vacuo. The crude product was purified by prep-HPLC to give the product.

¹H NMR (400 MHz, DMSO) δ 8.13 (s, 2H), 7.43 (ddd, J=12.3, 11.5, 7.2 Hz, 2H), 7.21 (dd, J=16.7, 7.9 Hz, 2H), 6.77 (dd, J=43.2, 20.0 Hz, 2H), 4.85-4.70 (m, 2H), 4.30 (t, J=5.3 Hz, 1H), 4.18 (s, 1H), 4.06-3.95 (m, 2H), 3.18 (s, 3H), 2.14 (d, J=19.8 Hz, 3H).

### Example 1-79 Preparation of Compound I-79

FIG. 79 illustrates the synthetic scheme of compound I-79. As shown in FIG. 79, the specific synthesis step is as follows:

### Step 1: N-(4-(5-acetyl-2-(4-fluorophenyl)-4,5,6,7-tetrahydropyrazolo[1,5-a]pyrazin-3-yl)pyridin-2-yl)methanesulfonamide

To a solution 1-(3-(2-aminopyridin-4-yl)-2-(4-fluorophenyl)-6,7-dihydropyrazolo[1,5-a]pyrazin-5(4H)-yl)ethan-1-one (150 mg, 0.46 mmol) in DCM (10 ML), triethylamine (129.59 mg, 1.280 mmol) and methanesulfonyl chloride (0.337 mL) was added at 0° C. The mixture was stirred at room temperature for 16 h. LCMS showed product peak. The reaction was washed with water and extracted with DCM. The organic layer was dried over Na₂SO₄. The solution was concentrated in vacuo. The crude product was purified by prep-HPLC to give the product. ¹H NMR (400 MHz, DMSO) δ 8.250-8.12 (m, 1H), 7.53-7.36 (m, 2H), 7.27-7.19 (m, 2H), 6.90-6.65 (m, 2H), 4.79 (d, J=31.2, 2H), 4.30 (t, J=5.3 Hz, 1H), 4.20-4.15 (m, 1H), 4.09-3.93 (m, 2H), 3.21 (s, 3H), 2.13 (d, J=19.6 Hz, 3H).

### Example 1-88 Preparation of Compound I-80

FIG. 80 illustrates the synthetic scheme of compound I-80. As shown in FIG. 80, the specific synthesis step is as follows:

### Step 1: N-(4-(5-benzyl-2-(4-fluorphenyl)-4,5,6,7-tetrahydropyrazolo[1,5-a]pyrazin-3-yl)pyridin-2-yl)-2-(4-fluorphenyl) acetamide

To a solution of 4-[2-(4-fluorophenyl)-5-(1-methylphenyl)-4H,6H,7Hpyrazolo[1,5-a]pyrazin-3-yl]pyridin-2-amine (500 mg, 1.25 mmol) in DCM (20 mL), 2-(4-fluorophenyl) acetyl chloride (258.6 mg, 1.5 mmol) and triethylamine (189.5 mg, 1.87 mmol) was added. The reaction mixture was stirred at 25° C. for 15 h. The reaction mixture was concentrated under pressure to give product. Chemical Formula: calculated for (M+H+) C₃₂F₂₇N₅O: 535.60, Found: 536.1.

¹H NMR (400 MHz, DMSO) δ 10.73 (s, 1H), 8.19 (d, J=5.2 Hz, 1H), 7.89 (s, 1H), 7.42-7.06 (m, 13H), 6.73 (dd, J=5.2, 1.5 Hz, 1H), 4.17 (t, J=5.3 Hz, 2H), 3.78-3.60 (m, 6H), 2.96 (t, J=5.4 Hz, 2H).

### Step 2: 2-(4-fluorophenyl)-N-(4-(2-(4-fluorophenyl)-4,5,6,7-tetrahydropyrazolo[1,5-a]pyrazin-3-yl)pyridin-2-yl) acetamide (3)

To a solution of 2-(4-fluorophenyl)-N-{4-[2-(4-fluorophenyl)-5-(1-methylphenyl)-4H,6H,7H-pyrazolo[1,5-a]pyrazin-3-yl]pyridin-2-yl}acetamide (380 mg, 0.745 mmol) in MeOH (20 mL), Pd/C (80 mg) was added. The reaction mixture was stirred at 25° C. under H₂ for 15 h. The solution was filtered, filtrate was collected to give product. Chemical Formula: calculated for (M+H+) C₂₅H₂₁F₂N₅O: 445.47, Found: 446.0.

### Step 3: 2-(4-fluorophenyl)-N-(4-(2-(4-fluorophenyl)-5-methyl-4,5,6,7-tetrahydropyrazolo[1,5-a]pyrazin-3-yl)pyridin-2-yl) acetamide

To a solution of 2-(4-fluorophenyl)-N-{4-[2-(4-fluorophenyl)-4H,5H,6H,7H-pyrazolo[1,5-a]pyrazin-3-yl]pyridin-2-yl}acetamide (200 mg, 0.45 mmol) in THF (15 mL), HCHO (40.5 mg, 1.35 mmol) and sodium triacetoxyborohydride (142.7 mg, 0.674 mmol) was added. The reaction mixture was stirred at 60° C. for 15 h. The solution was filtered, filtrate was collected. The reaction mixture was concentrated under pressure. The residue was purified via Genal-Prep-HPLC and give the product.

¹H NMR (400 MHz, DMSO) δ 10.74 (s, 1H), 8.22 (d, J=5.1 Hz, 1H), 7.90 (s, 1H), 7.45-7.28 (m, 4H), 7.15 (ddd, J=8.9, 6.5, 4.3 Hz, 4H), 6.82-6.71 (m, 1H), 4.18 (s, 2H), 3.69 (s, 3H), 3.17 (s, 2H), 2.91 (s, 2H), 2.39 (s, 2H).

### Example 1-81 Preparation of Compound I-81

FIG. 81 illustrates the synthetic scheme of compound I-81. As shown in FIG. 81, the specific synthesis step is as follows:

### Step 1:tert-butyl (4-(5-acetyl-2-(4-fluorophenyl)-4,5,6,7-tetrahydropyrazolo[1,5-a]pyrazin-3-yl)pyridin-2-yl)carbamate

To a solution of tert-butyl (4-(2-(4-fluorophenyl)-4,5,6,7-tetrahydropyrazolo[1,5-a]pyrazin-3-yl)pyridin-2-yl) carbamate (200 mg, 0.489 mmol) in DCM (10 mL) at 0° C., Et₃N (74.2 mg, 0.733 mmol) was added and acetyl chloride (57.5 mg, 0.733 mmol) in DCM (2 mL) was added drop by drop. The mixture was stirred at 0° C. for 1 h. The reaction mixture was quenched with H₂O and the organic layer was concentrated under pressure. The residue was purified via Prep-TLC (PE/EtOAc)=(5:1) and obtained the product.

Chemical Formula: calculated for (M+H⁺) C₂₄H₂₆FN₅O₃: 451.20, Found: 452.25.

### Step 2: 1-(3-(2-aminopyridin-4-yl)-2-(4-fluorophenyl)-6,7-dihydropyrazolo[1,5-a]pyrazin-5(4H)-yl)ethan-1-one

To a solution of tert-butyl (4-(5-acetyl-2-(4-fluorophenyl)-4,5,6,7-tetrahydropyrazolo[1,5-a]pyrazin-3-yl)pyridin-2-yl) carbamate (200 mg, 0.443 mmol) in DCM (10 mL) at 0° C., TFA (75.8 mg, 0.665 mmol) in DCM (2 mL) was added drop by drop. The mixture was stirred at 0° C. for 1 h. The reaction mixture was quenched with the saturated aqueous solution of NaHCO₃ (10 ml) and the organic layer was concentrated under pressure. The residue was purified via Prep-TLC (PE/EtOAc)=(3:1) and obtained the product. Chemical Formula: calculated for (M+H⁺) C₁₉H₁₈FN₅O: 351.15, Found: 352.18.

### Step 3:1-(2-(4-fluorophenyl)-3-(2-(phenethylamino)pyridin-4-yl)-6,7-dihydropyrazolo[1,5-a]pyrazin-5(4H)-yl)ethan-1-one

To a solution of 1-(3-(2-aminopyridin-4-yl)-2-(4-fluorophenyl)-6,7-dihydropyrazolo[1,5-a]pyrazin-5(4H)-yl)ethan-1-one (100 mg, 0.285 mmol) in dioxane (20 mL), Cs₂CO₃ (185.7 mg, 0.57 mmol), (2-bromoethyl)benzene (52.4 mg, 0.285 mmol), Pd₂(dba)₃ (25.6 mg, 0.028 mmol) and x-Phos (1.34 mg, 0.028 mmol) was added. The reaction mixture was stirred at 100° C. for 8 h. The reaction mixture was concentrated under pressure. The residue was purified via Prep-TLC (PE/EtOAc)=(3:1). The residue was purified via Genal-Prep-HPLC and obtained the product. Chemical Formula: calculated for (M+H⁺) C₂₇H₂₆FN₅O: 455.21, Found: 456.25.

### Example 1-82 Preparation of Compound I-82

FIG. 82 illustrates the synthetic scheme of compound I-82. As shown in FIG. 82, the specific synthesis step is as follows:

### Step 1:2-(4-fluorophenyl)propynoyl chloride (2)

To a solution of 2-(4-fluorophenyl)propanoic acid (500 mg, 2.97 mmol) in DCM (30 mL), oxalyl dichloride (1.13 g, 8.9 mmol) were added. The mixture was stirred at 25° C. for 6 h. The reaction mixture was concentrated under pressure. Chemical Formula: calculated for (M+CH3O+) C10H11FO2: 182.19, Found: 182.8.

### Step 2:N-(4-(5-acetyl-2-(4-fluorophenyl)-4,5,6,7-tetrahydropyrazolo[1,5-a]pyrazin-3-yl)pyridin-2-yl)-2-(4-fluorophenyl)-N-(2-(4-fluorophenyl)propanoyl)propanamide (3)

To a solution of 1-(3-(2-aminopyridin-4-yl)-2-(4-fluorophenyl)-6,7-dihydropyrazolo[1,5-a]pyrazin-5(4H)-yl)ethan-1-one (500 mg, 1.42 mmol) in DCM (15 mL) at 0° C., Et3N (216 mg, 2.13 mmol) and 2-(4-fluorophenyl)propanoyl chloride (398.32 mg, 2.13 mmol) in DCM (5 mL) were added. The mixture was stirred at 0° C. for 1 h. The reaction mixture was concentrated under pressure. Chemical Formula: calculated for (M+H+) C₃₇H₃₂F₃N₅O₃: 651.69, Found: 651.8.

### Step 3:N-(4-(5-acetyl-2-(4-fluorophenyl)-4,5,6,7-tetrahydropyrazolo[1,5-a]pyrazin-3-yl)pyridin-2-yl)-2-(4-fluorophenyl)propanamide

To a solution of N-(4-(5-acetyl-2-(4-fluorophenyl)-4,5,6,7-tetrahydropyrazolo[1,5-a]pyrazin-3-yl)pyridin-2-yl)-2-(4-fluorophenyl)-N-(2-(4-fluorophenyl)propanoyl)propanamide (700 mg, 1.07 mmol) in MeOH (20 mL), K₂CO₃ (299.87 mg, 2.15 mmol) was added. The mixture was stirred at 25° C. for 16 h. The reaction mixture was concentrated under pressure. Chemical Formula: calculated for (M+H+) C₂₈H₂₅F₂N₅O₂: 501.54, Found: 501.8. (Compound I-82) ¹H NMR (400 MHz, DMSO) δ 10.72 (d, J=11.6 Hz, 1H), 8.23 (t, J=5.9 Hz, 1H), 7.97 (s, 1H), 7.53-7.26 (m, 4H), 7.26-7.01 (m, 4H), 7.00-6.67 (m, 1H), 4.76 (d, J=27.8 Hz, 2H), 4.23 (dt, J=48.3, 5.2 Hz, 2H), 4.02 (dd, J=8.0, 4.3 Hz, 3H), 2.25-1.95 (m, 3H), 1.36 (t, J=6.1 Hz, 3H). (Compound I-82) ¹H NMR (400 MHz, DMSO) δ 10.71 (d, J=11.5 Hz, 1H), 8.23 (t, J=5.9 Hz, 1H), 7.96 (a, 1H), 7.39 (ddd, J=10.3, 9.5, 5.7 Hz, 4H), 7.25-7.05 (m, 4H), 6.96-6.63 (m, 1H), 4.75 (d, J=28.3 Hz, 2H), 4.34-4.10 (m, 2H), 4.02 (d, J=5.4 Hz, 3H), 2.26-1.97 (m, 3H), 1.36 (t, J=6.1 Hz, 3H).

### Example 1-83 Preparation of Compound I-83

FIG. 83 illustrates the synthetic scheme of compound I-83. As shown in FIG. 83, the specific synthesis step is as follows:

### Step 1:N-(4-(2-(4-fluorophenyl)-5-methyl-4,5,6,7-tetrahydropyrazolo[1,5-a]pyrazin-3-yl)pyridin-2-yl)pivalamide

To a solution of 4-(2-(4-fluorophenyl)-5-methyl-4,5,6,7-tetrahydropyrazolo[1,5-a]pyrazin-3-yl)pyridin-2-amine (200 mg, 0.619 mmol) in DCM (10 ml) at 0° C., Et₃N (93.9 mg, 0.926 mmol) was added and pivaloyl chloride (111.2 mg, 0.926 mmol) in DCM (2 ml) was added drop by drop. The mixture was stirred at 0° C. for 1 h. The reaction mixture was quenched with H₂O and the organic layer was concentrated under pressure. The residue was purified via Genal-Prep-HPLC and obtained the product.

Chemical Formula: calculated for (M+H⁺) C₂₃H₂₆FN₅O: 407.21, Found: 408.25.

### Example 1-84 Preparation of Compound I-84

FIG. 84 illustrates the synthetic scheme of compound I-84. As shown in FIG. 84, the specific synthesis step is as follows:

### Step 1:5-benzyl-2-(4-fluorophenyl)-3-(2-(2,2,2-trifluoroethyl)pyridin-4-yl)-4,5,6,7-tetrahydropyrazolo[1,5-a]pyrazine

To a solution of 5-benzyl-2-(4-fluorophenyl)-3-(4,4,5,5-tetramethyl-1,3,2-dioxaborolan-2-yl)-4,5,6,7-tetrahydropyrazolo[1,5-a]pyrazine (200 mg, 0.462 mmol) in THF (20 mL) and H₂O (4 ml), K₂CO₃ (140.3 mg, 1.02 mmol), 4-bromo-2-(2,2,2-trifluoroethyl)pyridine (110.4 mg, 0.462 mmol), Pd(PPh₃)₄ (26.6 mg, 0.023 mmol) was added. The reaction mixture was stirred at 100° C. for 8 h. The reaction mixture was concentrated under pressure. The residue was purified via Prep-TLC (PE/EtOAc)=(5:1) and obtained the product.

Chemical Formula: calculated for (M+H⁺) C₂₆H₂₂F₄N₄: 466.18, Found: 467.25.

### Step 2:2-(4-fluorophenyl)-3-(2-(2,2,2-trifluoroethyl)pyridin-4-yl)-4,5,6,7-tetrahydropyrazolo[1,5-a]pyrazine

HCOONH₄ (270.5 mg, 4.29 mmol) and Pd/C (20 mg) were added to a solution of 5-benzyl-2-(4-fluorophenyl)-3-(2-(2,2,2-trifluoroethyl)pyridin-4-yl)-4,5,6,7-tetrahydropyrazolo[1,5-a]pyrazine (200 mg, 0.429 mmol) in MeOH (10 ml). The mixture was stirred at 60° C. for 3 h. The solution was filtered, filtrate was collected and the solution was concentrated under pressure and used without further purification.

Chemical Formula: calculated for (M+H⁺) C₁₉H₁₆F₄N₄: 376.13, Found: 377.24.

### Step 3:2-(4-fluorophenyl)-5-methyl-3-(2-(2,2,2-trifluoroethyl)pyridin-4-yl)-4,5,6,7-tetrahydropyrazolo[1,5-a]pyrazine

To a solution of 2-(4-fluorophenyl)-3-(2-(2,2,2-trifluoroethyl)pyridin-4-yl)-4,5,6,7-tetrahydropyrazolo[1,5-a]pyrazine (150 mg, 0.399 mmol) in DCM (10 ml) at 0° C., Et₃N (60.5 mg, 0.598 mmol) was added and CH₃I (62.3 mg, 0.439 mmol) in DCM (2 ml) was added drop by drop. The mixture was stirred at 0° C. for 1 h. The reaction mixture was quenched with H₂O and the organic layer was concentrated under pressure. The residue was purified via Genal-Prep-HPLC and obtained the product.

Chemical Formula: calculated for (M+H⁺) C₂₀H₁₈F₄N₄: 390.15, Found: 391.20.

### Example 1-85 Preparation of Compound I-85

FIG. 85 illustrates the synthetic scheme of compound I-85. As shown in FIG. 85, the specific synthesis step is as follows:

### Step 1:2-((4-(2-(4-fluorophenyl)-5-methyl-4,5,6,7-tetrahydropyrazolo[1,5-a]pyrazin-3-yl)pyridin-2-yl)methyl)oxazole

To a solution of 2-(4-fluorophenyl)-5-methyl-3-(4,4,5,5-tetramethyl-1,3,2-dioxaborolan-2-yl)-4,5,6,7-tetrahydropyrazolo[1,5-a]pyrazine (200 mg, 0.56 mmol) in THF (20 mL) and H₂O (4 ml), K₂CO₃ (170 mg, 1.23 mmol), 2-((4-bromopyridin-2-yl)methyl)oxazole (133.3 mg, 0.56 mmol), Pd(PPh₃)₄ (32.3 mg, 0.028 mmol) was added. The reaction mixture was stirred at 80° C. for 8 h. The reaction mixture was concentrated under pressure. The residue was purified via Prep-TLC (PE/EtOAc)=(4:1) and obtained the crude product. The crude product was purified via Genal-Prep-HPLC to obtain the product.

Chemical Formula: calculated for (M+H⁺) C₂₂H₂₀FN₅O: 389.17, Found: 390.21.

### Example 1-86 Preparation of Compound I-86

FIG. 86 illustrates the synthetic scheme of compound I-86. As shown in FIG. 86, the specific synthesis step is as follows:

### Step 1:3-(2-((1H-imidazol-2-yl)methyl)pyridin-4-yl)-2-(4-fluorophenyl)-5-methyl-4,5,6,7-tetrahydropyrazolo[1,5-a]pyrazine

To a solution of 2-(4-fluorophenyl)-5-methyl-3-(4,4,5,5-tetramethyl-1,3,2-dioxaborolan-2-yl)-4,5,6,7-tetrahydropyrazolo[1,5-a]pyrazine (200 mg, 0.56 mmol) in THF (20 mL) and H₂O (4 ml), K₂CO₃ (170 mg, 1.23 mmol), 2-((1H-imidazol-2-yl)methyl)-4-bromopyridine (132.7 mg, 0.56 mmol), Pd(PPh₃)₄ (32.3 mg, 0.028 mmol) was added. The reaction mixture was stirred at 80° C. for 8 h. The reaction mixture was concentrated under pressure. The residue was purified via Prep-TLC (PF/EtOAc)=(4:1) and obtained the crude product. The crude product was purified via Genal-Prep-HPLC to obtain the product.

Chemical Formula: calculated for (M+H⁺) C₂₂H₂₁FN₆: 388.18, Found: 388.45.

### Example 1-87 Preparation of Compound I-87

FIG. 87 illustrates the synthetic scheme of compound I-87. As shown in FIG. 87, the specific synthesis step is as follows:

### Step 1:5-benzyl-2-(4-fluorophenyl)-3-(2-(trifluoromethyl)pyridin-4-yl)-4,5,6,7-tetrahydropyrazolo[1,5-a]pyrazine

To a solution of 5-benzyl-2-(4-fluorophenyl)-3-(4,4,5,5-tetramethyl-1,3,2-dioxaborolan-2-yl)-4,5,6,7-tetrahydropyrazolo[1,5-a]pyrazine (200 mg, 0.462 mmol) in THF (20 mL), K₂CO₃ (140.3 mg, 1.02 mmol), 4-bromo-2-(trifluoromethyl)pyridine (103.9 mg, 0.462 mmol), Pd₂(dba)₃ (21 mg, 0.023 mmol) and S-Phos (9.43 mg, 0.023 mmol) was added. The reaction mixture was stirred at 100° C. for 8 h. The reaction mixture was concentrated under pressure. The residue was purified via Prep-TLC (PE/EtOAc)=(5:1) and obtained the product.

Chemical Formula: calculated for (M+H⁺) C₂₅H₂₀F₄N₄: 452.16, Found: 453.25.

### Step 2: 2-(4-fluorophenyl)-3-(2-(trifluoromethyl)pyridin-4-yl)-4,5,6,7-tetrahydropyrazolo[1,5-a]pyrazine

HCOONH₄ (278.6 mg, 4.42 mmol) and Pd/C (20 mg) were added to a solution of 5-benzyl-2-(4-fluorphenyl)-3-(2-(trifluoromethyl)pyridin-4-yl)-4,5,6,7-tetrahydropyrazolo[1,5-a]pyrazine (200 mg, 0.442 mmol) in MeOH (10 ml). The mixture was stirred at 60° C. for 3 h. The solution was filtered, filtrate was collected and the solution was concentrated under pressure and used without further purification.

Chemical Formula: calculated for (M+H⁺) C₁₈H₁₄F₄N₄: 362.12, Found: 363.14.

### Step 3:1-(2-(4-fluorophenyl)-3-(2-(trifluoromethyl)pyridin-4-yl)-6,7-dihydropyrazolo[1,5-a]pyrazin-5(4H)-yl)ethan-1-one

To a solution of 2-(4-fluorophenyl)-3-(2-(trifluoromethyl)pyridin-4-yl)-4,5,6,7-tetrahydropyrazolo[1,5-a]pyrazine (150 mg, 0.414 mmol) in DCM (10 ml) at 0° C., Et₃N (62.9 mg, 0.621 mmol) was added and acetyl chloride (38.7 mg, 0.497 mmol) in DCM (2 ml) was added drop by drop. The mixture was stirred at 0° C. for 1 h. The reaction mixture was quenched with H₂O and the organic layer was concentrated under pressure. The residue was purified via Genal-Prep-HPLC and obtained the product.

Chemical Formula: calculated for (M+H⁺) C₂₀H₁₆F₄N₄O: 404.13, Found: 405.20.

### Example 1-88 Preparation of Compound I-98

FIG. 88 illustrates the synthetic scheme of compound I-88. As shown in FIG. 88, the specific synthesis step is as follows:

### Step 1:5-benzyl-3-(2-(difluoromethyl)pyridin-4-yl)-2-(4-fluorophenyl)-4,5,6,7-tetrahydropyrazolo[1,5-a]pyrazine

To a solution of 5-benzyl-2-(4-fluorophenyl)-3-(4,4,5,5-tetramethyl-1,3,2-dioxaborolan-2-yl)-4,5,6,7-tetrahydropyrazolo[1,5-a]pyrazine (200 mg, 0.462 mmol) in THF (20 mL), K₂CO₃ (140.3 mg, 1.02 mmol), 4-bromo-2-(difluoromethyl)pyridine (95.6 mg, 0.462 mmol), Pd₂(dba)₃ (21 mg, 0.023 mmol) and S-Phos (9.43 mg, 0.023 mmol) was added. The reaction mixture was stirred at 100° C. for 8 h. The reaction mixture was concentrated under pressure. The residue was purified via Prep-TLC (PE/EtOAc)=(5:1) and obtained the product.

Chemical Formula: calculated for (M+H⁺) C₂₅H₂₁F₃N₄: 434.17, Found: 435.25.

### Step 2: 3-(2-(difluoromethyl)pyridin-4-yl)-2-(4-fluorophenyl)-4,5,6,7-tetrahydropyrazolo[1,5-a]pyrazine

HCOONH₄ (278.6 mg, 4.6 mmol) and Pd/C (20 mg) were added to a solution of 5-benzyl-3-(2-(difluoromethyl)pyridin-4-yl)-2-(4-fluorophenyl)-4,5,6,7-tetrahydropyrazolo[1,5-a]pyrazine (200 mg, 0.46 mmol) in MeOH (10 ml). The mixture was stirred at 60° C. for 3 h. The solution was filtered, filtrate was collected and the solution was concentrated under pressure and used without further purification.

Chemical Formula: calculated for (M+H⁺) C₁₈H₁₄F₄N₄: 344.12, Found: 345.14.

### Step 3:1-(3-(2-(difluoromethyl)pyridin-4-yl)-2-(4-fluorophenyl)-6,7-dihydropyrazolo[1,5-a]pyrazin-5(4H)-yl)ethan-1-one

To a solution of 3-(2-(difluoromethyl)pyridin-4-yl)-2-(4-fluorophenyl)-4,5,6,7-tetrahydropyrazolo[1,5-a]pyrazine (150 mg, 0.436 mmol) in DCM (10 ml) at 0° C., Et₃N (66.2 mg, 0.654 mmol) was added and acetyl chloride (40.8 mg, 0.523 mmol) in DCM (2 ml) was added drop by drop. The mixture was stirred at 0° C. for 1 h. The reaction mixture was quenched with H₂O and the organic layer was concentrated under pressure. The residue was purified via Genal-Prep-HPLC and obtained the product.

Chemical Formula: calculated for (M+H⁺) C₂₀H₁₇F₃N₄O: 386.14, Found: 387.16.

### Example 1-89 Preparation of Compound I-89

FIG. 89 illustrates the synthetic scheme of compound I-89. As shown in FIG. 89, the specific synthesis step is as follows:

### Step 1: N-(4-(5-benzyl-2-(4-fluorophenyl)-4,5,6,7-tetrahydropyrazolo[1,5-a]pyrazin-3-yl) pyridin-2-yl)cyclopropanecar-boxamide (2)

To a solution of 4-[2-(4-fluorophenyl)-5-(1-methylphenyl)-4H,6H,7H-pyrazolo[1,5-a]pyrazin-3-yl]pyridin-2-amine (500 mg, 1.4982 mmol) in DCM (10 mL), then was added cyclopropanecarbonyl chloride (187.94 mg, 1.7978 mmol), DIPEA (386.54 mg, 2.9964 mmol), The reaction mixture was stirred at 25° C. for 2 h. Then was added MeOH (10 mL), then was added K₂CO₃ (257.18 mg, 1.8636 mmol), The reaction mixture was stirred at 25° C. for 2 h. The reaction was complete detected by LCMS. The reaction mixture was filtered and concentrated under pressure. The crude material was added to a silica gel column and was eluted with PE/EtOAc (1:1). Chemical Formula: calculated for (M+H⁺) C₂₈H₂₆FN₅O: 467.2, Found: 467.7.

### Step 2: N-(4-(2-(4-fluorophenyl)-4,5,6,7-tetrahydropyrazolo[1,5-a]pyrazin-3-yl)pyridin-2-yl)cyclopropanecarboxamide (3)

To a solution of N-(4-(5-benzyl-2-(4-fluorophenyl)-4,5,6,7-tetrahydropyrazolo[1,5-a]pyrazin-3-yl)pyridin-2-yl)cyclopropanecarboxamide (480 mg, 1.0244 mmol) in EA/MeOH (10 mL), then was added Pd/C (200 mg), HCl (1 mL), under H₂, the reaction mixture was stirred at 25° C. for 2 h. The reaction was complete detected by lcms. The solution was filtered, filtrate was collected. The reaction mixture was concentrated under pressure. The organic phase was washed with 2 M sodium carbonate solution (5 mL). The residue was extracted with EA (10 mL*3). The combined organic layers were washed with brine (10 mL), dried over sodium sulfate, filtered and concentrated to give the product. Chemical Formula: calculated for (M+H⁺) C₂₁H₂₀FN₅O: 377.2, Found: 377.8.

### Step 3: N-(4-(2-(4-fluorophenyl)-5-methyl-4,5,6,7-tetrahydropyrazolo[1,5-a]pyrazin-3-yl)pyridin-2-yl)cyclopropanecarboxamide

To a solution of N-(4-(2-(4-fluorophenyl)-4,5,6,7-tetrahydropyrazolo[1,5-a]pyrazin-3-yl)pyridin-2-yl)cyclopropanecarboxamide (120 mg, 0.3180 mmol) in THF (5 mL), then was added HCHO (47.70 mg, 1.59 mmol), The reaction mixture was stirred at 70° C. for 14 h. Then sodium triacetoxyborohydride (80.90 mg, 0.3816 mmol) was added. The reaction mixture was stirred at 25° C. for 2 h. The reaction was complete detected by LCMS. The reaction mixture was concentrated under pressure. The organic phase was washed with H₂O (5 mL). The residue was extracted with EA (10 mL*3). The combined organic layers were washed with brine (10 mL), dried over sodium sulfate, filtered and concentrated to give the product. The residue was purified via Genal-Prep-HPLC and give the product.

¹H NMR (400 MHz, DMSO) δ 10.81 (s, 1H), 8.21 (d, J=5.2 Hz, 1H), 7.92 (s, 1H), 7.43-7.33 (m, 2H), 7.17 (t, J=8.8 Hz, 2H), 6.75 (dd, J=5.2, 1.6 Hz, 1H), 4.18 (t, J=5.4 Hz, 2H), 3.59 (s, 2H), 2.92 (t, J=5.4 Hz, 2H), 2.40 (s, 3H), 2.02-1.95 (m, 11H), 0.84-0.71 (m, 4H).

### Example 1-90 Preparation of Compound I-90

FIG. 90 illustrates the synthetic scheme of compound I-90. As shown in FIG. 90, the specific synthesis step is as follows:

### Step 1: 2-(tert-butoxy)-N-(4-(2-(4-fluorophenyl)-5-methyl-4,5,6,7-tetrahydropyrazolo[1,5-a]pyrazin-3-yl)pyridin-2-yl) acetamide

To a solution of 4-(2-(4-fluorophenyl)-5-methyl-4,5,6,7-tetrahydropyrazolo[1,5-a]pyrazin-3-yl)pyridin-2-amine (200 mg, 0.619 mmol) in DCM (20 mL), DIPEA (160 mg, 1.238 mmol), 2-(tert-butoxy) acetic acid (81.8 mg, 0.619 mmol), and HATU (235.4 mg, 0.619 mmol) was added. The reaction mixture was stirred at r.t. for 4 h. The reaction mixture was quenched with H₂O and the organic layer was concentrated under pressure. The residue was purified via Prep-TLC (PE/EtOAc)=(5:1) and obtained the product.

Chemical Formula: calculated for (M+H⁺) C₂₄H₂₈FN₅O₂: 437.22, Found: 438.25.

### Step 2:N-(4-(2-(4-fluorophenyl)-5-methyl-4,5,6,7-tetrahydropyrazolo[1,5-a]pyrazin-3-yl)pyridin-2-yl)-2-hydroxyacetamide

To a solution of 2-(tert-butoxy)-N-(4-(2-(4-fluorophenyl)-5-methyl-4,5,6,7-tetrahydropyrazolo[1,5-a]pyrazin-3-yl) pyridin-2-yl) acetamide (150 mg, 0.343 mmol) in DCM (10 ml) at 0° C., HCl (1N, 0.686 mmol) was added. The mixture was stirred at r.t. for 1 h. The reaction mixture was quenched with the saturated aqueous solution of NaHCO₃ (5 ml) and the organic layer was concentrated under pressure. The residue was purified via Genal-Prep-HPLC and obtained the product.

Chemical Formula: calculated for (M+H⁺) C₂₀H₂₀FN₅O₂: 381.16, Found: 382.24.

### Example 1-91 Preparation of Compound I-91

FIG. 91 illustrates the synthetic scheme of compound I-91. As shown in FIG. 91, the specific synthesis step is as follows:

### Step 1:3-(4-(2-(4-fluorophenyl)-5-methyl-4,5,6,7-tetrahydropyrazolo[1,5-a]pyrazin-3-yl)pyridin-2-yl)-1,1-dimethylurea

To a solution of 4-(2-(4-fluorophenyl)-5-methyl-4,5,6,7-tetrahydropyrazolo[1,5-a]pyrazin-3-yl)pyridin-2-amine (200 mg, 0.619 mmol) in DCM (10 ml) at 0° C., Et₃N (93.9 mg, 0.926 mmol) was added and dimethylcarbamic chloride (99.1 mg, 0.926 mmol) in DCM (2 ml) was added drop by drop. The mixture was stirred at r.t. for 2 h. The reaction mixture was quenched with H₂O and the organic layer was concentrated under pressure. The residue was purified via Genal-Prep-HPLC and obtained the product.

Chemical Formula: calculated for (M+H⁺) C₂₁H₂₃FN₆O: 394.19, Found: 395.23.

### Example 1-92 Preparation of Compound I-92

FIG. 92 illustrates the synthetic scheme of compound I-92. As shown in FIG. 92, the specific synthesis step is as follows:

### Step 1:1-(4-(2-(4-fluorophenyl)-5-methyl-4,5,6,7-tetrahydropyrazolo[1,5-a]pyrazin-3-yl)pyridin-2-yl)-3-methylurea

To a solution of 4-(2-(4-fluorophenyl)-5-methyl-4,5,6,7-tetrahydropyrazolo[1,5-a]pyrazin-3-yl)pyridin-2-amine (200 mg, 0.619 mmol) in DCM (10 ml) at 0° C., Et₃N (93.9 mg, 0.926 mmol) was added and methylcarbamic chloride (86.1 mg, 0.926 mmol) in DCM (2 ml) was added drop by drop. The mixture was stirred at r.t. for 2 h. The reaction mixture was quenched with H₂O and the organic layer was concentrated under pressure. The residue was purified via Genal-Prep-HPLC and obtained the product.

Chemical Formula: calculated for (M+H⁺) C₂₀H₂₁FN₆O: 380.18, Found: 381.21.

### Example 1-93 Preparation of Compound I-93

FIG. 93 illustrates the synthetic scheme of compound I-93. As shown in FIG. 93, the specific synthesis step is as follows:

### Step 1:N-(4-(2-(4-fluorophenyl)-5-methyl-4,5,6,7-tetrahydropyrazolo[1,5-a]pyrazin-3-yl)pyridin-2-yl)pyrimidin-4-amine

To a solution of 4-(2-(4-fluorophenyl)-5-methyl-4,5,6,7-tetrahydropyrazolo[1,5-a]pyrazin-3-yl)pyridin-2-amine (100 mg, 0.309 mmol) in dioxane (20 mL), Cs₂CO₃ (221.2 mg, 0.681 mmol), 4-bromopyrimidine (48.8 mg, 0.309 mmol), Pd₂(dba)₃ (27.4 mg, 0.03 mmol) and x-Phos (14.3 mg, 0.03 mmol) was added. The reaction mixture was stirred at 100° C. for 8 h. The reaction mixture was concentrated under pressure. The residue was purified via Prep-TLC (PF/EtOAc)=(2:1). The residue was purified via Genal-Prep-HPLC and obtained the product. Chemical Formula: calculated for (M+H⁺) C₂₂H₂₀FN₇: 401.18, Found: 402.25.

### Example 1-94 Preparation of Compound I-94

FIG. 94 illustrates the synthetic scheme of compound I-94. As shown in FIG. 94, the specific synthesis step is as follows:

### Step 1: N-(4-(5-benzyl-2-(4-fluorophenyl)-4,5,6,7-tetrahydropyrazolo[1,5-a]pyrazin-3-yl)pyridin-2-yl)-2-(4-fluorophenylacetamide

To a solution of 4-[2-(4-fluorophenyl)-5-(1-methylphenyl)-4H,6H,7Hpyrazolo[1,5-a]pyrazin-3-yl]pyridin-2-amine (500 mg, 1.25 mmol) in DCM (20 mL), 2-(4-fluorophenyl) acetyl chloride (258.6 mg, 1.5 mmol) and triethylamine (189.5 mg, 1.87 mmol) was added. The reaction mixture was stirred at 25° C. for 15 h. The reaction mixture was concentrated under pressure to give product. Chemical Formula: calculated for (M+H+) C₃₂H₂₇F₂N₅O: 535.60, Found: 536.1.

¹H NMR (400 MHz, DMSO) δ 10.73 (s, 1H), 8.19 (d, J=5.2 Hz, 1H), 7.89 (s, 1H), 7.42-7.06 (m, 13H), 6.73 (dd, J=5.2, 1.5 Hz, 1H), 4.17 (t, J=5.3 Hz, 2H), 3.78-3.60 (m, 6H), 2.96 (t, J=5.4 Hz, 2H).

### Example 1-95 Preparation of Compound I-95

FIG. 95 illustrates the synthetic scheme of compound I-95. As shown in FIG. 95, the specific synthesis step is as follows:

### Step 1: N-(4-(5-benzyl-2-(4-fluorophenyl-4,5,6,7-tetrahydropyrazolo[1,5-a]pyrazin-3-yl)pyridin-2-yl) acetamide

To a solution of 4-(5-benzyl-2-(4-fluorophenyl)-4,5,6,7-tetrahydropyrazolo[1,5-a]pyrazin-3-yl)pyridin-2-amine (200 mg, 0.50 mmol) in DCM (6 mL) at 0° C., Et₃N (15.8 mg, 0.75 mmol) was added and acetyl chloride (58 mg, 0.75 mmol) in DCM (2 mL) was added drop by drop. The mixture was stirred at 0° C. for 1 h. The reaction mixture was concentrated under pressure. The residue was purified via Prep-TLC PE/EA (5:1) and give the product. Chemical Formula: calculated for (M+H⁺) C₂₆H₂₄FN₅O: 441.51, Found: 441.8.

### Example 1-96 Preparation of Compound I-96

FIG. 96 illustrates the synthetic scheme of compound I-96. As shown in FIG. 96, the specific synthesis step is as follows:

### Step 1: 1-(2-(4-fluorphenyl)-3-(2-((2,2,2-trifluoroethyl)amino) pyridin-4-yl)-6,7-dihydropyrazolo[1,5-a]pyrazin-5 (4H)-yl)ethan-1-one

To a solution of 1-(3-(2-aminopyridin-4-yl)-2-(4-fluorophenyl)-6,7-dihydropyrazolo[1,5-a]pyrazin-5(4H)-yl)ethan-1-one (280 mg, 0.7969 mmol) in THF (5 mL), then was added LiHMDS (199.62 mg, 1.1953 mmol) at 0° C. The reaction mixture was stirred at 0° C. for 30 mins. Then 2,2,2-trifluoroethyl trifluoromethanesulfonate (277.44 mg, 1.1953 mmol) was added. The reaction mixture was stirred at 25-70° C. for 2.5 h. The reaction mixture was concentrated under pressure. The organic phase was washed with H₂O (5 mL). The residue was extracted with EA (10 mL*3). The combined organic layers were washed with brine (10 mL), dried over sodium sulfate, filtered and concentrated to give the product. The residue was purified via Genal-Prep-HPLC and give the product.

¹H NMR (400 MHz, MeOD) δ 7.97-7.91 (m, 1H), 7.46-7.38 (m, 2H), 7.08 (t, J=8.8 Hz, 2H), 6.49-6.39 (m, 2H), 4.84 (d, J=7.8 Hz, 2H), 4.33 (t, J=5.4 Hz, 1H), 4.23 (d, J=5.2 Hz, 1H), 4.16-4.01 (m, 4H), 2.21 (d, J=30.7 Hz, 3H).

### Example 1-97 Preparation of Compound I-97

FIG. 97 illustrates the synthetic scheme of compound I-97. As shown in FIG. 97, the specific synthesis step is as follows:

### Step 1: N-(4-(2-(4-fluorophenyl)-5-propionyl-4,5,6,7-tetrahydropyrazolo[1,5-a]pyrazin-3-yl)pyridin-2-yl)-N-propionylpropanamide (2)

To a solution of 4-[2-(4-fluorophenyl)-4H,5H,6H,7H-pyrazolo[1,5-a]pyrazin-3-yl]pyridin-2-amine (500 mg, 1.61 mmol) in DCM (20 mL), propanoyl chloride (299 mg, 3.23 mmol) and triethylamine (327 mg, 3.23 mmol) was added. The reaction mixture was stirred at 25° C. for 15 h. The reaction mixture was concentrated under pressure to give product. Chemical Formula: calculated for (M+H⁺) C₂₆H₂₅FN₅O₃: 477.54, Found: 478.1.

### Step 2: N-(4-(2-(4-fluorophenyl)₅-propionyl-4,5,6,7-tetrahydropyrazolo[1,5-a]pyrazin-3-yl)pyridin-2-yl)propionamide

To a solution of N-{4-[2-(4-fluorophenyl)-5-propanoyl-4H,6H,7H-pyrazolo[1,5-a]pyrazin-3-yl]pyridin-2-yl}-N propionylpropanamide (500 mg, 1.05 mmol) in MeOH (20 mL), potassium carbonate (289 mg, 2.09 mmol) was added. The reaction mixture was stirred at 25° C. for 15 h. The reaction mixture was concentrated under pressure. The residue was purified via Genal-Prep-HPLC and give the product. ¹H NMR (400 MHz, DMSO) δ 10.49 (s, 1H), 8.23 (d, J=5.0 Hz, 1H), 8.00 (s, 1H), 7.40 (dd, J=8.5, 5.6 Hz, 2H), 7.19 (t, J=8.9 Hz, 2H), 6.89-6.70 (m, 1H), 4.74 (s, 2H), 4.23 (d, J=41.1 Hz, 2H), 4.04 (s, 2H), 2.37 (dt, J=16.6, 8.4 Hz, 4H), 1.03 (dd, J=12.7, 5.1 Hz, 6H).

### Example 1-98 Preparation of Compound I-98

FIG. 98 illustrates the synthetic scheme of compound I-98. As shown in FIG. 98, the specific synthesis step is as follows:

### Step 1: N-(4-(2-(4-fluorophenyl)-5-isobutyryl-4,5,6,7-tetrahydropyrazolo[1,5-a]pyrazin-3-yl)pyridin-2-yl)-N-isobutyrylisobutyramide (2)

To a solution of 4-[2-(4-fluorophenyl)-4H,5H,6H,7H-pyrazolo[1,5-a]pyrazin-3-yl]pyridin-2-amine (500 mg, 1.61 mmol) in DCM (20 mL), propanoyl chloride (344 mg, 3.23 mmol) and triethylamine (327 mg, 3.23 mmol) was added. The reaction mixture was stirred at 25° C. for 15 h. The reaction mixture was concentrated under pressure to give product. Chemical Formula: calculated for (M+H⁺) C₂₉H₃₄FN₅O₃: 519.62, Found: 520.1.

### Step 2: N-(4-(2-(4-fluorophenyl)-5-propionyl-4,5,6,7-tetrahydropyrazolo[1,5-a]pyrazin-3-yl)pyridin-2-yl)propionamide

To a solution of N-{4-[2-(4-fluorophenyl)-5-propanoyl-4H,6H,7H-pyrazolo[1,5-a]pyrazin-3-yl]pyridin-2-yl}-N-propionylpropanamide (500 mg, 0.989 mmol) in MeOH (20 mL). Potassium carbonate (273 mg, 1.98 mmol) was added

The reaction mixture was stirred at 25° C. for 15 h. reaction mixture was concentrated under pressure. The residue was purified via Genal-Prep-HPLC and give the product.

¹H NMR (400 MHz, DMSO) δ 10.57 (s, 1H), 8.24 (s, 1H), 8.00 (s, 1H), 7.41 (dd, J=8.7, 5.6 Hz, 2H), 7.19 (t, J=8.9 Hz, 2H), 6.79 (d, J=23.3 Hz, 1H), 4.82 (d, J=53.6 Hz, 2H), 4.15 (dd, J=59.4, 35.4 Hz, 4H), 3.03 (d, J=6.5 Hz, 1H), 2.82-2.70 (m, 1H), 1.06 (dd, J=11.3, 6.8 Hz, 12H).

### Example 1-99 Preparation of Compound I-9

FIG. 99 illustrates the synthetic scheme of compound I-99. As shown in FIG. 99, the specific synthesis step is as follows:

### Step 1: N-(4-(2-(4-fluorophenyl)-4,5,6,7-tetrahydropyrazolo[1,5-a]pyrazin-3-yl)pyridin-2-yl) acetamide

HCOONH₄ (598 mg, 9.49 mmol) and Pd/C (210 mg) were added to a solution of N-(4-(5-benzyl-2-(4-fluorophenyl)-4,5,6,7-tetrahydropyrazolo[1,5-a]pyrazin-3-yl)pyridin-2-yl) acetamide (210 mg, 0.47 mmol) in MeOH (10 mL). The mixture was stirred at 60° C. for 3 h. The solution was filtered, filtrate was collected and the solution was concentrated under pressure. Chemical Formula: calculated for (M+H⁺) C₁₉H₁₈FN₅: 351.39, Found: 351.8.

### Example 1-1N Preparation of Compound I-100

FIG. 100 illustrates the synthetic scheme of compound I-100. As shown in FIG. 100, the specific synthesis step is as follows:

### Step 1:N-(4-(5-benzyl-2-(4-fluorophenyl)-4,5,6,7-tetrahydropyrazolo[1,5-a]pyrazin-3-yl)pyridin-2-yl)₂-methylbutanamide (2)

To a solution of 4-(5-benzyl-2-(4-fluorophenyl)-4,5,6,7-tetrahydropyrazolo[1,5-a]pyrazin-3-yl)pyridin-2-amine (800 mg, 2 mmol) in DCM (15 mL) at 0° C., Et₃N (243 mg, 204 mmol) and 2-methylbutanoyl chloride (289.04 mg, 204 mmol) in DCM (5 mL) was added. The mixture was stirred at 0° C. for 1 h. The reaction mixture was concentrated under pressure. Chemical Formula: calculated for (M+H⁺) C₂₉H₃₀FN₅O: 483.59, Found: 483.8.

### Step 2:N-(4-(2-(4-fluorophenyl)-4,5,6,7-tetrahydropyrazolo[1,5-a]pyrazin-3-yl)pyridin-2-yl)-2-methylbutanamide (3)

To a solution of N-(4-(5-benzyl-2-(4-fluorophenyl)-4,5,6,7-tetrahydropyrazolo[1,5-a]pyrazin-3-yl)pyridin-2-yl)-2-methylbutanamide (900 mg, 1.9 mmol) in MeOH (30 mL), Pd/C (90 mg) and 1 M HCl (0.5 mL) were added. The mixture was stirred at 60° C. for 16 h under hydrogen atmosphere. The solution was filtered, filtrate was collected and concentrated under pressure. Chemical Formula: calculated for (M+H⁺) C₂H₂₄FN₅O: 393.47, Found: 393.8.

### Step 3: N-(4-(2-(4-fluorophenyl)-5-methyl-4,5,6,7-tetrahydropyrazolo[1,5-a]pyrazin-3-yl)pyridin-2-yl)-2-methylbutanamide

To a solution of N-(4-(2-(4-fluorophenyl)-4,5,6,7-tetrahydropyrazolo[1,5-a]pyrazin-3-yl)pyridin-2-yl)-2-methylbutanamide (700 mg, 1.78 mmol) in MeOH (20 mL), acetaldehyde (157 mg, 3.56 mmol), ZnCl₂ (485 mg, 3.56 mmol) were added. The mixture was stirred at 25° C. for 1 h. Then, NaBH₃CN (224.17 mg, 3.56 mmol) was added, the reaction was stirred at 25° C. for 1 h. The reaction mixture was concentrated under pressure. Chemical Formula: calculated for (M+H⁺) C₂₃H₂₆FN₅O: 407.49, Found: 407.8. ¹H NMR (400 MHz, DMSO) δ 10.44 (s, 1H), 8.19 (t, J=11.1 Hz, 1H), 7.96 (s, 1H), 7.48-7.28 (m, 2H), 7.22-7.08 (m, 2H), 6.77 (dd, J=5.2, 1.5 Hz, 1H), 4.19 (t, J=5.4 Hz, 2H), 3.63 (s, 2H), 2.93 (t, J=5.4 Hz, 2H), 2.64-2.51 (m, 1H), 2.41 (s, 3H), 1.64-1.22 (m, 2H), 1.04 (d, J=6.8 Hz, 3H), 0.82 (t, J=7.4 Hz, 3H).

### Example 1-191 Preparation of Compound I-101

FIG. 101 illustrates the synthetic scheme of compound I-101. As shown in FIG. 101, the specific synthesis step is as follows:

### Step 1: 1-(2-(4-fluorophenyl)-3-(2-(pyridin-4-ylamino)pyridin-4-yl)-6,7-dihydropyrazolo[1,5-a]pyrazin-5(4H)-yl)ethan-1-one

To a solution of 1-[3-(2-aminopyridin-4-yl)-2-(4-fluorophenyl)-4H,6H,7H-pyrazolo[1,5-a]pyrazin-5-yl]ethanone (300 mg, 0.85 mmol) in dioxane (20 mL), Cs₂CO₃ (556 mg, 1.7 mmol), 4-iodopyridine (210 mg, 1.02 mmol), Pd₂(dba)₃ (78 mg, 0.085 mmol) and x-Phos (40.7 mg, 0.085 mmol) was added. The reaction mixture was stirred at 100° C. for 15 h. reaction mixture was concentrated under pressure. The residue was purified via Genal-Prep-HPLC and give the product.

¹H NMR (400 MHz, DMSO) δ 9.56 (d, J=16.1 Hz, 1H), 8.26 (dd, J=16.5, 5.6 Hz, 3H), 8.17 (s, 1H), 7.62 (d, J=4.8 Hz, 2H), 7.44 (dd, J=8.7, 5.5 Hz, 2H), 7.23 (t, J=8.9 Hz, 2H), 6.84-6.61 (m, 2H), 4.79 (d, J=21.2 Hz, 2H), 4.28 (dd, J=30.1, 24.7 Hz, 2H), 4.07-3.98 (m, 2H), 2.14 (d, J=23.0 Hz, 3H).

### Example 1-112 Preparation of Compound I-102

FIG. 102 illustrates the synthetic scheme of compound I-102. As shown in FIG. 102, the specific synthesis step is as follows:

### Step 1: N-(4-(5-benzyl-2-(4-fluorophenyl)-4,5,6,7-tetrahydropyrazolo[1,5-a]pyrazin-3-yl)pyridin-2-yl)isobutyramide (2)

To a solution of 4-[2-(4-fluorophenyl)-5-(1-methylphenyl)-4H,6H,7H-pyrazolo[1,5-a]pyrazin-3-yl]pyridin-2-amine (200 mg, 0.4994 mmol) in DCM (10 mL), then was added 2-methylpropanoyl chloride (79.82 mg, 0.7491 mmol), DIPEA (128.85 mg, 0.9988 mmol). The reaction mixture was stirred at 25° C. for 2 h. Then MeOH (5 mL) and K₂CO₃ (102.09 mg, 0.7398 mmol) was added. The reaction mixture was stirred at 25° C. for 2 h. The reaction was complete detected by lcms. The reaction mixture was filtered and concentrated under pressure. The crude material was added to a silica gel column and was eluted with PE/EtOAc (1:1). Chemical Formula: calculated for (M+H⁺) C₂₈H₂₈FN₅O: 469.2, Found: 469.7.

### Step 2: N-(4-(2-(4-fluorophenyl)-4,5,6,7-tetrahydropyrazolo[1,5-a]pyrazin-3-yl)pyridin-2-yl)isobutyramide

To a solution of N-(4-(5-benzyl-2-(4-fluorophenyl)-4,5,6,7-tetrahydropyrazolo[1,5-a]pyrazin-3-yl)pyridin-2-yl)isobutyramide (200 mg, 0.425 mmol) in EA/MeOH (10 mL), then Pd/C (100 mg) and HCl (1 mL) was added under H₂. The reaction mixture was stirred at 25° C. for 2 h. The reaction was complete detected by loins. The solution was filtered, filtrate was collected. The reaction mixture was concentrated under pressure. The organic phase was washed with 2 M sodium carbonate solution (5 mL). The residue was extracted with EA (10 mL*3). The combined organic layers were washed with brine (10 mL), dried over sodium sulfate, filtered and concentrated to give the product. Chemical Formula: calculated for (M+H⁺) C₂₁H₂₂FN₅O: 379.2, Found: 379.8.

### Step 3: N-(4-(2-(4-fluorophenyl)₅-methyl-4,5,6,7-tetrahydropyrazolo[1,5-a]pyrazin-3-yl) pyridin-2-yl)isobutyramide

To a solution of N-(4-(2-(4-fluorophenyl)-4,5,6,7-tetrahydropyrazolo[1,5-a]pyrazin-3-yl)pyridin-2-yl)isobutyramide (120 mg, 0.3163 mmol) in THF (10 mL), then was added HCHO (94.89 mg, 3.1630 mmol), The reaction mixture was stirred at 70° C. for 14 h. then was added Sodium triacetoxyborohydride (80.47 mg, 0.3795 mmol), The reaction mixture was stirred at 25° C. for 2 h. The reaction was complete detected by lcms. The reaction mixture was concentrated under pressure. The organic phase was washed with H₂O (5 mL). The residue was extracted with EA (10 mL*3). The combined organic layers were washed with brine (10 mL), dried over sodium sulfate, filtered and concentrated to give the product. The residue was purified via Genal-Prep-HPLC and give the product.

¹H NMR (400 MHz, DMSO) δ 10.44 (s, 1H), 8.20 (d, J=5.2 Hz, 1H), 7.95 (s, 1H), 7.43-7.34 (m, 2H), 7.16 (dd, J=12.4, 5.6 Hz, 2H), 6.75 (dd, J=5.2, 1.6 Hz, 1H), 4.19 (s, 2H), 3.61 (s, 2H), 2.93 (s, 2H), 2.73 (dt, J=13.6, 6.8 Hz, 1H), 2.41 (s, 3H), 1.06 (d, J=6.8 Hz, 6H).

### Example 1-193 Preparation of Compound I-103

FIG. 103 illustrates the synthetic scheme of compound I-103. As shown in FIG. 103, the specific synthesis step is as follows:

### Step 1: N-(4-(5-benzyl-2-(4-fluorophenyl)-4,5,6,7-tetrahydropyrazolo[1,5-a]pyrazin-3-yl)pyridin-2-yl)propionamide (2)

To a solution of 4-[2-(4-fluorophenyl)-5-(1-methylphenyl)-4H,6H,7H-pyrazolo[1,5-a]pyrazin-3-yl]pyridin-2-amine (200 mg, 0.4994 mmol) in DCM (10 mL), then propanoyl chloride (69.31 mg, 0.7491 mmol) and DIPEA (128.85 mg, 0.9988 mmol) was added. The reaction mixture was stirred at 25° C. for 2 h. Then MeOH (5 mL) and K₂CO₃ (107.70 mg, 0.7804 mmol) was added. The reaction mixture was stirred at 25° C. for 2 h. The reaction was complete detected by lcms. The reaction was complete detected by lcms. The reaction mixture was filtered and concentrated under pressure. The crude material was added to a silica gel column and was eluted with PE/EtOAc (1:1). Chemical Formula: calculated for (M+H⁺) C₂₇H₂₆FN₅O: 455.2, Found: 455.7.

### Step 2: N-(4-(2-(4-fluorophenyl)-4,5,6,7-tetrahydropyrazolo[1,5-a]pyrazin-3-yl)pyridin-2-yl)propionamide

To a solution of N-(4-(5-benzyl-2-(4-fluorophenyl)-4,5,6,7-tetrahydropyrazolo[1,5-a]pyrazin-3-yl)pyridin-2-yl)propionamide (200 mg, 0.4381 mmol) in EA/MeOH (10 mL), then Pd/C (100 mg) and HCl (1 mL) was added under H₂. The reaction mixture was stirred at 25° C. for 2 h. The reaction was complete detected by lcms. The solution was filtered, filtrate was collected. The reaction mixture was concentrated under pressure. The organic phase was washed with 2 M sodium carbonate solution (5 mL). The residue was extracted with EA (10 mL*3). The combined organic layers were washed with brine (10 mL), dried over sodium sulfate, filtered and concentrated to give the product. Chemical Formula: calculated for (M+H⁺) C₂₀H₂₀FN₅O: 365.2, Found: 365.8.

### Step 3: N-(4-(2-(4-fluorophenyl)-5-methyl-4,5,6,7-tetrahydropyrazolo[1,5-a]pyrazin-3-yl) pyridin-2-yl)propionamide

To a solution of N-(4-(2-(4-fluorophenyl)-4,5,6,7-tetrahydropyrazolo[1,5-a]pyrazin-3-yl)pyridin-2-yl)propionamide (120 mg, 0.3284 mmol) in THF (5 mL), then HCHO (98.52 mg, 3.2840 mmol) was added. The reaction mixture was stirred at 70° C. for 14 h. Then sodium triacetoxyborohydride (83.54 mg, 0.3940 mmol) was added. The reaction mixture was stirred at 25° C. for 2 h. The reaction was complete detected by lcms. The reaction mixture was concentrated under pressure. The organic phase was washed with H₂O (5 mL). The residue was extracted with EA (10 mL*3). The combined organic layers were washed with brine (10 mL), dried over sodium sulfate, filtered and concentrated to give the product. The residue was purified via Genal-Prep-HPLC and give the product.

¹H NMR (400 MHz, DMSO) δ 10.44 (s, 1H), 8.20 (d, J=5.2 Hz, 1H), 7.95 (s, 1H), 7.43-7.35 (m, 2H), 7.21-7.13 (m, 2H), 6.75 (dd, J=5.2, 1.6 Hz, 1H), 4.19 (t, J=5.6 Hz, 2H), 3.61 (s, 2H), 2.92 (t, J=5.6 Hz, 2H), 2.41 (s, 3H), 2.39-2.32 (m, 2H), 1.03 (t, J=7.6 Hz, 3H).

### Example 1-104 Preparation of Compound I-104

FIG. 104 illustrates the synthetic scheme of compound I-104. As shown in FIG. 104, the specific synthesis step is as follows:

### Step 1:1-(4-bromopyridin-2-yl)propan-2-one (2)

To a solution of 4-bromo-2-methylpyridine (3 g, 17.4 mmol) in THF (30 mL), LDA (13 mL, 26 mmol, 2N) was added. The reaction mixture was stirred at −78° C. for 1 h. N-methoxy-N-methylacetamide (2.69 g, 26 mmol) was added. The reaction mixture was stirred at 25° C. for 15 h. The reaction mixture was concentrated under pressure. Chemical Formula: calculated for (M+H⁺) C₈H₈BrN₄O: 214.06, Found: 213.8.

### Step 2:1-(4(5-benzyl-2-(4-fluorophenyl)-4,5,6,7-tetrahydropyrazolo[1,5-a]pyrazin-3-yl)pyridin-2-yl)propan-2-one (3)

1-(4-bromopyridin-2-yl)propan-2-one (2 g, 9.3 mmol), potassium carbonate (2.57 g, 18.6 mmol) and Tetrakis(triphenylphosphine)Palladium (1 g, 0.9 mmol) was added to a solution of 2-(4-fluorophenyl)-5-(1-methylphenyl)-3-(4,4,5,5-tetramethyl-1,3,2-dioxaborolan-2-yl)-4H,6H,7H-pyrazolo[1,5-a]pyrazine (6 g, 13.9 mmol) in dioxane (30 mL) and water (5 mL). The reaction mixture was stirred at 100° C. for 18 h. The reaction mixture was concentrated under pressure. Chemical Formula: calculated for (M+H⁺) C₂₇H₂₅FN₄O: 440.52, Found: 440.8.

### Step 3:5-benzyl-2-(4-fluorophenyl)-3-(2-((2-methyl-1,3-dioxolan-2-yl)methyl)pyridin-4-yl)-4,5,6,7-tetrahydropyrazolo[1,5-a]pyrazine (4)

To a solution of 1-(4-(5-benzyl-2-(4-fluorophenyl)-4,5,6,7-tetrahydropyrazolo[1,5-a]pyrazin-3-yl)pyridin-2-yl)propan-2-one (500 mg, 1.13 mmol) in toluene (20 mL), ethylene glycol (702 mg, 11.3 mmol) and PTS (32 mg, 0.23 mmol) were added. The mixture was stirred at 130° C. for 4 h. The reaction mixture was concentrated under pressure. Chemical Formula: calculated for (M+H⁺) C₂₉H₂₉FN₄O₂: 484.58, Found: 484.8.

### Step 4:2-(4-fluorophenyl)-3-(2-4(2-methyl-1,3-dioxolan-2-yl)methyl)pyridin-4-yl)-4,5,6,7-tetrahydropyrazolo[1,5-a]pyrazine (5)

To a solution of 5-benzyl-2-(4-fluorophenyl)-3-(2-((2-methyl-1,3-dioxolan-2-yl)methyl)pyridin-4-yl)-4,5,6,7-tetrahy dropyrazolo[1,5-a]pyrazine (450 mg, 0.93 mmol) in EA (20 mL), Pd/C (50 mg) was added. The mixture was stirred at 60° C. for 4 h under hydrogen atmosphere. The solution was filtered, filtrate was collected and the concentrated under pressure. Chemical Formula: calculated for (M+H⁺) C₂₂H₂₃FN₄O₂: 394.45, Found: 394.8.

### Step 5:1-(2-(4-fluorophenyl)-3-(2-((2-methyl-1,3-dioxolan-2-yl)methyl)pyridin-4-yl)-6,7-dihydropyrazolo[1,5-a]pyrazin-5(4H)-yl)ethan-1-one (6)

To a solution of 2-(4-fluorophenyl)-3-(2-((2-methyl-1,3-dioxolan-2-yl)methyl)pyridin-4-yl)-4,5,6,7-tetrahydropyrazolo[1,5-a]pyrazine (400 mg, 1.1 ml) in DCM (12 mL) at 0° C., acetyl chloride (96 mg, 1.22 mmol) in DCM (3 mL) and Et₃N (123.14 mg, 1.22 mmol) were added. The mixture was stirred at 0° C. for 1 h. The reaction mixture was concentrated under pressure. Chemical Formula: calculated for (M+H⁺) C₂₄H₂₅FN₄O₃: 436.49, Found: 436.8.

### Step 6: 1-(4-(5-acetyl-2-(4-fluorophenyl)-4,5,6,7-tetrahydropyrazolo[1,5-a]pyrazin-3-yl)pyridin-2-yl)propan-2-one

To a solution of 1-(2-(4-fluorophenyl)-3-(2-((2-methyl-1,3-dioxolan-2-yl)methyl)pyridin-4-yl)-6,7-dihydropyrazolo[1,5-a]pyrazin-5(4H)-yl)ethan-1-one (200 mg, 0.46 mmol) in THF (5 mL), 3 M HCl (5 mL) was added. The mixture was stirred at 25° C. for 16 h. The reaction mixture was concentrated under pressure. The mixture was adjusted to pH=7 with NaHCO₃. The residue was extracted with EA. The reaction mixture was concentrated under pressure. Chemical Formula: calculated for (M+H⁺) C₂₂H₂₁FN₄O₂: 392.43, Found: 392.8. ¹H NMR (400 MHz, DMSO) δ 8.46 (d, J=5.1 Hz, 1H), 7.48-7.30 (m, 2H), 7.18 (dd, J=12.4, 5.5 Hz, 2H), 7.14-6.98 (m, 2H), 4.79 (d, J=30.4 Hz, 2H), 4.41-4.12 (m, 2H), 4.11-3.94 (m, 2H), 3.88 (s, 2H), 2.12 (t, J=11.1 Hz, 6H).

### Example 1-165 Preparation of Compound I-105

FIG. 105 illustrates the synthetic scheme of compound I-105. As shown in FIG. 105, the specific synthesis step is as follows:

### Step 1:3-bromo-2-(4-fluorophenyl)-5-methyl-4,5,6,7-tetrahydropyrazolo[1,5-a]pyrazine (2)

To a solution of 3-bromo-2-(4-fluorophenyl)-4,5,6,7-tetrahydropyrazolo[1,5-a]pyrazine (2.00 g, 6.75 mmol) in THF (20 mL), paraformaldehyde (204.20 mg, 6.80 mmol) was added in. The mixture was stirred at 25° C. for 30 min. Then sodium cyanoborohydride (848.34 mg, 13.5 mmol) was added in at 0° C. The reaction was stirred at 25° C. for 2 h. The reaction was quenched with water and was extracted with EA twice time, the organic phase was combined and concentrated under pressure. The residue was purified via Prep-TLC and give the product as off-white solid (1.58 g, 75.3%)

Chemical Formula: calculated for (M+H+)C₁₃H₁₃BrFN₃: 310.17, Found:311.

### Step 2: 4-(2-(4-fluorophenyl)-5-methyl-4,5,6,7-tetrahydropyrazolo[1,5-a]pyrazin-3-yl)pyridin-2-amine (3)

To a solution of 3-bromo-2-(4-fluorophenyl)-5-methyl-4,5,6,7-tetrahydropyrazolo[1,5-a]pyrazine (1.50 g, 4.84 mmol) in Dioxane (20 mL) and Water (2 ml), 4-(3,3,4,4-tetramethylborolan-1-yl)pyridin-2-amine (1.26 g, 5.81 mmol), Pd (dppf)C₁₂ (150 mg, 10% w/w) and K₃PO₄ (2.05 g, 9.68 mmol) were added in. The mixture was stirred at 90° C. under N₂ atmosphere for 4 h. The reaction mixture was diluted with water, and the water phase was extracted with EA twice time, the organic phase was combined and concentrated under pressure. The residue was purified via Prep-TLC and give the product as yellow solid (1.10 g, 70.6%) 17951 Chemical Formula: calculated for (M+H+)C₁₈H₁₈FN₅: 323.38, Found:324.

### Step 3: N-(4-(2-(4-fluorophenyl)-5-methyl-4,5,6,7-tetrahydropyrazolo[1,5-a]pyrazin-3-yl)pyridin-2-yl)-2-(piperidin-1-yl) acetamide

To a solution of 4-(2-(4-fluorophenyl)-5-methyl-4,5,6,7-tetrahydropyrazolo[1,5-a]pyrazin-3-yl)pyridin-2-amine (50 mg, 0.15 mmol) in DCM (7 mL), TEA (30.36 mg, 0.30 mmol) was added in and then 2-(piperidin-1-yl) acetyl chloride (27.48 mg, 0.17 mmol) was dropped in at 0° C. The mixture was stirred at 25° C. for 2 h. The reaction mixture was diluted with water, and the water phase was extracted with DCM twice time, the organic phase was combined and concentrated under pressure. The residue was purified via Genal-Prep-HPLC and give the product as white solid (35.72 mg, 51.5%)

Chemical Formula: calculated for (M+H+) C₂₅H₂₉FN₆O: 448.55, Found:449.

### Example 1-196 Preparation of Compound I-106

FIG. 106 illustrates the synthetic scheme of compound I-106. As shown in FIG. 106, the specific synthesis step is as follows:

### Step 1: N-(4-(2-(4-fluorophenyl-5-methyl-4,5,6,7-tetrahydropyrazolo[1,5-a]pyrazin-3-yl)pyridin-2-yl)-2-(pyrrolidin-1-yl) acetamide

To a solution of 4-(2-(4-fluorophenyl)-5-methyl-4,5,6,7-tetrahydropyrazolo[1,5-a]pyrazin-3-yl)pyridin-2-amine (50 mg, 0.15 mmol) in DCM (6 mL), TEA (30.36 mg, 0.30 mmol) was added in and then 2-(pyrrolidin-1-yl) acetyl chloride (25.09 mg, 0.17 mmol) was dropped in at 0° C. The mixture was stirred at 25° C. for 2 h. The reaction mixture was diluted with water, and the water phase was extracted with DCM twice time, the organic phase was combined and concentrated under pressure. The residue was purified via Genal-Prep-HPLC and give the product as white solid (31.04 mg, 46.2%)

Chemical Formula: calculated for (M+H+) C₂₄H₂₇FN₆O: 434.52, Found:435.

### Example 1-167 Preparation of Compound I-107

FIG. 107 illustrates the synthetic scheme of compound I-107. As shown in FIG. 107, the specific synthesis step is as follows:

### Step 1: N-(4-(2-(4-fluorophenyl)-5-methyl-4,5,6,7-tetrahydropyrazolo[1,5-a]pyrazin-3-yl)pyridin-2-yl)-2 tetrahydro-2H-pyran-4-yl) acetamide

To a solution of 4-(2-(4-fluorophenyl)-5-methyl-4,5,6,7-tetrahydropyrazolo[1,5-a]pyrazin-3-yl)pyridin-2-amine (50 mg, 0.15 mmol) in DCM (6 mL), TEA (30.36 mg, 0.30 mmol) was added in and then 2-(tetrahydro-2H-pyran-4-yl) acetyl chloride (27.64 mg, 0.17 mmol) was dropped in at 0° C. The mixture was stirred at 25° C. for 2 h. The reaction mixture was diluted with water, and the water phase was extracted with DCM twice time, the organic phase was combined and concentrated under pressure. The residue was purified via Genal-Prep-HPLC and give the product as white solid (37.46 mg, 53.9%)

Chemical Formula: calculated for (M+H+) C₂₅H₂₅FN₅O₂: 449.53, Found:450.

### Example 1-108 Preparation of Compound I-108

FIG. 108 illustrates the synthetic scheme of compound I-108. As shown in FIG. 108, the specific synthesis step is as follows:

### Step 1: N-(4-(2-(4-fluorophenyl)-5-methyl-4,5,6,7-tetrahydropyrazolo[1,5-a]pyrazin-3-yl)pyridin-2-yl)-2-(thiazol-4-yl) acetamide

To a solution of 4-(2-(4-fluorophenyl)-5-methyl-4,5,6,7-tetrahydropyrazolo[1,5-a]pyrazin-3-yl)pyridin-2-amine (50 mg, 0.15 mmol) in DCM (5 mL), TEA (30.36 mg, 0.3 mmol) was added in and then 2-(thiazol-4-yl) acetyl chloride (29.09 mg, 0.18 mmol) was dropped in at 0° C. The mixture was stirred at 25° C. for 2 h. The reaction mixture was diluted with water, and the water phase was extracted with DCM twice time, the organic phase was combined and concentrated under pressure. The residue was purified via Genal-Prep-HPLC and give the product as white solid (22.75 mg, 32.8%)

Chemical Formula: calculated for (M+H+) C₂₃H₂₁FN₆OS: 448.52, Found: 449.

### Example 1-109 Preparation of Compound I-109

FIG. 109 illustrates the synthetic scheme of compound I-109. As shown in FIG. 109, the specific synthesis step is as follows:

### Step 1: N-(4-(2-(4-fluorophenyl-5-methyl-4,5,6,7-tetrahydropyrazolo[1,5-a]pyrazin-3-yl)pyridin-2-yl)-2-(furan-3-yl) acetamide

To a solution of 4-(2-(4-fluorophenyl)-5-methyl-4,5,6,7-tetrahydropyrazolo[1,5-a]pyrazin-3-yl)pyridin-2-amine (50 mg, 0.15 mmol) in DCM (6 mL), TEA (30.36 mg, 0.30 mmol) was added in and then 2-(furan-3-yl) acetyl chloride (26.02 mg, 0.18 mmol) was dropped in at 0° C. The mixture was stirred at 25° C. for 2 h. The reaction mixture was diluted with water, and the water phase was extracted with DCM twice time, the organic phase was combined and concentrated under pressure. The residue was purified via Genal-Prep-HPLC and give the product as white solid (32.09 mg, 48.1%).

Chemical Formula: calculated for (M+H+) C₂₄H₂₂FN₃O₂:431.47, Found:432.

### Example 1-116 Preparation of Compound I-110

FIG. 110 illustrates the synthetic scheme of compound I-110. As shown in FIG. 110, the specific synthesis step is as follows:

### Step 1: N-(4-(2-(4-fluorophenyl)-5-methyl-4,5,6,7-tetrahydropyrazolo[1,5-a]pyrazin-3-yl)pyridin-2-yl)-2-(thiophen-2-yl) acetamide

To a solution of 4-(2-(4-fluorophenyl)-5-methyl-4,5,6,7-tetrahydropyrazolo[1,5-a]pyrazin-3-yl)pyridin-2-amine (50 mg, 0.15 mmol) in DCM (7 mL), TEA (30.36 mg, 0.30 mmol) was added in and then 2-(thiophen-2-yl) acetyl chloride (28.91 mg, 0.18 mmol) was dropped in at 0° C. The mixture was stirred at 25° C. for 2 h. The reaction mixture was diluted with water, and the water phase was extracted with DCM twice time, the organic phase was combined and concentrated under pressure. The residue was purified via Genal-Prep-HPLC and give the product as white solid (22.49 mg, 32.5%).

Chemical Formula: calculated for (M+H+) C₂₄H₂₂FN₃OS:447.53, Found:448.

### Example 1-111 Preparation of Compound I-111

FIG. 111 illustrates the synthetic scheme of compound I-111. As shown in FIG. 111, the specific synthesis step is as follows:

### Step 1: N-(4-(2-(4-fluorophenyl)-5-methyl-4,5,6,7-tetrahydropyrazolo[1,5-a]pyrazin-3-yl)pyridin-2-yl)-2-(furan-2-yl) acetamide

To a solution of 4-(2-(4-fluorophenyl)-5-methyl-4,5,6,7-tetrahydropyrazolo[1,5-a]pyrazin-3-yl)pyridin-2-amine (50 mg, 0.15 mmol) in DCM (6 mL), TEA (30.36 mg, 0.30 mmol) was added in and then 2-(furan-2-yl) acetyl chloride (26.02 mg, 0.18 mmol) was dropped in at 0° C. The mixture was stirred at 25° C. for 2 h. The reaction mixture was diluted with water, and the water phase was extracted with DCM twice time, the organic phase was combined and concentrated under pressure. The residue was purified via Genal-Prep-HPLC and give the product as white solid (33.56 mg, 50.3%).

Chemical Formula: calculated for (M+H+) C₂₄H₂₂FN₅O₂:431.47, Found:432.

### Example 1-112 Preparation of Compound I-112

FIG. 112 illustrates the synthetic scheme of compound I-112. As shown in FIG. 112, the specific synthesis step is as follows:

### Step 1: N-(4-(2-(4-fluorophenyl)-5-methyl-4,5,6,7-tetrahydropyrazolo[1,5-a]pyrazin-3-yl)pyridin-2-yl)-2-(1H-pyrrol-1-yl) acetamide

To a solution of 4-(2-(4-fluorophenyl)-5-methyl-4,5,6,7-tetrahydropyrazolo[1,5-a]pyrazin-3-yl)pyridin-2-amine (50 mg, 0.15 mmol) in DCM (5 mL), TEA (30.36 mg, 0.30 mmol) was added in and then 2-(1H-pyrrol-1-yl) acetyl chloride (24.41 mg, 0.17 mmol) was dropped in at 0° C. The mixture was stirred at 25° C. for 2 h. The reaction mixture was diluted with water, and the water phase was extracted with DCM twice time, the organic phase was combined and concentrated under pressure. The residue was purified via Genal-Prep-HPLC and give the product as white solid (34.74 mg, 52.2%).

Chemical Formula: calculated for (M+H+) C₂₄H₂₃FN₆O:430.49, Found:431.

### Example 1-113 Preparation of Compound I-113

FIG. 113 illustrates the synthetic scheme of compound I-113. As shown in FIG. 113, the specific synthesis step is as follows:

### Step 1:1-(2-(4-fluorophenyl)-3-(isoxazololo[4,5-b]pyridin-7-yl)-6,7-dihydropyrazolo[1,5-b]pyrazin-5(4H)-yl) ethan-1-one (2)

To a solution of 1-(3-bromo-2-(4-fluorophenyl)-6,7-dihydropyrazolo[1,5-a]pyrazin-5(4H)-yl)ethan-1-one (35 mg, 0.10 mmol) in Dioxane (3 mL) and Water (0.5 ml) 7-(3,3,4,4-tetramethylborolan-1-yl)isoxazolo[4,5-b]pyridine (26.63 mg, 0.11 mmol), Pd(dppf)Cl₂ (3.5 mg, 10% w/w)) and K₃PO₄ (42.45 mg, 0.20 mmol) were added in. The mixture was stirred at 90° C. under N₂ atmosphere for 4 h. The reaction mixture was diluted with water, and the water phase was extracted with EA twice time, the organic phase was combined and concentrated under pressure. The residue was purified via Prep-TLC and give the product as light brown solid (24.88 mg, 63.7%)

Chemical Formula: calculated for (M+H+) C₂₀H₁₆FN₅O₂: 377.38, Found:378.

### Step 2:7-(5-acetyl-2-(4-fluorophenyl)-4,5,6,7-tetrahydropyrazolo[1,5-a]pyrazin-3-yl)-2-methylisoxazolo[4,5-b]pyridin-2-ium

To a solution of 1-(2-(4-fluorophenyl)-3-(isoxazolo[4,5-b]pyridin-7-yl)-6,7-dihydropyrazolo[1,5-a]pyrazin-5(4H)-yl) ethan-1-one (20 mg, 0.05 mmol) in DMF (2 mL), Cs₂CO₃ (32.58 mg, 0.1 mmol) was added in and then iodomethane (9.94 mg, 0.07 mmol) was dropped in at 0° C. The mixture was stirred at 65° C. for 2 h. The reaction mixture was diluted with water, and the water phase was extracted with EA twice time, the organic phase was combined and concentrated under pressure. The residue was purified via Genal-Prep-HPLC and give the product as white solid (11.64 mg, 42.3%)

Chemical Formula: calculated for (M+H+)C₂₁H₁₉FN₅O₂: 392.41, Found:393.

### Example 1-114 Preparation of Compound I-114

FIG. 114 illustrates the synthetic scheme of compound I-114. As shown in FIG. 114, the specific synthesis step is as follows:

### Step 1: N-(4-(5-benzyl-2-(4-fluorophenyl)-4,5,6,7-tetrahydropyrazolo[1,5-a]pyrazin-3-yl)pyridin-2-yl)-2-cyclohexylacetamide (2)

To a solution of 4-(5-benzyl-2-(4-fluorophenyl)-4,5,6,7-tetrahydropyrazolo[1,5-a]pyrazin-3-yl)pyridin-2-amine (150 mg, 0.38 mmol) in DCM (10 ml), TEA (76.90 mg, 0.76 mmol) was added in. Then 2-cyclohexylacetyl chloride (61.04 mg, 0.38 mmol) was dropped in at 0° C. The reaction mixture was stirred at 25° C. for 10 h. The reaction mixture was diluted with water, and the water phase was extracted with DCM twice time, the organic phase was combined and concentrated under pressure. The residue was purified via Prep-TLC and give the product as brown solid (165.57 mg, 84.2%)

Chemical Formula: calculated for (M+H+): C₃₂H₃₄FN₅O, 523.66, Found:524.

### Step 2: 2-cyclohexyl-N-(4-(2-(4-fluorophenyl)-4,5,6,7-tetrahydropyrazolo[1,5-a]pyrazin-3-yl)pyridin-2-yl) acetamide (3)

To a solution of N-(4-(5-benzyl-2-(4-fluorophenyl)-4,5,6,7-tetrahydropyrazolo[1,5-a]pyrazin-3-yl)pyridin-2-yl)-2-cyclohexylacetamide (150 mg, 0.29 mmol) in MeOH (10 mL), Pd/C (15 mg, 10% w/w) was added in. The mixture was stirred at 45° C. for 8 h under H₂ atmosphere. The reaction mixture was filtrated, The filtrate liquid was concentrated under pressure. A dark solid was obtained (99.47 mg, 80.1%)

Chemical Formula: calculated for (M+H+):C₂₅H₂₅FN₅O, 433.53, Found:434.

### Step 3: 2-cyclohexyl-N-(4-(2-(4-fluorophenyl)-5-propionyl-4,5,6,7-tetrahydropyrazolo[1,5-a]pyrazin-3-yl)pyridin-2-yl) acetamide

To a solution of 2-cyclohexyl-N-(4-(2-(4-fluorophenyl)-4,5,6,7-tetrahydropyrazolo[1,5-a]pyrazin-3-yl)pyridin-2-yl) a cetamide (50 mg, 0.12 mmol) in DMF (5 mL), Cs₂CO₃ (78.20 mg, 0.24 mmol) and then propionyl chloride (13.88 mg, 0.15 mmol) was dropped in. The mixture was stirred at 25° C. for 12 h. The reaction mixture was diluted with water, and the water phase was extracted with EA twice time, the organic phase was combined and concentrated under pressure. The residue was purified viaGenal-Prep-HPLC and give the product as white solid (25.69 mg, 45.5%)

Chemical Formula: calculated for (M+H+)C₂₈H₃₂FN₅O₂:489.60, Found:490.

### Example 1-115 Preparation of Compound I-115

FIG. 115 illustrates the synthetic scheme of compound I-115. As shown in FIG. 115, the specific synthesis step is as follows:

### Step 1:2-cyclohexyl-N-(4-(2-(4-fluorophenyl)-5-methyl-4,5,6,7-tetrahydropyrazolo[1,5-a]pyrazin-3-yl)pyridin-2-yl) acetamide

To a solution of 2-cyclohexyl-N-(4-(2-(4-fluorophenyl)-4,5,6,7-tetrahydropyrazolo[1,5-a]pyrazin-3-yl)pyridin-2-yl) a cetamide (40 mg, 0.09 mmol) in THF (4 mL), paraformaldehyde (3.00 mg, 0.10 mmol) was added in and the mixture was stirred at 25° C. for 30 min. Then sodium cyanoborohydride (11.31 mg, 0.18 mmol) was added in at 0° C. The mixture was stirred at 25° C. for 2 h. The reaction was quenched with water, and was extracted with EA twice time, the organic phase was combined and concentrated under pressure. The residue was purified viaGenal-Prep-HPLC and give the product as white solid (14.54 mg, 35.2%)

Chemical Formula: calculated for (M+H+)C₂₆H₃₀FN₅O:447.56, Found:448.

### Example 1-116 Preparation of Compound I-116

FIG. 116 illustrates the synthetic scheme of compound I-116. As shown in FIG. 116, the specific synthesis step is as follows:

### Step 1:1-(3-(2-aminopyridin-4-yl)-2-(4-fluorophenyl)-6,7-dihydropyrazolo[1,5-a]pyrazin-5(4H)-yl)ethan-1-one (2)

To a solution of 1-(3-bromo-2-(4-fluorophenyl)-6,7-dihydropyrazolo[1,5-a]pyrazin-5(4H)-yl)ethan-1-one (110 mg, 0.33 mmol) in Dioxane (10 mL) and Water (1 ml), 4-(3,3,4,4-tetramethylborolan-1-yl)pyridin-2-amine (75.65 mg, 0.35 mmol), Pd(dppf)Cl₂ (1 mg, 10% w/w)) and K₃PO₄ (215.04 mg, 0.66 mmol) were added in. The mixture was stirred at 90° C. under N₂ atmosphere for 3 h. The reaction mixture was diluted with water, and the water phase was extracted with EA twice time, the organic phase was combined and concentrated under pressure. The residue was purified via Prep-TLC and give the product as often-white solid (76.69 mg, 67.1%)

Chemical Formula: calculated for (M+H+): C₁₉H₁₈FN₅O, 351.39, Found:352.

### Step 2: N-(4-(5-acetyl-2-(4-fluorophenyl)-4,5,6,7-tetrahydropyrazolo[1,5-a]pyrazin-3-yl)pyridin-2-yl)-2-(thiophen-2-yl) acetamide

To a solution of 1-(3-(2-aminopyridin-4-yl)-2-(4-fluorophenyl)-6,7-dihydropyrazolo[1,5-a]pyrazin-5(4H)-yl)ethan-1-one (35 mg, 0.10 mmol) in DMF (4 mL), Cs₂CO₃ (65.16 mg, 0.20 mmol) and then 2-(thiophen-2-yl) acetyl chloride (17.67 mg, 0.11 mmol) was dropped in. The mixture was stirred at 25° C. for 12 h. The reaction mixture was diluted with water, and the water phase was extracted with EA twice time, the organic phase was combined and concentrated under pressure. The residue was purified viaGenal-Prep-HPLC and give the product as white solid (21.60 mg, 45.6%)

Chemical Formula: calculated for (M+H+) C₂₅H₂₂FN₅O₂S: 475.54, Found: 476.

### Example 1-117 Preparation of Compound I-117

FIG. 117 illustrates the synthetic scheme of compound I-117. As shown in FIG. 117, the specific synthesis step is as follows:

### Step 1: N-(4-(5-acetyl-2-(4-fluorophenyl)-4,5,6,7-tetrahydropyrazolo[1,5-a]pyrazin-3-yl)pyridin-2-yl)-2-(furan-2-yl) acetamide

To a solution of 1-(3-(2-aminopyridin-4-yl)-2-(4-fluorophenyl)-6,7-dihydropyrazolo[1,5-a]pyrazin-5(4H)-yl)ethan-1-one (35 mg, 0.10 mmol) in DMF (4 mL), Cs₂CO₃ (65.16 mg, 0.20 mmol) and then 2-(furan-2-yl) acetyl chloride (15.90 mg, 0.11 mmol) was dropped in. The mixture was stirred at 25° C. for 12 h. The reaction mixture was diluted with water, and the water phase was extracted with EA twice time, the organic phase was combined and concentrated under pressure. The residue was purified viaGenal-Prep-HPLC and give the product as white solid (17.71 mg, 38.7%).

Chemical Formula: calculated for (M+H+):C₂₅H₂₂FN₅O₃, 459.48 Found:460.

### Example 1-118 Preparation of Compound I-118

FIG. 118 illustrates the synthetic scheme of compound I-118. As shown in FIG. 118, the specific synthesis step is as follows:

### Step 1: N-(4-(5-acetyl-2-(4-fluorophenyl)-4,5,6,7-tetrahydropyrazolo[1,5-a]pyrazin-3-yl)pyridin-2-yl)-2-(furan-3-yl) acetamide

To a solution of 1-[3-(2-aminopyridin-4-yl)-2-(4-fluorophenyl)-4H,6H,7H-pyrazolo[1,5-a]pyrazin-5-yl]ethanone [100 mg, 0.2846 mmol] in pyridine [5 mL], then was added 2-(furan-3-yl) acetic acid [43 mg, 0.3415 mmol], HATU [118.963 mg, 0.3130 mmol], The reaction mixture was stirred at 100° C. for 16 h. The reaction was complete detected by lcms. The solution was filtered, filtrate was collected. The reaction was quenched by H₂O (50 mL), The mixture was concentrated. The mixture was extracted with EA (50 mL*3). The combined organic layers were washed with brine (50 mL), dried over sodium sulfate, filtered and concentrated to give the crude product. The reaction mixture was concentrated under pressure. The residue was purified via Genal-Prep-HPLC and give the product.

### Example 1-119 Preparation of Compound I-119

FIG. 119 illustrates the synthetic scheme of compound I-119. As shown in FIG. 119, the specific synthesis step is as follows:

### Step 1: N-(4-(5-acetyl-2-(4-fluorophenyl)-4,5,6,7-tetrahydropyrazolo[1,5-a]pyrazin-3-yl)pyridin-2-yl)-2-(tetrahydro-2H-pyran-4-yl) acetamide

To a solution of 1-[3-(2-aminopyridin-4-yl)-2-(4-fluorophenyl)-4H,6H,7H-pyrazolo[1,5-a]pyrazin-5-yl]ethanone [100 mg, 0.2846 mmol] in pyridine [5 mL], then was added 2-(tetrahydro-2H-pyran-4-yl) acetic acid [49 mg, 0.3415 mmol], HATU [118.963 mg, 0.3130 mmol]. The reaction mixture was stirred at 100° C. for 16 h. The reaction was complete detected by lcms. The solution was filtered, filtrate was collected. The reaction was quenched by H₂O (50 mL), The mixture was concentrated. The mixture was extracted with EA (50 mL*3). The combined organic layers were washed with brine (50 mL), dried over sodium sulfate, filtered and concentrated to give the crude product. The reaction mixture was concentrated under pressure. The residue was purified via Genal-Prep-HPLC and give the product.

### Example 1-120 Preparation of Compound I-120

FIG. 120 illustrates the synthetic scheme of compound I-120. As shown in FIG. 120, the specific synthesis step is as follows:

### Step 1: N-(4-(5-acetyl-2-(4-fluorophenyl)-4,5,6,7-tetrahydropyrazolo[1,5-a]pyrazin-3-yl)pyridin-2-yl)-2-(thiazol-4-yl) acetamide

To a solution of 1-[3-(2-aminopyridin-4-yl)-2-(4-fluorophenyl)-4H,6H,7H-pyrazolo[1,5-a]pyrazin-5-yl]ethanone [100 mg, 0.2846 mmol] in pyridine [5 mL], then was added 2-(thiazol-4-yl) acetic acid [49 mg, 0.3415 mmol], HATU [118.963 mg, 0.3130 mmol], The reaction mixture was stirred at 100° C. for 16 h. The reaction was complete detected by lcms. The solution was filtered, filtrate was collected. The reaction was quenched by H₂O (50 mL), The mixture was concentrated. The mixture was extracted with EA (50 mL*3). The combined organic layers were washed with brine (50 mL), dried over sodium sulfate, filtered and concentrated to give the crude product. The reaction mixture was concentrated under pressure. The residue was purified via Genal-Prep-HPLC and give the product.

### Example 1-121 Preparation of Compound I-121

FIG. 121 illustrates the synthetic scheme of compound I-121. As shown in FIG. 121, the specific synthesis step is as follows:

### Step 1: N-(4-(2-(4-fluorophenyl)-5-propionyl-4,5,6,7-tetrahydropyrazolo[1,5-a]pyrazin-3-yl)pyridin-2-yl)pentanamide

To a solution of 1-(3-(2-aminopyridin-4-yl)-2-(4-fluorophenyl)-6,7-dihydropyrazolo[1,5-a]pyrazin-5(4H)-yl)propan-1-one [100 mg, 0.2846 mmol] in pyridine [5 mL], then was added pentanoic acid [35 mg, 0.3415 mmol], HATU [118.963 mg, 0.3130 mmol], The reaction mixture was stirred at 100° C. for 16 h. The reaction was complete detected by lcms. The solution was filtered, filtrate was collected. The reaction was quenched by H₂O (50 mL), The mixture was concentrated. The mixture was extracted with EA (50 mL*3). The combined organic layers were washed with brine (50 mL), dried over sodium sulfate, filtered and concentrated to give the crude product. The reaction mixture was concentrated under pressure. The residue was purified via Genal-Prep-HPLC and give the product.

### Example 1-122 Preparation of Compound I-122

FIG. 122 illustrates the synthetic scheme of compound I-122. As shown in FIG. 122, the specific synthesis step is as follows:

### Step 1: N-(4-(2-(4-fluorophenyl-5-methyl-4,5,6,7-tetrahydropyrazolo[1,5-a]pyrazin-3-yl)pyridin-2-yl)pentanamide

To a solution of 4-(2-(4-fluorophenyl)-5-methyl-4,5,6,7-tetrahydropyrazolo[1,5-a]pyrazin-3-yl)pyridin-2-amine [100 mg, 0.309 mmol] in pyridine [5 mL], then was added pentanoic acid [35 mg, 0.3415 mmol], HATU [118.963 mg, 0.3130 mmol], The reaction mixture was stirred at 100 for 16 h. The reaction was complete detected by lams. The solution was filtered, filtrate was collected. The reaction was quenched by H2O (50 mL), The mixture was concentrated. The mixture was extracted with EA (50 mL*3). The combined organic layers were washed with brine (50 mL), dried over sodium sulfate, filtered and concentrated to give the crude product. The reaction mixture was concentrated under pressure. The residue was purified via Genal-Prep-HPLC and give the product.

### Example 1-123 Preparation of Compound I-123

FIG. 123 illustrates the synthetic scheme of compound I-123. As shown in FIG. 123, the specific synthesis step is as follows:

### Step 1: N-(4-(5-acetyl-2-(4-fluorophenyl)-4,5,6,7-tetrahydropyrazolo[1,5-a]pyrazin-3-yl)pyridin-2-yl)-2-(piperidin-1-yl) acetamide

To a solution of 1-[3-(2-aminopyridin-4-yl)-2-(4-fluorophenyl)-4H,6H,7H-pyrazolo[1,5-a]pyrazin-5-yl]ethanone [100 mg, 0.2846 mmol] in pyridine [5 mL], then was added 2-(piperidin-1-yl) acetic acid [49 mg, 0.3415 mmol], HATU [118.963 mg, 0.3130 mmol], The reaction mixture was stirred at 100° C. for 16 h. The reaction was complete detected by lcms. The solution was filtered, filtrate was collected. The reaction was quenched by H₂O (50 mL), The mixture was concentrated. The mixture was extracted with EA (50 mL*3). The combined organic layers were washed with brine (50 mL), dried over sodium sulfate, filtered and concentrated to give the crude product. The reaction mixture was concentrated under pressure. The residue was purified via Genal-Prep-HPLC and give the product.

### Example 1-124 Preparation of Compound I-124

FIG. 124 illustrates the synthetic scheme of compound I-124. As shown in FIG. 124, the specific synthesis step is as follows:

### Step 1: N-(4-(5-acetyl-2-(4-fluorophenyl)-4,5,6,7-tetrahydropyrazolo[1,5-a]pyrazin-3-yl)pyridin-2-yl)-2-(pyrrolidin-1-yl) acetamide

To a solution of 1-[3-(2-aminopyridin-4-yl)-2-(4-fluorophenyl)-4H,6H,7H-pyrazolo[1,5-a]pyrazin-5-yl]ethanone [100 mg, 0.2846 mmol] in pyridine [5 mL], then was added 2-(pyrrolidin-1-yl) acetic acid [44 mg, 0.3415 mmol], HATU [118.963 mg, 0.3130 mmol], The reaction mixture was stirred at 100° C. for 16 h. The reaction was complete detected by lcms. The solution was filtered, filtrate was collected. The reaction was quenched by H₂O (50 mL), The mixture was concentrated. The mixture was extracted with EA (50 mL*3). The combined organic layers were washed with brine (50 mL), dried over sodium sulfate, filtered and concentrated to give the crude product. The reaction mixture was concentrated under pressure. The residue was purified via Genal-Prep-HPLC and give the product.

### Example 1-125 Preparation of Compound I-125

FIG. 125 illustrates the synthetic scheme of compound I-125. As shown in FIG. 125, the specific synthesis step is as follows:

### Step 1: N-(4-(5-acetyl-2-(4-fluorophenyl)-4,5,6,7-tetrahydropyrazolo[1,5-a]pyrazin-3-yl)pyridin-2-yl)-2-(1H-pyrrol-1-yl) acetamide

To a solution of 1-[3-(2-aminopyridin-4-yl)-2-(4-fluorophenyl)-4H,6H,7H-pyrazolo[1,5-a]pyrazin-5-yl]ethanone [100 mg, 0.2846 mmol] in pyridine [5 mL], then was added 2-(1H-pyrrol-1-yl) acetic acid [44 mg, 0.3415 mmol], HATU [118.963 mg, 0.3130 mmol], The reaction mixture was stirred at 100° C. for 16 h. The reaction was complete detected by lcms. The solution was filtered, filtrate was collected. The reaction was quenched by H₂O (50 mL), The mixture was concentrated. The mixture was extracted with EA (50 mL*3). The combined organic layers were washed with brine (50 mL), dried over sodium sulfate, filtered and concentrated to give the crude product. The reaction mixture was concentrated under pressure. The residue was purified via Genal-Prep-HPLC and give the product.

### Example 1-126 Preparation of Compound I-126

FIG. 126 illustrates the synthetic scheme of compound I-126. As shown in FIG. 126, the specific synthesis step is as follows:

### Step 1: tert-butyl 2-(4-chloropyridin-2-yl-1H-pyrrole-1-carboxylate (2)

To a solution of 2-bromo-4-chloropyridine (2 g, 10.4 mmol) in 1,4-Dioxane/H₂O=5:1 (36 mL), S(1-(tert-butoxycarbonyl)-1H-pyrrol-2-yl)boronic acid (1.1 g, 5.2 mmol), K₃PO₄ (1.27 g, 10.4 mmol) and Pd(PPh₃)₄ (1.2 g, 1 mmol) were added. The mixture was stirred at 60° C. for 3 h under nitrogen atmosphere. The reaction mixture was concentrated under pressure. The crude material was added to a silica gel column and was eluted with PE/EtOAc (10:1). Chemical Formula: calculated for (M+H⁺) C₁₄H₁₅ClN₂O₂: 278.74, Found: 278.8.

### Step 2: 1-(3-(2-(1H-pyrrol-2-yl)pyridin-4-yl)-2-(4-fluorophenyl)-6,7-dihydropyrazolo[1,5-a]pyrazin-5(4H)-yl)ethan-1-one

To a solution of tert-butyl 2-(4-chloropyridin-2-yl)-1H-pyrrole-1-carboxylate (500 mg, 1.78 mmol) in EtOH/Toluene=4:1 (10 mL), 1-(2-(4-fluorophenyl)-3-(4,4,5,5-tetramethyl-1,3,2-dioxaborolan-2-yl)-6,7-dihydropyrazolo[1,5-a]pyrazin-5(4H)-yl)ethan-1-one (826.29 mg, 2.14 mmol), 2 M Na₂CO₃ aqueous solution 0.5 mL and Pd(dppf)Cl₂ (261.57 mg, 0.0.36 mmol) were added. The mixture was stirred at 100° C. for 1 h under nitrogen atmosphere on microwave. The crude material was added to a silica gel column and was eluted with DCM/MeOH (10:1). Chemical Formula: calculated for (M+H⁺) C₂₃H₂₀FN₅O: 401.45, Found: 401.8. ¹H NMR (400 MHz, DMSO) δ 11.50 (s, 1H), 8.43 (d, J=5.0 Hz, 1H), 7.46 (dd, J=27.2, 21.1 Hz, 3H), 7.20 (t, J=8.8 Hz, 2H), 7.07-6.73 (m, 2H), 6.61 (d, J=15.3 Hz, 1H), 6.11 (d, J=2.8 Hz, 1H), 4.83 (d, J=30.6 Hz, 2H), 4.50-4.11 (m, 2H), 4.02 (d, J=5.4 Hz, 2H), 2.13 (d, J=20.8 Hz, 3H).

### Example 1-127 Preparation of Compound I-127

FIG. 127 illustrates the synthetic scheme of compound I-127. As shown in FIG. 127, the specific synthesis step is as follows:

### Step 1: tert-butyl 2-(4-chloropyridin-2-yl)-5-methyl-1H-pyrrole-1-ecarboxylate (2)

To a solution of 2-bromo-4-chloropyridine (2 g, 10.4 mmol) in 1,4-Dioxane/H₂O=5:1 (36 mL), (1-(tert-butoxycarbonyl)-5-methyl-1H-pyrrol-2-yl)boronic acid (1.2 g, 5.2 mmol), K₃PO₄ (1.27 g, 10.4 mmol) and Pd(PPh₃)₄ (1.2 g, 1 mmol) were added. The mixture was stirred at 60° C. for 3 h under nitrogen atmosphere. The reaction mixture was concentrated under pressure. The crude material was added to a silica gel column and was eluted with PE/EtOAc (10:1). Chemical Formula: calculated for (M+H⁺) C₁₅H₁₇ClN₂O₂: 293.76, Found: 293.8.

### Step 2: 1-(2-(4-fluorophenyl)-3-(2-(5-methyl-1H-pyrrol-2-yl)pyridin-4-yl)-6,7-dihydropyrazolo[1,5-a]pyrazin-5(4H)-yl)ethan-1-one

To a solution of tert-butyl 2-(4-chloropyridin-2-yl)-5-methyl-1H-pyrrole-1-carboxylate (500 mg, 1.78 mmol) in EtOH/Toluene=4:1 (10 mL), 1-(2-(4-fluorophenyl)-3-(4,4,5,5-tetramethyl-1,3,2-dioxaborolan-2-yl)-6,7-dihydropyrazolo[1,5-a]pyrazin-5(4H)-yl)ethan-1-one (826.29 mg, 2.14 mmol), 2 M Na₂CO₃ aqueous solution 0.5 mL and Pd(dppf)Cl₂ (261.57 mg, 0.0.36 mmol) were added. The mixture was stirred at 100° C. for 1 h under nitrogen atmosphere on microwave. The crude material was added to a silica gel column and was eluted with DCM/MeOH (10:1). Chemical Formula: calculated for (M+H⁺) C₂₄H₂₂FN₅O: 416.47, Found: 416.8.

### Example 1-128 Preparation of Compound I-128

FIG. 128 illustrates the synthetic scheme of compound I-128. As shown in FIG. 128, the specific synthesis step is as follows:

### Step 1: N-(4-(5-acetyl-2-(4-fluorophenyl)-4,5,6,7-tetrahydropyrazolo[1,5-a]pyrazin-3-yl)pyridin-2-yl)-3-oxabicyclo[3.1.0]hexane-6-carboxamide

To a solution of 1-[3-(2-aminopyridin-4-yl)-2-(4-fluorophenyl)-4H,6H,7H-pyrazolo[1,5-a]pyrazin-5-yl]ethanone [100 mg, 0.2846 mmol] in pyridine [5 mL], then was added 3-oxabicyclo[3.1.0]hexane-6-carboxylic acid [44 mg, 0.3415 mmol], HATU [118.963 mg, 0.3130 mmol], The reaction mixture was stirred at 100° C. for 16 h. The reaction was complete detected by lcms. The solution was filtered, filtrate was collected. The reaction was quenched by H₂O (50 mL), The mixture was concentrated. The mixture was extracted with EA (50 mL*3). The combined organic layers were washed with brine (50 mL), dried over sodium sulfate, filtered and concentrated to give the crude product. The reaction mixture was concentrated under pressure. The residue was purified via Genal-Prep-HPLC and give the product.

### Example 1-129 Preparation of Compound I-129

FIG. 129 illustrates the synthetic scheme of compound I-129. As shown in FIG. 129, the specific synthesis step is as follows:

### Step 1: 1-(3-(2-aminopyridin-4-yl)-2-(4-fluorophenyl)-6,7-dihydropyrazolo[1,5-a]pyrazin-5(4H)-yl)ethane-1-thione

To a solution of 4-[2-(4-fluorophenyl)-5-methyl-4H,6H,7H-pyrazolo[1,5-a]pyrazin-3-yl]pyridin-2-amine (300 mg, 0.9277 mmol) in DMF (10 mL), then 3-oxabicyclo[3.1.0]hexane-6-carboxylic acid (178.29 mg, 1.3915 mmol), HATU (528.79 mg, 1.3915 mmol), DIEA (359.02 mg, 2.7831 mmol) was added. The reaction mixture was stirred at 25° C. for 16 h. The reaction was complete detected by lcms. The organic phase was washed with water (10 mL). The residue was extracted with EA (5 mL*3). Solvent was dried over sodium sulphate and dried under a stream of nitrogen in the Radleys blowdown apparatus to give the crude product. The solution was filtered, filtrate was collected. The reaction mixture was concentrated under pressure. The residue was purified via Genal-Prep-HPLC and give the product.

¹H NMR (400 MHz) δ 10.68 (s, 1H), 8.17 (d, J=5.2 Hz, 1H), 7.86 (s, 1H), 7.38-7.32 (m, 2H), 7.13 (ddd, J=8.8, 5.8, 2.4 Hz, 2H), 6.73 (dd, J=5.2, 1.6 Hz, 1H), 4.15 (t, J=5.4 Hz, 2H), 3.77 (d, J=8.6 Hz, 2H), 3.65-3.52 (m, 4H), 2.88 (t, J=5.5 Hz, 2H), 2.39-2.29 (m, 3H), 2.04-2.01 (m, 2H), 1.88 (t, J=3.0 Hz, 1H).

### Example 1-138 Preparation of Compound I-130

FIG. 130 illustrates the synthetic scheme of compound I-130. As shown in FIG. 130, the specific synthesis step is as follows:

### Step 1: 2-(4-fluorophenyl)-4,5,6,7-tetrahydropyrazolo[1,5-a]pyrazine

Pd/C (100 mg) were added to a solution of 5-benzyl-3-bromo-2-(4-fluorophenyl)-4,5,6,7-tetrahydropyrazolo[1,5-a]pyrazine (2.0 g, 5.178 mmol) in MeOH (50 ml). The mixture was stirred at 60° C. for 3 h. The solution was filtered, filtrate was collected and the solution was concentrated under pressure and used without further purification.

Chemical Formula: calculated for (M+H⁺) C₁₉H₁₇BrFN₃: 385.06, Found: 386.24.

### Step 2: 2-(4-fluorophenyl)-5-(methylsulfonyl)-4,5,6,7-tetrahydropyrazolo[1,5-a]pyrazine

To a solution of 2-(4-fluorophenyl)-4,5,6,7-tetrahydropyrazolo[1,5-a]pyrazine (1.2 g, 5.527 mmol) in DCM (50 ml) at 0° C., MsCl (661 mg, 5.8 mmol) in DCM (5 ml) was added drop by drop and the mixture was stirred 3 h. SM was disappear on TLC. The reaction mixture was added the saturated aqueous solution of NaHCO₃ (10 ml) and extracted with DCM (20 ml). Then the organic layer was concentrated under pressure. The residue was purified via Prep-TLC (PF/EtOAc)=(2:1) and obtained the product.

Chemical Formula: calculated for (M+H⁺) C₁₃H₁₄FN₃O₂S: 295.08, Found: 296.21.

### Step 3: 3-bromo-2-(4-fluorophenyl)-5-(methylsulfonyl)-4,5,6,7-tetrahydropyrazolo[1,5-a]pyrazine

To a solution of 2-(4-fluorophenyl)-5-(methylsulfonyl)-4,5,6,7-tetrahydropyrazolo[1,5-a]pyrazine (1.2 g, 4.067 mmol) in acetone (50 ml), NBS (723.8 mg, 4.067 mmol) was added at r.t. The reaction mixture was stirred at r.t. for 15 h. SM was disappear on TLC. The reaction mixture was washed with H₂O (100 ml) and extracted with DCM (200 ml). Then the organic layer was concentrated under pressure. The residue was purified via Prep-TLC (PE/EtOAc)=(2:1) and obtained the product.

Chemical Formula: calculated for (M+H⁺) C₁₃H₁₃BrFN₃O₂S: 372.99, Found: 374.23.

### Step 4: 2-(4-fluorophenyl)-5-(methylsulfonyl)-3-(4,4,5,5-tetramethyl-1,3,2-dioxaborolan-2-yl)-4,5,6,7-tetrahydropyrazolo[1,5-a]pyrazine

To a solution of 3-bromo-2-(4-fluorophenyl)-5-(methylsulfonyl)-4,5,6,7-tetrahydropyrazolo[1,5-a]pyrazine (1.3 g, 3.485 mmol) in THF (50 ml), n-BuLi (3.66 mmol) was added at −78° C. The reaction mixture was stirred at −78° C. for 1 h and 2-isopropoxy-4,4,5,5-tetramethyl-1,3,2-dioxaborolane (973 mg, 5.228 mmol) in THF (10 ml) was added drop by drop. Then the mixture was warmed to r.t. After 3 h, SM was disappear on TLC. The reaction mixture was quenched with the saturated aqueous solution of NH₄Cl (10 ml) to PH=7 and extracted with DCM (200 ml). Then the organic layer was concentrated under pressure. The residue was purified via Prep-TLC (PE/EtOAc)=(2:1) and obtained the product.

Chemical Formula: calculated for (M+H⁺) C₁₉H₂₅BFN₃O₄S: 421.16, Found: 422.32.

### Step 5: 2-(4-fluorophenyl)-3-(2-(methyl-d3)pyridin-4-yl)-5-(methylsulfonyl)-4,5,6,7-tetrahydropyrazolo[1,5-a]pyrazine

To a solution of 2-(4-fluorophenyl)-5-(methylsulfonyl)-3-(4,4,5,5-tetramethyl-1,3,2-dioxaborolan-2-yl)-4,5,6,7-tetra-hydropyrazolo[1,5-a]pyrazine (1.2 g, 2.849 mmol) in THF/H₂O (50 ml/10 ml), 4-bromo-2-(methyl-d3)pyridine (495.75 mg, 2.849 mmol), Pd(dppf)Cl₂ (20.85 mg, 0.02849 mmol), AcOK (615.12 mg, 6.268 mmol) was added at r.t in N₂. The reaction mixture was refluxed for 8 h. The reaction mixture was extracted with DCM (200 ml). Then the organic layer was concentrated under pressure. The residue was purified via Prep-TLC (PE/EtOAc)=(1:1). The residue was purified via Genal-Prep-HPLC and obtained the product.

Chemical Formula: calculated for (M+H⁺) C₁₉H₁₆D₃FN₄O₂S: 389.14, Found: 390.20.

### Example 1-131 Preparation of Compound I-131

FIG. 131 illustrates the synthetic scheme of compound I-131. As shown in FIG. 131, the specific synthesis step is as follows:

### Step 1: 1-(2-(4-fluorophenyl)-6,7-dihydropyrazolo[1,5-a]pyrazin-5(4H)-yl)ethan-1-one

To a solution of 2-(4-fluorophenyl)-4,5,6,7-tetrahydropyrazolo[1,5-a]pyrazine (1.2 g, 5.527 mmol) in DCM (50 ml) at 0° C., acetyl chloride (455.3 mg, 5.8 mmol) in DCM (5 ml) was added drop by drop and the mixture was stirred 3 h. SM was disappear on TLC. The reaction mixture was added the saturated aqueous solution of NaHCO₃ (10 ml) and extracted with DCM (20 ml). Then the organic layer was concentrated under pressure. The residue was purified via Prep-TLC (PE/EtOAc)=(2:1) and obtained the product.

Chemical Formula: calculated for (M+H⁺) C₁₄H₁₄FN₅O: 259.11, Found: 260.23.

### Step 2: 1-(3-bromo-2-(4-fluorophenyl)-6,7-dihydropyrazolo[1,5-a]pyrazin-5(4H)-yl)ethan-1-one

To a solution of 1-(2-(4-fluorophenyl)-6,7-dihydropyrazolo[1,5-a]pyrazin-5(4H)-yl)ethan-1-one (1.2 g, 4.631 mmol) in acetone (50 ml), NBS (824.27 mg, 4.631 mmol) was added at r.t. The reaction mixture was stirred at r.t. for 15 h. SM was disappear on TLC. The reaction mixture was washed with H₂O (100 ml) and extracted with DCM (200 ml). Then the organic layer was concentrated under pressure. The residue was purified via Prep-TLC (PE/EtOAc)=(2:1) and obtained the product.

Chemical Formula: calculated for (M+H⁺) C₁₄H₁₃BrFN₅O: 337.02, Found: 338.25.

### Step 3: 1-(2-(4-fluorophenyl)-3-(4,4,5,5-tetramethyl-1,3,2-dioxaborolan-2-yl)-6,7-dihydropyrazolo[1,5-a]pyrazin-5(4H)-yl)ethan-1-one

To a solution of 1-(3-bromo-2-(4-fluorophenyl)-6,7-dihydropyrazolo[1,5-a]pyrazin-5(4H)-yl)ethan-1-one (1.3 g, 3.857 mmol) in THF (50 ml), n-BuLi (4.05 mmol) was added at −78° C. The reaction mixture was stirred at −78° C. for 1 h and 2-isopropoxy-4,4,5,5-tetramethyl-1,3,2-dioxaborolane (1.076 g, 5.786 mmol) in THF (10 ml) was added drop by drop. Then the mixture was warmed to r.t. After 3 h, SM was disappear on TLC. The reaction mixture was quenched with the saturated aqueous solution of NH₄Cl (10 ml) to PH=7 and extracted with DCM (200 ml). Then the organic layer was concentrated under pressure. The residue was purified via Prep-TLC (PE/EtOAc)=(2:1) and obtained the product.

Chemical Formula: calculated for (M+H⁺) C₂₀H₂₅BFN₃O₃: 385.20, Found: 386.25.

### Step 4: 1-(2-(4-fluorophenyl)-3-(2-(methyl-d3)pyridin-4-yl)-6,7-dihydropyrazolo[1,5-a]pyrazin-5(4H)-yl)ethan-1-one

To a solution of 1-(2-(4-fluorophenyl)-3-(4,4,5,5-tetramethyl-1,3,2-dioxaborolan-2-yl)-6,7-dihydropyrazolo[1,5-a]pyrazin-5(4H)-yl)ethan-1-one (1.1 g, 2.856 mmol) in THF/H₂O (50 ml/10 ml), 4-bromo-2-(methyl-d3)pyridine (496.86 mg, 2.856 mmol), Pd(dppf)Cl₂ (20.9 mg, 0.02856 mmol), AcOK (616.63 mg, 6.283 mmol) was added at r.t. in N₂. The reaction mixture was refluxed for 8 h. The reaction mixture was extracted with DCM (200 ml). Then the organic layer was concentrated under pressure. The residue was purified via Prep-TLC (PE/EtOAc)=(1:1). The residue was purified via Genal-Prep-HPLC and obtained the product.

Chemical Formula: calculated for (M+H⁺) C₂₀H₁₆D₃FN₄O: 353.17, Found: 354.24.

### Example 1-132 Preparation of Compound I-132

FIG. 132 illustrates the synthetic scheme of compound I-132. As shown in FIG. 132, the specific synthesis step is as follows:

### Step 1: (4-fluorophenyl)(2-(4-fluorophenyl)-6,7-dihydropyrazolo[1,5-a]pyrazin-5(4H)-yl)methanone

To a solution of 2-(4-fluorophenyl)-4,5,6,7-tetrahydropyrazolo[1,5-a]pyrazine (1.2 g, 5.527 mmol) in DCM (50 ml) at 0° C., 4-fluorobenzoyl chloride (916.4 mg, 5.8 mmol) in DCM (5 ml) was added drop by drop and the mixture was stirred 3 h. SM was disappear on TLC. The reaction mixture was added the saturated aqueous solution of NaHCO₃ (10 ml) and extracted with DCM (20 ml). Then the organic layer was concentrated under pressure. The residue was purified via Prep-TLC (PE/EtOAc)=(2:1) and obtained the product.

Chemical Formula: calculated for (M+H⁺) C₁₉H₁₅F₂N₃O: 339.12, Found: 340.24.

### Step 2: (3-bromo-2-(4-fluorophenyl)-6,7-dihydropyrazolo[1,5-a]pyrazin-5(4H)-yl)(4-fluorophenyl)methanone

To a solution of (4-fluorophenyl)(2-(4-fluorophenyl)-6,7-dihydropyrazolo[1,5-a]pyrazin-5(4H)-yl)methanone (1.2 g, 3.539 mmol) in acetone (50 ml), NBS (629.8 mg, 3.539 mmol) was added at r.t. The reaction mixture was stirred at r.t. for 15 h. SM was disappear on TLC. The reaction mixture was washed with H₂O (100 ml) and extracted with DCM (200 ml). Then the organic layer was concentrated under pressure. The residue was purified via Prep-TLC (PE/EtOAc)=(2:1) and obtained the product.

Chemical Formula: calculated for (M+H⁺) C₁₉H₁₄BrF₂N₃O: 417.03, Found: 418.24.

### Step 3: (4-fluorophenyl)(2-(4-fluorophenyl)-3-(4,4,5,5-tetramethyl-1,3,2-dioxaborolan-2-yl)-6,7-dihydropyrazolo[1,5-a]pyrazin-5(4H)-yl)methanone

To a solution of (3-bromo-2-(4-fluorophenyl)-6,7-dihydropyrazolo[1,5-a]pyrazin-5(4H)-yl)(4-fluorophenyl) methanone (1.3 g, 3.117 mmol) in THF (50 ml), n-BuLi (3.273 mmol) was added at −78° C. The reaction mixture was stirred at −78° C. for 1 h and 2-isopropoxy-4,4,5,5-tetramethyl-1,3,2-dioxaborolane (869.64 mg, 4.676 mmol) in THF (10 ml) was added drop by drop. Then the mixture was warmed to r.t. After 3 h, SM was disappear on TLC. The reaction mixture was quenched with the saturated aqueous solution of NH₄Cl (10 ml) to PH=7 and extracted with DCM (200 ml). Then the organic layer was concentrated under pressure. The residue was purified via Prep-TLC (PE/EtOAc)=(2:1) and obtained the product.

Chemical Formula: calculated for (M+H⁺) C₂₅H₂₆BF₂N₃O₃: 465.20, Found: 466.32.

### Step 4: (4-fluorophenyl)(2-(4-fluorophenyl)-3-(2-(methyl-d3)pyridin-4-yl)-6,7-dihydropyrazolo[1,5-a]pyrazin-5(4H)-yl)methanone

To a solution of (4-fluorphenyl)(2-(4-fluorophenyl)-3-(4,4,5,5-tetramethyl-1,3,2-dioxaborolan-2-yl)-6,7-dihydropyrazolo[1,5-a]pyrazin-5(4H)-yl)methanone (1.1 g, 2.365 mmol) in THF/H₂O (50 ml/10 ml), 4-bromo-2-(methyl-d3)-pyridine (411.44 mg, 2.365 mmol), Pd(dppf)Cl₂ (17.3 mg, 0.02365 mmol), AcOK (510.62 mg, 5.203 mmol) was added at r.t. in N₂. The reaction mixture was refluxed for 8 h. The reaction mixture was extracted with DCM (200 ml). Then the organic layer was concentrated under pressure. The residue was purified via Prep-TLC (PF/EtOAc)=(1:1). The residue was purified via Genal-Prep-HPLC and obtained the product.

Chemical Formula: calculated for (M+H⁺) C₂₅H₁₇D₃F₂N₄O: 433.18, Found: 434.26.

### Example 1-133 Preparation of Compound I-133

FIG. 133 illustrates the synthetic scheme of compound I-133. As shown in FIG. 133, the specific synthesis step is as follows:

### Step 1: (2-(4-fluorophenyl)-6,7-dihydropyrazolo[1,5-a]pyrazin-5(4H)-yl)(pyridin-3-yl)methanone

To a solution of 2-(4-fluorophenyl)-4,5,6,7-tetrahydropyrazolo[1,5-a]pyrazine (1.2 g, 5.527 mmol) in DCM (50 ml) at 0° C., nicotinoyl chloride (817.8 mg, 5.8 mmol) in DCM (5 ml) was added drop by drop and the mixture was stirred 3 h. SM was disappear on TLC. The reaction mixture was added the saturated aqueous solution of NaHCO₃ (10 ml) and extracted with DCM (20 ml). Then the organic layer was concentrated under pressure. The residue was purified via Prep-TLC (PE/EtOAc)=(2:1) and obtained the product.

Chemical Formula: calculated for (M+H⁺) C₁₈H₁₅FN₄O: 322.12, Found: 323.24.

### Step 2: (3-bromo-2-(4-fluorophenyl)-6,7-dihydropyrazolo[1,5-a]pyrazin-5(4H)-yl)(pyridin-3-yl)methanone

To a solution of (2-(4-fluorophenyl)-6,7-dihydropyrazolo[1,5-a]pyrazin-5(4H)-yl)(pyridin-3-yl)methanone (1.2 g, 3.725 mmol) in acetone (50 ml), NBS (663.03 mg, 3.725 mmol) was added at r.t. The reaction mixture was stirred at r.t. for 15 h. SM was disappear on TLC. The reaction mixture was washed with H₂O (100 ml) and extracted with DCM (200 ml). Then the organic layer was concentrated under pressure. The residue was purified via Prep-TLC (PE/EtOAc)=(2:1) and obtained the product.

Chemical Formula: calculated for (M+H⁺) C₁₈H₁₄BrFN₄O: 400.03, Found: 401.26.

### Step 3: (2-(4-fluorophenyl)-3-(4,4,5,5-tetramethyl-1,3,2-dioxaborolan-2-yl)-6,7-dihydropyrazolo[1,5-a]pyrazin-5(4H)-yl) (pyridin-3-yl)methanone

To a solution of (3-bromo-2-(4-fluorophenyl)-6,7-dihydropyrazolo[1,5-a]pyrazin-5(4H)-yl)(pyridin-3-yl)methanone (1.3 g, 3.25 mmol) in THF (50 ml), n-BuLi (3.412 mmol) was added at −78° C. The reaction mixture was stirred at −78° C. for 1 h and 2-isopropoxy-4,4,5,5-tetramethyl-1,3,2-dioxaborolane (906.75 mg, 4.875 mmol) in THF (10 ml) was added drop by drop. Then the mixture was warmed to r.t. After 3 h, SM was disappear on TLC. The reaction mixture was quenched with the saturated aqueous solution of NH₄Cl (10 ml) to PH=7 and extracted with DCM (200 ml). Then the organic layer was concentrated under pressure. The residue was purified via Prep-TLC (PE/EtOAc)=(2:1) and obtained the product.

Chemical Formula: calculated for (M+H⁺) C₂₄H₂₆BFN₄O₃: 448.21, Found: 449.35.

### Step 4: (2-(4-fluorophenyl)-3-(2-(methyl-d3)pyridin-4-yl)-6,7-dihydropyrazolo[1,5-a]pyrazin-5(4H)-yl)(pyridin-3-yl)methanone

To a solution of (2-(4-fluorophenyl)-3-(4,4,5,5-tetramethyl-1,3,2-dioxaborolan-2-yl)-6,7-dihydropyrazolo[1,5-a]pyrazin-5(4H)-yl) (pyridin-3-yl)methanone (1.1 g, 2.454 mmol) in THF/H₂O (50 ml/10 ml), 4-bromo-2-(methyl-d3)-pyridine (427 mg, 2.454 mmol), Pd(dppf)Cl₂ (17.9 mg, 0.02454 mmol), AcOK (529.84 mg, 5.399 mmol) was added at r.t. in N₂. The reaction mixture was refluxed for 8 h. The reaction mixture was extracted with DCM (200 ml). Then the organic layer was concentrated under pressure. The residue was purified via Prep-TLC (PE/EtOAc)=(1:1). The residue was purified via Genal-Prep-HPLC and obtained the product.

Chemical Formula: calculated for (M+H⁺) C₂₄H₁₇D₃FN₅O: 416.18, Found: 417.25.

### Example 1-134 Preparation of Compound I-134

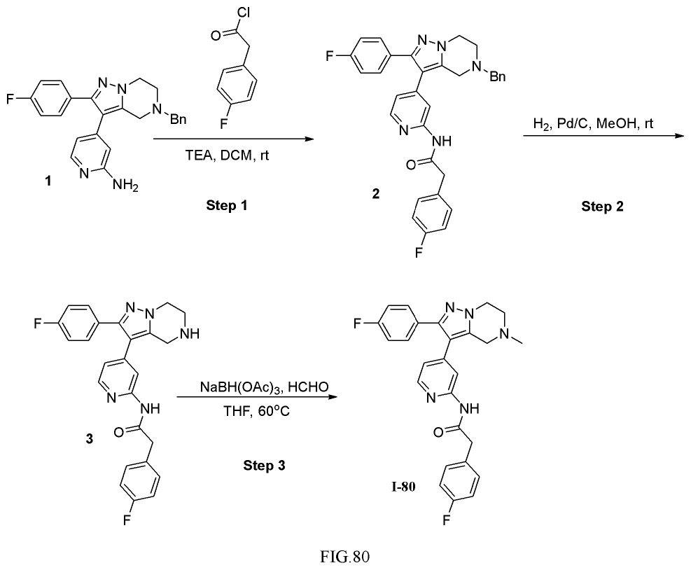

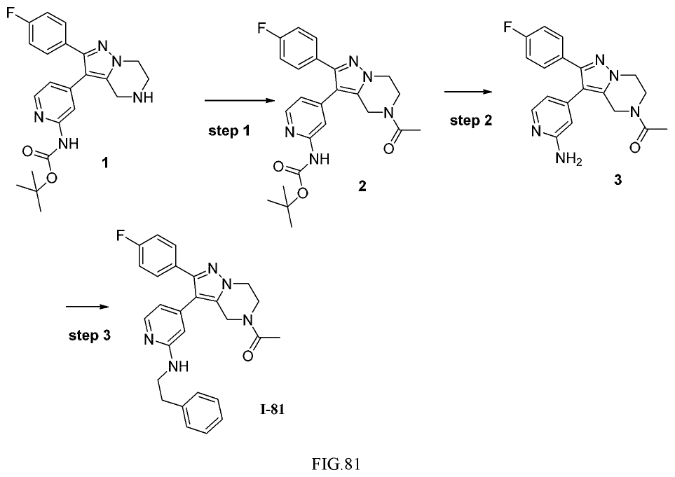

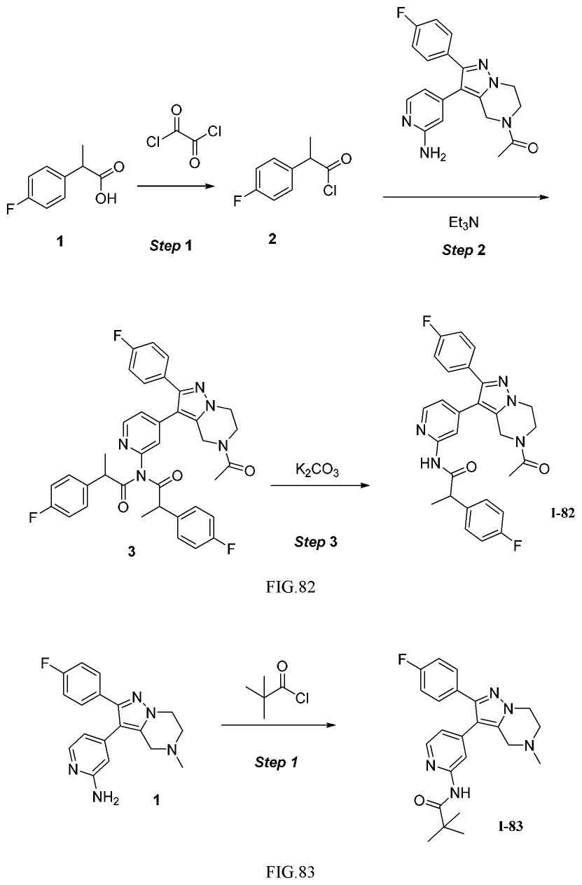

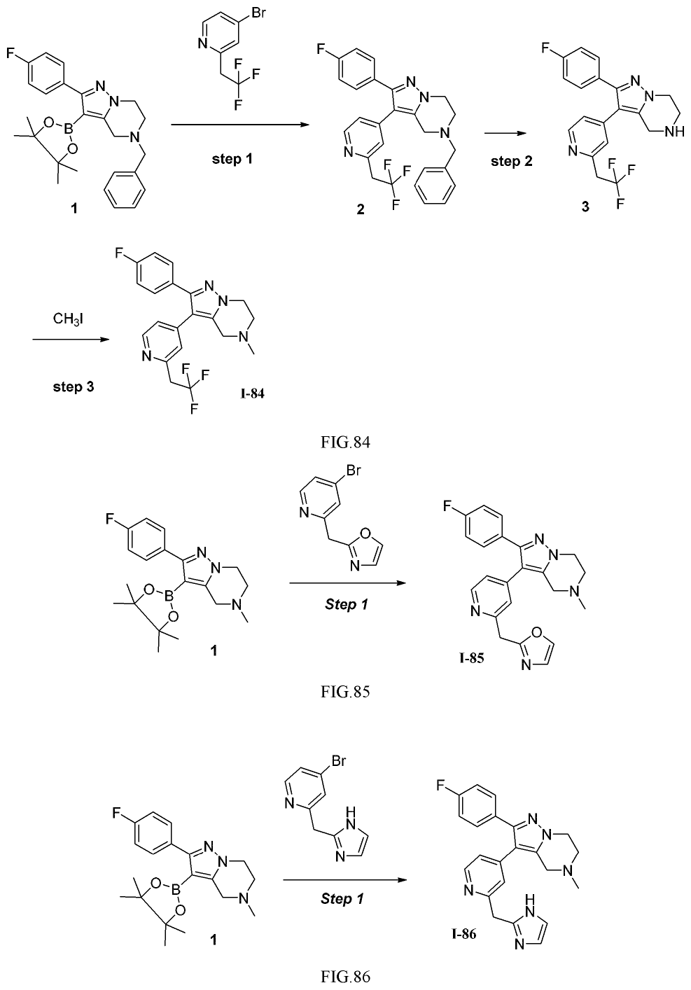

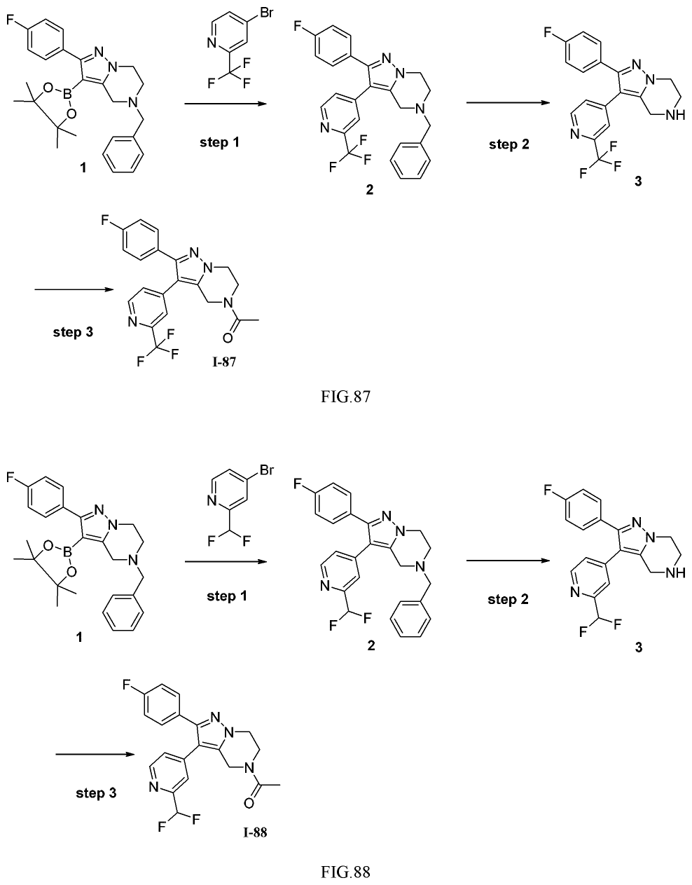

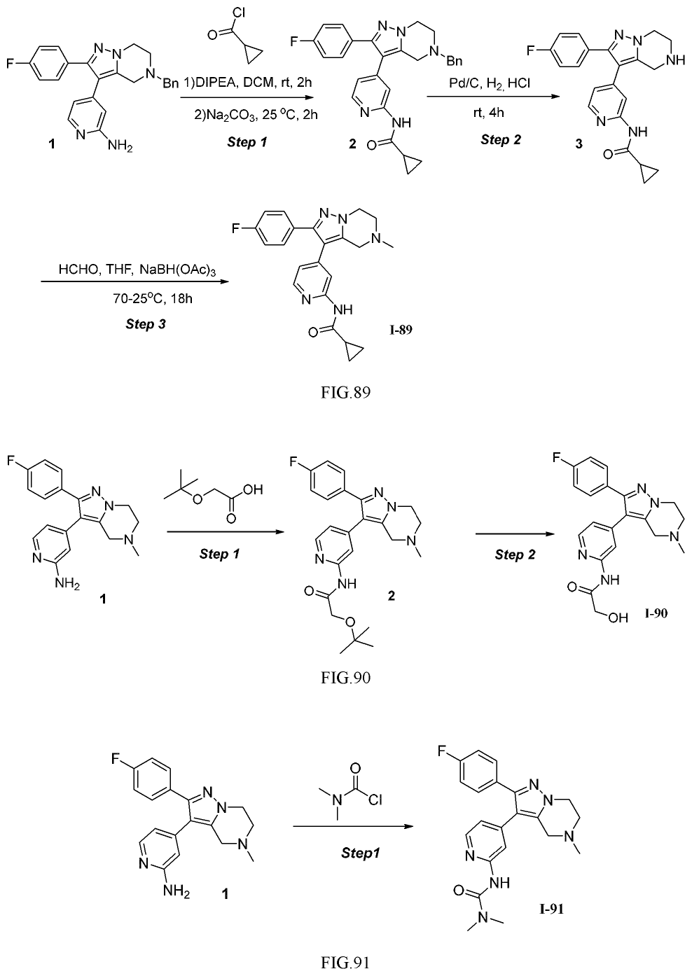

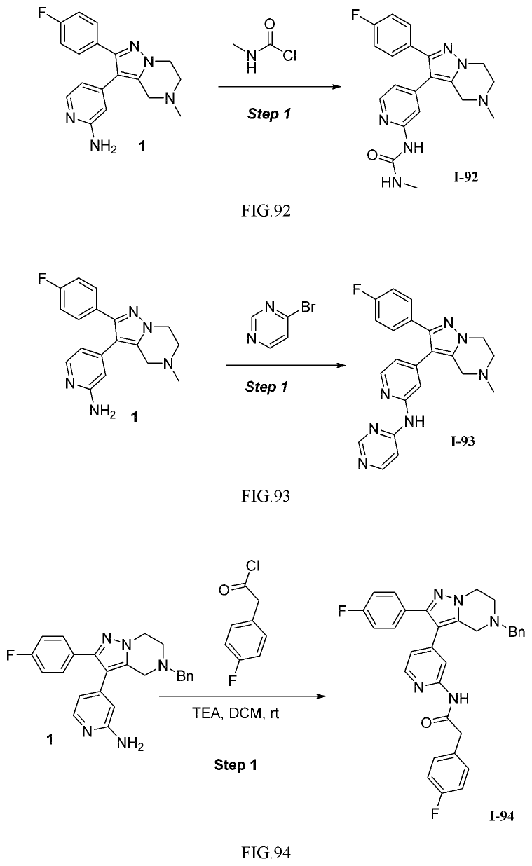

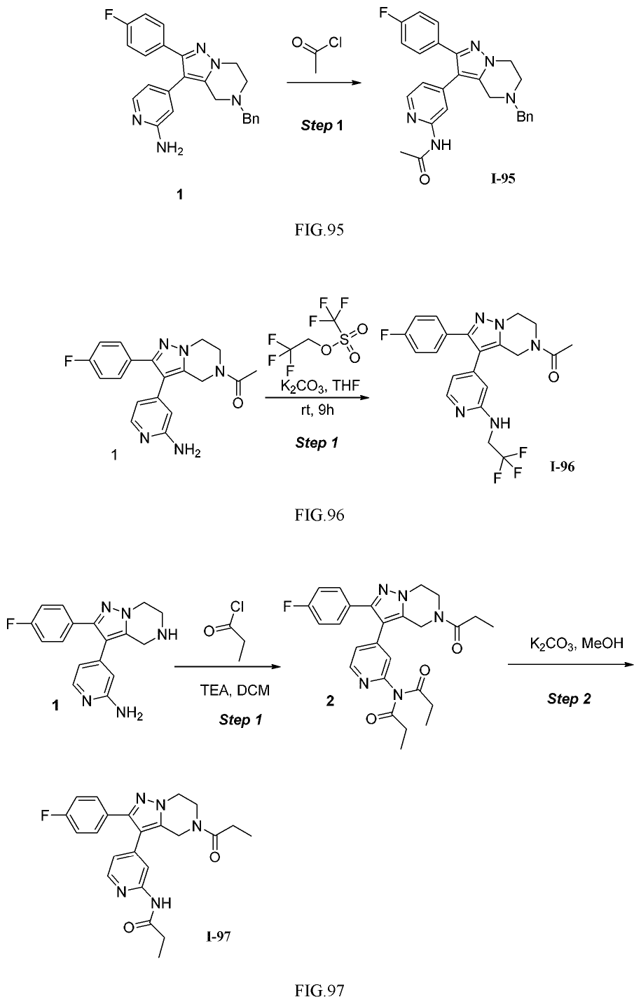

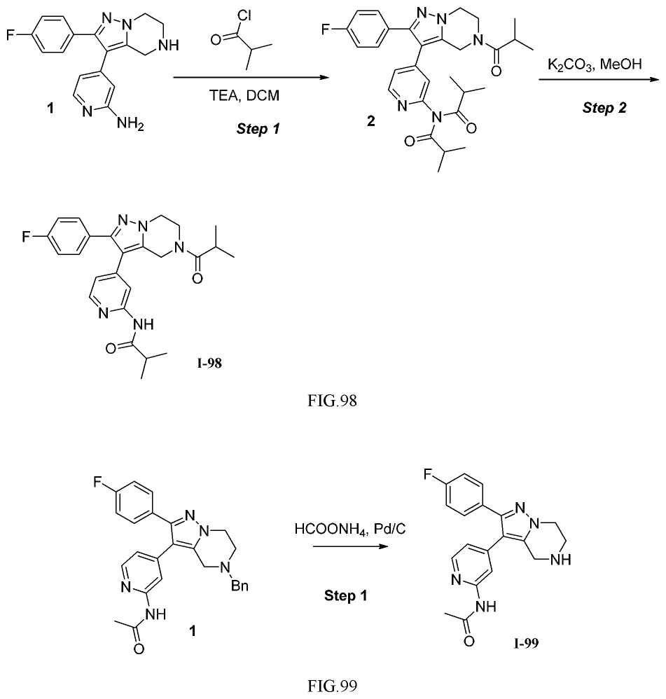

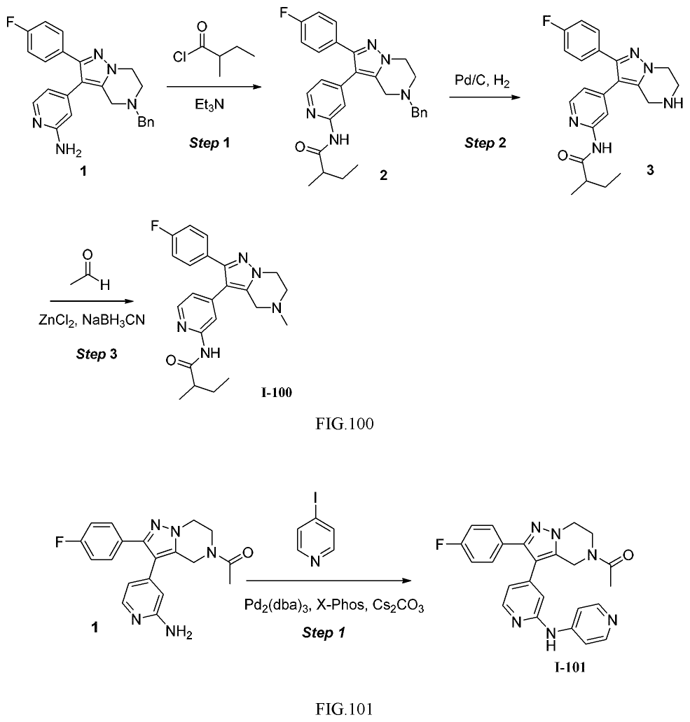

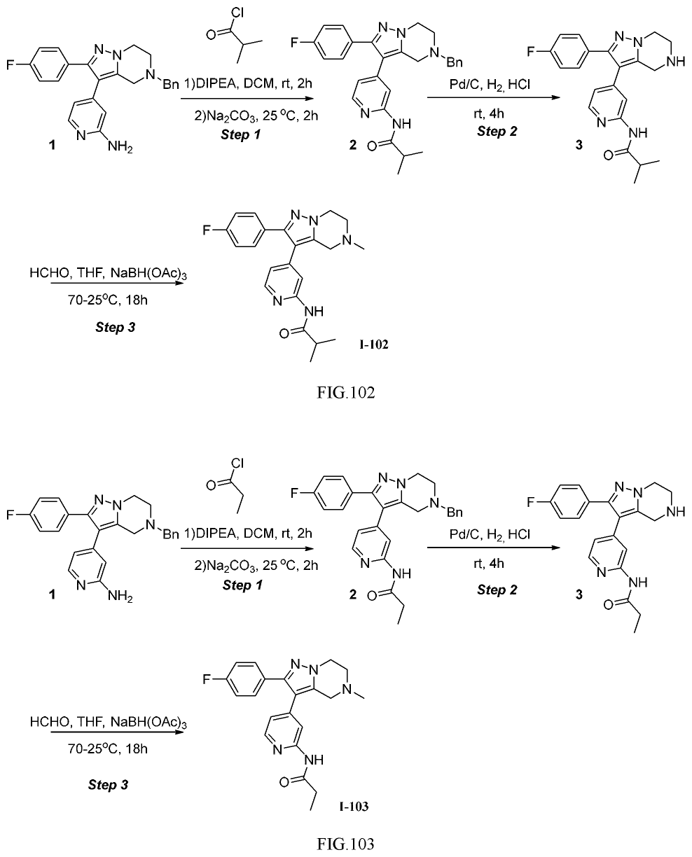

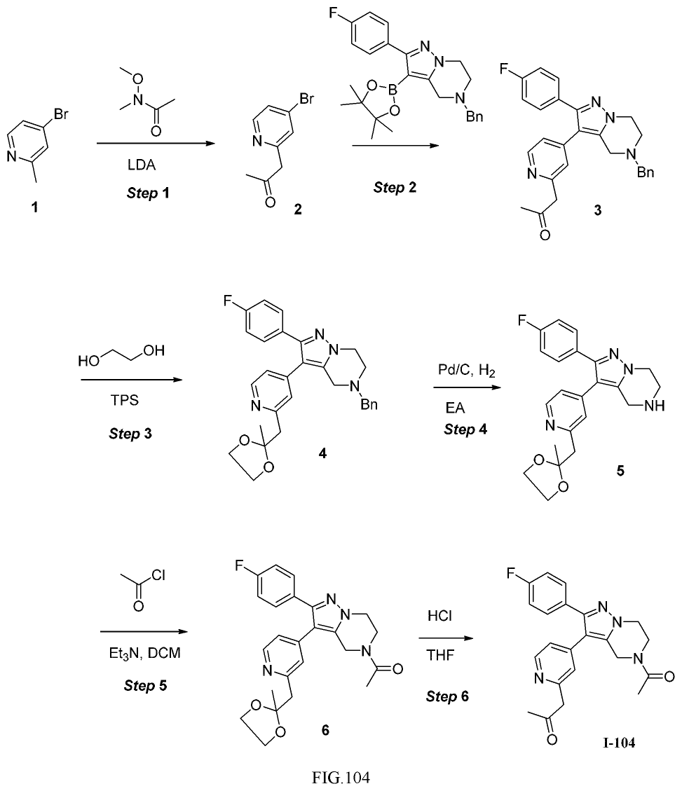

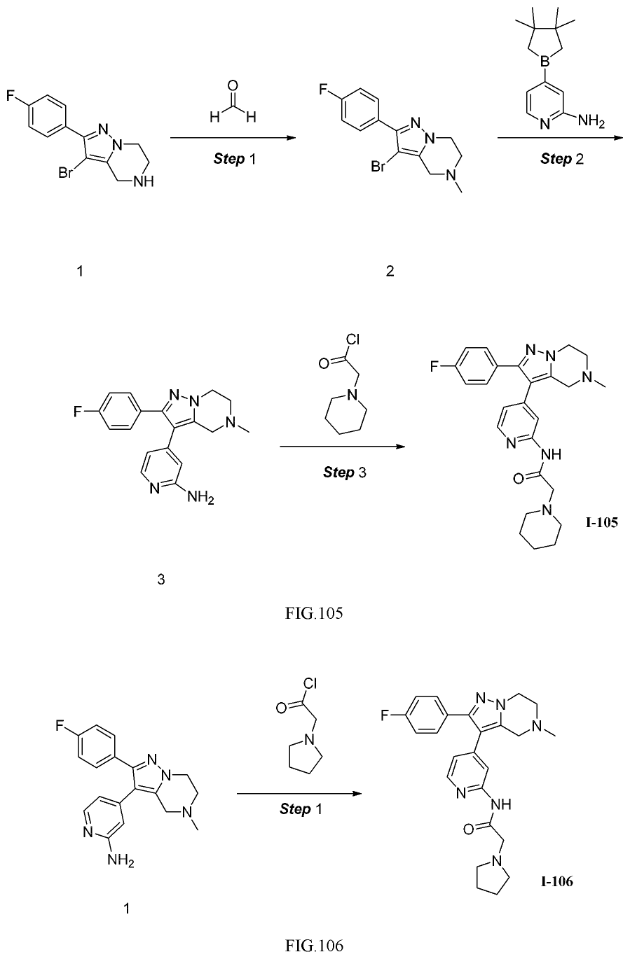

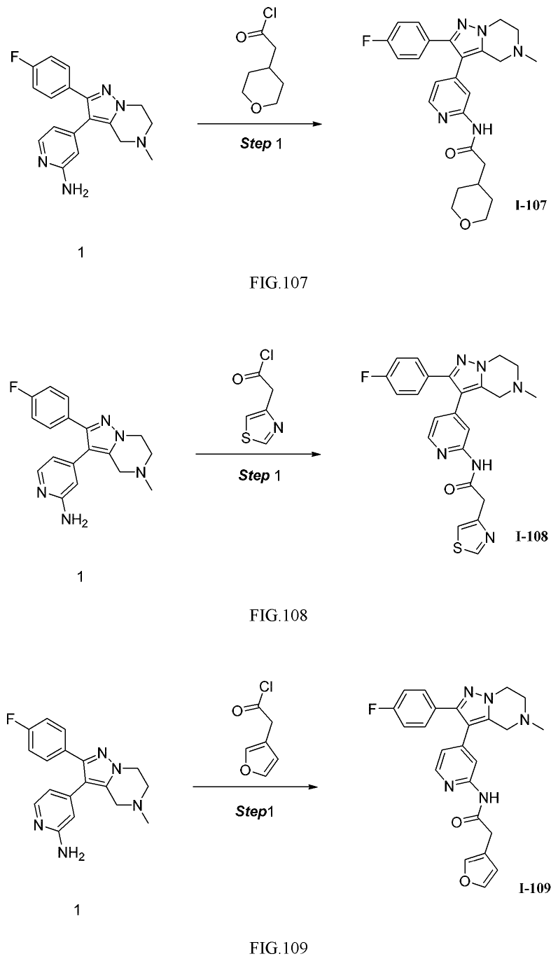

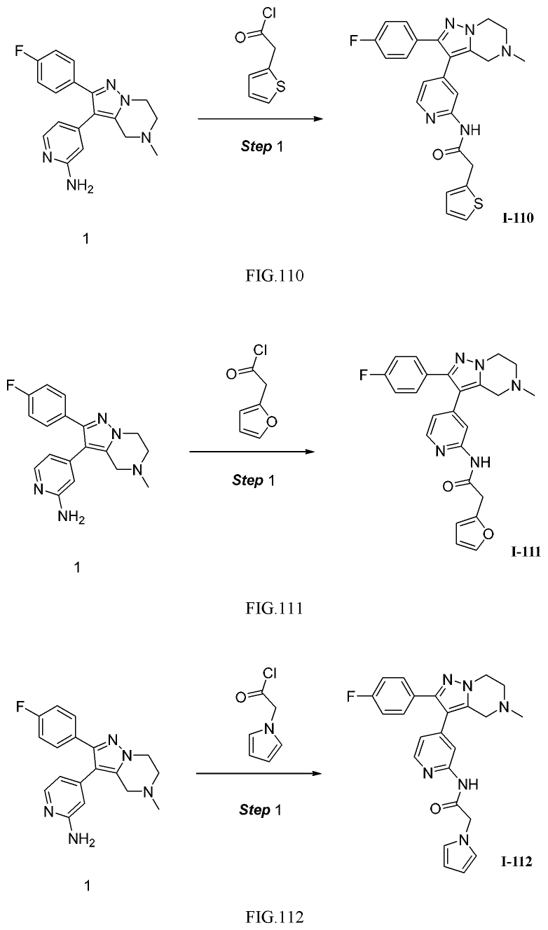

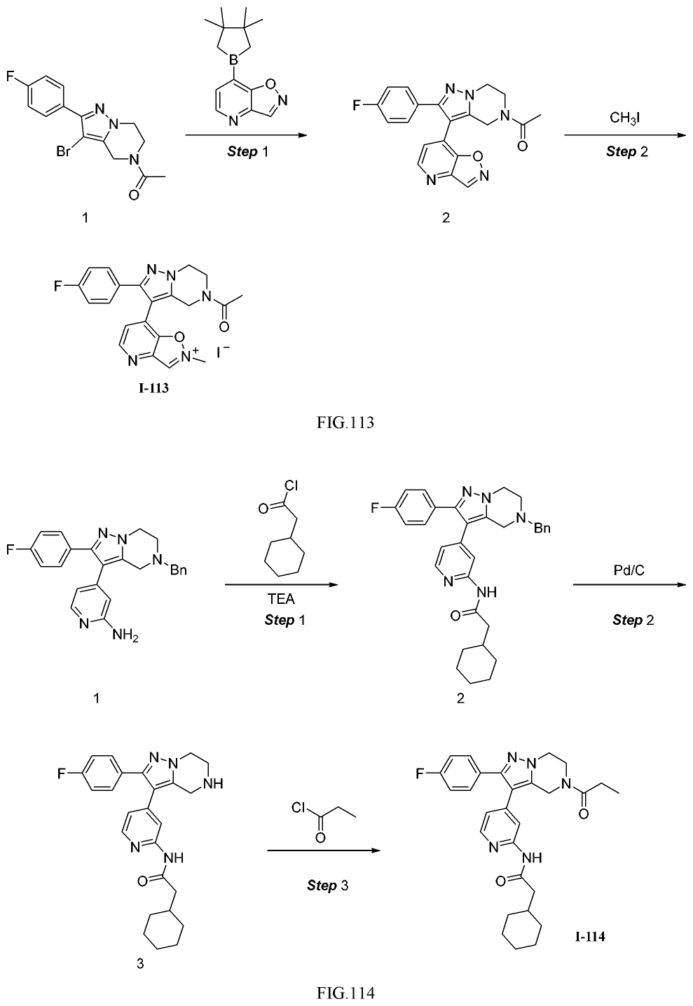

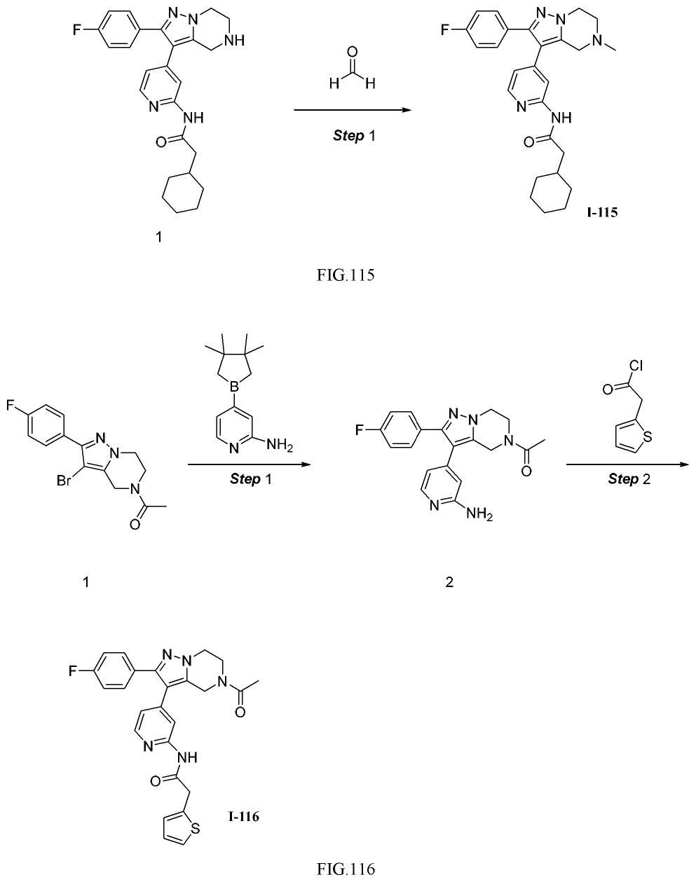

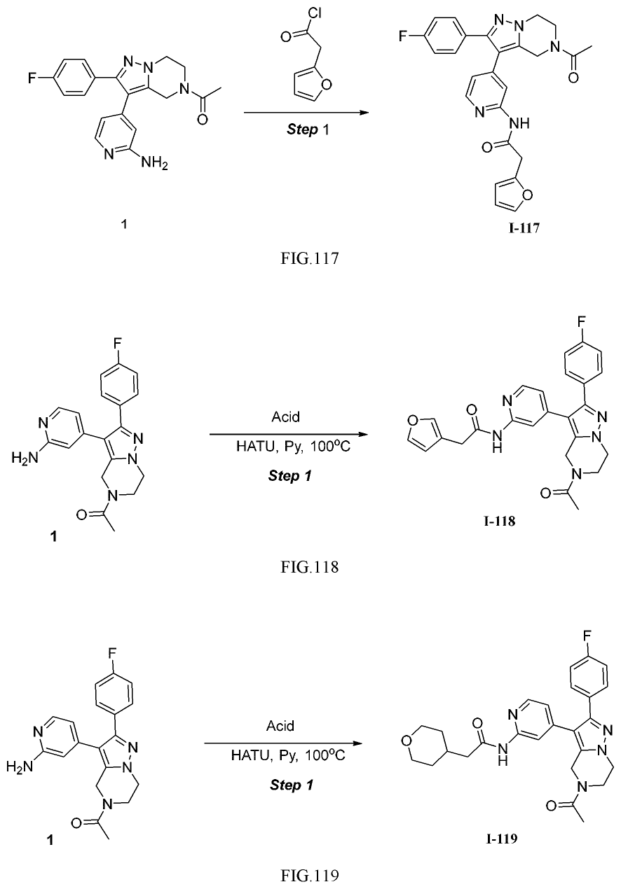

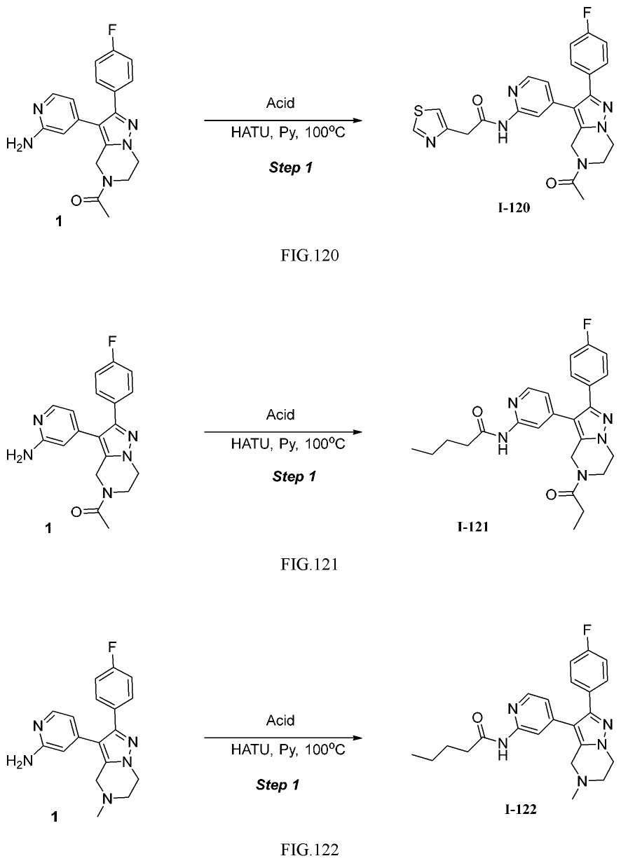

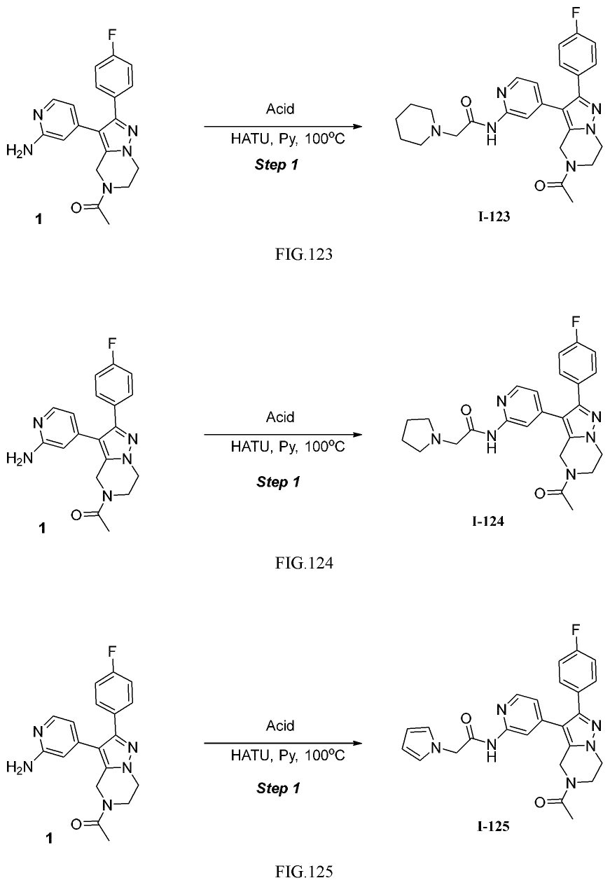

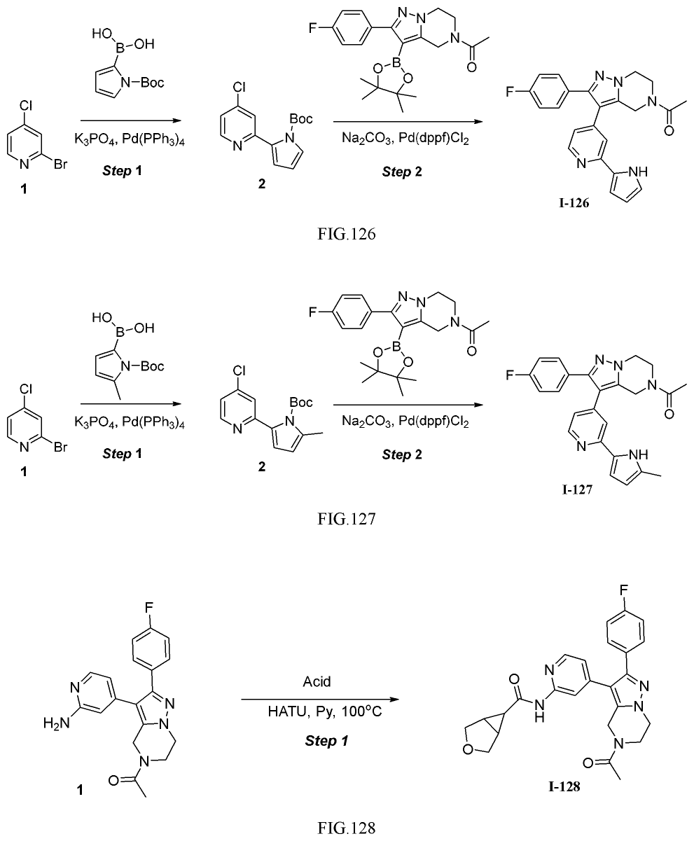

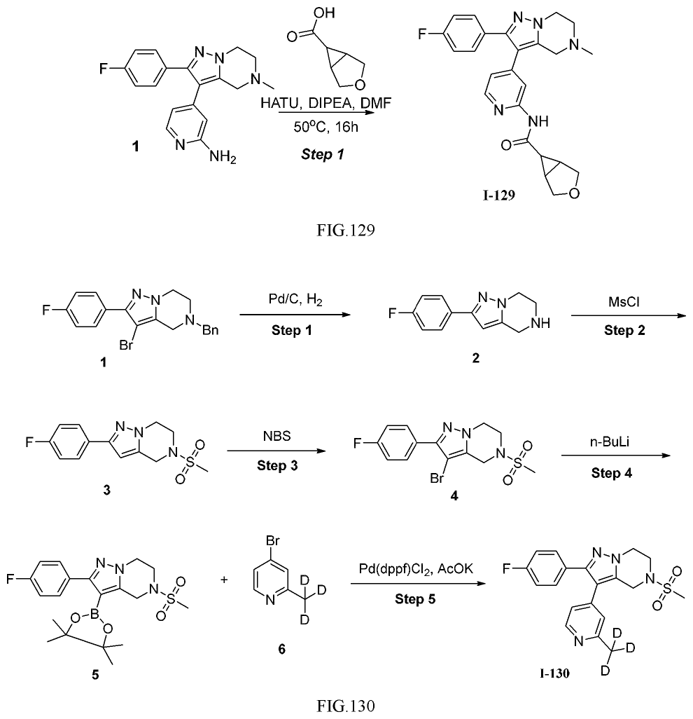

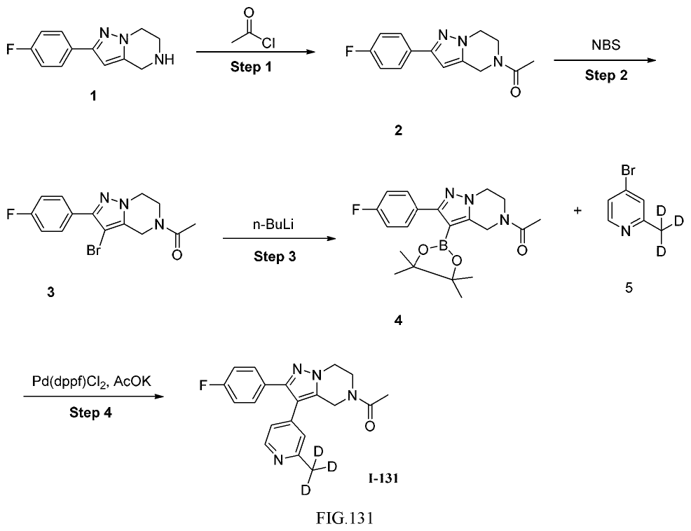

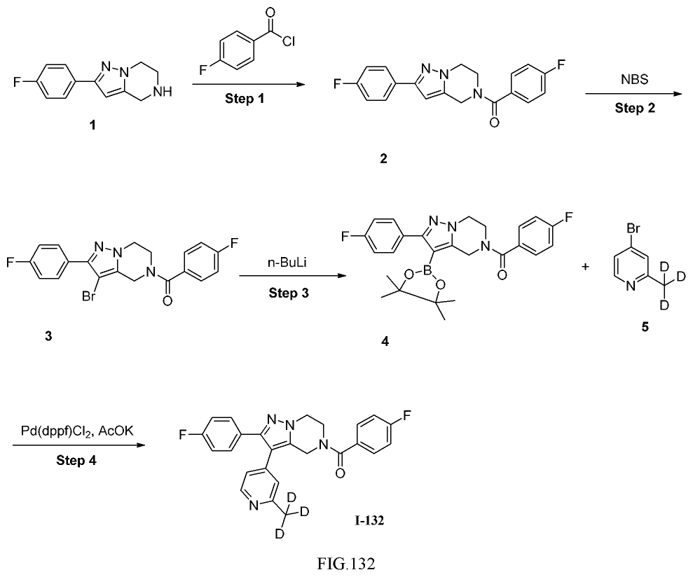

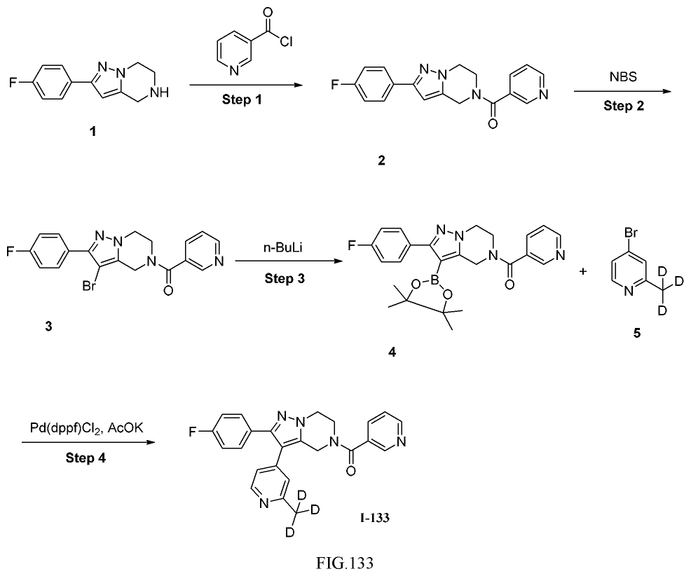

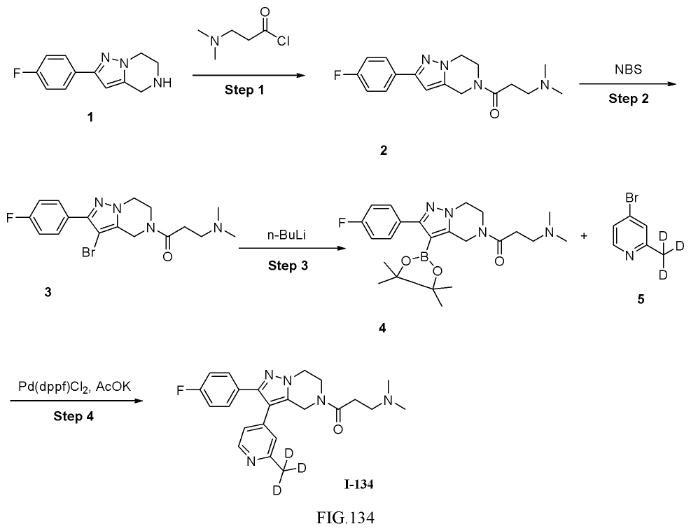

FIG. 134 illustrates the synthetic scheme of compound I-134. As shown in FIG. 134, the specific synthesis step is as follows:

### Step 1: 3-(dimethylamino)-1-(2-(4-fluorophenyl)-6,7-dihydropyrazolo[1,5-a]pyrazin-5(4H)-yl)propan-1-one

To a solution of 2-(4-fluorophenyl)-4,5,6,7-tetrahydropyrazolo[1,5-a]pyrazine (1.2 g, 5.527 mmol) in DCM (50 ml) at 0° C., 3-(dimethylamino)propanoyl chloride (783.29 mg, 5.8 mmol) in DCM (5 ml) was added drop by drop and the mixture was stirred 3 h. SM was disappear on TLC. The reaction mixture was added the saturated aqueous solution of NaHCO₃ (10 ml) and extracted with DCM (20 ml). Then the organic layer was concentrated under pressure. The residue was purified via Prep-TLC (PE/EtOAc)=(2:1) and obtained the product.

Chemical Formula: calculated for (M+H⁺) C₁₇H₂₁FN₄O: 316.17, Found: 317.24.

### Step 2: 1-(3-bromo-2-(4-fluorophenyl)-6,7-dihydropyrazolo[1,5-a]pyrazin-5(4H)-yl)-3-(dimethylamino)propan-1-one

To a solution of 3-(dimethylamino)-1-(2-(4-fluorophenyl)-6,7-dihydropyrazolo[1,5-a]pyrazin-5(4H)-yl)propan-1-one (1.2 g, 3.795 mmol) in acetone (50 ml), NBS (675.43 mg, 3.795 mmol) was added at r.t. The reaction mixture was stirred at r.t. for 15 h. SM was disappear on TLC. The reaction mixture was washed with H₂O (100 ml) and extracted with DCM (200 ml). Then the organic layer was concentrated under pressure. The residue was purified via Prep-TLC (PE/EtOAc)=(2:1) and obtained the product.

Chemical Formula: calculated for (M+H⁺) C₁₇H₂₀BrFN₄O: 394.08, Found: 395.21.

### Step 3: 3-(dimethylamino)-1-(2-(4-fluorophenyl)-3-(4,4,5,5-tetramethyl-1,3,2-dioxaborolan-2-yl)-6,7-dihydropyrazolo[1,5-a]pyrazin-5(4H)-yl)propan-1-one

To a solution of 1-(3-bromo-2-(4-fluorophenyl)-6,7-dihydropyrazolo[1,5-a]pyrazin-5(4H)-yl)-3-(dimethylamino) propan-1-one (1.3 g, 3.299 mmol) in THF (50 ml), n-BuLi (3.464 mmol) was added at −78° C. The reaction mixture was stirred at −78° C. for 1 h and 2-isopropoxy-4,4,5,5-tetramethyl-1,3,2-dioxaborolane (920.37 mg, 4.948 mmol) in THF (10 ml) was added drop by drop. Then the mixture was warmed to r.t. After 3 h, SM was disappear on TLC. The reaction mixture was quenched with the saturated aqueous solution of NH₄Cl (10 ml) to PH=7 and extracted with DCM (200 ml). Then the organic layer was concentrated under pressure. The residue was purified via Prep-TLC (PE/EtOAc)=(2:1) and obtained the product.

Chemical Formula: calculated for (M+H⁺) C₂₃H₃₂BFN₄O₃: 442.26, Found: 443.35.

### Step 4: 3-(dimethylamino)-1-(2-(4-fluorophenyl)-3-(2-(methyl-d3)pyridin-4-yl)-6,7-dihydropyrazolo[1,5-a]pyrazin-5(4H)-yl) propan-1-one

To a solution of 3-(dimethylamino)-1-(2-(4-fluorophenyl)-3-(4,4,5,5-tetramethyl-1,3,2-dioxaborolan-2-yl)-6,7-dihydropyrazolo[1,5-a]pyrazin-5(4H)-yl)propan-1-one (1.1 g, 2.487 mmol) in THF/H₂O (50 ml/10 ml), 4-bromo-2-(methyl-d3)-pyridine (432.78 mg, 2.487 mmol), Pd(dppf)Cl₂ (18.2 mg, 0.02487 mmol), AcOK (536.96 mg, 5.471 mmol) was added at r.t. in N₂. The reaction mixture was refluxed for 8 h. The reaction mixture was extracted with DCM (200 ml). Then the organic layer was concentrated under pressure. The residue was purified via Prep-TLC (PE/EtOAc)=(1:1). The residue was purified via Genal-Prep-HPLC and obtained the product.

Chemical Formula: calculated for (M+H⁺) C₂₃H₂₃D₃FN₅O: 410.23, Found: 411.35.

### Example 2 Assay of Biological Activities

The CK1δ kinase assay was performed with a buffer (40 μL, pH 7.5) containing 50 mM Trig, 10 mM MgCl₂, 1 mM dithiothreitol, 100 μg/mL BSA with 10 μM ATP, 2 nM wild type CK1δ, and 42 μM peptide substrate PLSRTLpSVASLPGL (Flotow et al., 1990) in the presence of 1 μL of a CK1δ inhibitor (e.g., a compound of the present application) or 4% DMSO (e.g., as control). The reaction mixture was incubated for 85 min at 25° C.; detection was carried out as described for the Kinase-Glo Assay (Promega). Luminescent output was measured on the Perkin Elmer Envision plate reader (PerkinElmer, Waltham, MA).

Bmall-dLuc or Per2-dLuc U2OS cells were suspended in the culture medium (DMEM supplemented with 10% fetal bovine serum, 0.29 mg/mL L-glutamine, 100 units/mL penicillin, and 100 mg/mL streptomycin) and plated onto 96-well white solid-bottom plates at 200 μL (10,000 cells) per well. After 2 days, 100 μL of the explant medium (DMEM supplemented with 2% B27, 10 mM HEPES, 0.38 mg/mL sodium bicarbonate, 0.29 mg/mL L-glutamine, 100 units/mL penicillin, 100 mg/mL streptomycin, 0.1 mg/mL gentamicin, and 1 mM luciferin, pH 7.2) was dispensed to each well, followed by the application of 1 μL of a compound of the present application (dissolved in DMSO; final concentration was 0.7% in DMSO). The plate was covered with an optically clear film and set to microplate reader (Infinite M200, Tecan). The luminescence was recorded every 1 h for 3-4 days. The period parameter was obtained from the luminescence rhythm by curve fitting program CellulaRhythm or MultiCycle (Actimetrics), both of which generated similar results.

The CK1δ inhibition results (IC50) are summarized in Table 2.

TABLE 2

| | CK1δ |
|---|---|
| Compound | (IC50, nM) |
| I-1 | 1789 |
| I-2 | 1259 |
| I-3 | 2750 |
| I-4 | 2642 |
| I-5 | 5080 |
| I-6 | 1026 |
| I-7 | 369.4 |
| I-8 | 2550 |
| I-9 | 1654 |
| I-10 | 650.5 |
| I-11 | 2496 |
| I-13 | 102.8 |
| I-14 | 5981 |
| I-15 | 8515 |
| I-16 | 1699 |
| I-17 | 12049 |
| I-18 | 410.1 |
| I-19 | 502.2 |
| I-20 | 677.9 |
| I-21 | 363.7 |
| I-22 | 1774 |
| I-23 | 1049 |
| I-24 | 346 |
| I-58 | 247.3 |
| I-59 | 4506 |
| I-60 | 14462 |
| I-61 | 183.7 |
| I-62 | 21.35 |
| I-63 | 28909 |
| I-64 | 334.1 |
| I-65 | 13271 |
| I-66 | 41.75 |
| I-67 | 5059 |
| I-68 | 32.68 |
| I-69 | 37.43 |
| I-70 | 120.5 |
| I-134 | 1789 |

The CK1δ inhibition results (EC50) are summarized in Table 3.

TABLE 3

| | CK1δ |
|---|---|
| Compound | (EC50, μM) |
| I-1 | 18.9 |
| I-3 | 55 |
| I-4 | 57 |
| I-5 | 58 |
| I-6 | 7.4 |
| I-7 | 2.2 |
| I-8 | 10 |
| I-9 | 5.8 |
| I-10 | 3.2 |
| I-11 | 6.5 |
| I-12 | 20 |
| I-13 | 26 |
| I-14 | 24 |
| I-15 | 24 |
| I-16 | 5.2 |
| I-17 | 5.1 |
| I-19 | 6 |
| I-20 | 2.9 |
| I-21 | 0.4 |
| I-22 | 9.9 |
| I-23 | 12.7 |
| I-24 | 0.4 |
| I-25 | 0.67 |
| I-26 | 0.4 |
| I-27 | 0.5 |
| I-28 | 0.6 |
| I-29 | 0.6 |
| I-30 | 0.5 |
| I-31 | 0.3 |
| I-32 | 0.8 |
| I-33 | 0.5 |
| I-40 | 0.7 |
| I-41 | 0.6 |
| I-42 | 0.4 |
| I-44 | 0.8 |
| I-46 | 1.2 |
| I-49 | 1.3 |
| I-50 | 0.2 |
| I-56 | 0.6 |
| I-57 | 0.5 |
| I-58 | 0.6 |
| I-62 | 18 |
| I-64 | 0.7 |
| I-66 | 0.02 |
| I-68 | 1.9 |
| I-69 | 0.3 |
| I-70 | 1.4 |
| I-71 | 1 |
| I-72 | 0.01 |
| I-73 | 0.11 |
| I-74 | 1.6 |
| I-75 | 0.2 |
| I-76 | 1.0 |
| I-77 | 2.0 |
| I-80 | 0.1 |
| I-82 | 0.1 |
| I-89 | 0.7 |
| I-94 | 0.2 |
| I-95 | 1.7 |
| I-96 | 4.5 |
| I-97 | 0.9 |
| I-98 | 2.1 |
| I-99 | 0.7 |
| I-100 | 0.6 |
| I-101 | 2.2 |
| I-102 | 0.7 |
| I-103 | 0.5 |

### Example 3 Assay of Drug Transport

### 1. Preparation of Caco-2 Cells

1) 50 μL and 25 mL of cell culture medium were added to each well of the Transwell insert and reservoir, respectively. And then the HTS transwell plates were incubated at 37° C., 5% CO₂ for 1 hour before cell seeding.
2) Caco-2 cells were diluted to 6.86×10⁵ cells/mL with culture medium and 50 μL of cell suspension were dispensed into the filter well of the 96-well HTS Transwell plate. Cells were cultivated for 14-18 days in a cell culture incubator at 37° C., 5% CO₂, 95% relative humidity. Cell culture medium was replaced every other day, beginning no later than 24 hours after initial plating.

### 2. Preparation of Stock Solutions

10 mM stock solutions of test compounds were prepared in DMSO. The stock solutions of positive controls were prepared in DMSO at the concentration of 10 mM. Digoxin and propranolol were used as control compounds in this assay.

### 3. Assessment of Cell Monolayer Integrity

1) Medium was removed from the reservoir and each Transwell insert and replaced with prewarmed fresh culture medium.
2) Transepithelial electrical resistance (TEER) across the monolayer was measured using Millicell Epithelial Volt-Ohm measuring system (Millipore, USA).
3) The Plate was returned to the incubator once the measurement was done.

The TEER value was calculated according to the following equation:

TEER measurement (ohms)*Area of membrane (cm²)=TEER value (ohm·cm²)

TEER value should be greater than 230 ohm·cm², which indicates the well-qualified Caco-2 monolayer.

### 4. Assay Procedures

1) The Caco-2 plate was removed from the incubator and washed twice with pre-warmed HBSS (10 mM HEPES, pH 7.4), and then incubated at 37° C. for 30 minutes.
2) The stock solutions of control compounds and test compounds were diluted in DMSO to get 1 mM solutions and then diluted with HBSS (10 mM HEPES, pH 7.4) get 5 μM working solutions. The final concentration of DMSO in the incubation system was 0.5%.
3) To determine the rate of drug transport in the apical to basolateral direction. 125 μL of 5 μM working solution of control compound and test compounds were added to the Transwell insert (apical compartment), and transfer 50 μL sample (DO sample) immediately from the apical compartment to a new 96-well plate. Fill the wells in the receiver plate (basolateral compartment) with 235 μL of HBSS (10 mM HEPES, pH 7.4).
4) To determine the rate of drug transport in the basolateral to apical direction. 285 μL of 5 μM working solution of control compound and test compounds were to the receiver plate wells (basolateral compartment), and transfer 50 μL sample (DO sample) immediately from the basolateral compartment to a new 96-well plate. Fill the wells in the Transwell insert (apical compartment) with 75 μL of HBSS (10 mM HEPES, pH 7.4). The assay was performed in duplicate.
5) The plates were incubated at 37° C. for 2 hours.
6) At the end of the incubation, 50 μL samples from donor sides (apical compartment for Ap→Bl flux, and basolateral compartment for Bl→Ap) and receiver sides (basolateral compartment for Ap→Bl flux, and apical compartment for Bl→Ap) were transferred to wells of a new 96-well plate, followed by the addition of 4 volume of cold methanol containing appropriate internal standards (IS). Samples were Vortexed for 5 minutes and then centrifuged at 3,220 g for 40 minutes. An aliquot of 100 μL of the supernatant was mixed with an appropriate volume of ultra-pure water before LC-MS/MS analysis.
7) To determine the Lucifer Yellow leakage after 2 hour transport period, stock solution of Lucifer yellow was prepared in water and diluted with HBSS (10 mM HEPES, pH 7.4) to reach the final concentration of 100 μM. 100 μL of the Lucifer yellow solution was added to each Transwell insert (apical compartment), followed by filling the wells in the receiver plate (basolateral compartment) with 300 μL of HBSS (10 mM HEPES, pH 7.4). The plates were Incubated at 37° C. for 30 mins. 80 μL samples were removed directly from the apical and basolateral wells (using the basolateral access holes) and transferred to wells of new 96 wells plates. The Lucifer Yellow fluorescence (to monitor monolayer integrity) signal was measured in a fluorescence plate reader at 485 nM excitation and 530 nM emission.

### 5. Data Analysis

The apparent permeability coefficient (Pₐₚₚ), in units of centimeter per second, can be calculated for Caco-2 drug transport assays using the following equation:

$${P}_{app} = ( {V}_{A} \times {[ drug ]}_{acceptor} ) / ( Area \times Time \times {[ drug ]}_{initial , donor} )$$

Where V_{A} is the volume (in mL) in the acceptor well, Area is the surface area of the membrane (0.143 cm² for Transwell-96 Well Permeable Supports), and time is the total transport time in seconds.

The efflux ratio will be determined using the following equation:

$$Efflux Ratio = {P}_{app ( B - A )} / {P}_{app ( A - B )}$$

Where P_{app (B-A)} indicates the apparent permeability coefficient in basolateral to apical direction, and P_{app (A-B)} indicates the apparent permeability coefficient in apical to basolateral direction.

The recovery can be determined using the following equation:

$$Recovery \% = ( {V}_{A} \times {[ drug ]}_{acceptor} + {V}_{D} \times {[ drug ]}_{donor} ) / ( {V}_{D} \times {[ drug ]}_{initial , donor} )$$

Where V_{A} is the volume (in mL) in the acceptor well (0.235 mL for Ap→Bl flux, and 0.075 mL for Bl→Ap), V_{D} is the volume (in mL) in the donor well (0.075 mL for Ap→Bl flux, and 0.235 mL for Bl→Ap)

The leakage of Lucifer Yellow, in unit of percentage (%), can be calculated using the following equation:

$$\% LY leakage = 100 \times {[ LY ]}_{acceptor} / ( {[ LY ]}_{donor} + {[ LY ]}_{acceptor} )$$

LY leakage of <1% is acceptable to indicate the well-qualified Caco-2 monolayer.

The P_{app (B-A)}, P_{app (A-B)} and Efflux ratio are summarized in Table 4.

TABLE 4

| | P_{app (B-A)} | P_{app (A-B)} | |
|---|---|---|---|
| | (10⁻⁶, | (10⁻⁶, | Efflux |
| Compound | cm/s) | cm/s) | Ratio |
| Digoxin | 0.30 | 16.98 | 56.52 |
| Propranolol | 26.55 | 13.63 | 0.51 |
| I-7 | 20.27 | 14.70 | 0.73 |
| I-10 | 22.56 | 17.38 | 0.77 |
| I-21 | 19.9 | 40.0 | 2.0 |
| I-24 | 8.1 | 51.3 | 6.4 |
| I-25 | 4.40 | 34.75 | 7.90 |
| I-26 | 18.82 | 18.85 | 1.00 |
| I-27 | 19.21 | 18.41 | 0.96 |
| I-28 | 21.21 | 13.38 | 0.63 |
| I-29 | 8.36 | 8.00 | 0.96 |
| I-30 | 3.63 | 5.65 | 1.56 |
| I-31 | 3.55 | 31.60 | 8.90 |
| I-41 | 0.73 | 9.67 | 13.21 |
| I-46 | 11.72 | 39.26 | 3.35 |
| I-50 | 0.88 | 22.40 | 25.37 |
| I-57 | 29.4 | 43.7 | 1.5 |
| I-58 | 8.6 | 38.5 | 4.5 |
| I-64 | 2.1 | 33.1 | 15.6 |
| I-66 | 30.9 | 55.9 | 1.8 |
| I-70 | 6.97 | 10.62 | 1.52 |
| I-71 | 23.76 | 29.96 | 1.26 |
| I-72 | 3.36 | 14.53 | 4.33 |
| I-73 | 6.23 | 12.05 | 1.94 |
| I-75 | 7.53 | 17.40 | 2.31 |
| I-99 | 11.48 | 34.81 | 3.03 |
| I-101 | 0.41 | 28.02 | 68.88 |

### Example 4 Assay of Intrinsic Clearance

1. The master solution was prepared according to Table 5.

TABLE 5

| | Stock | | | | Final | |
|---|---|---|---|---|---|---|
| Reagent | Concentration | | Volume | | Concentration | |
| Phosphate buffer | 200 | mM | 200 | μL | 100 | mM |
| Ultra-pure H₂O | — | | 106 | μL | — | |
| MgCl₂ solution | 50 | mM | 40 | μL | 5 | mM |

2. Three separated experiments were performed as follows. a) With NADPH: 10 μL of 20 mg/mL liver microsomes and 40 μL of 10 mM NADPH were added to the incubations. The final concentrations of microsomes and NADPH were 0.5 mg/mL and 1 mM, respectively. b) Without NADPH: 10 μL of 20 mg/mL liver microsomes and 40 μL of ultra-pure H₂O were added to the incubations. The final concentration of microsomes was 0.5 mg/mL. c) Heat-inactivated microsomes without NADPH: 10 μL of 20 mg/mL heat-inactivated liver microsomes and 40 μL of ultra-pure H₂O were added to the incubations. The final concentration of microsomes was 0.5 mg/mL.
3. The reaction was started with the addition of 4 μL of 200 μM test compound solution or control compound solution at the final concentration of 2 μM and carried out at 37° C.
4. Aliquots of 50 μL were taken from the reaction solution at 0, 15, 30, 45 and 60 min. The reaction was stopped by the addition of 4 volumes of cold acetonitrile with IS (100 nM alprazolam, 200 nM labetalol, 200 nM caffeine and 2 μM ketoprofen). Samples were centrifuged at 3, 220 g for 40 minutes. Aliquot of 100 μL of the supernatant was mixed with 100 μL of ultra-pure H₂O and then used for LC-MS/MS analysis.
5. Data Analysis

All calculations were carried out using Microsoft Excel.

Peak areas were determined from extracted ion chromatograms. The slope value, k, was determined by linear regression of the natural logarithm of the remaining percentage of the parent drug vs. incubation time curve.

The in vitro half-life (in vitro t_{1/2}) was determined from the slope value:

in vitro t_{1/2}=−(0.693/k)

Conversion of the in vitro t_{1/2} (min) into the in vitro intrinsic clearance (in vitro CLᵢₙₜ, in μL/min/mg protein) was done using the following equation (mean of duplicate determinations):

$$in vitro {CL}_{int} = ( 0.693 * volume of incubation ( μ l ) ) / ( in vitro {t}_{1 / 2} * amount of proteins ( mg ) )$$

Conversion of the in vitro t_{1/2} (min) into the scale-up unbound intrinsic clearance (Scale-up CLᵢₙₜ, in mL/min/kg) was done using the following equation (mean of duplicate determinations):

The Scaling Factors for Intrinsic Clearance Prediction in Liver Microsomes are summarized in Table 6.

TABLE 6

| | Liver Weight | Microsomal | Liver blood | |
|---|---|---|---|---|
| | (g liver/kg | Concentration | flow (Q, | Scaling |
| Species | body weight)^{a} | (mg/g liver)^{b} | mL/min/kg)^{a} | Factor |
| Human | 25.7 | 48.8 | 20.7 | 1254.2 |
| Monkey | 30.0 | 50.0 | 43.6 | 1500.0 |
| Dog | 32.0 | 77.9 | 30.9 | 2492.8 |
| Rat | 40.0 | 44.8 | 55.2 | 1792.0 |
| Mouse | 88.0 | 50.0 | 90.0 | 4400.0 |

^{a}Iwatsubo et al, Davies and Morris, 1993, 10 (7) pp 1093-1095.
^{b}Barter et al, 2007, Curr Drug Metab, 8(1), pp 33-45; Iwatsubo et al, 1997, JPET, 283 pp 462-469.

The Papp (B-A), Papp (A-B) and Efflux ratio are summarized in Table 7.

TABLE 7

| | | | in vitro CLᵢₙₜ |
|---|---|---|---|
| | | in vitro t_{1/2} | (μL/min/mg |
| Compound | Species | (min) | protein) |
| I-7 | Human | 410.77 | 3.37 |
| | Mouse | 262.87 | 5.27 |
| I-9 | Human | 111.69 | 12.41 |
| | Mouse | 31.92 | 43.43 |
| I-10 | Human | 32.18 | 43.06 |
| | Mouse | 11.86 | 116.90 |
| I-11 | Human | 158.66 | 8.74 |
| | Mouse | 84.86 | 16.33 |
| I-21 | Human | 186.4 | 7.4 |
| | Mouse | 186.4 | 7.4 |
| I-24 | Human | 186.4 | 7.4 |
| | Mouse | 24.1 | 57.5 |
| I-25 | Mouse | 539.01 | 2.57 |
| I-26 | Human | 16.12 | 85.99 |
| | Mouse | 1.44 | 959.28 |
| I-27 | Human | 3.21 | 431.99 |
| | Mouse | 1.51 | 914.94 |
| I-28 | Human | 21.47 | 64.57 |
| I-28 | Mouse | 3.07 | 451.36 |
| I-29 | Human | 23.51 | 58.96 |
| | Mouse | 1.65 | 838.27 |
| I-30 | Human | 32.81 | 42.24 |
| | Mouse | 7.82 | 177.33 |
| I-31 | Human | 131.78 | 10.52 |
| | Mouse | 12.10 | 114.55 |
| I-33 | Human | 5.06 | 274.06 |
| | Mouse | 1.33 | 1041.32 |
| I-40 | Human | 26.43 | 52.45 |
| | Mouse | 2.61 | 531.67 |
| I-41 | Human | 163.38 | 8.48 |
| | Mouse | 89.50 | 15.49 |
| I-42 | Human | 29.22 | 47.44 |
| | Mouse | 1.42 | 974.75 |
| I-44 | Human | 278.31 | 4.98 |
| I-44 | Mouse | 67.51 | 20.53 |
| I-46 | Human | 168.55 | 8.22 |
| | Mouse | 6.21 | 223.27 |
| I-50 | Human | 102.38 | 13.54 |
| | Mouse | 39.45 | 35.14 |
| I-57 | Human | 19.3 | 71.9 |
| | Mouse | 5.0 | 277.3 |
| I-58 | Human | 186.4 | 7.4 |
| | Mouse | 79.9 | 17.4 |
| I-64 | Human | 186.4 | 7.4 |
| I-66 | Human | 66.3 | 20.9 |
| | Mouse | 17.5 | 79.4 |
| I-70 | Human | 72.60 | 19.09 |
| | Mouse | 15.67 | 88.43 |
| I-71 | Human | 101.93 | 13.60 |
| | Mouse | 5.87 | 236.30 |
| I-72 | Human | 111.10 | 12.48 |
| | Mouse | 23.87 | 58.05 |
| I-73 | Human | 102.25 | 13.56 |
| | Mouse | 95.84 | 14.46 |
| I-75 | Human | 95.19 | 14.56 |
| | Mouse | 17.18 | 80.67 |
| I-77 | Human | 55.05 | 25.18 |
| | Mouse | 9.14 | 151.59 |
| I-80 | Human | 12.33 | 112.40 |
| | Mouse | 2.51 | 552.00 |
| I-82 | Human | 84.11 | 16.48 |
| | Mouse | 3.06 | 453.44 |
| I-89 | Human | 14.69 | 94.35 |
| | Mouse | 2.09 | 664.59 |
| I-96 | Human | 370.14 | 3.74 |
| | Mouse | 138.47 | 10.01 |
| I-99 | Human | 265.58 | 5.22 |
| | Mouse | 68.00 | 20.38 |
| I-100 | Human | 20.32 | 68.22 |
| | Mouse | 2.98 | 465.16 |
| I-101 | Human | 2678.19 | 0.52 |
| | Mouse | 77.45 | 17.90 |
| I-102 | Human | 46.39 | 29.88 |
| | Mouse | 2.27 | 611.48 |
| I-103 | Human | 27.82 | 49.82 |
| | Mouse | 2.04 | 678.80 |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A compound having the structure of formula (I), 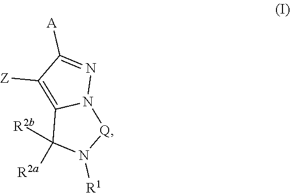
or a pharmaceutically acceptable salt, or prodrug thereof, or a solvate or hydrate of any of the forgoing, or a composition comprising any of the forgoing,
each A and Z is independently selected from the group consisting of optionally substituted C₆-C₁₄ aryl, and optionally substituted C₂-C₉ heteroaryl;
Q is selected from the group consisting of optionally substituted —CH₂—, optionally substituted —CH₂—CH₂—, optionally substituted —CH═CH—, and optionally substituted —CH₂—CH₂—CH₂—;
R¹ is selected from the group consisting of hydrogen, protium, deuterium, tritium, halogen, cyano, nitro, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl;
each R^{2a} and R^{2b} is independently absent or is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₃-C₁₀) aryl, and optionally substituted (C₂-C₉)heteroaryl,
or R^{2a} and R^{2b} combined with the atoms to which they are attached form an optionally substituted ring.

2. The compound of claim 1,
wherein, when R¹ is H, Z is not selected from the group consisting of: 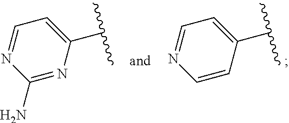 [SMILES: *c1ccnc(N)n1 ; *c1ccncc1]
when R¹ is —CH₃, Z is not selected from the group consisting of: 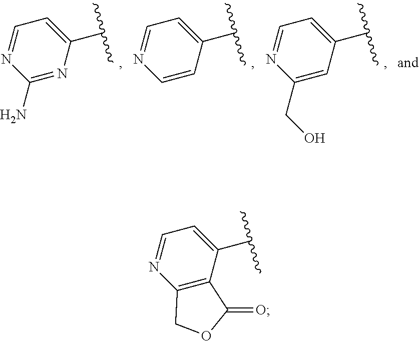
when R¹ is selected from the group consisting of:
—C(═O)—C₂H₅, —C(═O)—CH—(CH₃)₂, —C(═O)—CH₂—CN, —C(═O)—NH—CH₃, 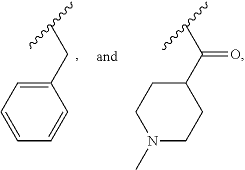
Z is not 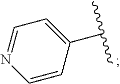 [SMILES: *c1ccncc1]
when R¹ is 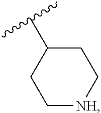 [SMILES: *C1CCNCC1]
Z is not 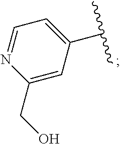 [SMILES: *c1ccnc(CO)c1]
when R¹ is 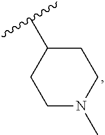 [SMILES: *C1CCN(C)CC1]
Z is not 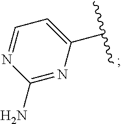 [SMILES: *c1ccnc(N)n1]
when R¹ is —C(═O)—CH₃, Z is not selected from the group consisting of:

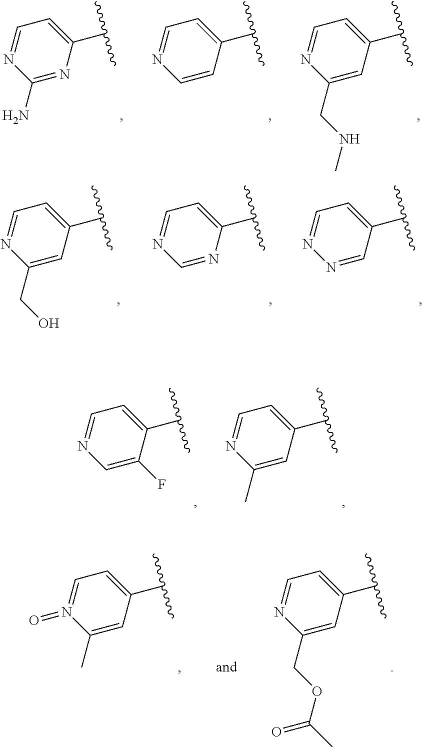

3. The compound of claim 1, wherein,
R¹ is selected from the group consisting of deuterium, optionally substituted-methyl-(C₁-C₉)heteroaryl, optionally substituted-halomethyl, optionally substituted deuterated methyl, optionally substituted-methyl-(C₃-C₁₀)carbocycle, optionally substituted-methyl-thio, optionally substituted (C₂-C₆)alkyl, optionally substituted —(C₁) acyl-(C₁-C₉)heteroaryl, optionally substituted —(C₁) acyl-(C₆-C₁₀) aryl, optionally substituted —(C₁) acyl-tetrahydrofuran, optionally substituted —(C₁) acyl-(C₃-C₁₀)carbocycle, optionally substituted-halo(C₂) acyl, optionally substituted —(C₂) acyl-hydroxy, optionally substituted (C₂)thioacyl, optionally substituted —(C₃) acyl-amino, optionally substituted —C(═O)—(CH₂)₂—CH₃, optionally substituted —(C₅-C₆) acyl, optionally substituted oxetane, optionally substituted tetrahydropyran, optionally substituted tetrahydrothiopyran, optionally substituted sulfonyl, optionally substituted phosphorous-containing group, optionally substituted silicon-containing group, optionally substituted thio, optionally substituted (C₁-C₆)alkenyl, optionally substituted (C₃-C₁₀)carbocycle, and optionally substituted (C₁-C₆)heteroaryl.

4. The compound of claim 1, wherein,
R¹ is selected from the group consisting of optionally substituted (C₂-C₆)alkyl, optionally substituted —(C₅-C₆) acyl, optionally substituted oxetane, optionally substituted tetrahydropyran, optionally substituted tetrahydrothiopyran, optionally substituted sulfonyl, optionally substituted thio, optionally substituted (C₂-C₆)alkenyl, optionally substituted (C₃-C₁₀)carbocycle, and optionally substituted (C₁-C₉)heteroaryl.

5. The compound of claim 1, wherein,
R¹ is optionally substituted (C₂-C₆)alkyl; or
R¹ is selected from the group consisting of optionally substituted oxetane, optionally substituted tetrahydropyran, and optionally substituted tetrahydrothiopyran; or
R¹ is optionally substituted sulfonyl; or
R¹ is optionally substituted thio; or
R¹ is optionally substituted (C₂-C₆)alkenyl; or
R¹ is optionally substituted (C₃-C₁₀)carbocycle; or
R¹ is optionally substituted (C₁-C₉)heteroaryl.

6. (canceled)

7. (canceled)

8. (canceled)

9. (canceled)

10. (canceled)

11. (canceled)

12. The compound of claim 1, wherein, said R¹ is substituted with one or more R³, each R³ is independently absent or is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl; or
each R³ is independently selected from the group consisting of hydrogen, deuterium, halogen, cyano, ═O, optionally substituted hydroxy, optionally substituted thio, optionally substituted amino, optionally substituted (C₁-C₆)alkyl optionally substituted (C₃-C₁₀)carbocycle, optionally substituted (C₂-C₉)heterocycle, optionally substituted (C₆-C₁₀) aryl, and optionally substituted (C₁-C₉)heteroaryl; or
each R³ is independently selected from the grow consisting of hydrogen, optionally substituted hydroxy, optionally substituted thio, optionally substituted amino, optionally substituted (C₁-C₆)alkyl, optionally substituted (C₃-C₁₀)carbocycle, optionally substituted (C₂-C₉)heterocycle, optionally substituted (C₃-C₁₀) aryl, and optionally substituted (C₁-C₉)heteroaryl; or
each R³ is independently selected from the group consisting of hydrogen, optionally substituted (C₁-C₆)alkyl, and optionally substituted (C₆-C₁₀) aryl; or
each R³ is independently selected from the group consisting of hydrogen, optionally substituted methyl, and optionally substituted phenyl.

13. (canceled)

14. (canceled)

15. (canceled)

16. (canceled)

17. The compound of claim 12, wherein, said R³ is substituted with one or more R⁴, each R⁴ is independently absent or is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl; or
each R⁴ is independently selected from the group consisting of hydrogen, halogen, optionally substituted (C₁-C₆) acyl, and optionally substituted (C₁-C₆)alkyl; or
each R⁴ is independently selected from the group consisting of hydrogen and halogen; or
each R⁴ is independently selected from the group consisting of hydrogen and F.

18. (canceled)

19. (canceled)

20. (canceled)

21. The compound of claim 1, wherein, said R^{2a} is hydrogen and said R^{2b} is hydrogen; or
said R^{2a} is ═O and said R^{2b} is absent.

22. (canceled)

23. The compound of claim 1, wherein, Q is optionally substituted —CH₂—CH₂—.

24. The compound of claim 1, wherein, said Z is optionally substituted C₆-C₁₄ heteroaryl; or
said Z is selected from the group consisting of optionally substituted pyridine and optionally substituted pyrimidine.

25. (canceled)

26. The compound of claim 1, wherein, said Z is substituted with one or more R⁵, each R⁵ is independently absent or is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₃-C₁₀) aryl, and optionally substituted (C₁-C₉)heteroaryl; or
each R⁵ is independently selected from the group consisting of hydrogen, optionally substituted amino, optionally substituted (C₁-C₆)alkyl, and optionally substituted (C₂-C₉)heterocycle; or
each R⁵ is independently selected from the group consisting of hydrogen, optionally substituted amino, and optionally substituted (C₁-C₆)alkyl; or
each R⁵ is independently selected from the group consisting of hydrogen, optionally substituted amino, and optionally substituted methyl.

27. (canceled)

28. (canceled)

29. (canceled)

30. The compound of claim 26, wherein, said R⁵ is substituted with one or more R⁶, each R⁶ is independently absent or is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl; or
each R⁶ is independently selected from the group consisting of hydrogen, optionally substituted hydroxy, optionally substituted (C₁-C₆) acyl, and optionally substituted (C₁-C₉)heteroaryl; or
each R⁶ is independently selected from the group consisting of hydrogen and optionally substituted (C₂-C₆) acyl.

31. (canceled)

32. (canceled)

33. The compound of claim 1, wherein, said A is optionally substituted C₆-C₁₄ aryl; or
said A is optionally substituted phenyl.

34. (canceled)

35. The compound of claim 1, wherein, said A is substituted with one or more R⁷, each R⁷ is independently absent or is independently selected from the group consisting of hydrogen, protium, deuterium, tritium, halogen, cyano, nitro, ═S, ═O, N₃, optionally substituted hydroxy, optionally substituted phosphorous-containing group, optionally substituted silicon-containing group, optionally substituted thio, optionally substituted amino, optionally substituted carboxyl, optionally substituted sulfonyl, optionally substituted sulfinyl, optionally substituted (C₁-C₆) acyl, optionally substituted (C₁-C₆)thioacyl, optionally substituted (C₁-C₆)alkyl, optionally substituted (C₂-C₆)alkenyl, optionally substituted (C₂-C₆)alkynyl, optionally substituted (C₃-C₁₀)carbocycle, optionally substituted (C₂-C₉)heterocycle, optionally substituted (C₆-C₁₀) aryl, and optionally substituted (C₁-C₉)heteroaryl; or
each R⁷ is halogen; or
each R⁷ is F.

36. (canceled)

37. (canceled)

38. The compound of claim 1, wherein, said compound is selected from the group consisting of:

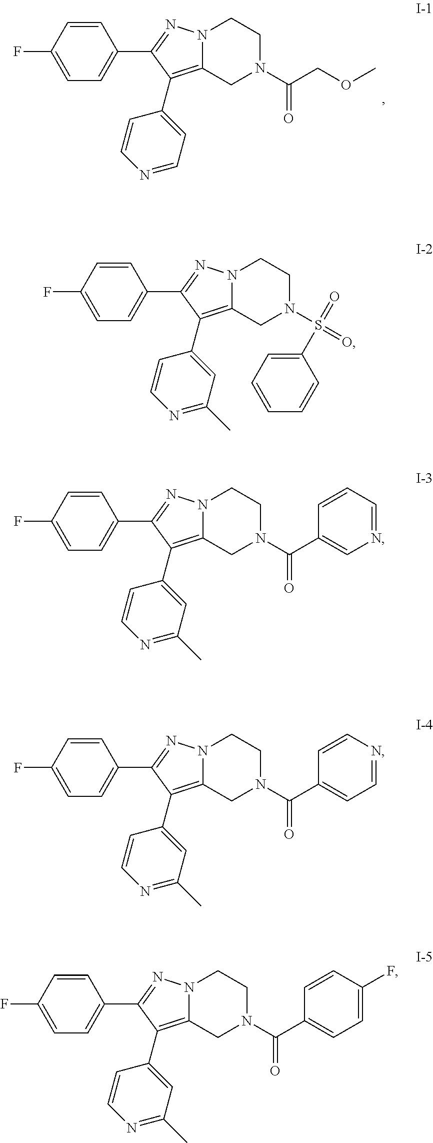 [SMILES: COCC(=O)N1CCn2nc(-c3ccc(F)cc3)c(-c3ccncc3)c2C1 ; Cc1cc(-c2c(-c3ccc(F)cc3)nn3c2CN(S(=O)(=O)c2ccccc2)CC3)ccn1 ; Cc1cc(-c2c(-c3ccc(F)cc3)nn3c2CN(C(=O)c2cccnc2)CC3)ccn1 ; Cc1cc(-c2c(-c3ccc(F)cc3)nn3c2CN(C(=O)c2ccncc2)CC3)ccn1 ; Cc1cc(-c2c(-c3ccc(F)cc3)nn3c2CN(C(=O)c2ccc(F)cc2)CC3)ccn1]

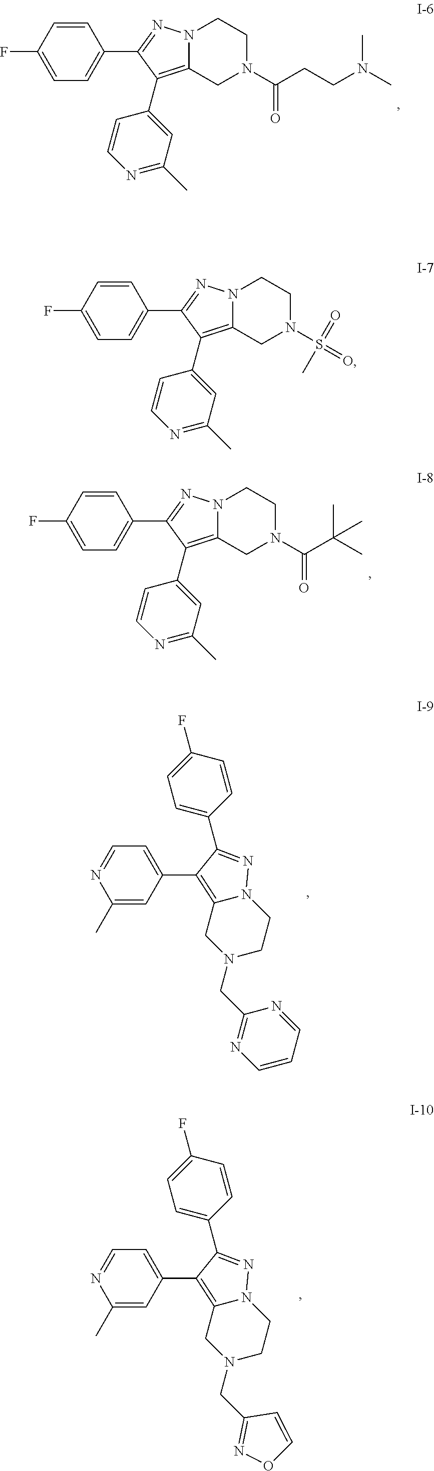

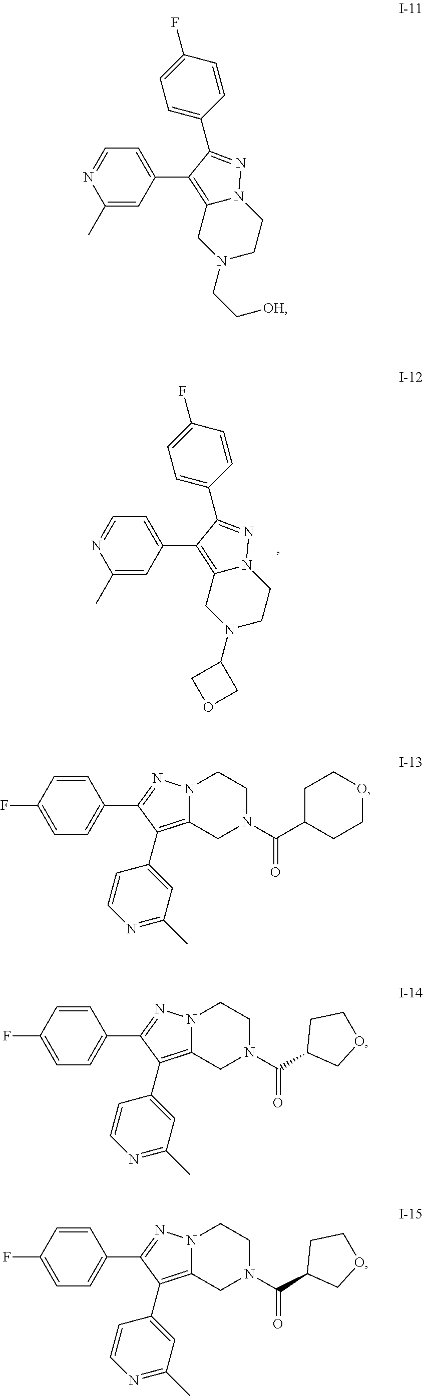 [SMILES: Cc1cc(-c2c(-c3ccc(F)cc3)nn3c2CN(CCO)CC3)ccn1 ; Cc1cc(-c2c(-c3ccc(F)cc3)nn3c2CN(C2COC2)CC3)ccn1 ; Cc1cc(-c2c(-c3ccc(F)cc3)nn3c2CN(C(=O)C2CCOCC2)CC3)ccn1 ; Cc1cc(-c2c(-c3ccc(F)cc3)nn3c2CN(C(=O)[C@@H]2CCOC2)CC3)ccn1 ; Cc1cc(-c2c(-c3ccc(F)cc3)nn3c2CN(C(=O)[C@H]2CCOC2)CC3)ccn1]

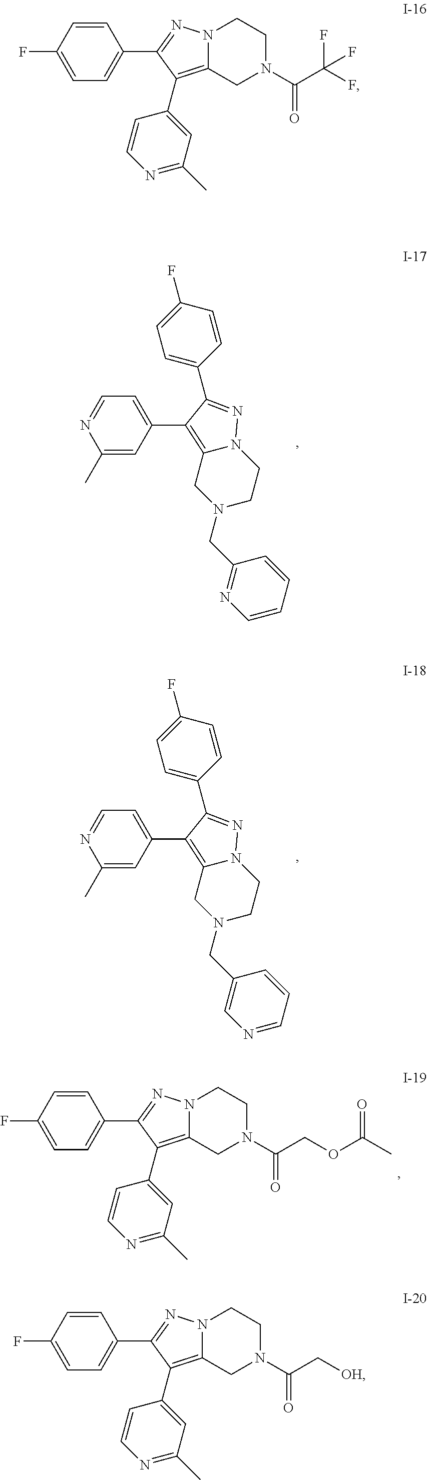 [SMILES: Cc1cc(-c2c(-c3ccc(F)cc3)nn3c2CN(C(=O)C(F)(F)F)CC3)ccn1 ; Cc1cc(-c2c(-c3ccc(F)cc3)nn3c2CN(Cc2ccccn2)CC3)ccn1 ; Cc1cc(-c2c(-c3ccc(F)cc3)nn3c2CN(Cc2cccnc2)CC3)ccn1 ; CC(=O)OCC(=O)N1CCn2nc(-c3ccc(F)cc3)c(-c3ccnc(C)c3)c2C1 ; Cc1cc(-c2c(-c3ccc(F)cc3)nn3c2CN(C(=O)CO)CC3)ccn1]

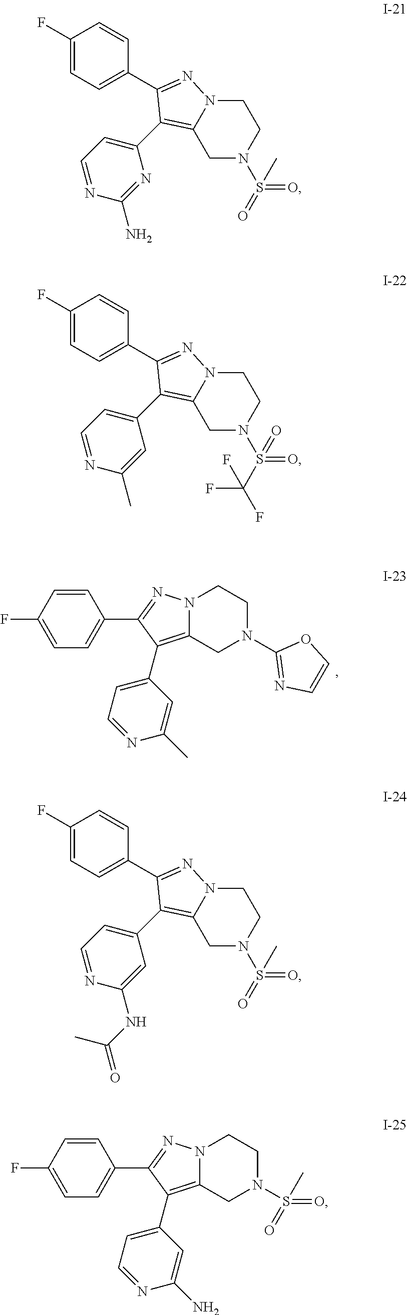 [SMILES: CS(=O)(=O)N1CCn2nc(-c3ccc(F)cc3)c(-c3ccnc(N)n3)c2C1 ; Cc1cc(-c2c(-c3ccc(F)cc3)nn3c2CN(S(=O)(=O)C(F)(F)F)CC3)ccn1 ; Cc1cc(-c2c(-c3ccc(F)cc3)nn3c2CN(c2ncco2)CC3)ccn1 ; CC(=O)Nc1cc(-c2c(-c3ccc(F)cc3)nn3c2CN(S(C)(=O)=O)CC3)ccn1 ; CS(=O)(=O)N1CCn2nc(-c3ccc(F)cc3)c(-c3ccnc(N)c3)c2C1]

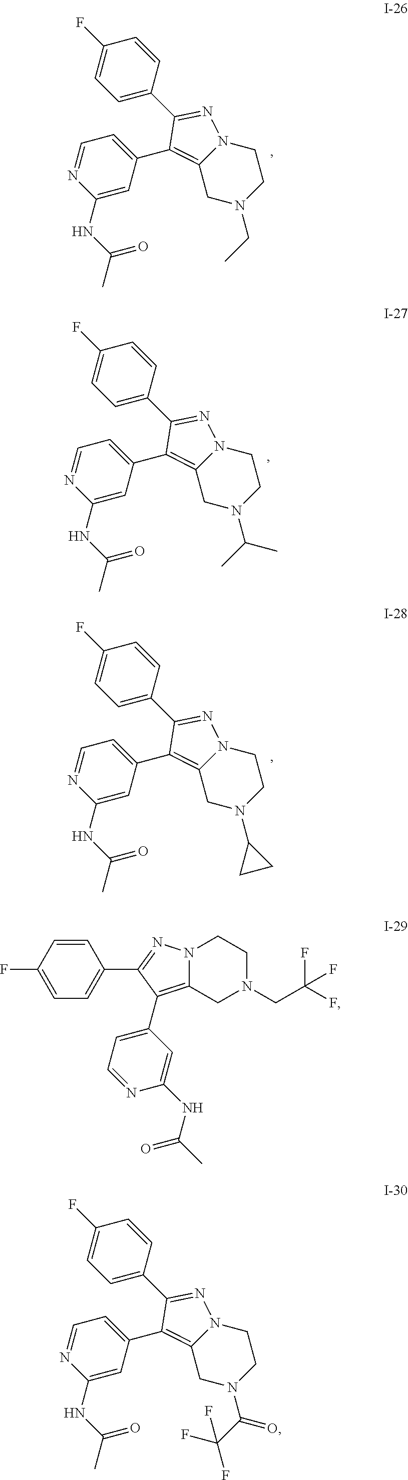

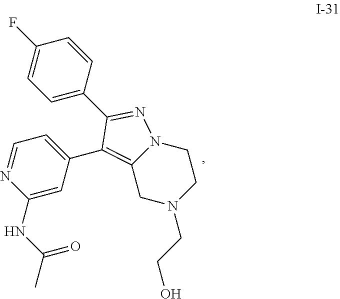 [SMILES: CC(=O)Nc1cc(-c2c(-c3ccc(F)cc3)nn3c2CN(CCO)CC3)ccn1]

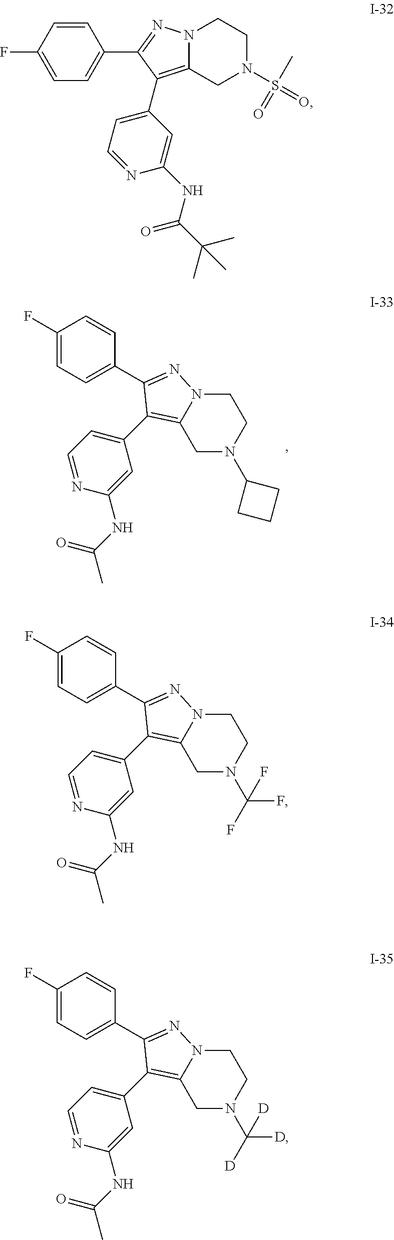

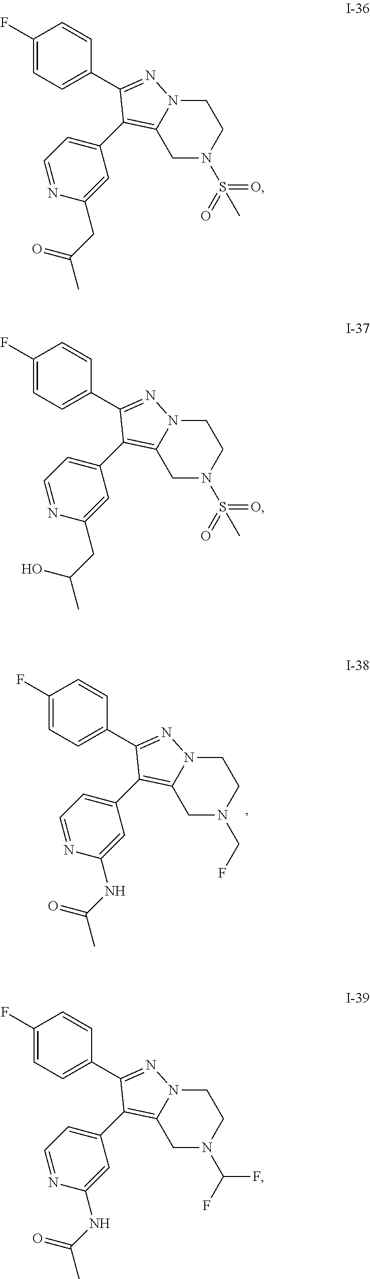 [SMILES: CC(=O)Cc1cc(-c2c(-c3ccc(F)cc3)nn3c2CN(S(C)(=O)=O)CC3)ccn1 ; CC(O)Cc1cc(-c2c(-c3ccc(F)cc3)nn3c2CN(S(C)(=O)=O)CC3)ccn1 ; CC(=O)Nc1cc(-c2c(-c3ccc(F)cc3)nn3c2CN(CF)CC3)ccn1 ; CC(=O)Nc1cc(-c2c(-c3ccc(F)cc3)nn3c2CN(C(F)F)CC3)ccn1]

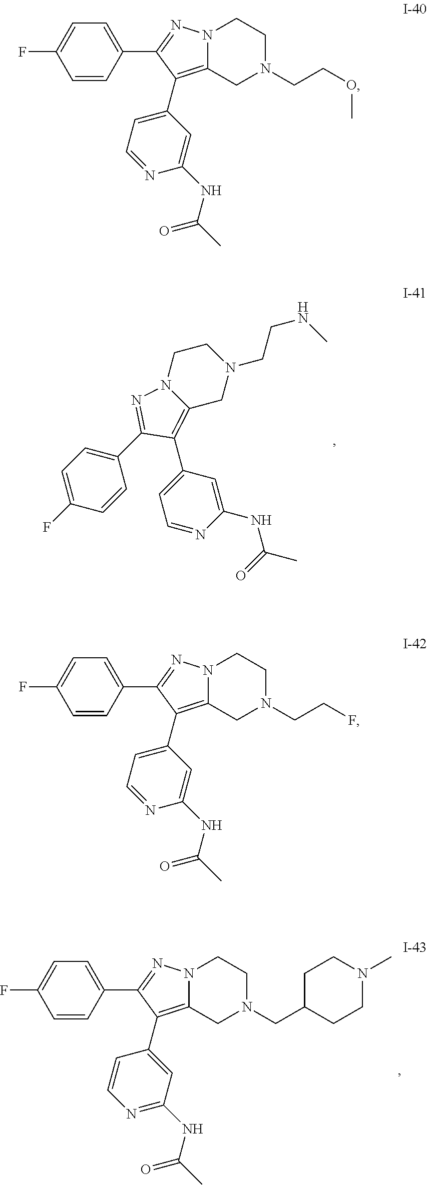 [SMILES: COCCN1CCn2nc(-c3ccc(F)cc3)c(-c3ccnc(NC(C)=O)c3)c2C1 ; CNCCN1CCn2nc(-c3ccc(F)cc3)c(-c3ccnc(NC(C)=O)c3)c2C1 ; CC(=O)Nc1cc(-c2c(-c3ccc(F)cc3)nn3c2CN(CCF)CC3)ccn1 ; CC(=O)Nc1cc(-c2c(-c3ccc(F)cc3)nn3c2CN(CC2CCN(C)CC2)CC3)ccn1]

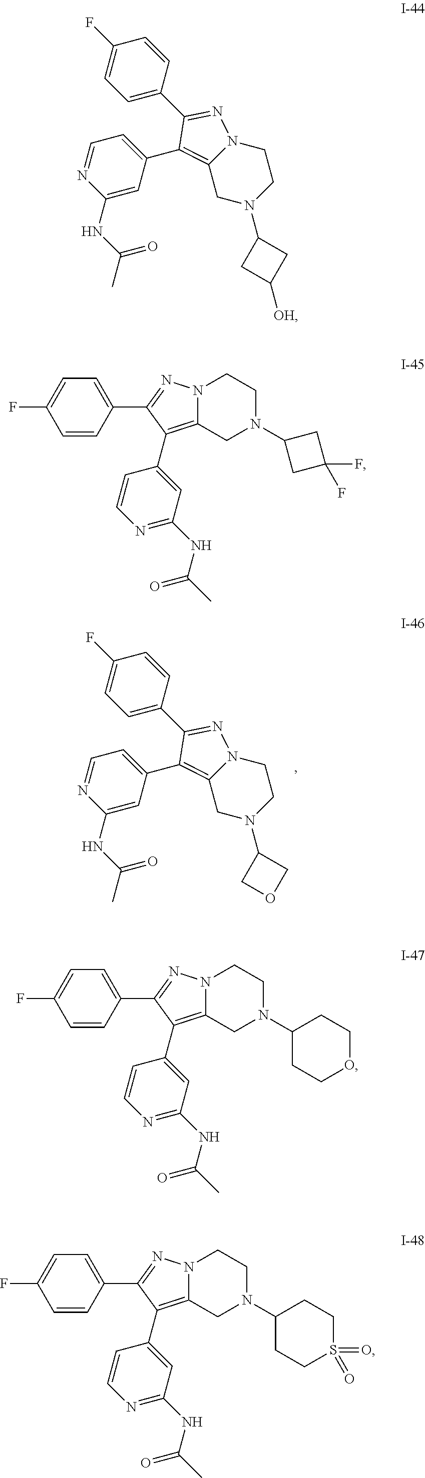 [SMILES: CC(=O)Nc1cc(-c2c(-c3ccc(F)cc3)nn3c2CN(C2CC(O)C2)CC3)ccn1 ; CC(=O)Nc1cc(-c2c(-c3ccc(F)cc3)nn3c2CN(C2CC(F)(F)C2)CC3)ccn1 ; CC(=O)Nc1cc(-c2c(-c3ccc(F)cc3)nn3c2CN(C2COC2)CC3)ccn1 ; CC(=O)Nc1cc(-c2c(-c3ccc(F)cc3)nn3c2CN(C2CCOCC2)CC3)ccn1 ; CC(=O)Nc1cc(-c2c(-c3ccc(F)cc3)nn3c2CN(C2CCS(=O)(=O)CC2)CC3)ccn1]

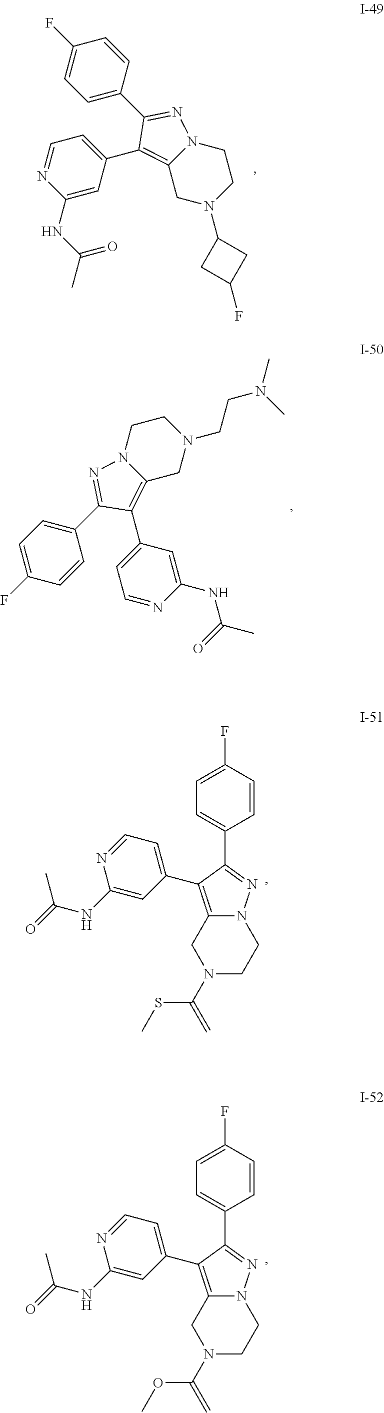 [SMILES: CC(=O)Nc1cc(-c2c(-c3ccc(F)cc3)nn3c2CN(C2CC(F)C2)CC3)ccn1 ; CC(=O)Nc1cc(-c2c(-c3ccc(F)cc3)nn3c2CN(CCN(C)C)CC3)ccn1 ; C=C(SC)N1CCn2nc(-c3ccc(F)cc3)c(-c3ccnc(NC(C)=O)c3)c2C1 ; C=C(OC)N1CCn2nc(-c3ccc(F)cc3)c(-c3ccnc(NC(C)=O)c3)c2C1]

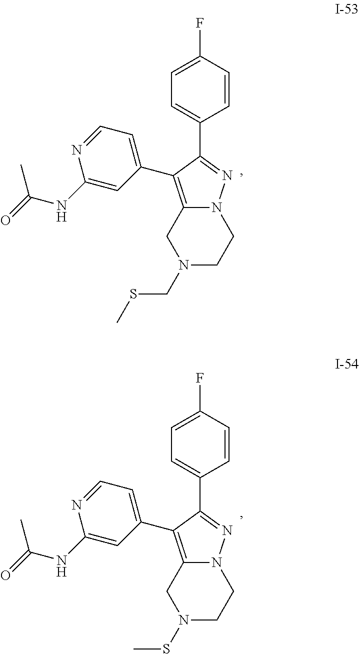 [SMILES: CSCN1CCn2nc(-c3ccc(F)cc3)c(-c3ccnc(NC(C)=O)c3)c2C1 ; CSN1CCn2nc(-c3ccc(F)cc3)c(-c3ccnc(NC(C)=O)c3)c2C1]

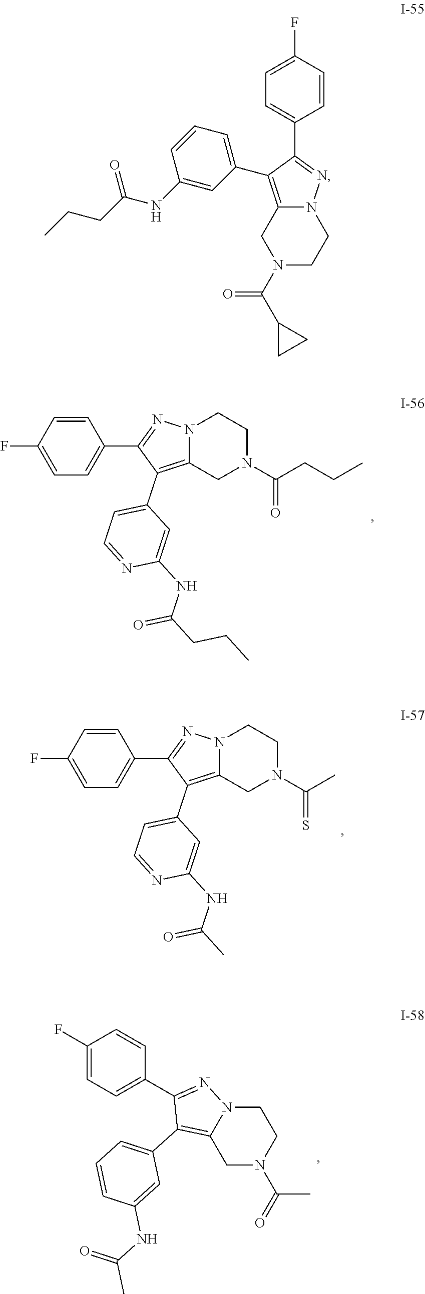 [SMILES: CCCC(=O)Nc1cccc(-c2c(-c3ccc(F)cc3)nn3c2CN(C(=O)C2CC2)CC3)c1 ; CCCC(=O)Nc1cc(-c2c(-c3ccc(F)cc3)nn3c2CN(C(=O)CCC)CC3)ccn1 ; CC(=O)Nc1cc(-c2c(-c3ccc(F)cc3)nn3c2CN(C(C)=S)CC3)ccn1 ; CC(=O)Nc1cccc(-c2c(-c3ccc(F)cc3)nn3c2CN(C(C)=O)CC3)c1]

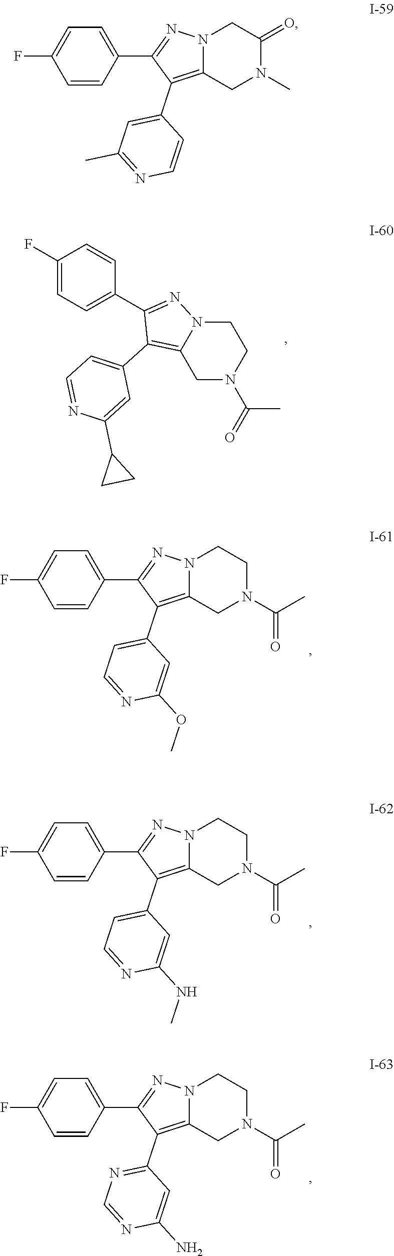 [SMILES: Cc1cc(-c2c(-c3ccc(F)cc3)nn3c2CN(C)C(=O)C3)ccn1 ; CC(=O)N1CCn2nc(-c3ccc(F)cc3)c(-c3ccnc(C4CC4)c3)c2C1 ; COc1cc(-c2c(-c3ccc(F)cc3)nn3c2CN(C(C)=O)CC3)ccn1 ; CNc1cc(-c2c(-c3ccc(F)cc3)nn3c2CN(C(C)=O)CC3)ccn1 ; CC(=O)N1CCn2nc(-c3ccc(F)cc3)c(-c3cc(N)ncn3)c2C1]

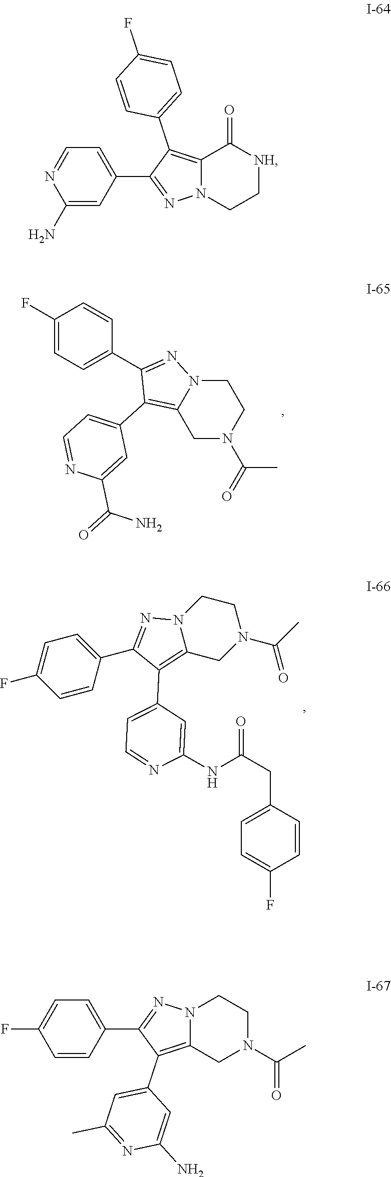 [SMILES: Nc1cc(-c2nn3c(c2-c2ccc(F)cc2)C(=O)NCC3)ccn1 ; CC(=O)N1CCn2nc(-c3ccc(F)cc3)c(-c3ccnc(C(N)=O)c3)c2C1 ; CC(=O)N1CCn2nc(-c3ccc(F)cc3)c(-c3ccnc(NC(=O)Cc4ccc(F)cc4)c3)c2C1 ; CC(=O)N1CCn2nc(-c3ccc(F)cc3)c(-c3cc(C)nc(N)c3)c2C1]

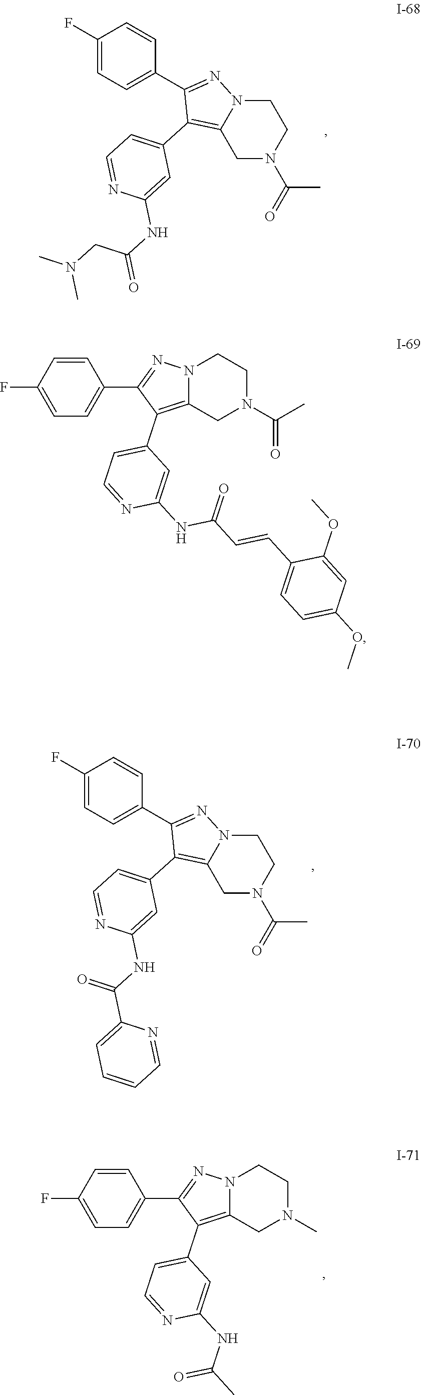 [SMILES: CC(=O)N1CCn2nc(-c3ccc(F)cc3)c(-c3ccnc(NC(=O)CN(C)C)c3)c2C1 ; COc1ccc(/C=C/C(=O)Nc2cc(-c3c(-c4ccc(F)cc4)nn4c3CN(C(C)=O)CC4)ccn2)c(OC)c1 ; CC(=O)N1CCn2nc(-c3ccc(F)cc3)c(-c3ccnc(NC(=O)c4ccccn4)c3)c2C1 ; CC(=O)Nc1cc(-c2c(-c3ccc(F)cc3)nn3c2CN(C)CC3)ccn1]

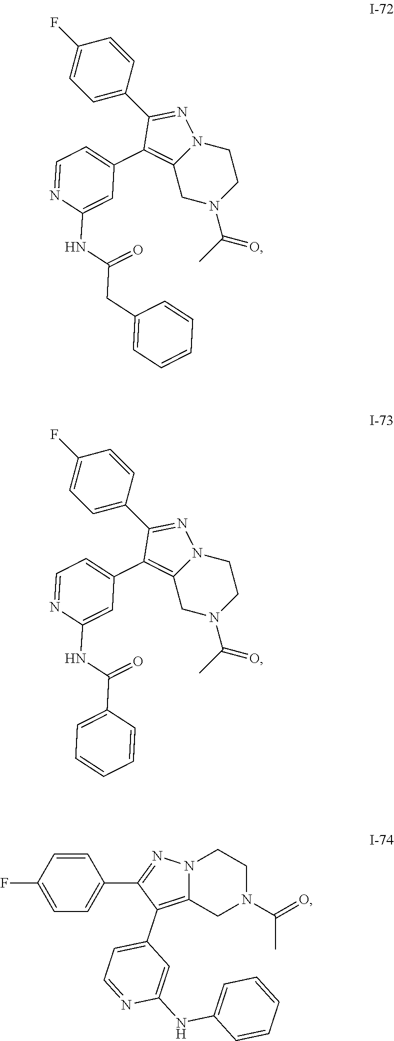 [SMILES: CC(=O)N1CCn2nc(-c3ccc(F)cc3)c(-c3ccnc(NC(=O)Cc4ccccc4)c3)c2C1 ; CC(=O)N1CCn2nc(-c3ccc(F)cc3)c(-c3ccnc(NC(=O)c4ccccc4)c3)c2C1 ; CC(=O)N1CCn2nc(-c3ccc(F)cc3)c(-c3ccnc(Nc4ccccc4)c3)c2C1]

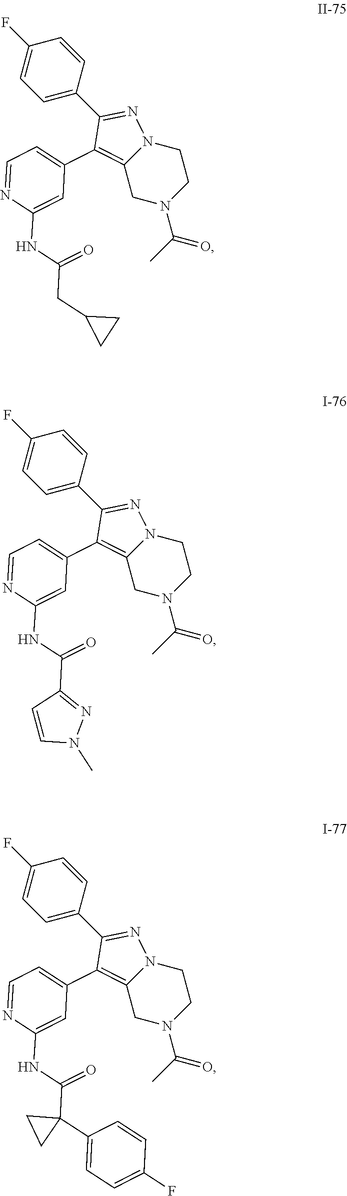 [SMILES: CC(=O)N1CCn2nc(-c3ccc(F)cc3)c(-c3ccnc(NC(=O)CC4CC4)c3)c2C1 ; CC(=O)N1CCn2nc(-c3ccc(F)cc3)c(-c3ccnc(NC(=O)c4ccn(C)n4)c3)c2C1 ; CC(=O)N1CCn2nc(-c3ccc(F)cc3)c(-c3ccnc(NC(=O)C4(c5ccc(F)cc5)CC4)c3)c2C1]

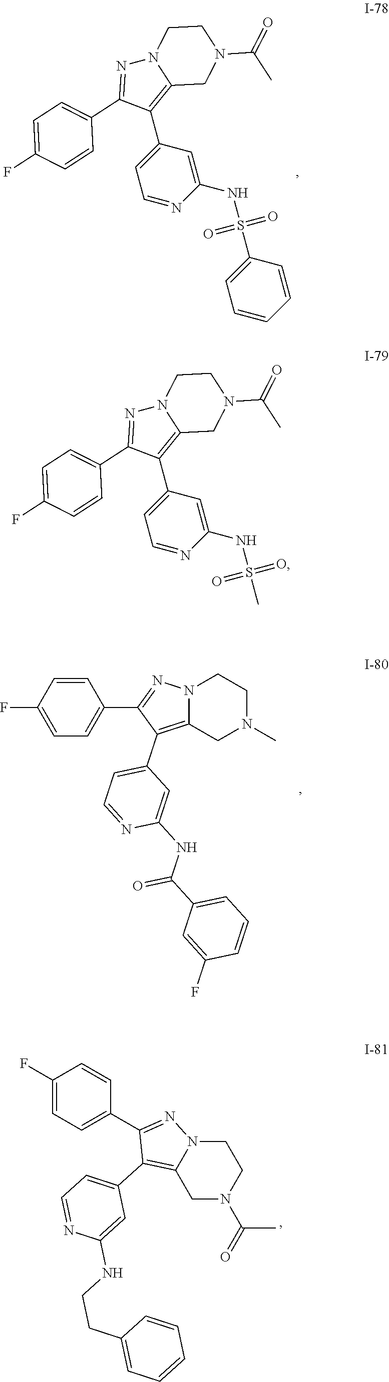 [SMILES: CC(=O)N1CCn2nc(-c3ccc(F)cc3)c(-c3ccnc(NS(=O)(=O)c4ccccc4)c3)c2C1 ; CC(=O)N1CCn2nc(-c3ccc(F)cc3)c(-c3ccnc(NS(C)(=O)=O)c3)c2C1 ; CN1CCn2nc(-c3ccc(F)cc3)c(-c3ccnc(NC(=O)c4cccc(F)c4)c3)c2C1 ; CC(=O)N1CCn2nc(-c3ccc(F)cc3)c(-c3ccnc(NCCc4ccccc4)c3)c2C1]

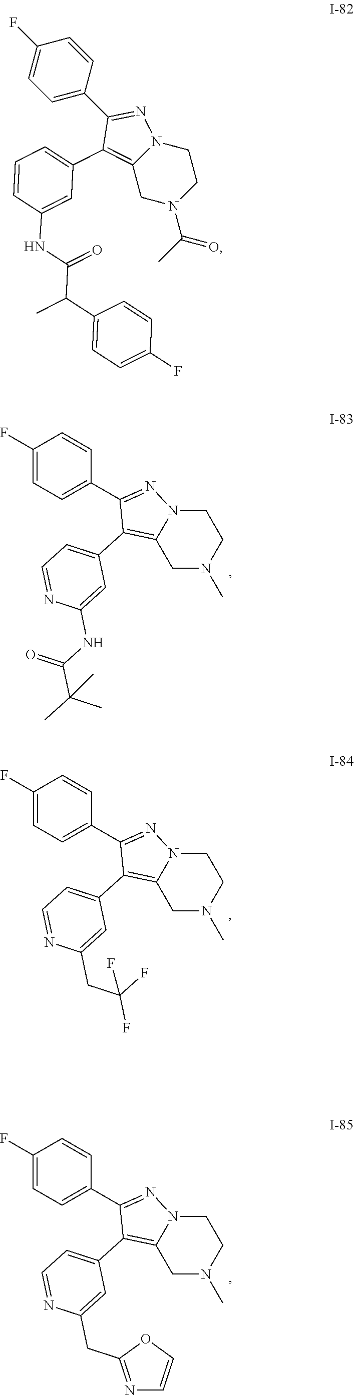 [SMILES: CC(=O)N1CCn2nc(-c3ccc(F)cc3)c(-c3cccc(NC(=O)C(C)c4ccc(F)cc4)c3)c2C1 ; CN1CCn2nc(-c3ccc(F)cc3)c(-c3ccnc(NC(=O)C(C)(C)C)c3)c2C1 ; CN1CCn2nc(-c3ccc(F)cc3)c(-c3ccnc(CC(F)(F)F)c3)c2C1 ; CN1CCn2nc(-c3ccc(F)cc3)c(-c3ccnc(Cc4ncco4)c3)c2C1]

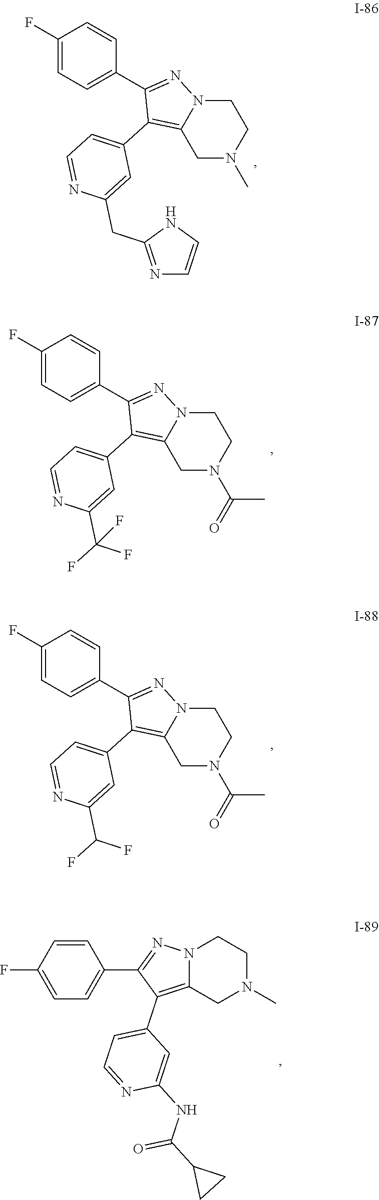 [SMILES: CN1CCn2nc(-c3ccc(F)cc3)c(-c3ccnc(Cc4ncc[nH]4)c3)c2C1 ; CC(=O)N1CCn2nc(-c3ccc(F)cc3)c(-c3ccnc(C(F)(F)F)c3)c2C1 ; CC(=O)N1CCn2nc(-c3ccc(F)cc3)c(-c3ccnc(C(F)F)c3)c2C1 ; CN1CCn2nc(-c3ccc(F)cc3)c(-c3ccnc(NC(=O)C4CC4)c3)c2C1]

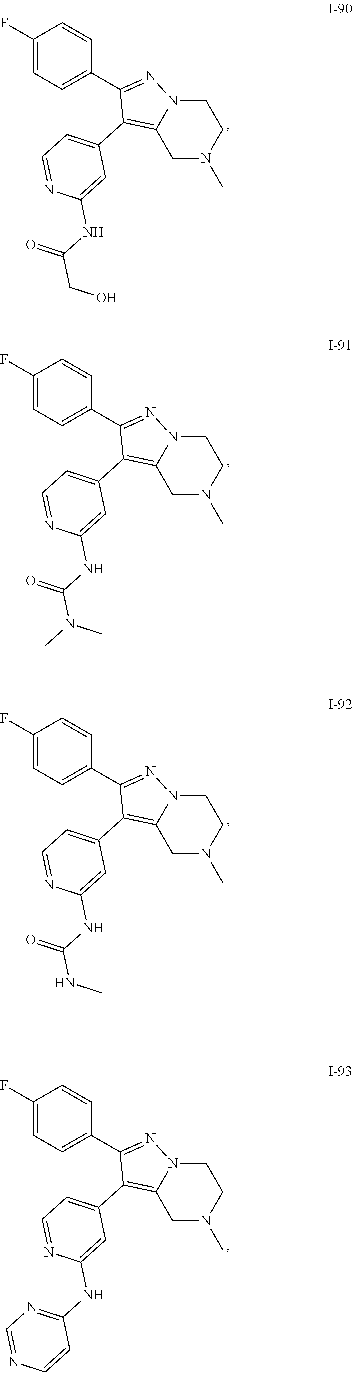 [SMILES: CN1CCn2nc(-c3ccc(F)cc3)c(-c3ccnc(NC(=O)CO)c3)c2C1 ; CN1CCn2nc(-c3ccc(F)cc3)c(-c3ccnc(NC(=O)N(C)C)c3)c2C1 ; CNC(=O)Nc1cc(-c2c(-c3ccc(F)cc3)nn3c2CN(C)CC3)ccn1 ; CN1CCn2nc(-c3ccc(F)cc3)c(-c3ccnc(Nc4ccncn4)c3)c2C1]

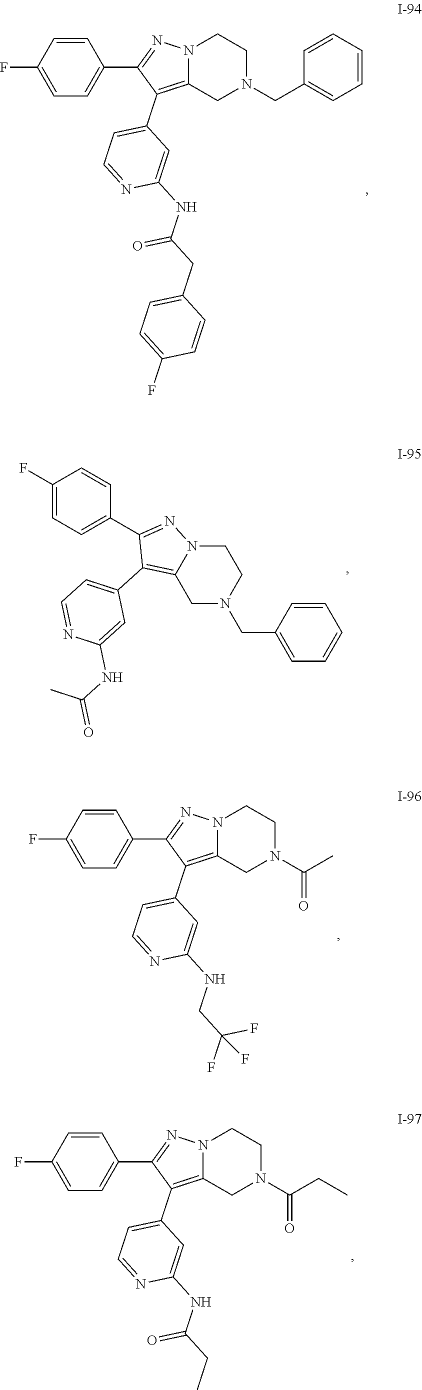 [SMILES: O=C(Cc1ccc(F)cc1)Nc1cc(-c2c(-c3ccc(F)cc3)nn3c2CN(Cc2ccccc2)CC3)ccn1 ; CC(=O)Nc1cc(-c2c(-c3ccc(F)cc3)nn3c2CN(Cc2ccccc2)CC3)ccn1 ; CC(=O)N1CCn2nc(-c3ccc(F)cc3)c(-c3ccnc(NCC(F)(F)F)c3)c2C1 ; CCC(=O)Nc1cc(-c2c(-c3ccc(F)cc3)nn3c2CN(C(=O)CC)CC3)ccn1]

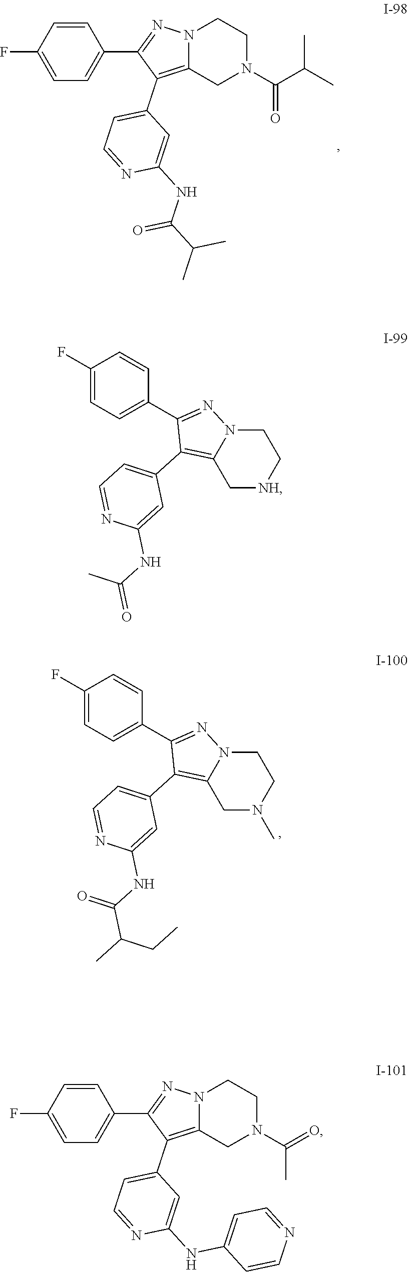 [SMILES: CC(C)C(=O)Nc1cc(-c2c(-c3ccc(F)cc3)nn3c2CN(C(=O)C(C)C)CC3)ccn1 ; CC(=O)Nc1cc(-c2c(-c3ccc(F)cc3)nn3c2CNCC3)ccn1 ; CCC(C)C(=O)Nc1cc(-c2c(-c3ccc(F)cc3)nn3c2CN(C)CC3)ccn1 ; CC(=O)N1CCn2nc(-c3ccc(F)cc3)c(-c3ccnc(Nc4ccncc4)c3)c2C1]

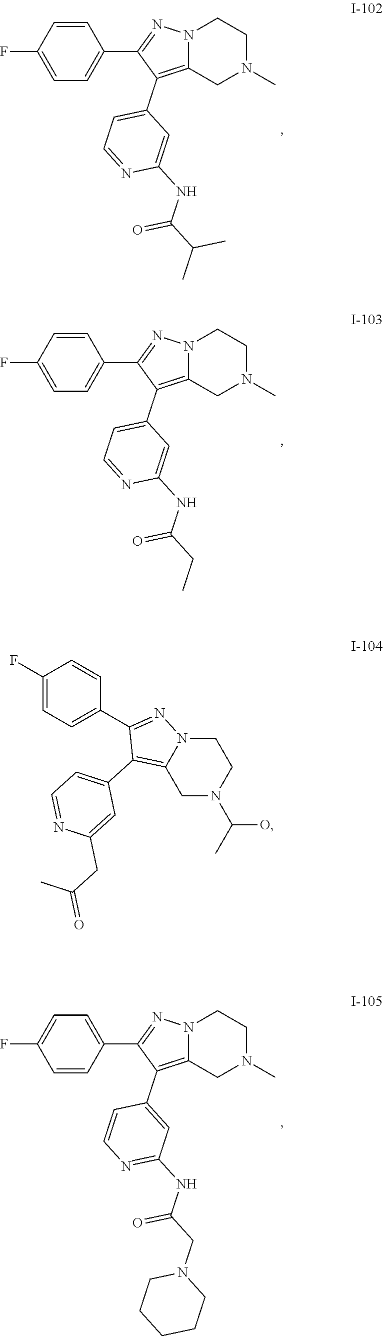

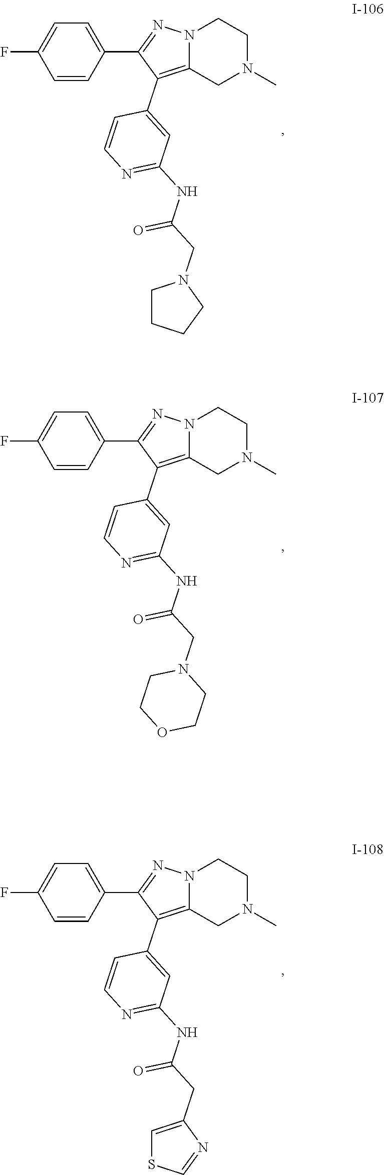 [SMILES: CN1CCn2nc(-c3ccc(F)cc3)c(-c3ccnc(NC(=O)CN4CCCC4)c3)c2C1 ; CN1CCn2nc(-c3ccc(F)cc3)c(-c3ccnc(NC(=O)CN4CCOCC4)c3)c2C1 ; CN1CCn2nc(-c3ccc(F)cc3)c(-c3ccnc(NC(=O)Cc4cscn4)c3)c2C1]

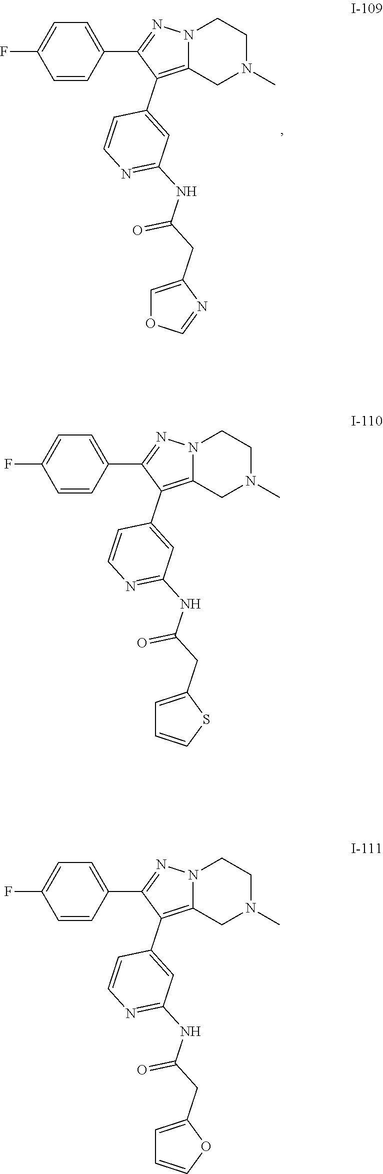 [SMILES: CN1CCn2nc(-c3ccc(F)cc3)c(-c3ccnc(NC(=O)Cc4cocn4)c3)c2C1 ; CN1CCn2nc(-c3ccc(F)cc3)c(-c3ccnc(NC(=O)Cc4cccs4)c3)c2C1 ; CN1CCn2nc(-c3ccc(F)cc3)c(-c3ccnc(NC(=O)Cc4ccco4)c3)c2C1]

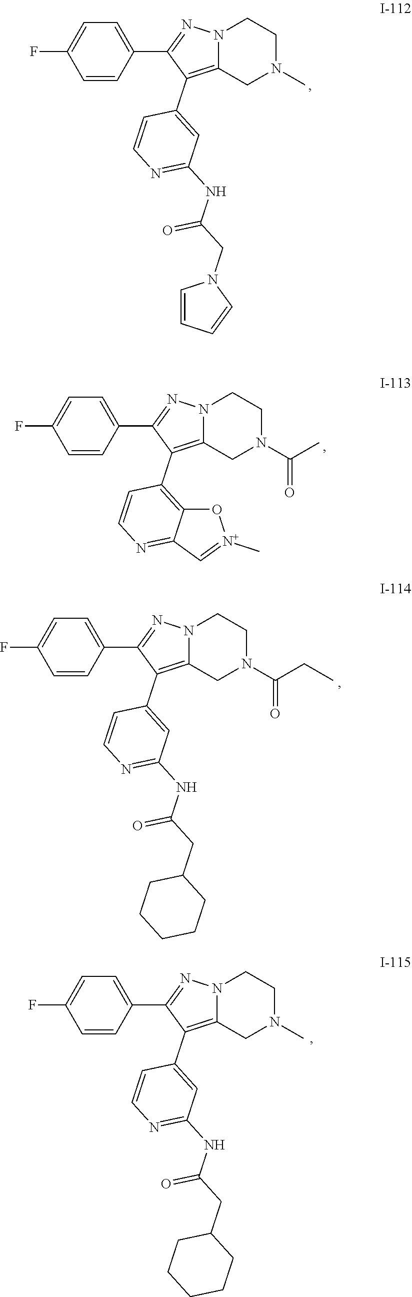

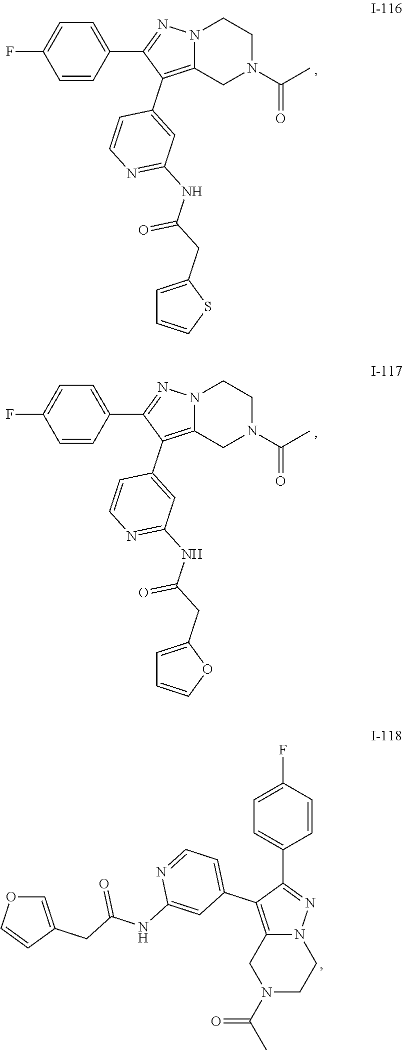 [SMILES: CC(=O)N1CCn2nc(-c3ccc(F)cc3)c(-c3ccnc(NC(=O)Cc4cccs4)c3)c2C1 ; CC(=O)N1CCn2nc(-c3ccc(F)cc3)c(-c3ccnc(NC(=O)Cc4ccco4)c3)c2C1 ; CC(=O)N1CCn2nc(-c3ccc(F)cc3)c(-c3ccnc(NC(=O)Cc4ccoc4)c3)c2C1]

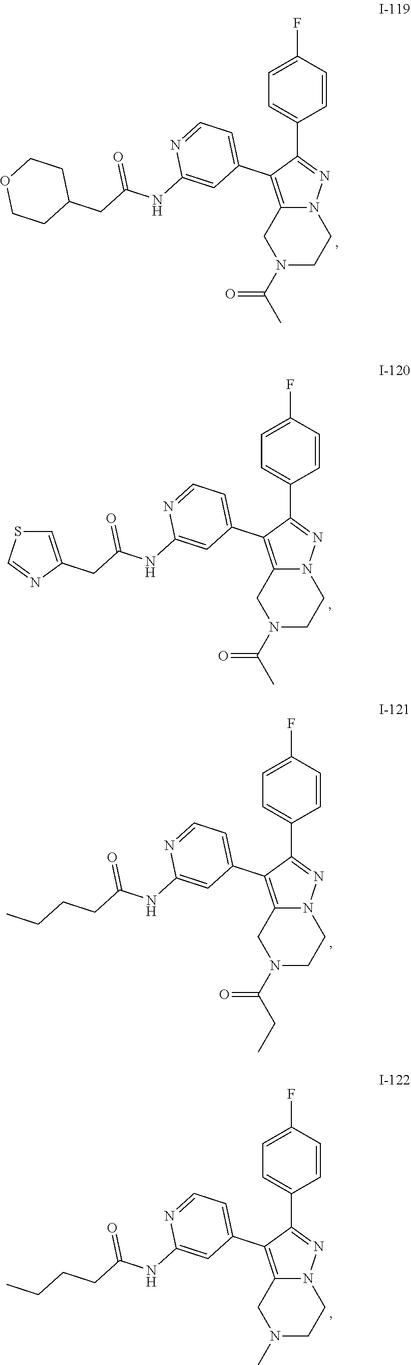

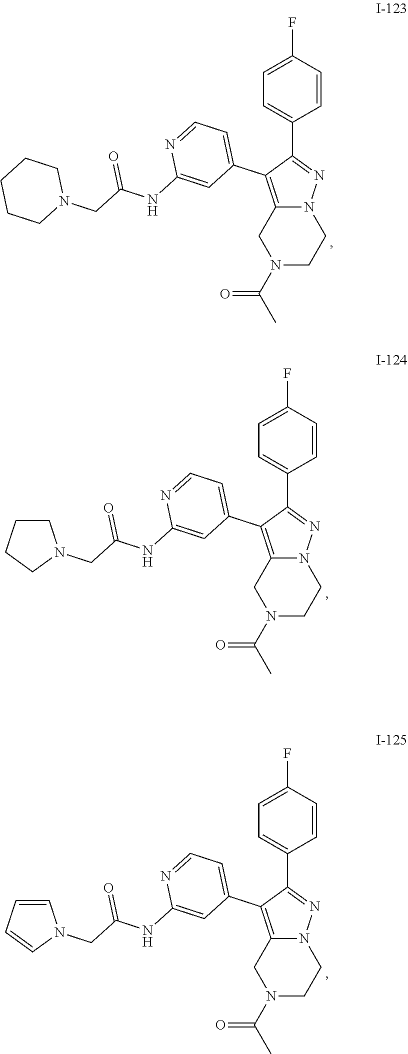 [SMILES: CC(=O)N1CCn2nc(-c3ccc(F)cc3)c(-c3ccnc(NC(=O)CN4CCCCC4)c3)c2C1 ; CC(=O)N1CCn2nc(-c3ccc(F)cc3)c(-c3ccnc(NC(=O)CN4CCCC4)c3)c2C1 ; CC(=O)N1CCn2nc(-c3ccc(F)cc3)c(-c3ccnc(NC(=O)Cn4cccc4)c3)c2C1]

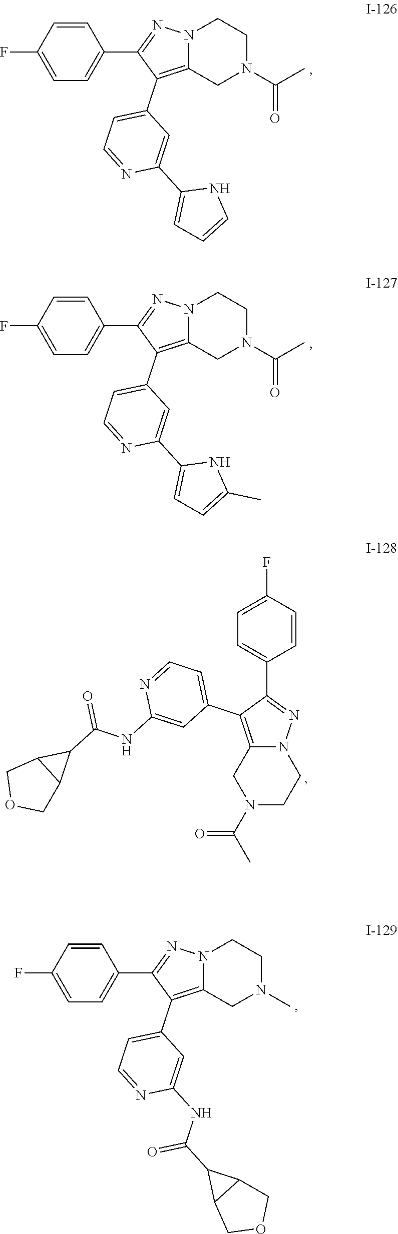 [SMILES: CC(=O)N1CCn2nc(-c3ccc(F)cc3)c(-c3ccnc(-c4ccc[nH]4)c3)c2C1 ; CC(=O)N1CCn2nc(-c3ccc(F)cc3)c(-c3ccnc(-c4ccc(C)[nH]4)c3)c2C1 ; CC(=O)N1CCn2nc(-c3ccc(F)cc3)c(-c3ccnc(NC(=O)C4C5COCC54)c3)c2C1 ; CN1CCn2nc(-c3ccc(F)cc3)c(-c3ccnc(NC(=O)C4C5COCC54)c3)c2C1]

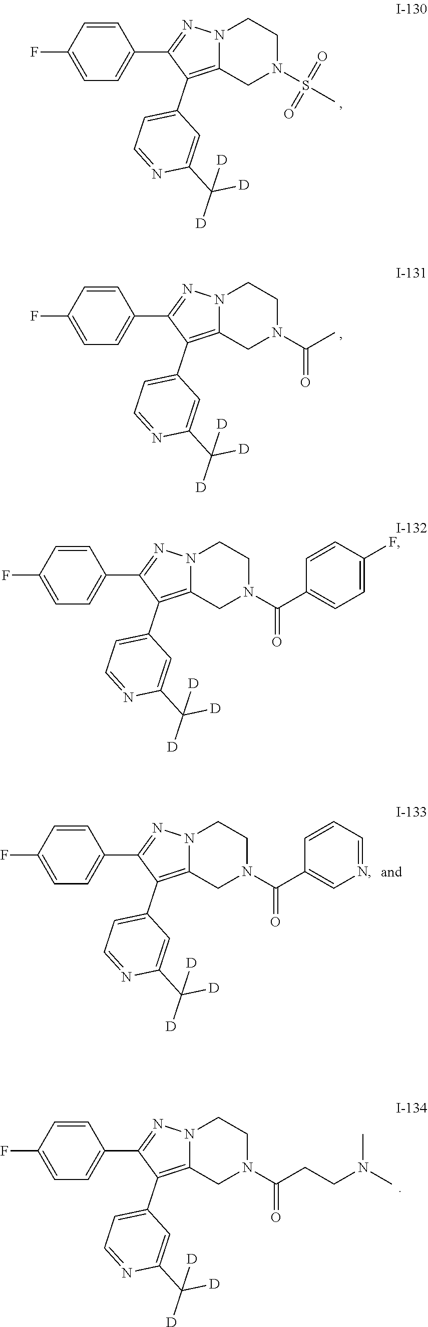 [SMILES: [2H]C([2H])([2H])c1cc(-c2c(-c3ccc(F)cc3)nn3c2CN(S(C)(=O)=O)CC3)ccn1 ; [2H]C([2H])([2H])c1cc(-c2c(-c3ccc(F)cc3)nn3c2CN(C(C)=O)CC3)ccn1 ; [2H]C([2H])([2H])c1cc(-c2c(-c3ccc(F)cc3)nn3c2CN(C(=O)c2ccc(F)cc2)CC3)ccn1 ; [2H]C([2H])([2H])c1cc(-c2c(-c3ccc(F)cc3)nn3c2CN(C(=O)c2cccnc2)CC3)ccn1 ; "and" ; [2H]C([2H])([2H])c1cc(-c2c(-c3ccc(F)cc3)nn3c2CN(C(=O)CCN(C)C)CC3)ccn1]

39. The compound of claim 1, wherein, the composition comprising optionally a pharmaceutically acceptable carrier.

40. A method for inhibiting casein kinase (CK) activity, said method comprising administering to a subject in need thereof an effective amount of the compound of claim 1, or a pharmaceutically acceptable salt, prodrug, or metabolite thereof, or a solvate or hydrate of any of the foregoing.

41. The method of claim 40, wherein said casein kinase (CK) is selected from the group consisting of casein kinase I alpha (CK1α), casein kinase I delta (CK1δ) and casein kinase I epsilon (CK1ε); or
said method is selected from the group consisting of an in vitro method, an ex vivo method, and an in vivo method.

42. (canceled)

43. A method for preventing and/or treating a disease or disorder, said method comprising administering to a subject in need thereof an effective amount of the compound of claim 1, or a pharmaceutically acceptable salt, prodrug, or metabolite thereof, or a solvate or hydrate of any of the foregoing.

44. The method of claim 43, wherein said disease or disorder is selected from the group consisting of neurological disease and psychiatric disease; or
said disease or disorder is selected from the group consisting of mood disorder, sleep disorder, and circadian disorder; or
said disease or disorder is selected from the group consisting of depressive disorder and bipolar disorder.

45. (canceled)

46. (canceled)
